(19) United States
(12) Patent Application Publication
(10) Pub. No.: US 2026/0255776 A1
(43) Pub. Date: Aug. 27, 2026

(54) ORGANIC COMPOUND, ORGANIC ELECTROLUMINESCENT DEVICE, AND ELECTRONIC APPARATUS

(71) Applicant: SHAANXI LIGHTE OPTOELECTRONICS MATERIAL CO., LTD., Xi'an, Shaanxi (CN)

(72) Inventors: Yingying WANG, Xi'an, Shaanxi (CN); Yingwen LI, Xi'an, Shaanxi (CN); Lei YANG, Xi'an, Shaanxi (CN)

(21) Appl. No.: 18/871,369

(22) PCT Filed: Sep. 19, 2023
(86) PCT No.: PCT/CN2023/119864

§ 371 Date: Dec. 3, 2024

(30) Foreign Application Priority Data
Apr. 26, 2023 (CN) 202310466053.1

(51) Int. Cl. H10K 50/11; C07B 59/00; C07D 407/12; H10K 85/30; H10K 85/60

(52) U.S. Cl. CPC H10K 50/11; C07B 59/002; C07D 407/12; H10K 85/342; H10K 85/626; H10K 85/633; H10K 85/636; H10K 85/6572; H10K 85/6574; H10K 85/6576; C07B 2200/05

(57) ABSTRACT

The present application relates to the technical field of organic electroluminescent materials, and provides an organic compound, an organic electroluminescent device, and an electronic apparatus. The organic compound of the present application has a structure as shown in formula 1, and the application of the organic compound to a hole auxiliary layer of the organic electroluminescent device can effectively improve the luminous efficiency of the device and prolong the service life.

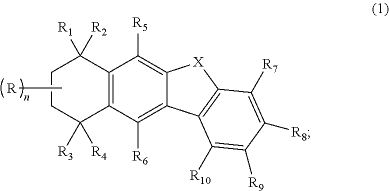

### CROSS-REFERENCE TO RELATED APPLICATIONS

This present disclosure claims the priority of Chinese patent application No. 2023104660531 filed on Apr. 26, 2023, which is incorporated herein by reference in its entirety as a part of this application.

### FIELD OF THE INVENTION

The present disclosure relates to the technical field of organic electroluminescent materials, and an organic compound, and an organic electroluminescent device comprising the same, and an electronic apparatus.

### BACKGROUND OF THE INVENTION

With the development of electronic technology and the progress of material science, the application range of electronic components used to realize electroluminescence, or photoelectric conversion is more and more extensive. This type of electronic components usually comprise a cathode and an anode disposed opposite to each other, and a functional layer disposed between the cathode and the anode. The functional layer is composed of multiple organic or inorganic film layers, and generally includes an energy conversion layer, a hole transport layer located between the energy conversion layer and the anode, and an electron transport layer located between the energy conversion layer and the cathode.

Taking organic electroluminescent devices as an example, they generally include an anode, a hole transport layer, an electroluminescent layer as an energy conversion layer, an electron transport layer, and a cathode stacked in sequence. When a voltage is applied to the anode and cathode, an electric field is generated between the two electrodes. Under the influence of the electric field, electrons on the cathode side move towards the electroluminescent layer, and holes on the anode side also move towards the luminescent layer. Electrons and holes combine in the electroluminescent layer to form excitons, which are in an excited state and release energy outward, thereby causing the electroluminescent layer to emit light externally.

Currently, the use of organic electroluminescent devices faces problems such as reduced luminous efficiency and shortened lifetime, leading to a decline in their performance. Therefore, there is still a need to continue developing new materials to further improve the performance of electronic components.

### SUMMARY OF THE INVENTION

In view of the above problems existing in the prior art, the objective of the present disclosure is to provide an organic compound, an organic electroluminescent device comprising the same, and an electronic apparatus. The organic compound is used in the organic electroluminescent device, such that the performance of the device can be improved.

According to a first aspect of the present disclosure, there is provided an organic compound having a structure shown in Formula 1:

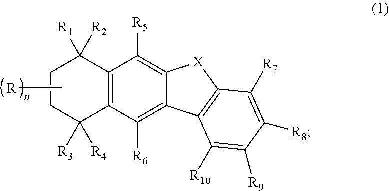

wherein, R₁, R₂, R₃, and R₄ are the same or different, and are each independently selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
one of R₅ and R₆ is a hydrogen, a deuterium, a halogen group, or a cyano, and the other is

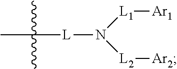

R₇, R₈, R₉, and R₁₀ are the same or different, and are each independently selected from a hydrogen, a deuterium, a halogen group, a cyano, an alkyl having 1 to 10 carbon atoms, an aryl having 6 to 20 carbon atoms, a heteroaryl having 3 to 20 carbon atoms, a deuterated alkyl having 1 to 10 carbon atoms, a deuterated aryl having 6 to 20 carbon atoms, and a deuterated heteroaryl having 3 to 20 carbon atoms;
optionally, R₇ and R₈, and/or R₈ and R₉, and/or R₉ and R₁₀, are interconnected to form a substituted or unsubstituted 5- to 13-membered ring;
substituent(s) on the 5- to 13-membered ring are the same or different, and are each independently selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
X is selected from C(R₁₁R₁₂), O, S, and N(R₁₃);
R₁₁ and R₁₂ are the same or different, and are each independently selected from a hydrogen, a deuterium, an alkyl having 1 to 10 carbon atoms, an aryl having 6 to 20 carbon atoms, a deuterated alkyl having 1 to 10 carbon atoms, and a deuterated aryl having 6 to 20 carbon atoms;
R₁₃ is selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
each R is independently selected from a deuterium, a halogen group, a cyano, an alkyl having 1 to 10 carbon atoms, an aryl having 6 to 20 carbon atoms, a deuterated alkyl having 1 to 10 carbon atoms, and a deuterated aryl having 6 to 20 carbon atoms;
n is the number of R, and n is selected from 0, 1, 2, 3, and 4;
L, L₁, and L₂ are the same or different, and are each independently selected from a single bond, a substituted or unsubstituted arylene having 6 to 30 carbon atoms, and a substituted or unsubstituted heteroarylene having 3 to 30 carbon atoms;
Ar₁ and Ar₂ are the same or different, and are each independently selected from a substituted or unsubstituted aryl having 6 to 40 carbon atoms, and a substituted or unsubstituted heteroaryl having 3 to 40 carbon atoms;
substituent(s) in Ar₁, Ar₂, L, L₁, and L₂ are the same or different, and are each independently selected from a deuterium, a cyano, a fluorine, an alkyl having 1 to 10 carbon atoms, a haloalkyl having 1 to 10 carbon atoms, a deuterated alkyl having 1 to 10 carbon atoms, an aryl having 6 to 20 carbon atoms, a haloaryl having 6 to 20 carbon atoms, a deuterated aryl having 6 to 20 carbon atoms, a heteroaryl having 3 to 20 carbon atoms, a haloheteroaryl having 3 to 20 carbon atoms, a deuterated heteroaryl having 3 to 20 carbon atoms, a cycloalkyl having 3 to 10 carbon atoms, a halocycloalkyl having 3 to 10 carbon atoms, a deuterated cycloalkyl having 3 to 10 carbon atoms, a triarylsilyl having 18 to 24 carbon atoms, and a trialkylsilyl having 3 to 12 carbon atoms; optionally, any two adjacent substituents in Ar₁ and Ar₂ form a saturated or unsaturated 5- to 13-membered ring.

According to a second aspect of the present disclosure, there is provided an organic electroluminescent device, comprising an anode and a cathode disposed opposite to each other, and a functional layer disposed between the anode and the cathode; the functional layer contains the organic compound as described above.

According to a third aspect of the present disclosure, there is provided an electronic apparatus, comprising the organic electroluminescent device described in the second aspect.

The present disclosure provides an organic compound, which is a kind of hole transport material. The parent nucleus of this compound is a tetrasubstituted cyclohexyl fused with a dibenzo-pentacyclic ring and connects a triarylamine group on the benzene ring fused with the cyclohexyl to form a triarylamine compound. In the compound of the present disclosure, the fused cycloalkyl can improve the interfacial properties and adhesion of the material; the introduction of triarylamine on the benzene ring fused with the cyclohexyl can ensure that the compound has a deep HOMO energy level while maintaining a high hole mobility; the four substituents are attached to specific substitution positions, such that the molecular morphology is distorted, thereby reducing the π-π stacking between molecules, effectively reducing the interaction between molecules, greatly improving the film forming properties of the compound, and thus improving the stability of the device. Using this compound as a hole auxiliary layer in organic electroluminescent devices can effectively reduce the operating voltage of the device, enhance the efficiency of the device, and increase the lifetime of the device.

### BRIEF DESCRIPTION OF THE DRAWINGS

The drawings are used for a further understanding of the present disclosure and constitute a part of the specification, and together with the following detailed description, are used to explain the present disclosure, but do not constitute a limitation of the present disclosure.

FIG. 1 is a schematic structural diagram of an organic electroluminescent device according to an embodiment of the present disclosure.

FIG. 2 is a schematic diagram of an electronic apparatus according to an embodiment of the present disclosure.

### REFERENCE SIGNS

100: Anode; 200: Cathode; 300: Functional layer; 310: Hole injection layer; 321: Hole transport layer; 322: Hole auxiliary layer; 330: Organic light-emitting layer; 340: Electron transport layer; 350: Electron injection layer; 400: Electronic apparatus.

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present disclosure more comprehensive and complete, and to convey the concepts of these exemplary embodiments fully to those of ordinary skill in the art. Features, structures, or characteristics described herein can be combined in one or more embodiment(s) in any suitable manner. In the following description, many specific details are provided to give a full understanding of the embodiments of the present disclosure.

In a first aspect, the present disclosure provides an organic compound having a structure shown in Formula 1:

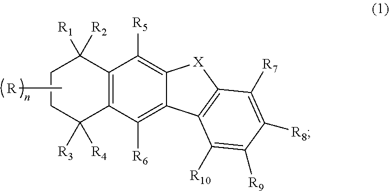

wherein, R₁, R₂, R₃, and R₄ are the same or different, and are each independently selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
one of R₅ and R₆ is a hydrogen, a deuterium, a halogen group, and a cyano, and the other is

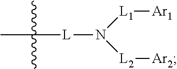

R₇, R₈, R₉, and R₁₀ are the same or different, and are each independently selected from a hydrogen, a deuterium, a halogen group, a cyano, an alkyl having 1 to 10 carbon atoms, an aryl having 6 to 20 carbon atoms, a heteroaryl having 3 to 20 carbon atoms, a deuterated alkyl having 1 to 10 carbon atoms, a deuterated aryl having 6 to 20 carbon atoms, and a deuterated heteroaryl having 3 to 20 carbon atoms;
optionally, R₇ and R₈, and/or R₈ and R₉, and/or R₉ and R₁₀, are interconnected to form a substituted or unsubstituted 5- to 13-membered ring;
substituent(s) on the 5- to 13-membered ring are the same or different, and are each independently selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
X is selected from C(R₁₁R₁₂), O, S, and N(R₁₃);
R₁₁ and R₁₂ are the same or different, and are each independently selected from a hydrogen, a deuterium, an alkyl having 1 to 10 carbon atoms, an aryl having 6 to 20 carbon atoms, a deuterated alkyl having 1 to 10 carbon atoms, and a deuterated aryl having 6 to 20 carbon atoms;
R₁₃ is selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
each R is independently selected from a deuterium, a halogen group, a cyano, an alkyl having 1 to 10 carbon atoms, an aryl having 6 to 20 carbon atoms, a deuterated alkyl having 1 to 10 carbon atoms, and a deuterated aryl having 6 to 20 carbon atoms;
n is the number of R, and n is selected from 0, 1, 2, 3, and 4;
L, L₁, and L₂ are the same or different, and are each independently selected from a single bond, a substituted or unsubstituted arylene having 6 to 30 carbon atoms, and a substituted or unsubstituted heteroarylene having 3 to 30 carbon atoms;
Ar₁ and Ar₂ are the same or different, and are each independently selected from a substituted or unsubstituted aryl having 6 to 40 carbon atoms, and a substituted or unsubstituted heteroaryl having 3 to 40 carbon atoms;
substituent(s) in Ar₁, Ar₂, L, L₁, and L₂ are the same or different, and are each independently selected from a deuterium, a cyano, a fluorine, an alkyl having 1 to 10 carbon atoms, a haloalkyl having 1 to 10 carbon atoms, a deuterated alkyl having 1 to 10 carbon atoms, an aryl having 6 to 20 carbon atoms, a haloaryl having 6 to 20 carbon atoms, a deuterated aryl having 6 to 20 carbon atoms, a heteroaryl having 3 to 20 carbon atoms, a haloheteroaryl having 3 to 20 carbon atoms, a deuterated heteroaryl having 3 to 20 carbon atoms, a cycloalkyl having 3 to 10 carbon atoms, a halocycloalkyl having 3 to 10 carbon atoms, a deuterated cycloalkyl having 3 to 10 carbon atoms, a triarylsilyl having 18 to 24 carbon atoms, and a trialkylsilyl having 3 to 12 carbon atoms; optionally, any two adjacent substituents in Ar₁ and Ar₂ form a saturated or unsaturated 5- to 13-membered ring.

In the present disclosure, when a plurality of groups are collectively listed with “and/or”, it indicates that all, some, or any one of the situations may occur.

In the present disclosure, the terms “optional” and “optionally” mean that the event or circumstance described later may or may not occur, and the description includes the situations where the event or circumstance occurs or does not occur. For example, “optionally, any two adjacent substituents in Ar₁ and Ar₂ form a saturated or unsaturated 5- to 13-membered ring” means that, two adjacent substituents can form a ring or not, which includes: the scenario where two adjacent substituents form a ring and the scenario where two adjacent substituents do not form a ring. For another example, “optionally, R₇ and R₈, and/or R₈ and R₉, and/or R₉ and R₁₀, form a saturated or unsaturated 5- to 13-membered ring” refers to any one or more set(s) of R₇ and R₈, and/or R₈ and R₉, and/or R₉ and R₁₀ are interconnected to form a ring, or R₇ to R₁₀ each independently exist, without forming a ring.

In the present disclosure, when it is mentioned that any two groups can form a ring, it refers to these groups forming a substituted or unsubstituted ring together with the atoms to which they are attached. For example, “R₇ and R₈, and/or R₈ and R₉, and/or R₉ and R₁₀, form a substituted or unsubstituted 5- to 13-membered ring” refers to R₇ and R₈ forming a substituted or unsubstituted 5- to 13-membered ring, and/or R₈ and R₉ forming a substituted or unsubstituted 5- to 13-membered ring, and/or R₉ and R₁₀ forming a substituted or unsubstituted 5- to 13-membered ring. The substituted or unsubstituted 5- to 13-membered ring include, but are not limited to, a benzene ring, a naphthalene ring, a cyclopentane, a cyclohexane, an adamantane, a deuterated benzene, a deuterated cyclohexane, a phenylcyclohexane, 1,1,4,4-tetramethylcyclohexane, 1,1,4,4-tetra(trideuteromethyl)cyclohexane, and so on.

In the present disclosure, when it is mentioned that any two adjacent substituents form a saturated or unsaturated ring, such as a saturated or unsaturated 5- to 13-membered ring, it includes a saturated carbocyclic ring, a saturated heterocyclic ring, a partially unsaturated carbocyclic ring, a partially unsaturated heterocyclic ring, an aromatic carbocyclic ring, and an aromatic heterocyclic ring. When n is used as a prefix for the ring, n is an integer indicating that the number of ring atoms in the ring is n. For example, a 5- to 13-membered ring refers to a ring with 5-13 ring atoms, including rings with 5, 6, 7, 8, 9, 10, 11, 12, and 13 ring atoms. A saturated or unsaturated 5- to 13-membered ring include, but are not limited to, a benzene ring, a naphthalene ring, a phenanthrene ring, an anthracene ring, a fluorene ring, a cyclopentane, a cyclohexane, an adamantane, and the like.

In the present disclosure, the descriptive expression “each . . . are independently” may be used interchangeably with the description expressions “ . . . are respectively independently” and “ . . . are each independently”, and all these expressions should be interpreted in a broad sense. They can not only mean that, in a different group, specific options expressed by the same symbol are mutual non-influence, but also mean that in same groups, specific options expressed by the same symbol are mutual non-influence. For example,

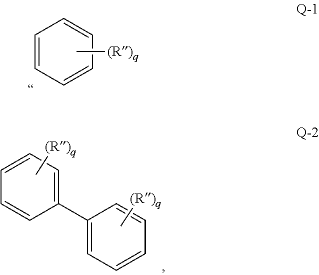

wherein each q is independently 0, 1, 2, or 3, and each R″ is independently selected from a hydrogen, a deuterium, a fluorine, and a chlorine” means that the benzene ring represented by Formula Q-1 has q substituents R″, and each R″ may be the same or different, with mutual non-influence between the options for each R″; and that each of benzene rings of the biphenyl represented by Formula Q-2 has q substituents R″, and the number q of R″ on each of the two benzene rings may be the same or different and each R″ may be the same or different, with mutual non-influence between the options for each R″.

In the present disclosure, such a term “substituted or unsubstituted” means that the functional group defined by this term may or may not have a substituent (hereinafter referred to as Rc for ease of description). For example, a “substituted or unsubstituted aryl” refers to an aryl with the substituent Rc or an aryl without a substituent. The aforementioned substituent, Rc, may be, such as, a deuterium, a halogen group, a cyano, an alkyl, a cycloalkyl, an aryl, a heteroaryl, a trialkylsilyl, a triarylsilyl, a haloalkyl, a deuterated alkyl, a deuterated aryl, a haloaryl, a haloheteroaryl, a deuterated heteroaryl, a halocycloalkyl, a deuterated cycloalkyl, etc. The number of substitutions can be one or more.

In the present disclosure, “more” refers to two or more, such as two, three, four, five, six, and so on.

The hydrogen atom in the structure of the compounds in the present disclosure includes various isotope atoms of hydrogen, such as hydrogen (H), deuterium (D), or tritium (T).

In the present disclosure, the number of carbon atoms of a substituted or unsubstituted functional group refers to the total number of carbon atoms. For example, if L₁ is a substituted arylene having 12 carbon atoms, the total number of carbon atoms in the arylene and its substituents is 12.

In the present disclosure, an aryl refers to an optional functional group or a substituent derived from an aromatic carbon ring. An aryl may be a monocyclic aryl (e.g., a phenyl) or a polycyclic aryl. In other words, an aryl may be a monocyclic aryl, a fused-ring aryl, a spiro-ring aryl, two or more monocyclic aryls linked by carbon-carbon bond, a monocyclic aryl and a fused-ring aryl linked by carbon-carbon bond, or two or more fused-ring aryls linked by carbon-carbon bond. That is, unless otherwise specified, two or more aromatic groups linked by carbon-carbon bond may also be regarded as an aryl in the present disclosure. Among them, a fused-ring aryl may include, for example, a bicyclic fused aryl (e.g., naphthyl), a tricyclic fused aryl (e.g., phenanthryl, fluorenyl, anthryl), etc. The aryl does not contain heteroatoms such as B, N, O, S, P, Se, and Si. Examples of an aryl may include, but are not limited to, a phenyl, a naphthyl, a fluorenyl, a spirobifluorenyl, an anthracenyl, a phenanthryl, a biphenyl, a terphenyl, a triphenylene, a perylenyl, a benzo[9,10]phenanthryl, a pyrenyl, a benzofluoranthryl, a chrysenyl, etc.

In the present disclosure, a terphenyl includes

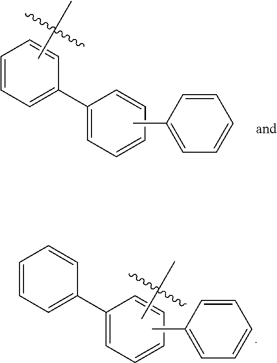

In the present disclosure, “arylene” involved refers to a divalent group formed by further removing one or more hydrogen atom(s) from an aryl.

In the present disclosure, the number of carbon atoms of a substituted or unsubstituted aryl (or arylene) may be 6, 8, 10, 12, 13, 14, 15, 16, 17, 18, 19, 20, 21, 22, 23, 24, 25, 26, 28, 30, 31, 33, 34, 35, 36, 38, 40, ect.

In the present disclosure, the substituted aryl (or arylene) can be an aryl (or arylene) in which one or more hydrogen atom(s) are replaced by a group such as a deuterium atom, a halogen group, a cyano, an aryl, a heteroaryl, a trialkylsilyl, a triarylsilyl, an alkyl, a cycloalkyl, a haloalkyl, a deuterated alkyl, a haloaryl, a deuterated aryl, a haloheteroaryl, a deuterated heteroaryl, a halocycloalkyl, a deuterated cycloalkyl, etc. It should be understood that the number of carbon atoms in the substituted aryl (or arylene) refers to the total number of carbon atoms in the aryl (or arylene) and the substituents on the aryl (or arylene). For example, a substituted aryl having 18 carbon atoms refers to the total number of carbon atoms in the aryl and its substituents being 18.

In the present disclosures, the aryl as a substituent includes, but is not limited to, a phenyl, a naphthyl, a phenanthryl, a biphenyl, a fluorenyl, a dimethylfluorenyl, an anthracenyl, a chrysenyl, triphenylene, terphenyl, etc.

In the present disclosure, the fluorenyl may be substituted by one or more substituent(s). In the case that the above fluorenyl are substituted, the substituted fluorenyl may be:

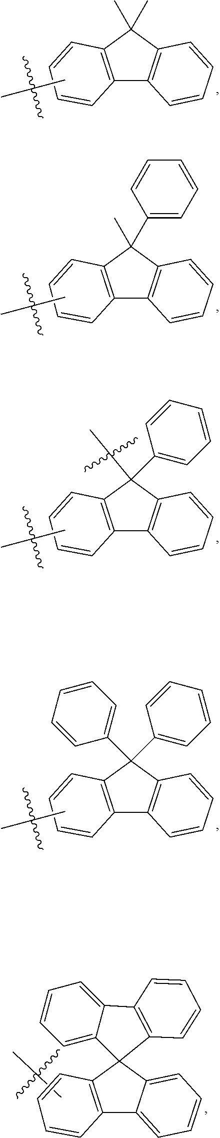

but is not limited thereto.

In the present disclosure, a “heteroaryl” refers to a monovalent aromatic ring containing 1, 2, 3, 4, 5, 6, or 7 heteroatoms or a derivative thereof. The heteroatoms may be one or more selected from B, O, N, P, Si, Se, and S. A heteroaryl may be a monocyclic heteroaryl or a polycyclic heteroaryl. In other words, a heteroaryl may be a single aromatic ring system, or multiple aromatic ring systems linked by carbon-carbon bond, with any of the aromatic ring systems being an aromatic monocyclic ring or an aromatic fused ring. For example, a heteroaryl may include, thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc, but not limited to thereto. Among them, the thienyl, the furyl, the phenanthrolinyl and the like are types of heteroaryl with a single aromatic ring system, while N-phenylcarbazolyl and N-pyridylcarbazolyl are types of heteroaryl with a polycyclic system linked by carbon-carbon bonds.

In the present disclosure, a “heteroarylene” involved refers to a divalent group formed by further removing one hydrogen atom from a heteroaryl.

In the present disclosure, the number of carbon atoms of a substituted or unsubstituted heteroaryl (or heteroarylene) may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 etc. In the present disclosure, the substituted heteroaryl (or heteroarylene) can be a heteroaryl (or heteroarylene) in which one or more hydrogen atom(s) are replaced by a group such as a deuterium atom, a halogen group, a cyano, an aryl, a heteroaryl, a trialkylsilyl, a triarylsilyl, an alkyl, a cycloalkyl, a haloalkyl, a deuterated alkyl, a haloaryl, a deuterated aryl, a haloheteroaryl, a deuterated heteroaryl, a halocycloalkyl, a deuterated cycloalkyl, etc. It should be understood that the number of carbon atoms in the substituted heteroaryl (or heteroarylene) refers to the total number of carbon atoms in the heteroaryl (or heteroarylene) and the substituents on the heteroaryl (or heteroarylene). For example, a substituted heteroaryl having 18 carbon atoms refers to the total number of carbon atoms in the heteroaryl and its substituents being 18.

In the present disclosure, the heteroaryl as a substituent includes, but is not limited to, a pyridyl, a carbazolyl, a quinolyl, an isoquinolyl, a phenanthrolinyl, a benzoxazolyl, a benzothiazolyl, a benzimidazolyl, a dibenzothienyl, a dibenzofuranyl, N-phenylcarbazolyl, etc.

In the present disclosure, an alkyl having 1 to 10 carbon atoms may include a straight-chain alkyl having 1 to 10 carbon atoms or a branched-chain alkyl having 3 to 10 carbon atoms. The number of carbon atoms of an alkyl may be for example 1, 2, 3, 4, 5, 6, 7, 8, 9, or 10. Specific examples of an alkyl include, but are not limited to, a methyl, an ethyl, a n-propyl, an isopropyl, a n-butyl, an isobutyl, a tert-butyl, a n-pentyl, an isopentyl, a neopentyl, n-hexyl, etc.

In the present disclosure, the number of carbon atoms of a cycloalkyl having 3 to 10 carbon atoms may be, for example, 3, 4, 5, 6, 7, 8, or 10. Specific examples of a cycloalkyl include, but are not limited to, a cyclopentyl, a cyclohexyl, an adamantyl, etc.

In the present disclosure, “deuterated” refers to at least one hydrogen (“H”) in a compound or a group is replaced by a deuterium (“D”). Specifically, a deuterated compound or a deuterated group may be a compound or a group in which one or more, or all of available hydrogen are replaced by a deuterium.

In the present disclosure, a halogen group may be for example, a fluorine, a chlorine, a bromine, or an iodine.

In the present disclosure, specific examples of a haloalkyl include, but are not limited to, a trifluoromethyl.

In the present disclosure, specific examples of a deuterated alkyl include, but are not limited to, a trideuterated methyl.

In the present disclosure, specific examples of a deuterated aryl include, but are not limited to, a perdeuterated phenyl, perdeuterated naphthyl.

In the present disclosure, specific examples of a haloaryl include, but are not limited to, a fluorophenyl.

In the present disclosure, specific examples of a deuterated heteroaryl include, but are not limited to a deuterated dibenzofuranyl, a deuterated pyridyl.

In the present disclosure, specific examples of a trialkylsilyl include, but are not limited to a trimethylsilyl.

In the present disclosure, specific examples of a triarylsilyl include, but are not limited to a triphenylsilyl.

In the present disclosure, a non-positioned bond is a single bond

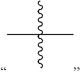

extending from the ring system involved in the non-positioned connection bond which represents that one end of the connection bond can connect to any position in the ring system through which the bond passes, and the other end connects to the rest of the compound molecule.

For example, as shown in Formula (f) below, the naphthyl represented by Formula (f) is connected to other positions of the molecule through two non-positioned bonds passing through the two rings, which indicates any of possible connection forms shown in Formulae (f-1) to (f-10):

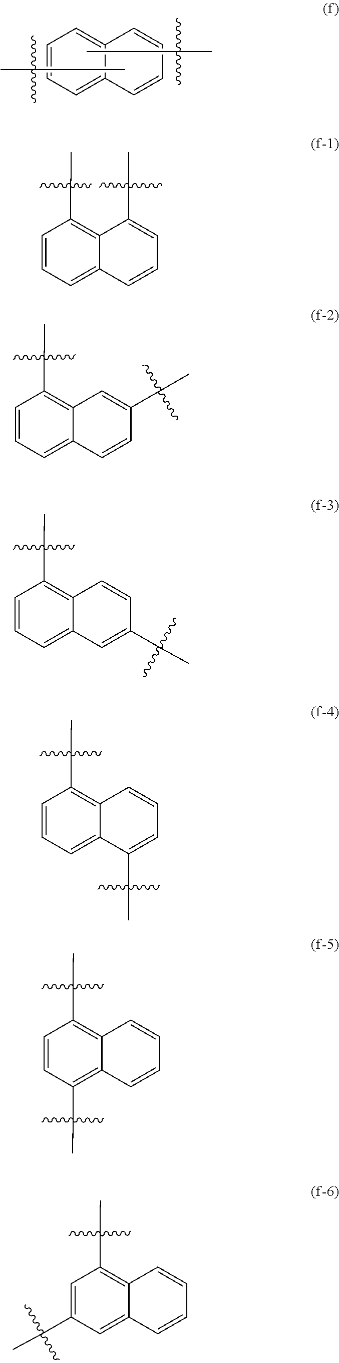

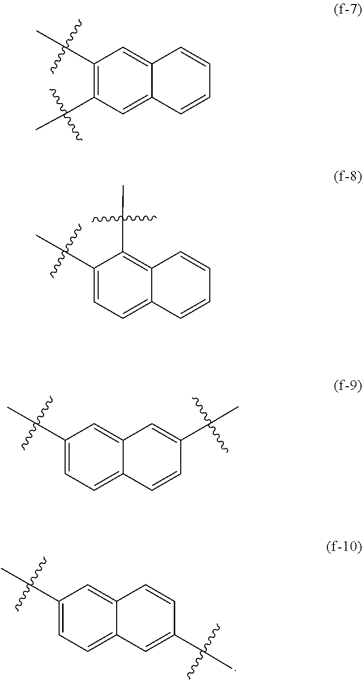 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl group represented by Formula (X′) is connected to other positions of the molecule via a non-positional connection bond extending from the middle of a side benzene ring, which indicates any of possible connection forms shown in Formulae (X′-1) to (X′-4):

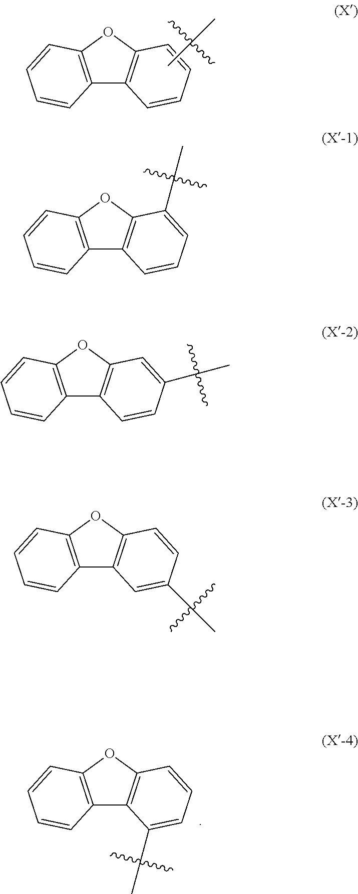

The non-positioned substituent in the present disclosure refers to a substituent connected by a single bond extending from the center of the ring system, indicating that the substituent can be connected to any possible position in the ring system. For example, as shown in Formula (Y) below, the substituent R′ represented by Formula (Y) is linked to a quinoline ring via a non-positioned connection bond, which indicates any of possible connecting mode shown in Formulae (Y-1) to (Y-7):

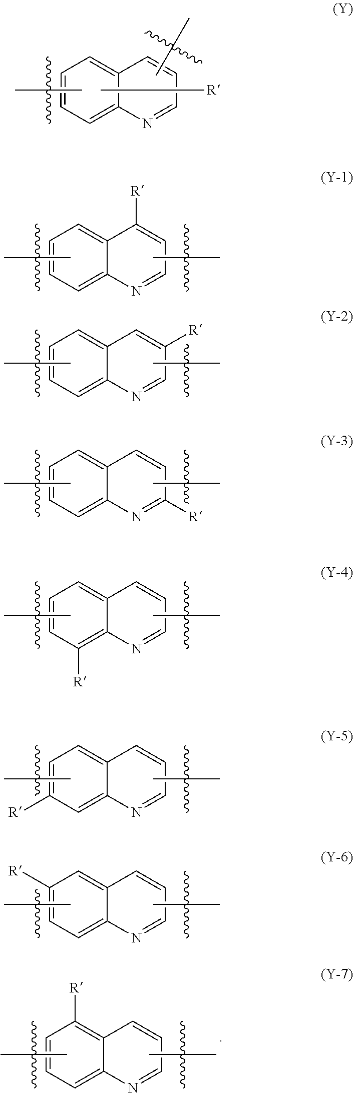

In some embodiments, Ar₁ and Ar₂ are the same or different, and are each independently selected from a substituted or unsubstituted aryl having 6 to 25 carbon atoms, a substituted or unsubstituted heteroaryl having 12 to 24 carbon atoms.

In some embodiments, Ar₁ and Ar₂ are each independently selected from a substituted or unsubstituted aryl having 6, 7, 8, 9, 10, 11, 12, 13, 14, 15, 16, 17, 18, 19, 20, 21, 22, 23, 24, and 25 carbon atoms, a substituted or unsubstituted heteroaryl having 12, 13, 14, 15, 16, 17, 18, 19, 20, 21, 22, 23, and 24 carbon atoms.

Optionally, substituent(s) in Ar₁ and Ar₂ are each independently selected from a deuterium, a cyano, a fluorine, an alkyl having 1 to 4 carbon atoms, a haloalkyl having 1 to 4 carbon atoms, a deuterated alkyl having 1 to 4 carbon atoms, a phenyl, a naphthyl, a heteroaryl having 5 to 12 carbon atoms, a cycloalkyl having 5 to 10 carbon atoms, a trimethylsilyl, and a triphenylsilyl.

Optionally, any two adjacent substituents in Ar₁ and Ar₂ form a cyclopentyl

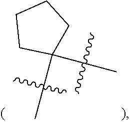 [SMILES: *C1(*)CCCC1]

a cyclohexyl

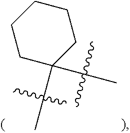 [SMILES: *C1(*)CCCCC1]

a phenyl

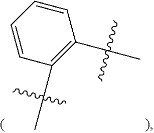 [SMILES: *c1ccccc1*]

and a fluorenyl

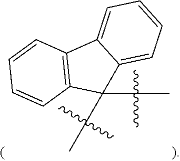 [SMILES: *C1(*)c2ccccc2-c2ccccc21]

In some embodiments, Ar₁ and Ar₂ are the same or different, and are each independently selected from a substituted or unsubstituted phenyl, a substituted or unsubstituted naphthyl, a substituted or unsubstituted biphenyl, a substituted or unsubstituted terphenyl, a substituted or unsubstituted phenanthryl, a substituted or unsubstituted anthracenyl, a substituted or unsubstituted triphenylene, a substituted or unsubstituted fluorenyl, a substituted or unsubstituted dibenzothienyl, a substituted or unsubstituted dibenzofuranyl, a substituted or unsubstituted carbazolyl, a substituted or unsubstituted spirobifluorenyl, a substituted or unsubstituted cyclopentane-spiro-fluorenyl, a substituted or unsubstituted cyclohexane-spiro-fluorenyl.

Optionally, substituent(s) Ar₁ and Ar₂ are the same or different, and are each independently selected from a deuterium, a fluorine, a cyano, a methyl, an ethyl, an isopropyl, a tert-butyl, a trifluoromethyl, a trideuterated methyl, a phenyl, a naphthyl, a carbazolyl, a trimethylsilyl, a triphenylsilyl, a cyclopentyl, and a cyclohexyl.

In some embodiments, Ar₁ and Ar₂ are the same or different, and are each independently selected from a substituted or unsubstituted group T, and the unsubstituted group T is selected from the group consisting of the following group:

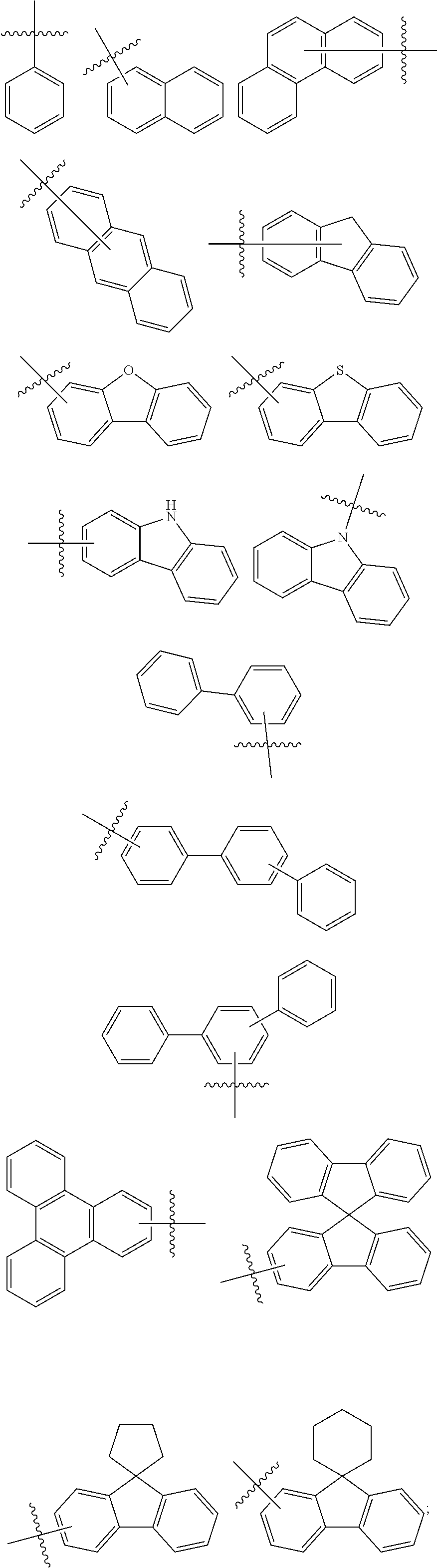

wherein, the substituted group T has one or more substituent(s), which are the same or different, and are each independently selected from a deuterium, a fluorine, a cyano, a methyl, an ethyl, an isopropyl, a tert-butyl, a trifluoromethyl, a trideuterated methyl, a trimethylsilyl, a triphenylsilyl, a phenyl, a naphthyl, a carbazolyl, a cyclopentyl, and a cyclohexyl.

In some embodiments, Ar₁ and Ar₂ are the same or different, and are each independently selected from the following groups:

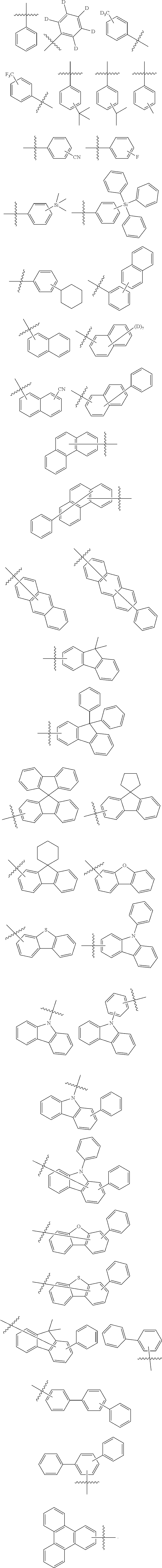

In some embodiments, Ar₁ and Ar₂ are the same or different, and are each independently selected from the following groups:

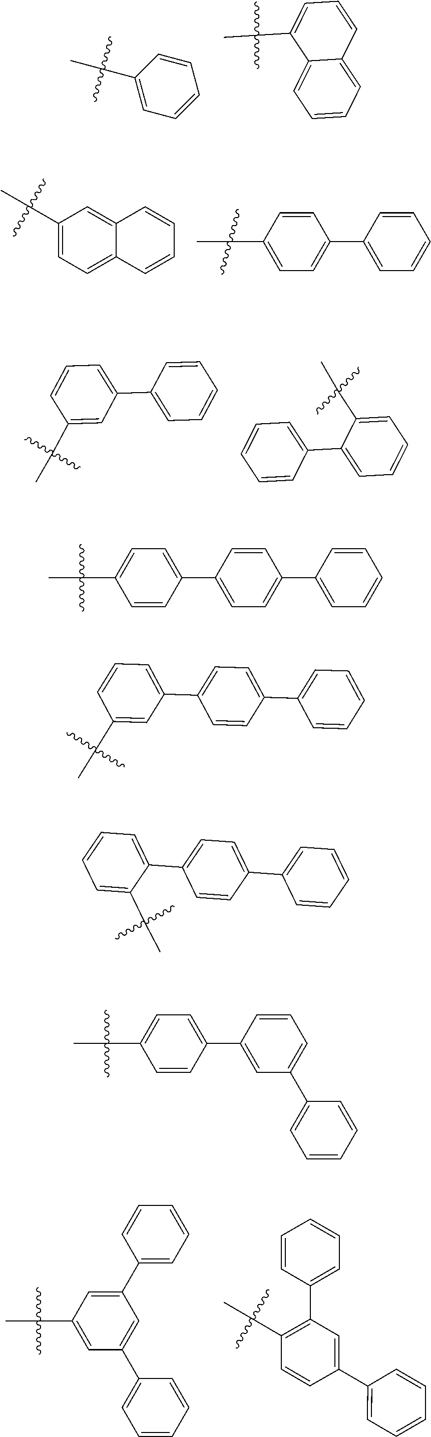

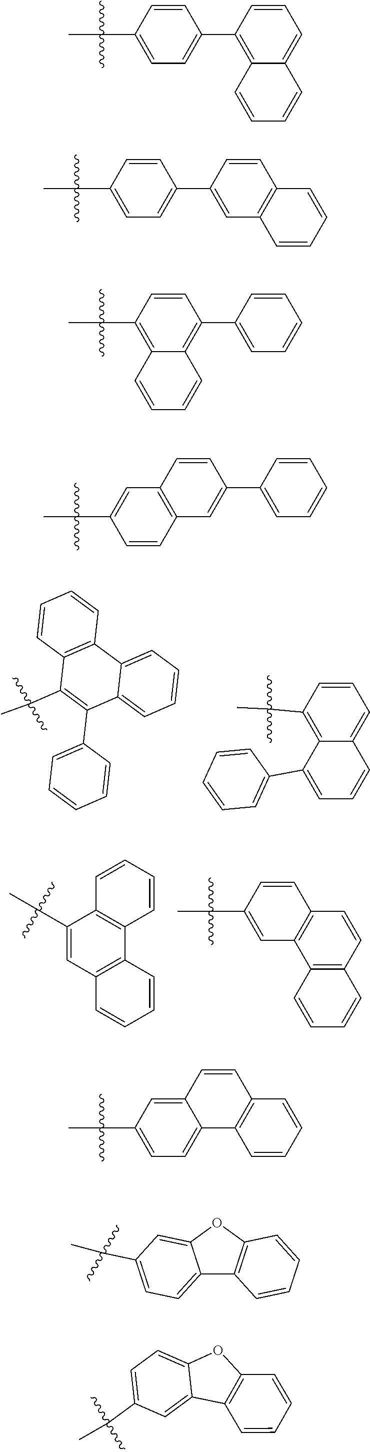

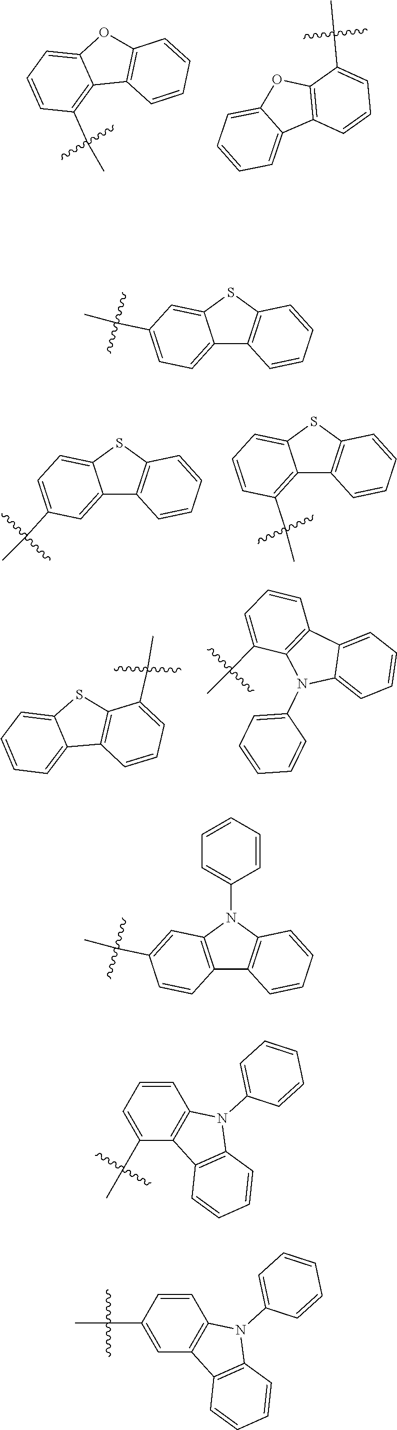

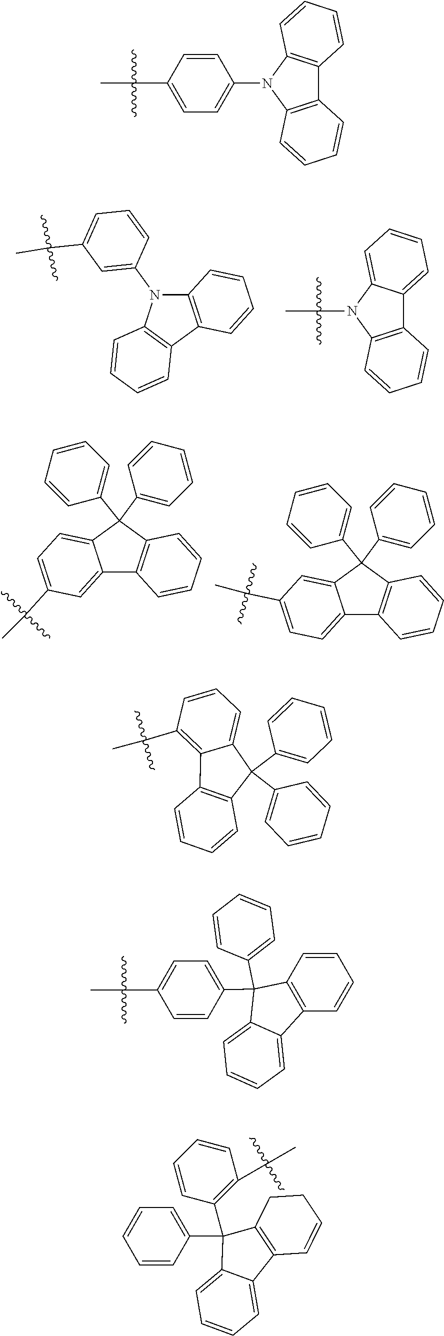

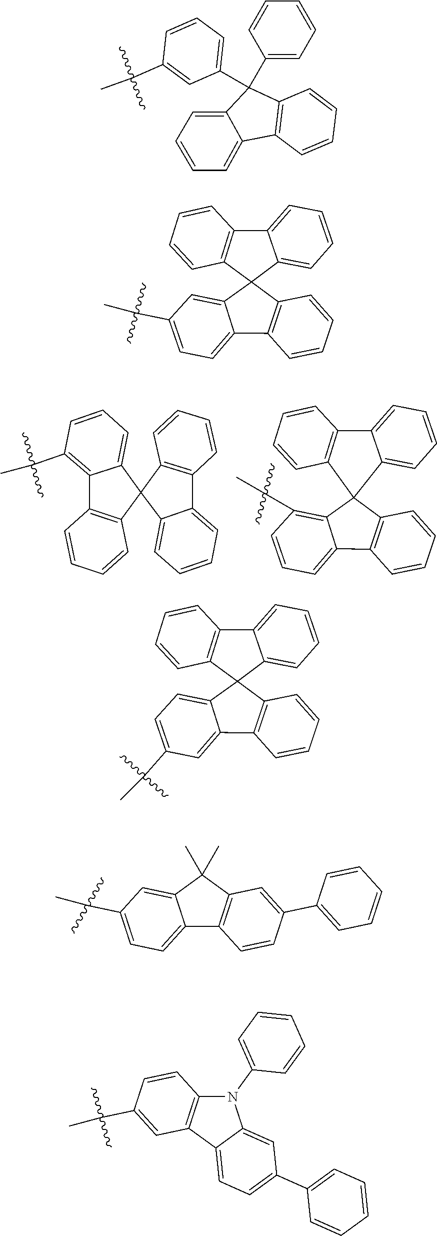

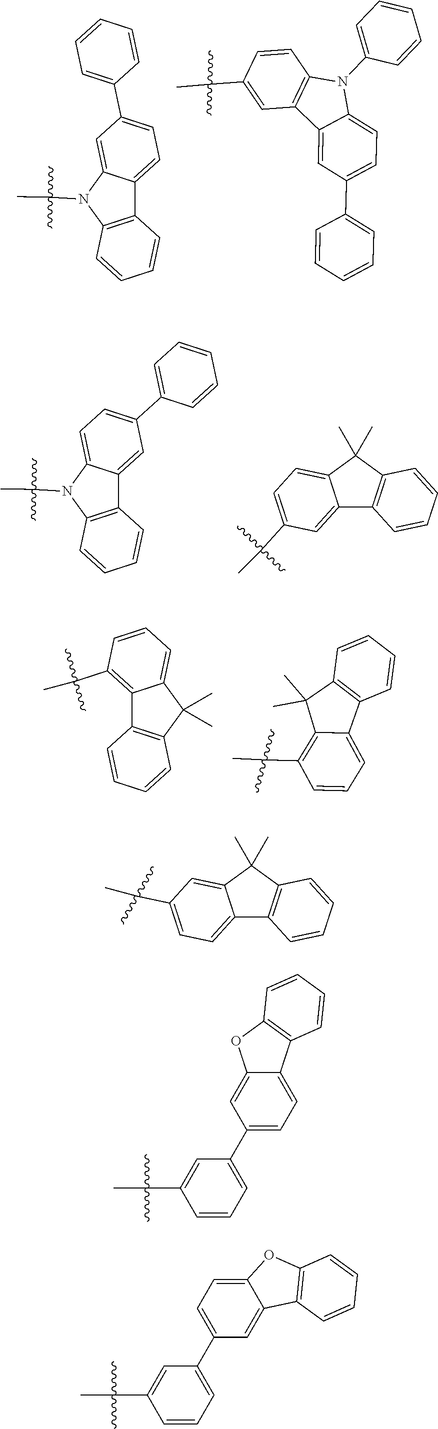

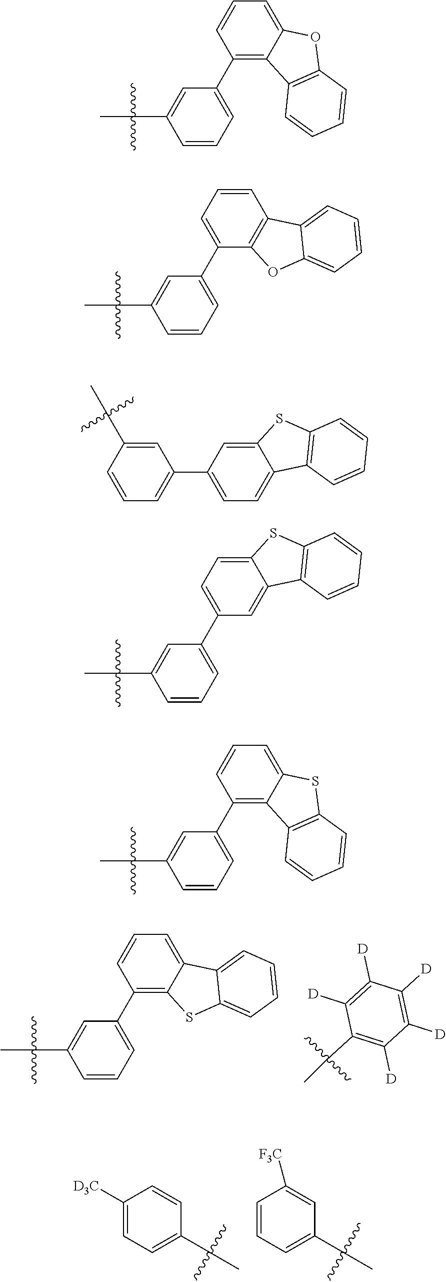

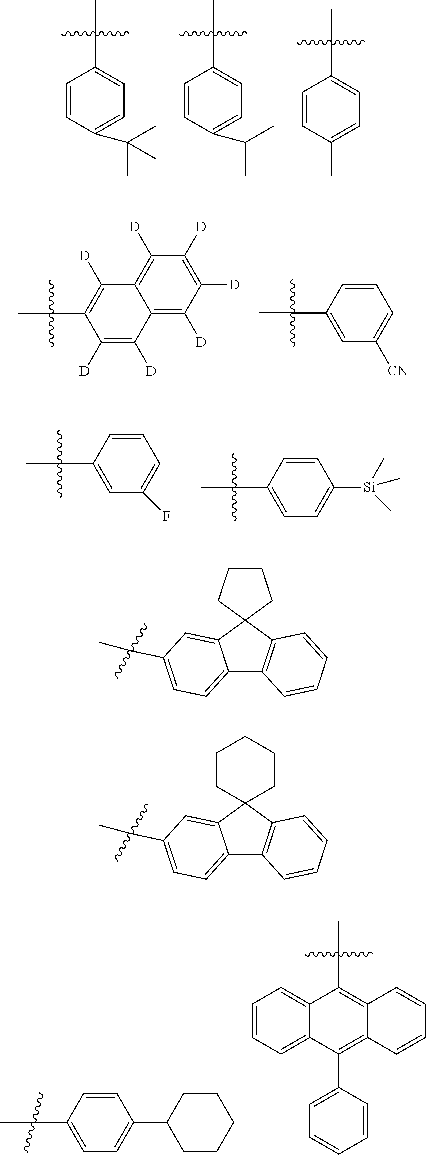

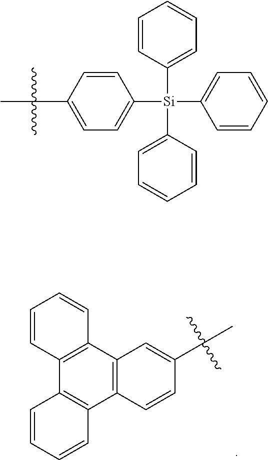 [SMILES: *c1ccc([Si](c2ccccc2)(c2ccccc2)c2ccccc2)cc1 ; *c1ccc2c3ccccc3c3ccccc3c2c1]

In some embodiments, L, L₁, and L₂ are the same or different, and are each independently selected from a single bond, a substituted or unsubstituted arylene having 6 to 18 carbon atoms, and a substituted or unsubstituted heteroarylene having 12 to 18 carbon atoms.

In some embodiments, L, L₁, and L₂ are the same or different, and are each independently selected from a single bond, a substituted or unsubstituted arylene having 6, 7, 8, 9, 10, 11, 12, 13, 14, 15, 16, 17, or 18 carbon atoms, or a substituted or unsubstituted heteroarylene having 12, 13, 14, 15, 16, 17, or 18 carbon atoms.

Optionally, substituent(s) in L, L₁, and L₂ are the same or different, and are each independently selected from a deuterium, a cyano, a halogen group, an alkyl having 1 to 4 carbon atoms, a haloalkyl having 1 to 4 carbon atoms, a deuterated alkyl having 1 to 4 carbon atoms, an aryl having 6 to 12 carbon atoms, a haloaryl having 6 to 12 carbon atoms, a deuterated aryl having 6 to 12 carbon atoms, a heteroaryl having 5 to 12 carbon atoms, a haloheteroaryl having 5 to 12 carbon atoms, and a deuterated heteroaryl having 5 to 12 carbon atoms.

In some embodiments, L, L₁, and L₂ are the same or different, and are each independently selected from a single bond, a substituted or unsubstituted phenylene, a substituted or unsubstituted naphthylene, a substituted or unsubstituted biphenylene, a substituted or unsubstituted terphenylene, a substituted or unsubstituted phenanthrylene, a substituted or unsubstituted dibenzothienylene, a substituted or unsubstituted dibenzofuranylene, and a substituted or unsubstituted carbazolylene.

Optionally, substituent(s) in L, L₁, and L₂ are the same or different, and are each independently selected from a deuterium, a fluorine, a cyano, a methyl, an ethyl, an isopropyl, a tert-butyl, a trifluoromethyl, a trideuterated methyl, a phenyl, and a naphthyl.

In some embodiments, L, L₁, and L₂ are the same or different, and each independently selected from a single bond, a substituted or unsubstituted group V, and the unsubstituted group V is selected from the group consisting of the following groups:

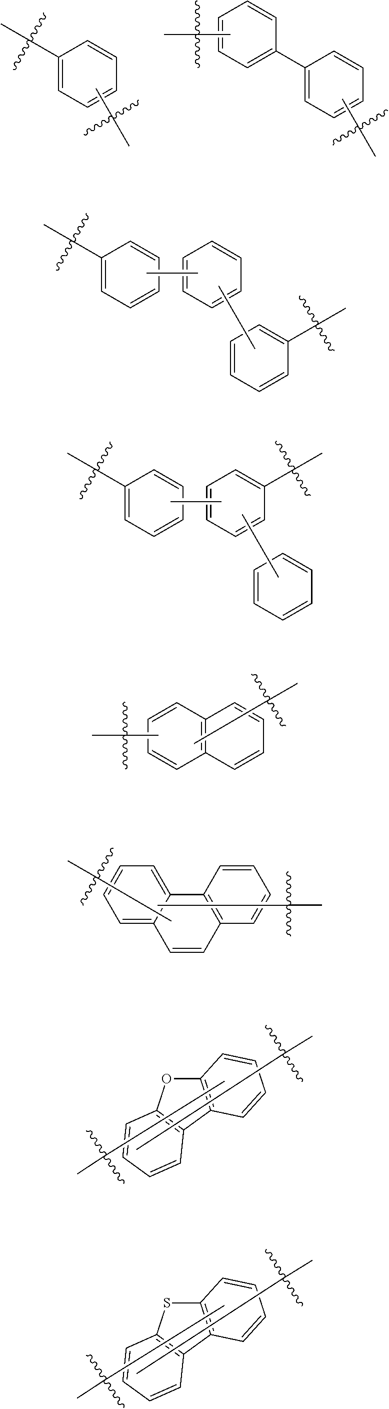

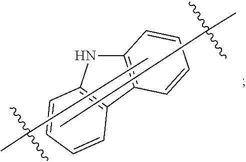

wherein, the substituted group V has one or more substituent(s), which are the same or different, and are each independently selected from a deuterium, a fluorine, a cyano, a methyl, an ethyl, an isopropyl, a tert-butyl, a trifluoromethyl, a trideuterated methyl, a phenyl, and a naphthyl.

In some embodiments, L is selected from a substituted or unsubstituted group V₁, and the unsubstituted group V₁ is selected from the group consisting of the following groups:

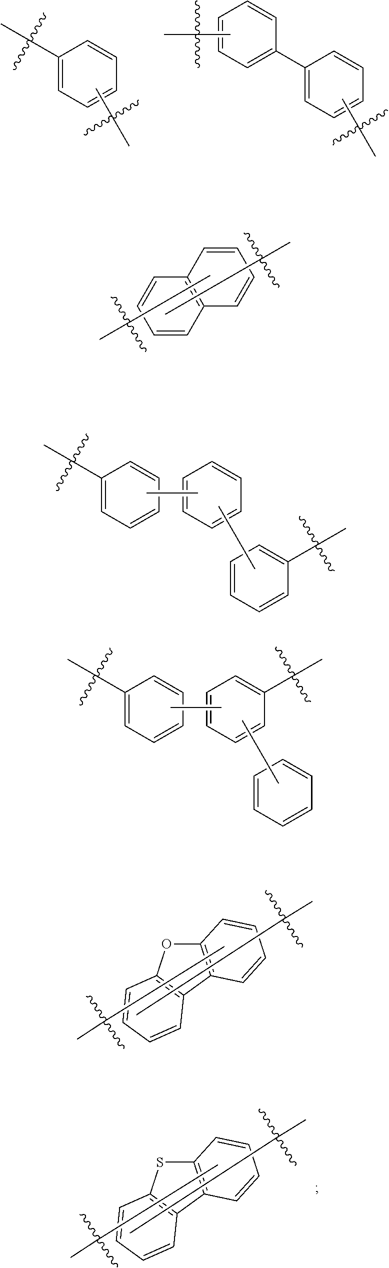

wherein, the substituted group V₁ has one or more substituent(s), which are the same or different, and are each independently selected from a deuterium, a fluorine, a cyano, a methyl, an ethyl, an isopropyl, a tert-butyl, a trifluoromethyl, a trideuterated methyl, a phenyl, and a naphthyl.

In some embodiments, L is selected from a single bond, and the group consisting of the following groups:

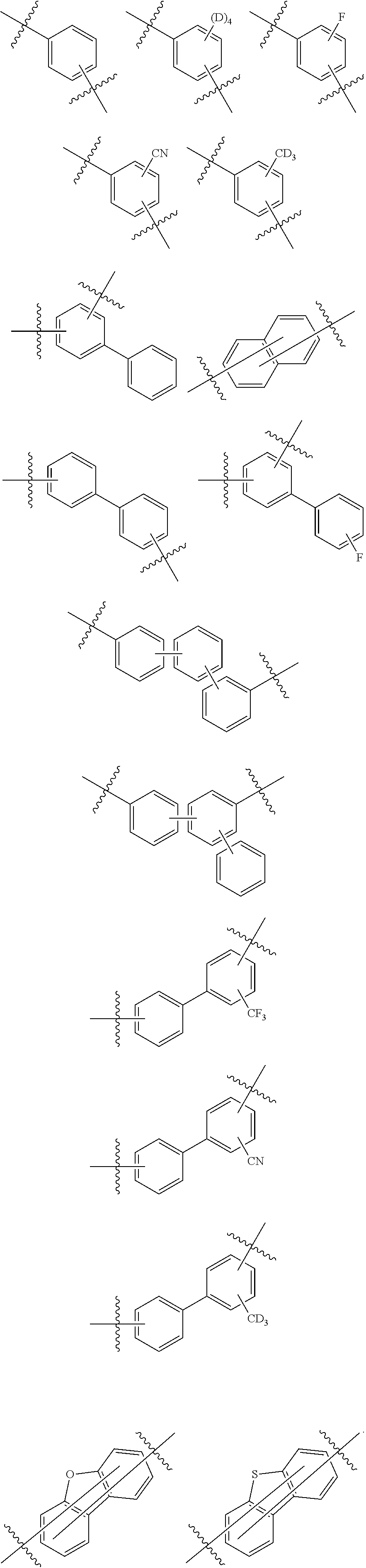

In some embodiments, L₁ and L₂ are each independently selected from a single bond, a substituted or unsubstituted group V₂, and the unsubstituted group V₂ is selected from the group consisting of the following groups:

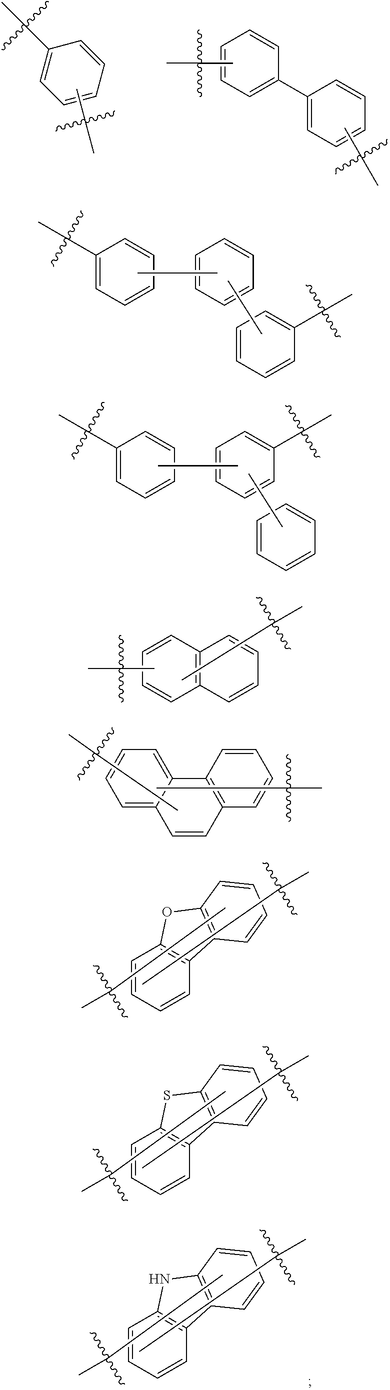

wherein, the substituted group V₂ has one or more substituent(s), which are the same or different, and are each independently selected from a deuterium, a fluorine, a cyano, a methyl, an ethyl, an isopropyl, a tert-butyl, a trifluoromethyl, a trideuterated methyl, a phenyl, and a naphthyl.

In some embodiments, L₁ and L₂ are each independently selected from a single bond and the group consisting of the following groups:

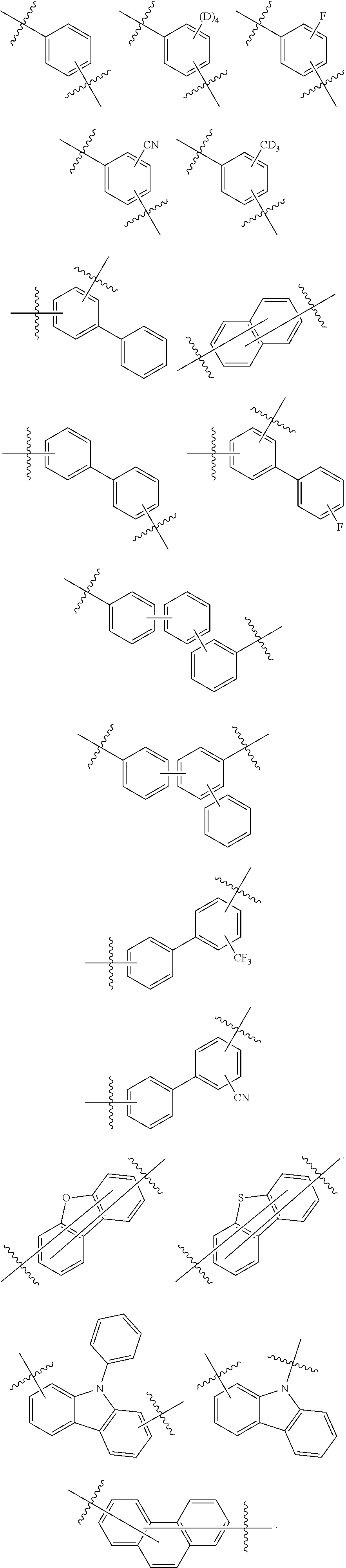

In some embodiments,

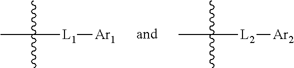

are each independently selected from

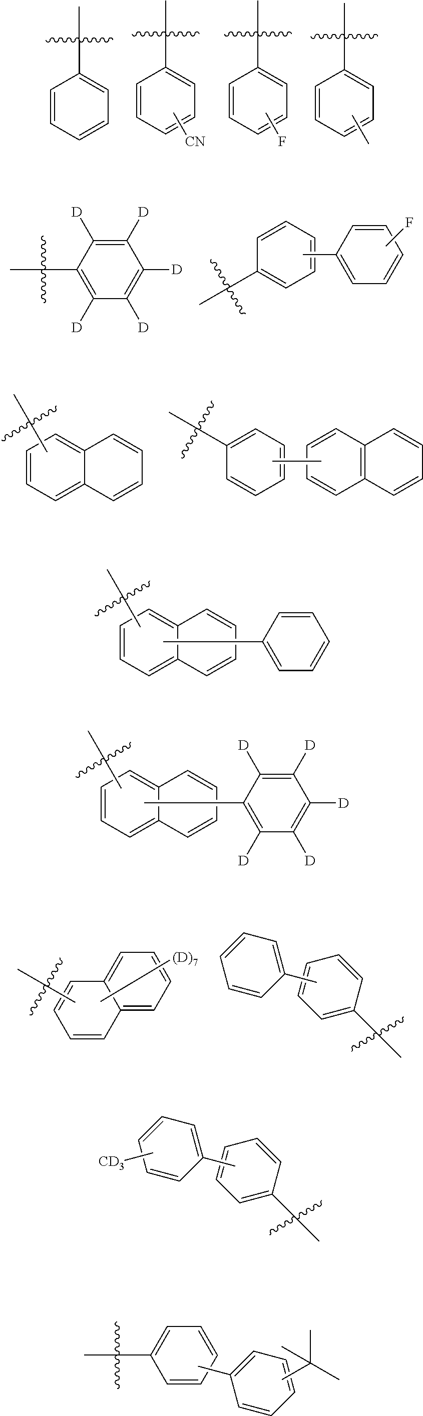

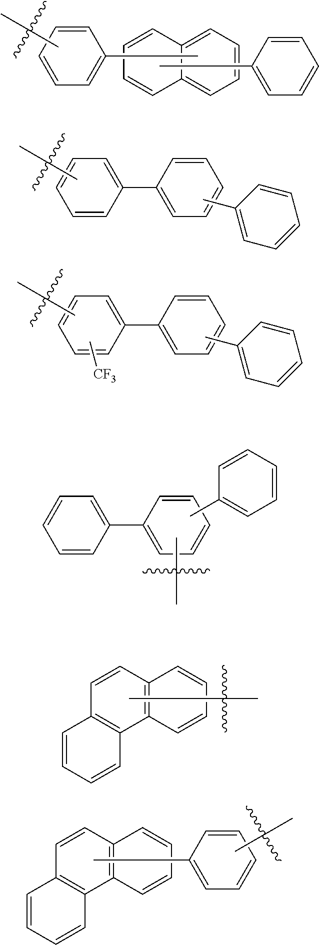

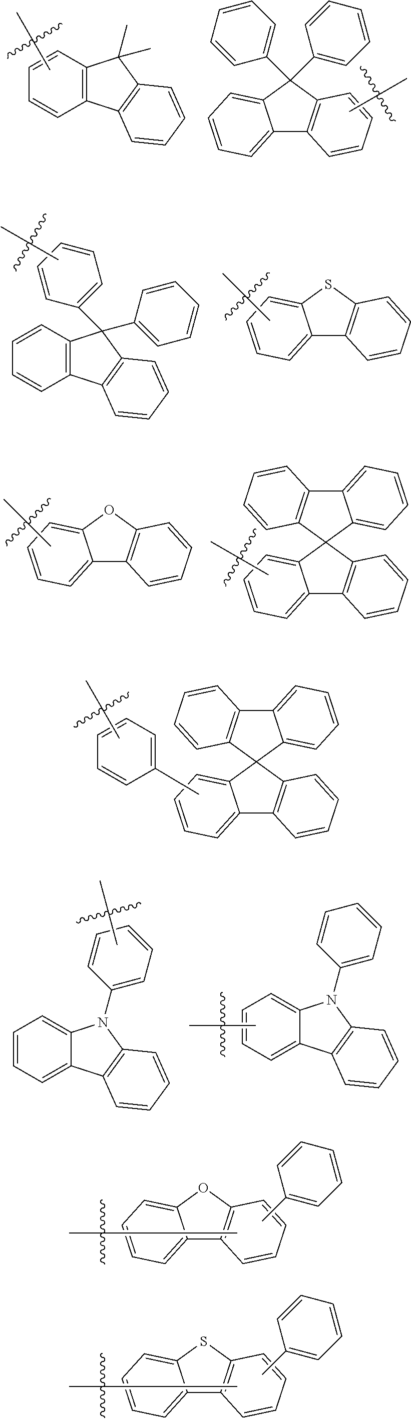

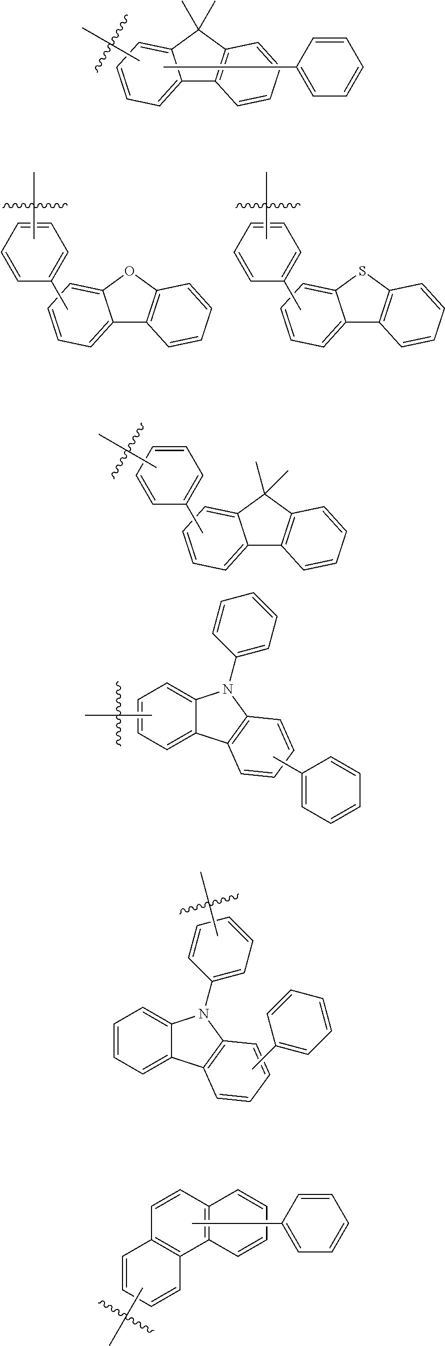

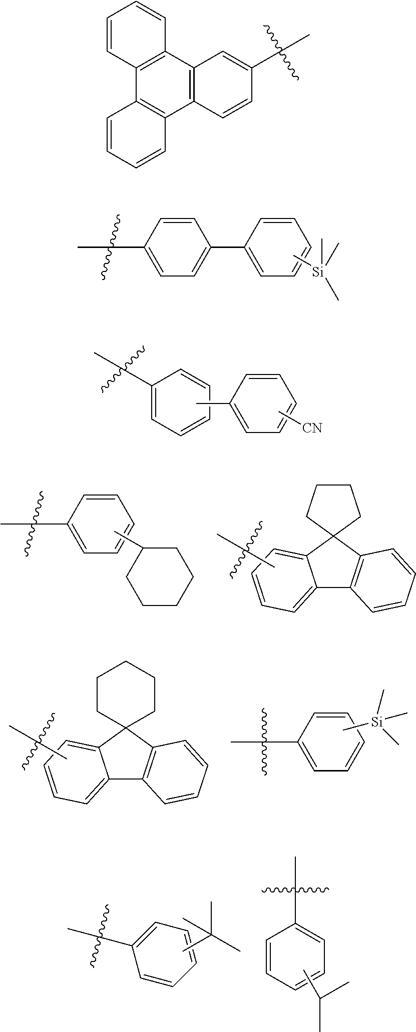

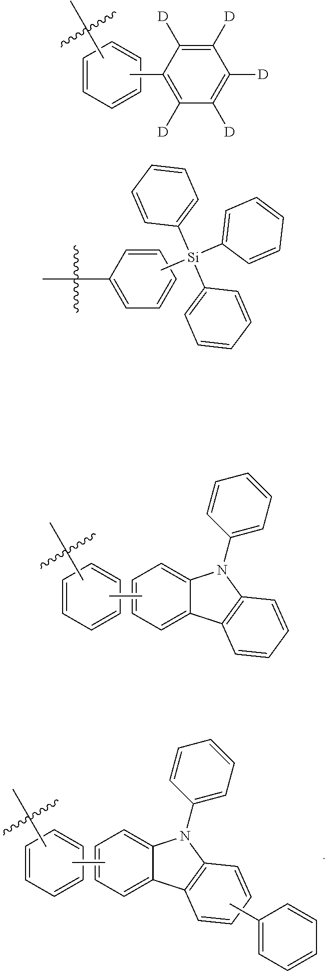

the following groups:

In some embodiments, R₁, R₂, R₃, and R₄ are the same or different, and are each independently selected from a methyl, a trideuterate methyl, a phenyl, and a penta-deuterated phenyl.

In some embodiments, one of R₅ and R₆ is selected from a hydrogen, a deuterium, a fluorine, and a cyano, and the other is

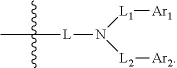

In some embodiments, R₇, R₈, R₉ and R₁₀ are the same or different, and are each independently selected from a hydrogen, a deuterium, a methyl, an ethyl, an isopropyl, a tert-butyl, a phenyl, a deuterated methyl, a deuterated ethyl, a deuterated isopropyl, a deuterated tert-butyl, and a deuterated phenyl.

Optionally, R₇ and R₈, and/or R₈ and R₉, and/or R₉ and R₁₀, are interconnected to form a substituted or unsubstituted cyclohexane.

In some embodiments, substituent(s) in the above cyclohexane are the same or different, and are each independently selected from a methyl, a trideuterated methyl, a phenyl, and a penta-deuterated phenyl.

In some embodiments, R₁₁ and R₁₂ are the same or different, and are each independently selected from a methyl and a phenyl.

In some embodiments, R₁₃ is selected from a phenyl, a naphthyl, and a biphenyl.

In some embodiments, each R is independently selected from a deuterium, a fluorine, a cyano, a methyl, a tert-butyl, and a phenyl.

In some embodiments, the compound represented by formula 1 is selected from the structures represented by formulae 2-1, 2-2, 2-3, and 2-4.

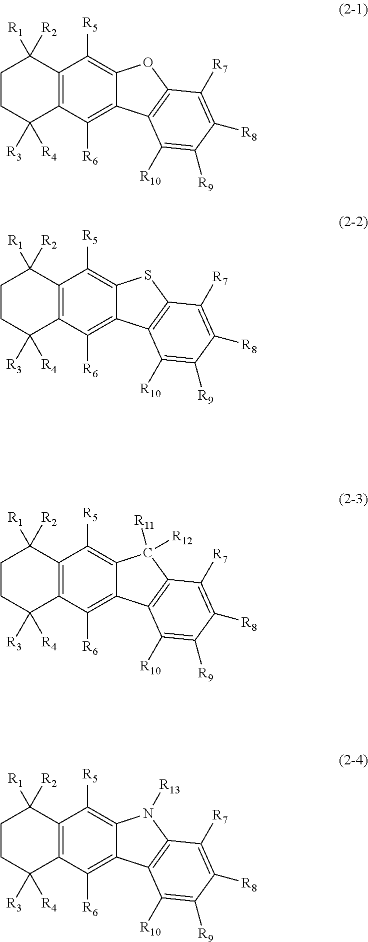

In some embodiments, the compound represented by formula 1 is selected from the structures represented by formulae 3-1, 3-2, 3-3, 3-4, 3-5, 3-6, 3-7, and 3-8.

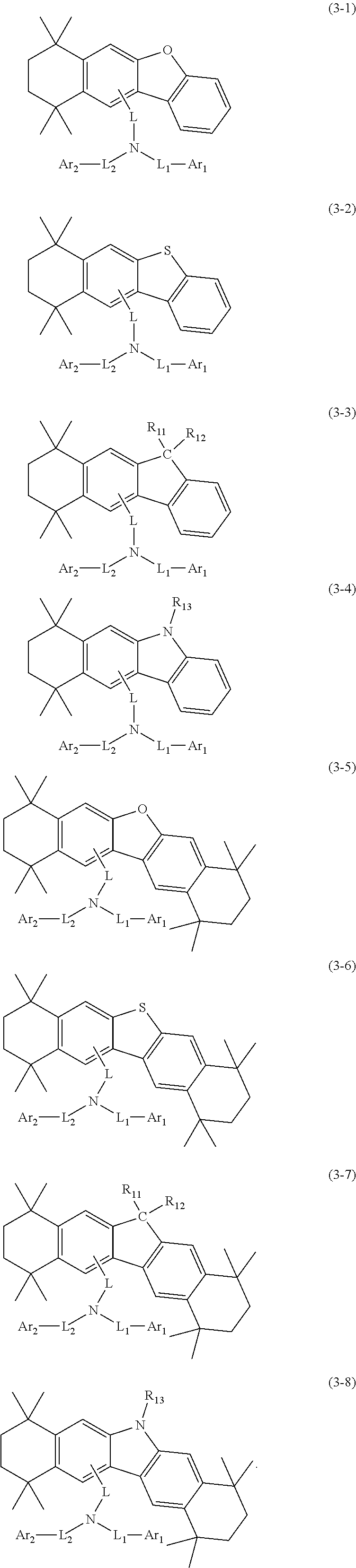

In some examples, the compound of the present disclosure is selected from the group consisting of the following compounds.

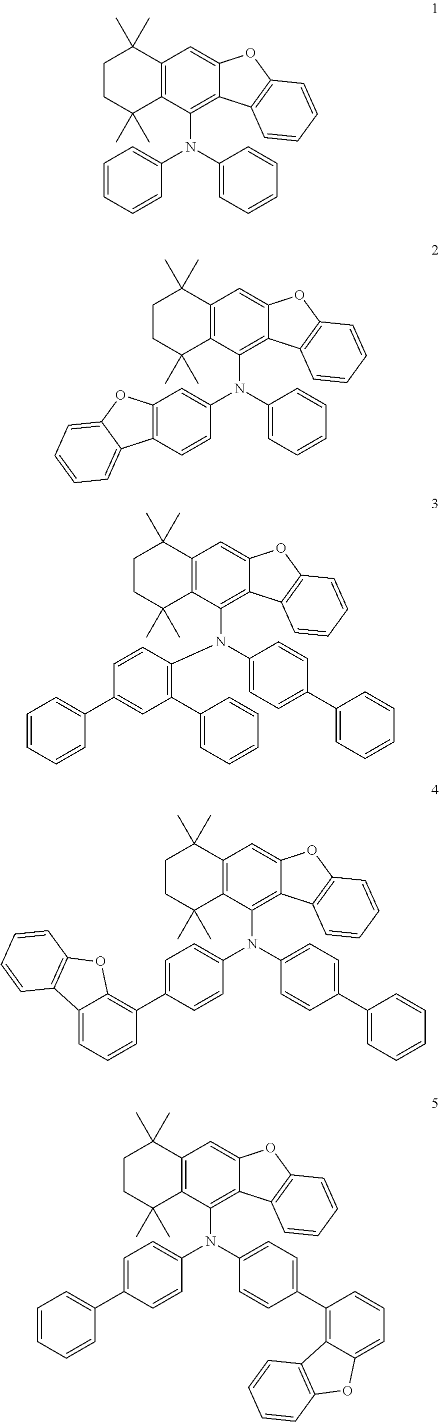 [SMILES: CC1(C)CCC(C)(C)c2c1cc1oc3ccccc3c1c2N(c1ccccc1)c1ccccc1 ; CC1(C)CCC(C)(C)c2c1cc1oc3ccccc3c1c2N(c1ccccc1)c1ccc2c(c1)oc1ccccc12 ; CC1(C)CCC(C)(C)c2c1cc1oc3ccccc3c1c2N(c1ccc(-c2ccccc2)cc1)c1ccc(-c2ccccc2)cc1-c1ccccc1 ; CC1(C)CCC(C)(C)c2c1cc1oc3ccccc3c1c2N(c1ccc(-c2ccccc2)cc1)c1ccc(-c2cccc3c2oc2ccccc23)cc1 ; CC1(C)CCC(C)(C)c2c1cc1oc3ccccc3c1c2N(c1ccc(-c2ccccc2)cc1)c1ccc(-c2cccc3oc4ccccc4c23)cc1]

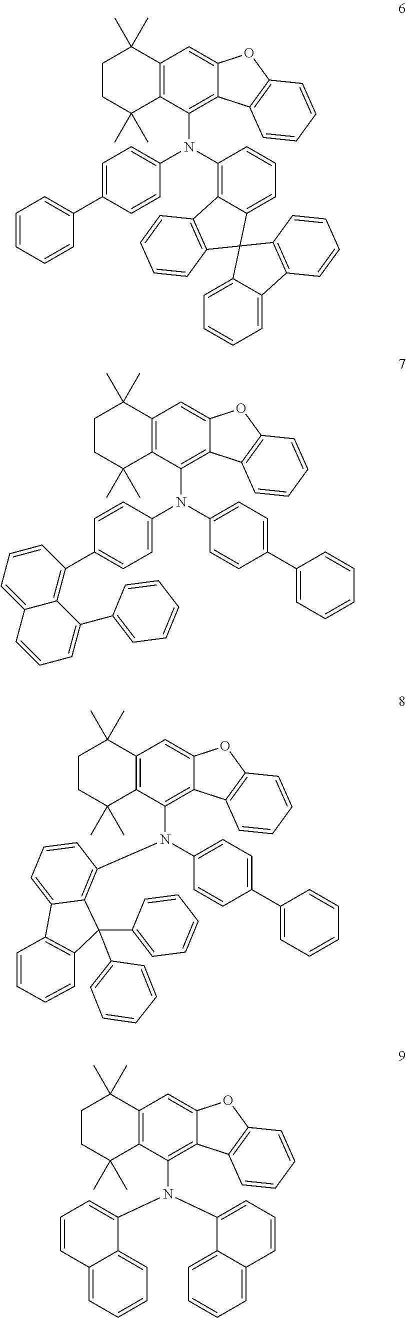

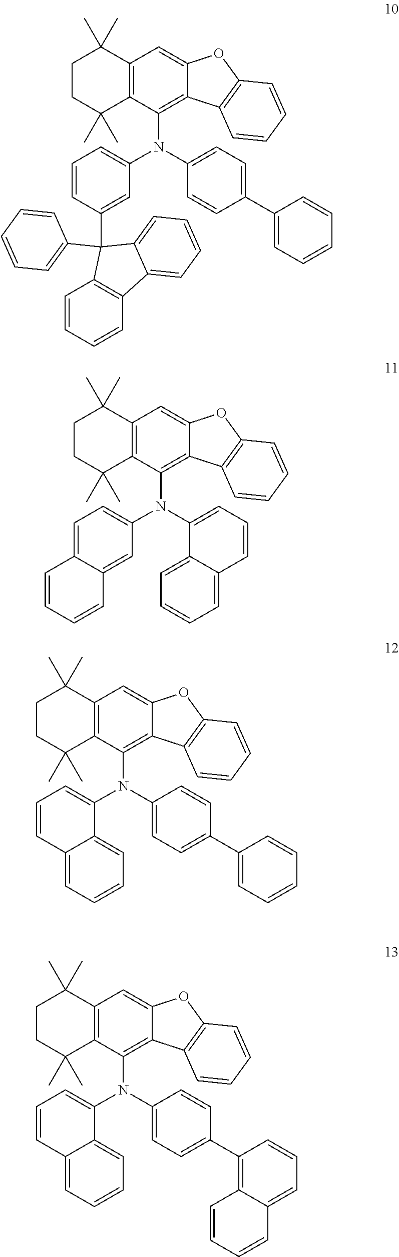

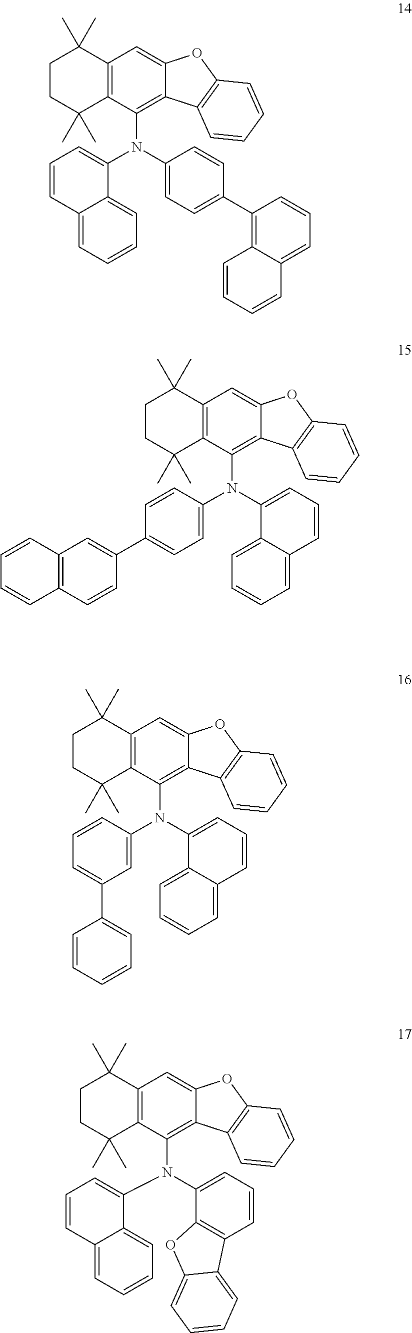 [SMILES: CC1(C)CCC(C)(C)c2c1cc1oc3ccccc3c1c2N(c1ccc(-c2cccc3ccccc23)cc1)c1cccc2ccccc12 ; CC1(C)CCC(C)(C)c2c1cc1oc3ccccc3c1c2N(c1ccc(-c2ccc3ccccc3c2)cc1)c1cccc2ccccc12 ; CC1(C)CCC(C)(C)c2c1cc1oc3ccccc3c1c2N(c1cccc(-c2ccccc2)c1)c1cccc2ccccc12 ; CC1(C)CCC(C)(C)c2c1cc1oc3ccccc3c1c2N(c1cccc2ccccc12)c1cccc2c1oc1ccccc12]

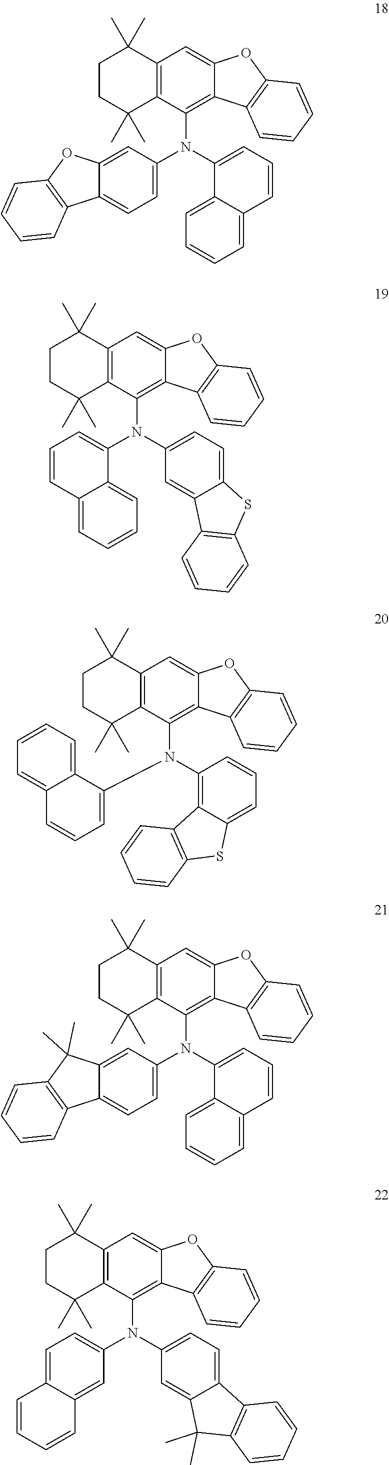

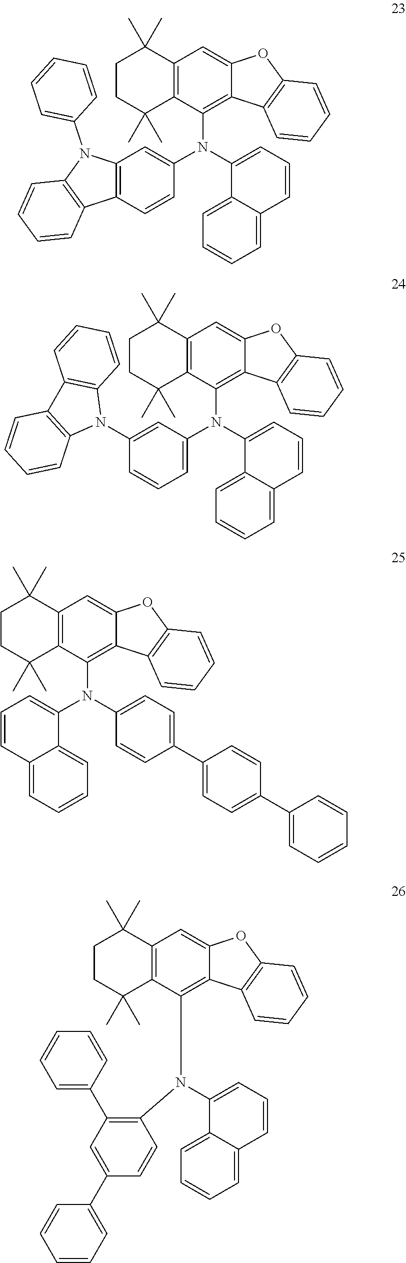 [SMILES: CC1(C)CCC(C)(C)c2c1cc1oc3ccccc3c1c2N(c1ccc2c3ccccc3n(-c3ccccc3)c2c1)c1cccc2ccccc12 ; CC1(C)CCC(C)(C)c2c1cc1oc3ccccc3c1c2N(c1cccc(-n2c3ccccc3c3ccccc32)c1)c1cccc2ccccc12 ; CC1(C)CCC(C)(C)c2c1cc1oc3ccccc3c1c2N(c1ccc(-c2ccc(-c3ccccc3)cc2)cc1)c1cccc2ccccc12 ; CC1(C)CCC(C)(C)c2c1cc1oc3ccccc3c1c2N(c1ccc(-c2ccccc2)cc1-c1ccccc1)c1cccc2ccccc12]

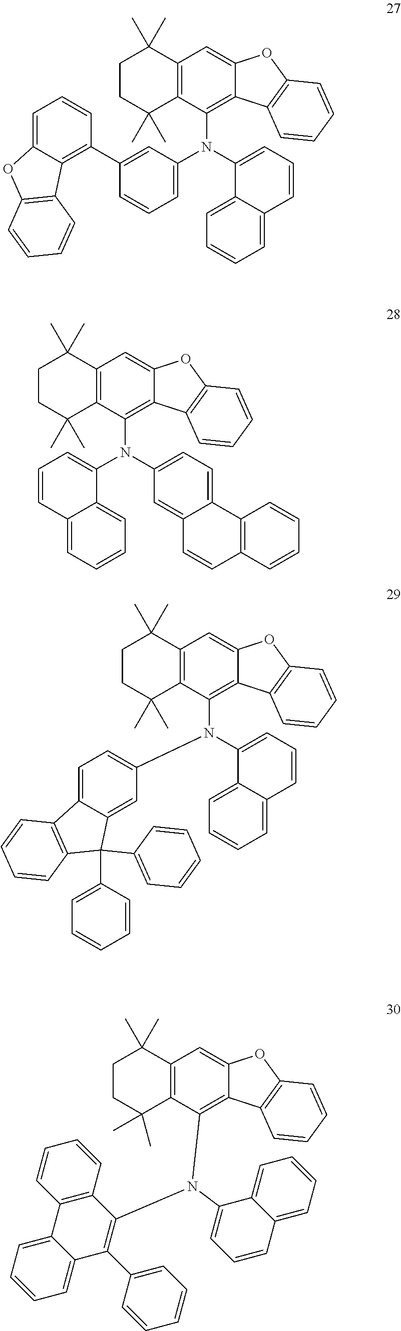 [SMILES: CC1(C)CCC(C)(C)c2c1cc1oc3ccccc3c1c2N(c1cccc(-c2cccc3oc4ccccc4c23)c1)c1cccc2ccccc12 ; CC1(C)CCC(C)(C)c2c1cc1oc3ccccc3c1c2N(c1ccc2c(ccc3ccccc32)c1)c1cccc2ccccc12 ; CC1(C)CCC(C)(C)c2c1cc1oc3ccccc3c1c2N(c1ccc2c(c1)C(c1ccccc1)(c1ccccc1)c1ccccc1-2)c1cccc2ccccc12 ; CC1(C)CCC(C)(C)c2c1cc1oc3ccccc3c1c2N(c1cccc2ccccc12)c1c(-c2ccccc2)c2ccccc2c2ccccc12]

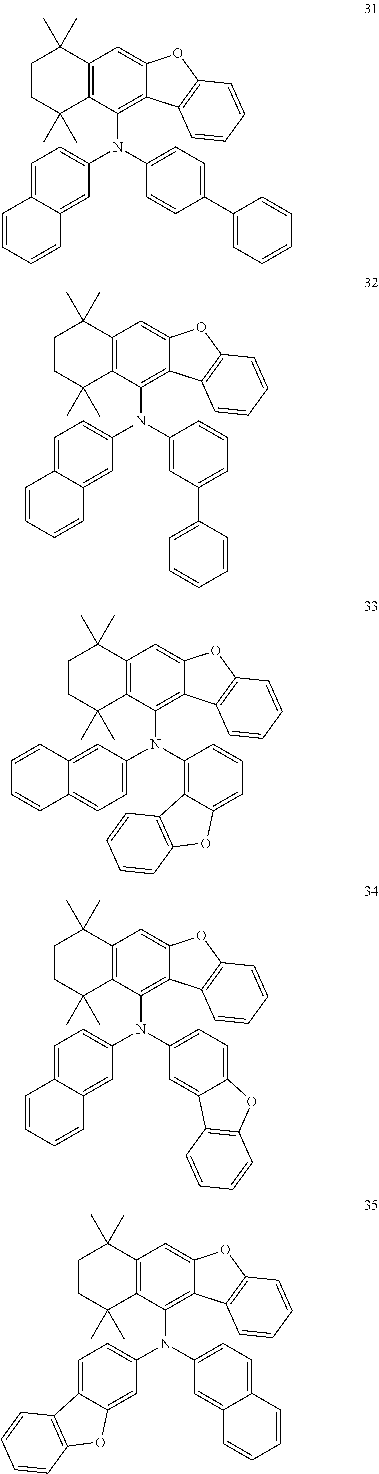

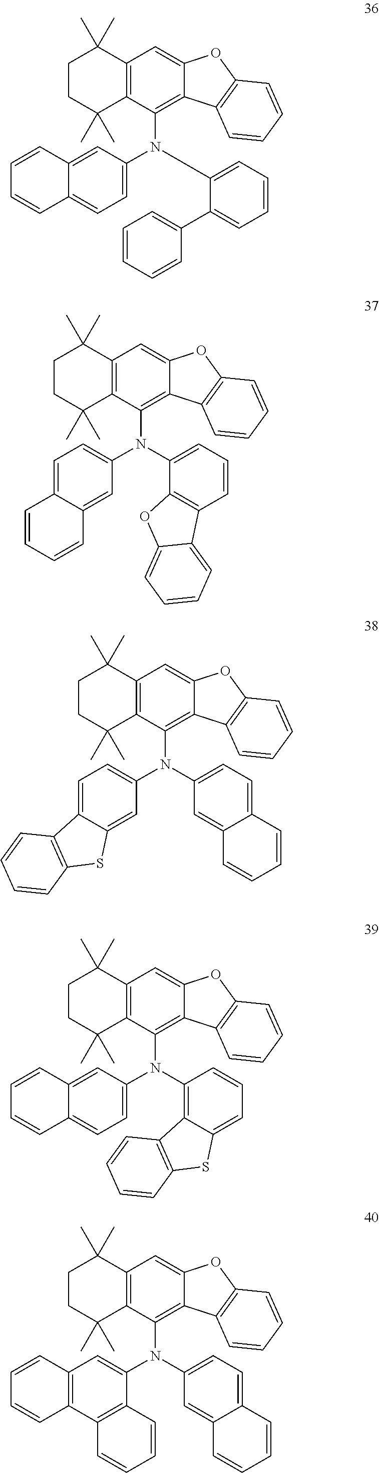

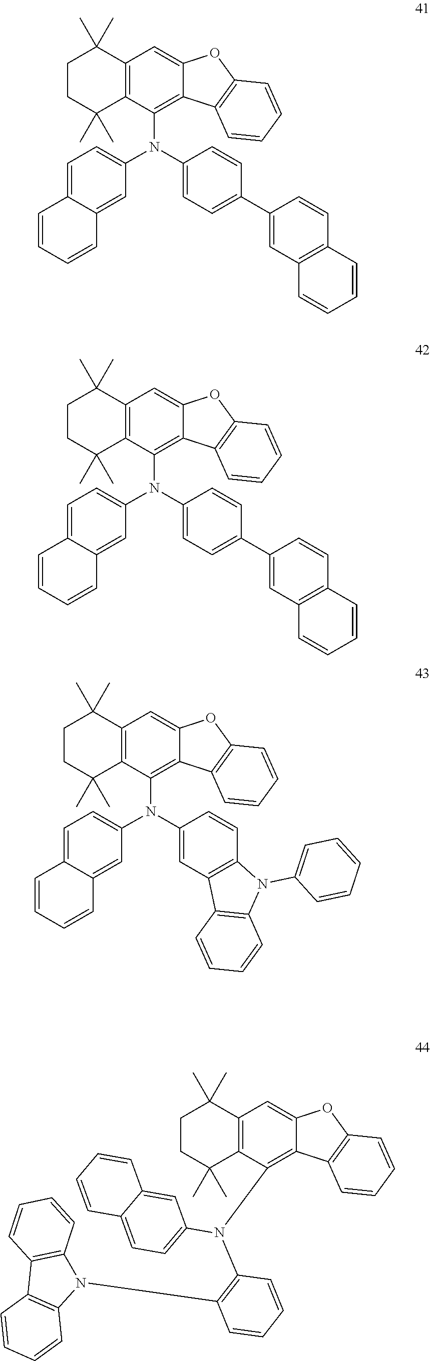 [SMILES: CC1(C)CCC(C)(C)c2c1cc1oc3ccccc3c1c2N(c1ccc(-c2ccc3ccccc3c2)cc1)c1ccc2ccccc2c1 ; CC1(C)CCC(C)(C)c2c1cc1oc3ccccc3c1c2N(c1ccc(-c2ccc3ccccc3c2)cc1)c1ccc2ccccc2c1 ; CC1(C)CCC(C)(C)c2c1cc1oc3ccccc3c1c2N(c1ccc2ccccc2c1)c1ccc2c(c1)c1ccccc1n2-c1ccccc1 ; CC1(C)CCC(C)(C)c2c1cc1oc3ccccc3c1c2N(c1ccc2ccccc2c1)c1ccccc1-n1c2ccccc2c2ccccc21]

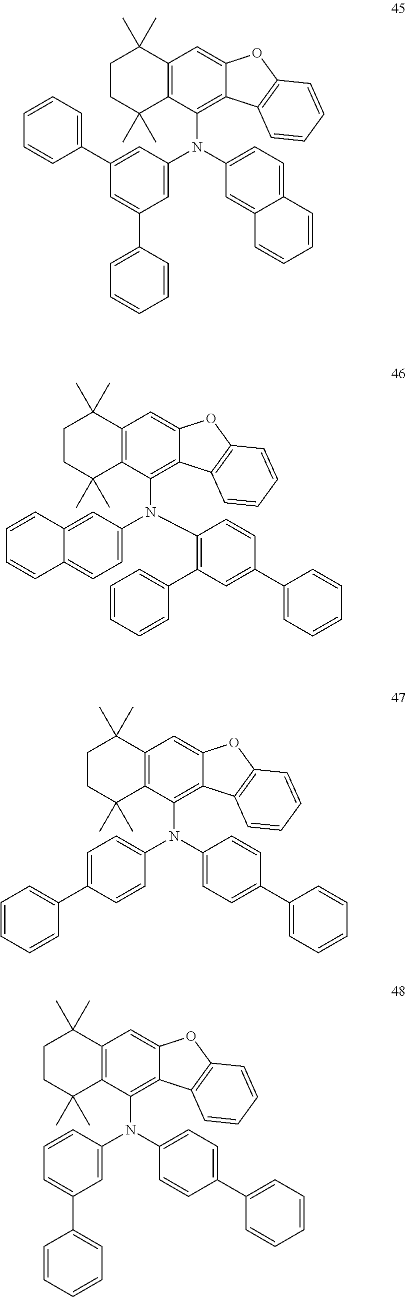 [SMILES: CC1(C)CCC(C)(C)c2c1cc1oc3ccccc3c1c2N(c1cc(-c2ccccc2)cc(-c2ccccc2)c1)c1ccc2ccccc2c1 ; CC1(C)CCC(C)(C)c2c1cc1oc3ccccc3c1c2N(c1ccc2ccccc2c1)c1ccc(-c2ccccc2)cc1-c1ccccc1 ; CC1(C)CCC(C)(C)c2c1cc1oc3ccccc3c1c2N(c1ccc(-c2ccccc2)cc1)c1ccc(-c2ccccc2)cc1 ; CC1(C)CCC(C)(C)c2c1cc1oc3ccccc3c1c2N(c1ccc(-c2ccccc2)cc1)c1cccc(-c2ccccc2)c1]

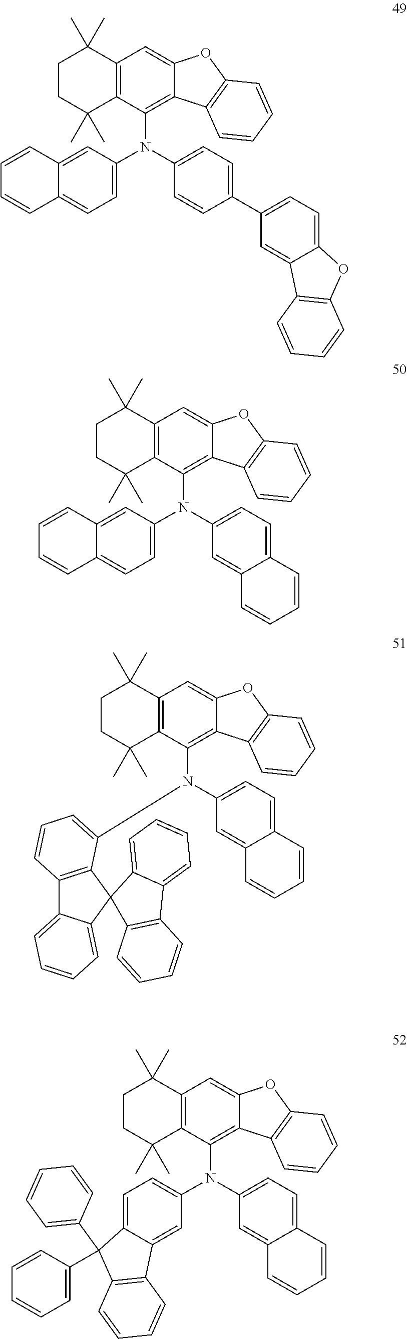 [SMILES: CC1(C)CCC(C)(C)c2c1cc1oc3ccccc3c1c2N(c1ccc(-c2ccc3oc4ccccc4c3c2)cc1)c1ccc2ccccc2c1 ; CC1(C)CCC(C)(C)c2c1cc1oc3ccccc3c1c2N(c1ccc2ccccc2c1)c1ccc2ccccc2c1 ; CC1(C)CCC(C)(C)c2c1cc1oc3ccccc3c1c2N(c1ccc2ccccc2c1)c1cccc2c1C1(c3ccccc3-c3ccccc31)c1ccccc1-2 ; CC1(C)CCC(C)(C)c2c1cc1oc3ccccc3c1c2N(c1ccc2c(c1)-c1ccccc1C2(c1ccccc1)c1ccccc1)c1ccc2ccccc2c1]

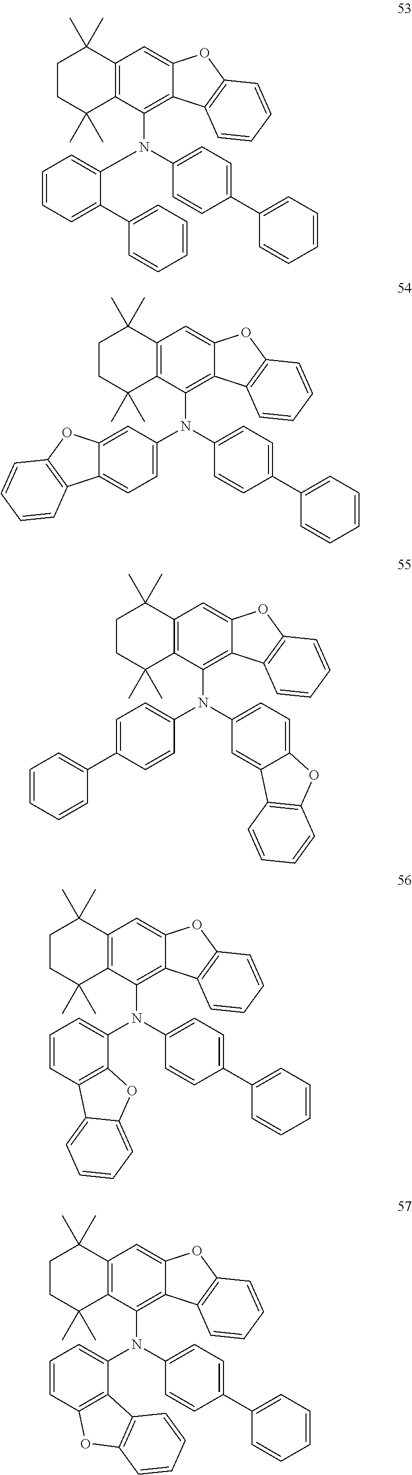

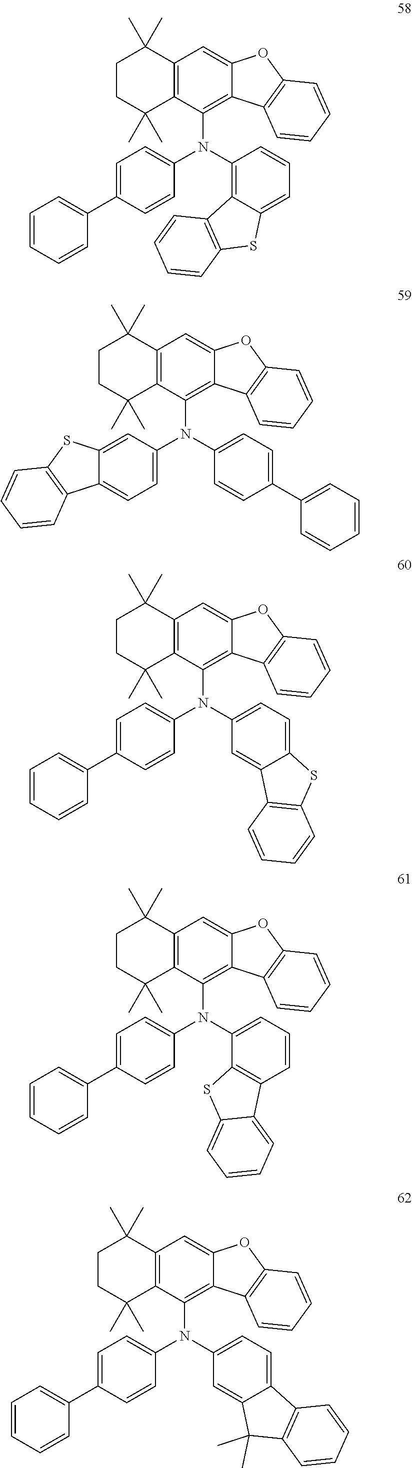

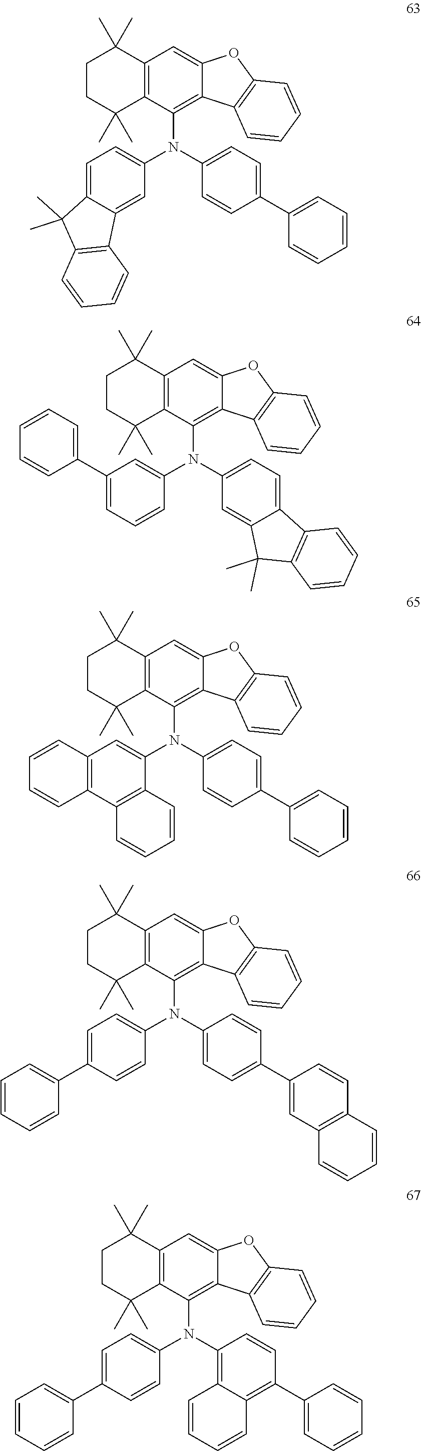

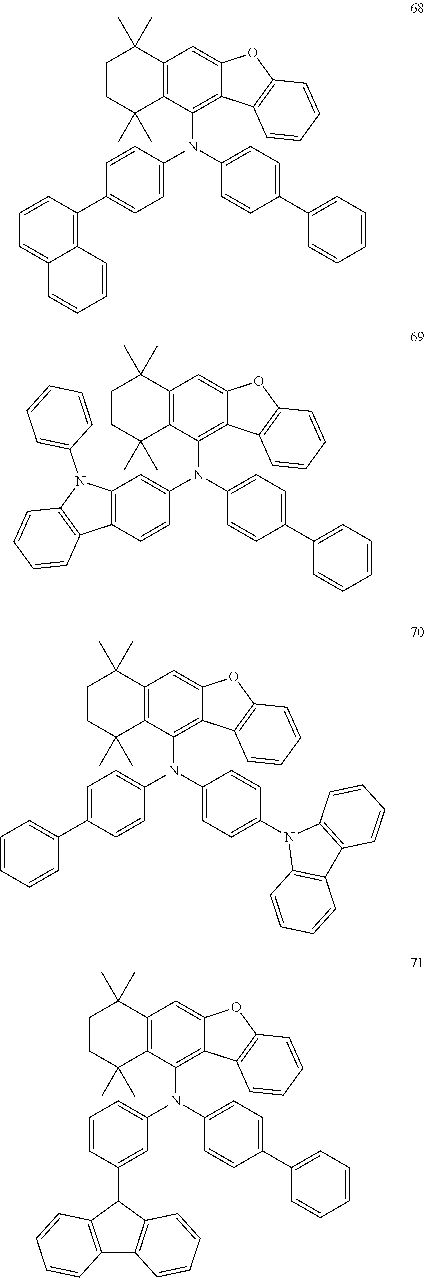 [SMILES: CC1(C)CCC(C)(C)c2c1cc1oc3ccccc3c1c2N(c1ccc(-c2ccccc2)cc1)c1ccc(-c2cccc3ccccc23)cc1 ; CC1(C)CCC(C)(C)c2c1cc1oc3ccccc3c1c2N(c1ccc(-c2ccccc2)cc1)c1ccc2c3ccccc3n(-c3ccccc3)c2c1 ; CC1(C)CCC(C)(C)c2c1cc1oc3ccccc3c1c2N(c1ccc(-c2ccccc2)cc1)c1ccc(-n2c3ccccc3c3ccccc32)cc1 ; CC1(C)CCC(C)(C)c2c1cc1oc3ccccc3c1c2N(c1ccc(-c2ccccc2)cc1)c1cccc(C2c3ccccc3-c3ccccc32)c1]

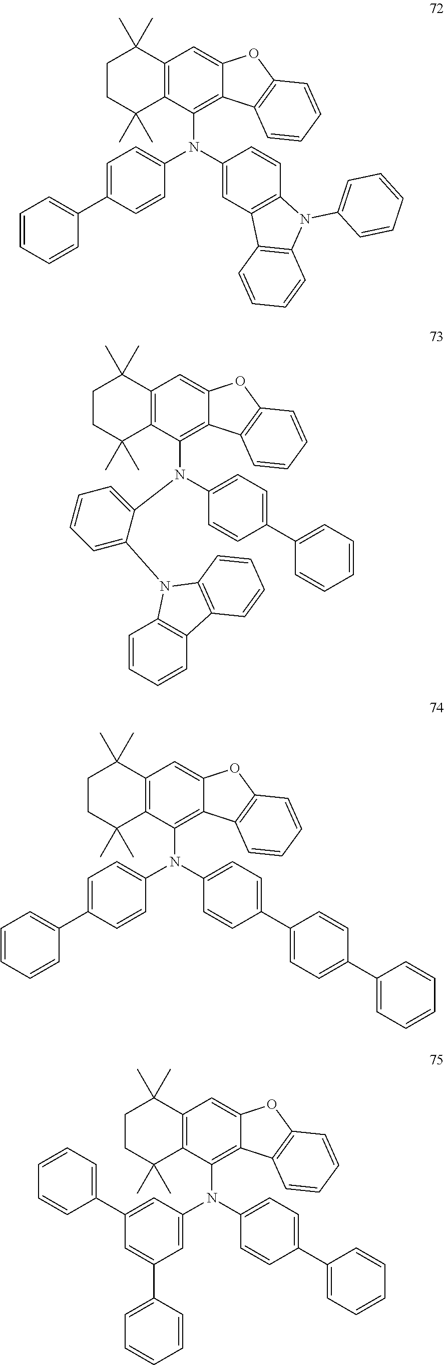 [SMILES: CC1(C)CCC(C)(C)c2c1cc1oc3ccccc3c1c2N(c1ccc(-c2ccccc2)cc1)c1ccc2c(c1)c1ccccc1n2-c1ccccc1 ; CC1(C)CCC(C)(C)c2c1cc1oc3ccccc3c1c2N(c1ccc(-c2ccccc2)cc1)c1ccccc1-n1c2ccccc2c2ccccc21 ; CC1(C)CCC(C)(C)c2c1cc1oc3ccccc3c1c2N(c1ccc(-c2ccccc2)cc1)c1ccc(-c2ccc(-c3ccccc3)cc2)cc1 ; CC1(C)CCC(C)(C)c2c1cc1oc3ccccc3c1c2N(c1ccc(-c2ccccc2)cc1)c1cc(-c2ccccc2)cc(-c2ccccc2)c1]

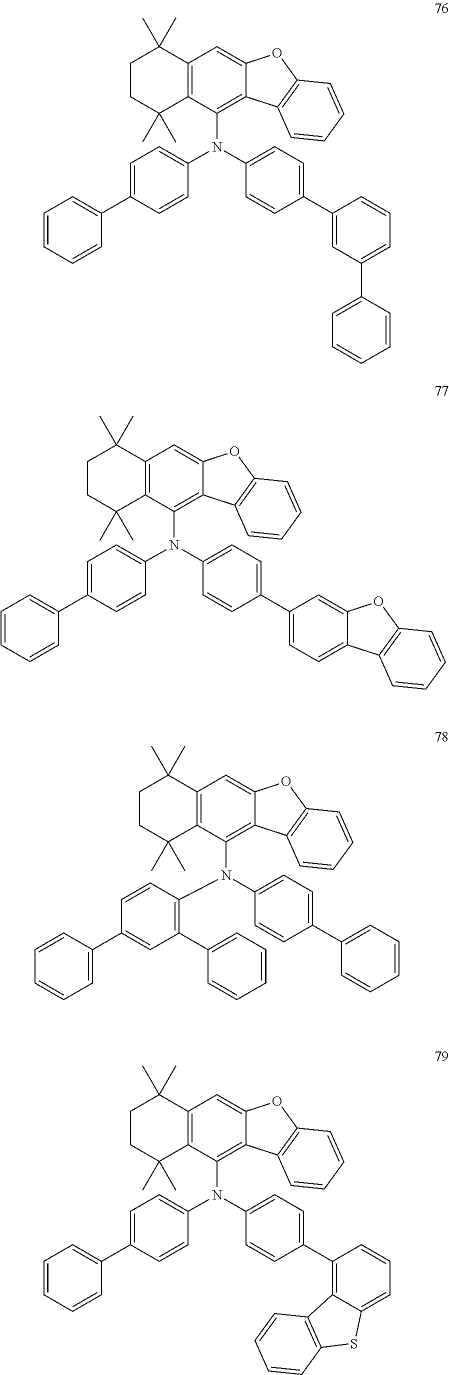 [SMILES: CC1(C)CCC(C)(C)c2c1cc1oc3ccccc3c1c2N(c1ccc(-c2ccccc2)cc1)c1ccc(-c2cccc(-c3ccccc3)c2)cc1 ; CC1(C)CCC(C)(C)c2c1cc1oc3ccccc3c1c2N(c1ccc(-c2ccccc2)cc1)c1ccc(-c2ccc3c(c2)oc2ccccc23)cc1 ; CC1(C)CCC(C)(C)c2c1cc1oc3ccccc3c1c2N(c1ccc(-c2ccccc2)cc1)c1ccc(-c2ccccc2)cc1-c1ccccc1 ; CC1(C)CCC(C)(C)c2c1cc1oc3ccccc3c1c2N(c1ccc(-c2ccccc2)cc1)c1ccc(-c2cccc3sc4ccccc4c23)cc1]

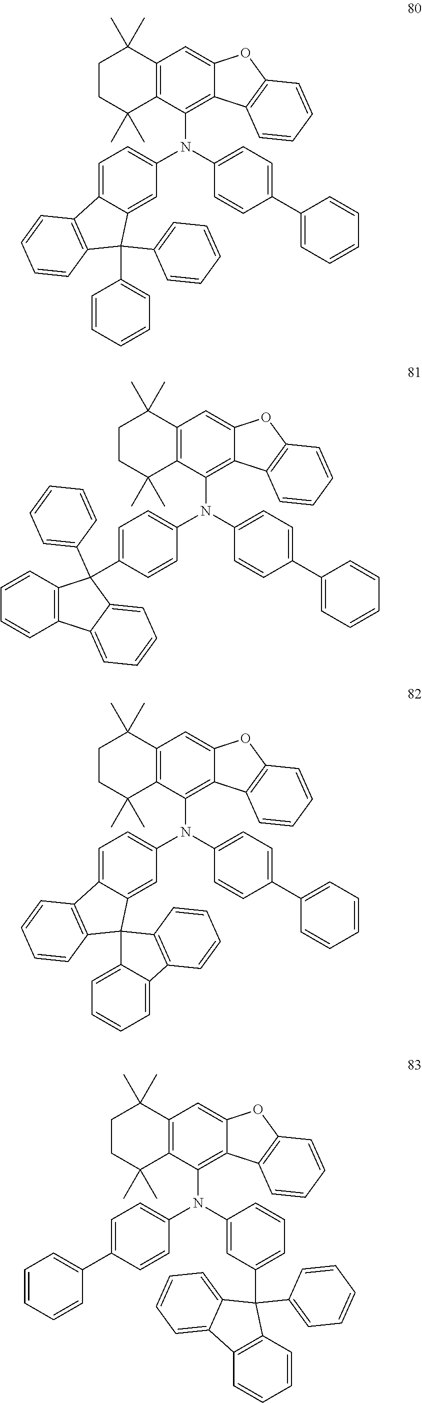

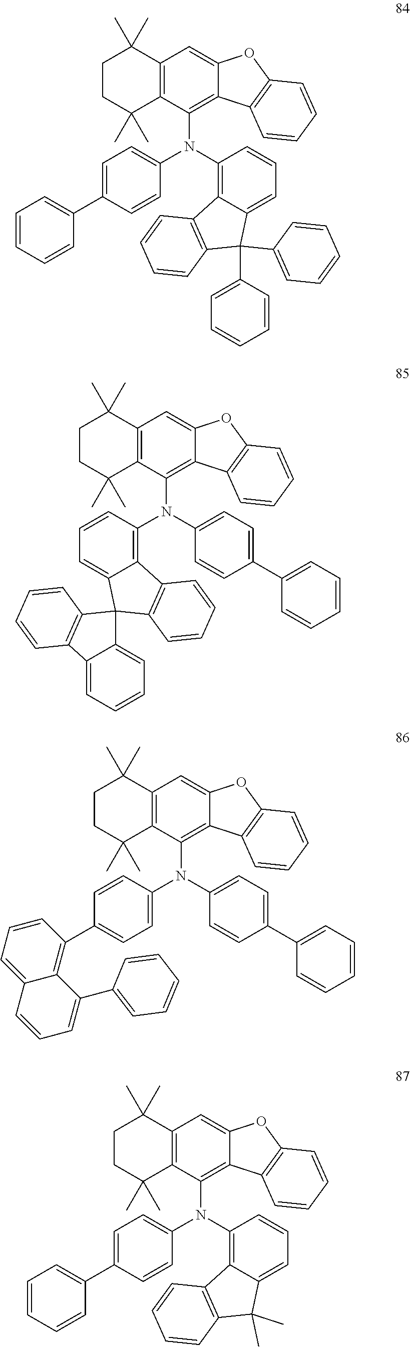

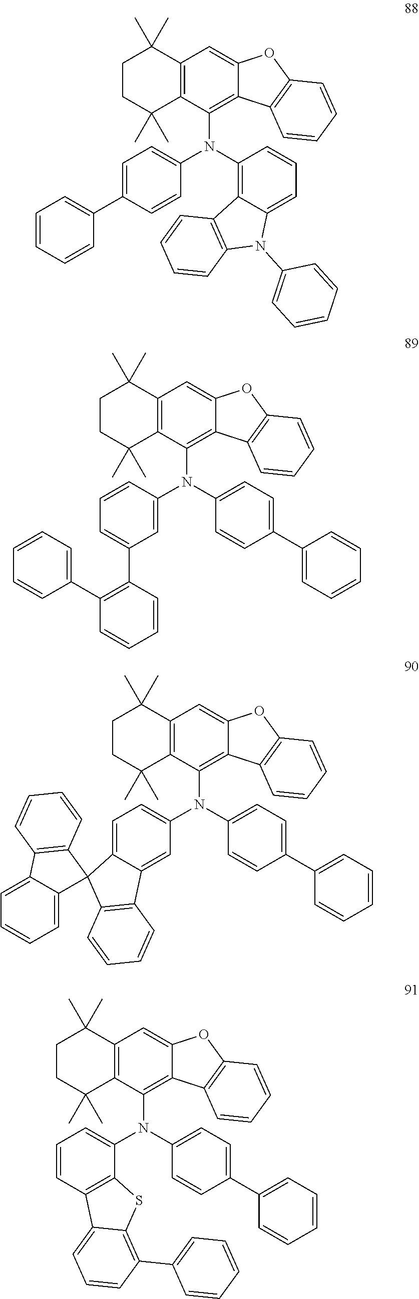

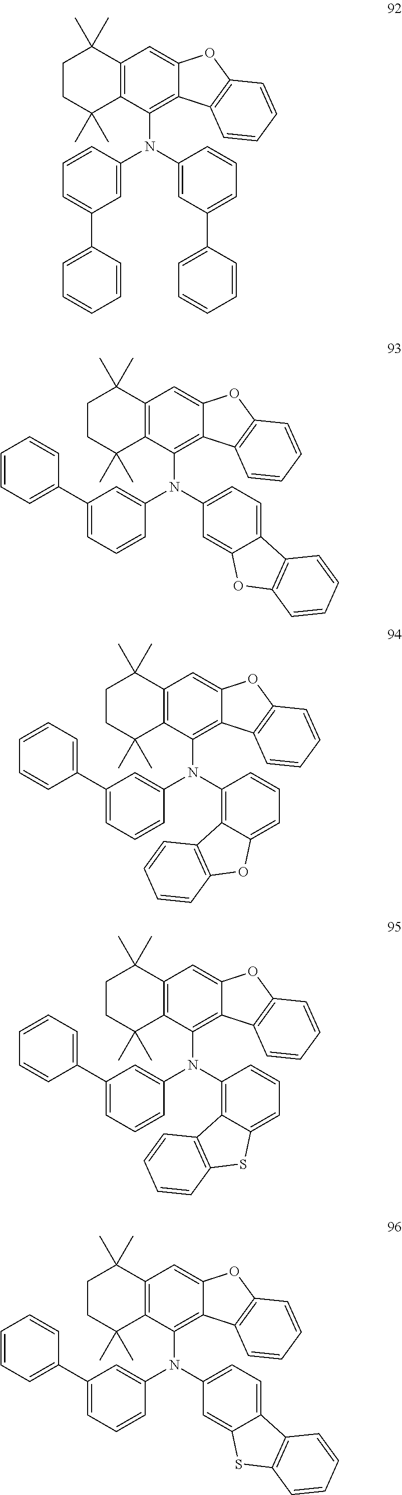

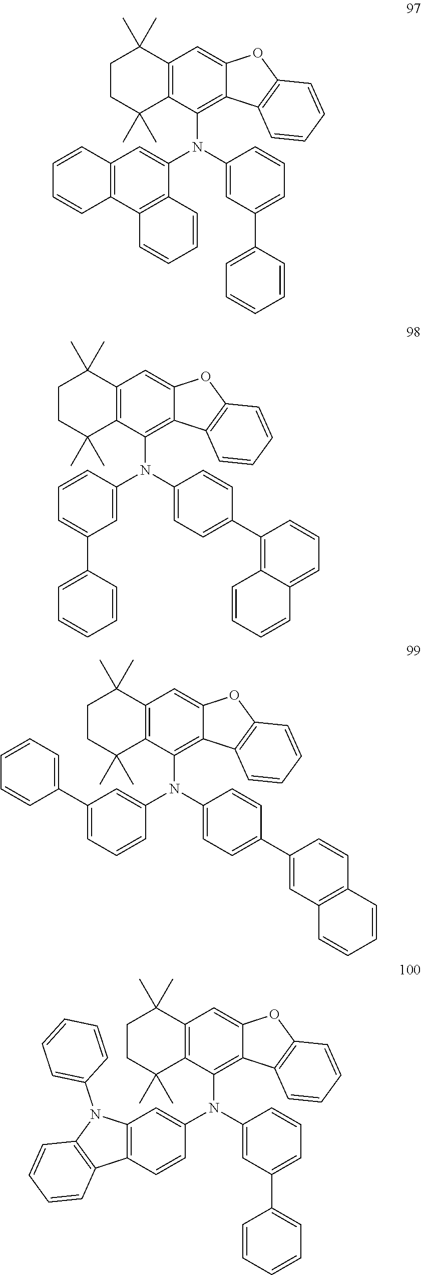

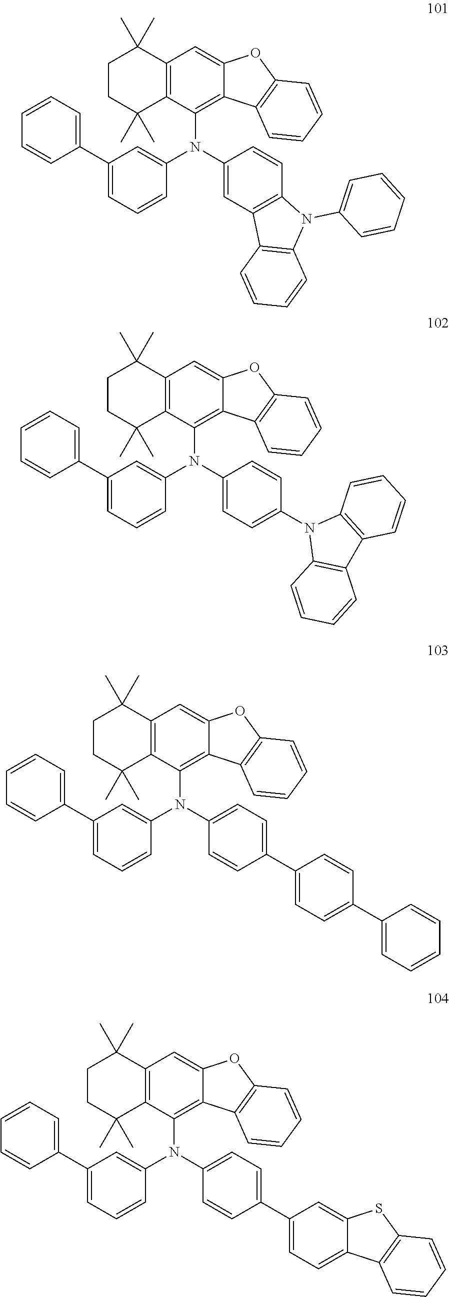 [SMILES: CC1(C)CCC(C)(C)c2c1cc1oc3ccccc3c1c2N(c1cccc(-c2ccccc2)c1)c1ccc2c(c1)c1ccccc1n2-c1ccccc1 ; CC1(C)CCC(C)(C)c2c1cc1oc3ccccc3c1c2N(c1ccc(-n2c3ccccc3c3ccccc32)cc1)c1cccc(-c2ccccc2)c1 ; CC1(C)CCC(C)(C)c2c1cc1oc3ccccc3c1c2N(c1ccc(-c2ccc(-c3ccccc3)cc2)cc1)c1cccc(-c2ccccc2)c1 ; CC1(C)CCC(C)(C)c2c1cc1oc3ccccc3c1c2N(c1ccc(-c2ccc3c(c2)sc2ccccc23)cc1)c1cccc(-c2ccccc2)c1]

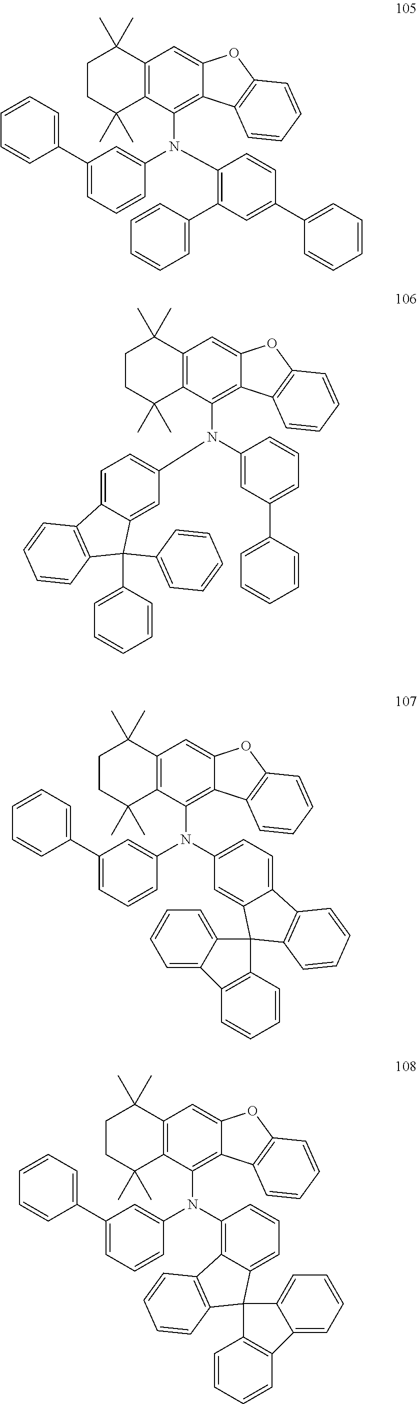

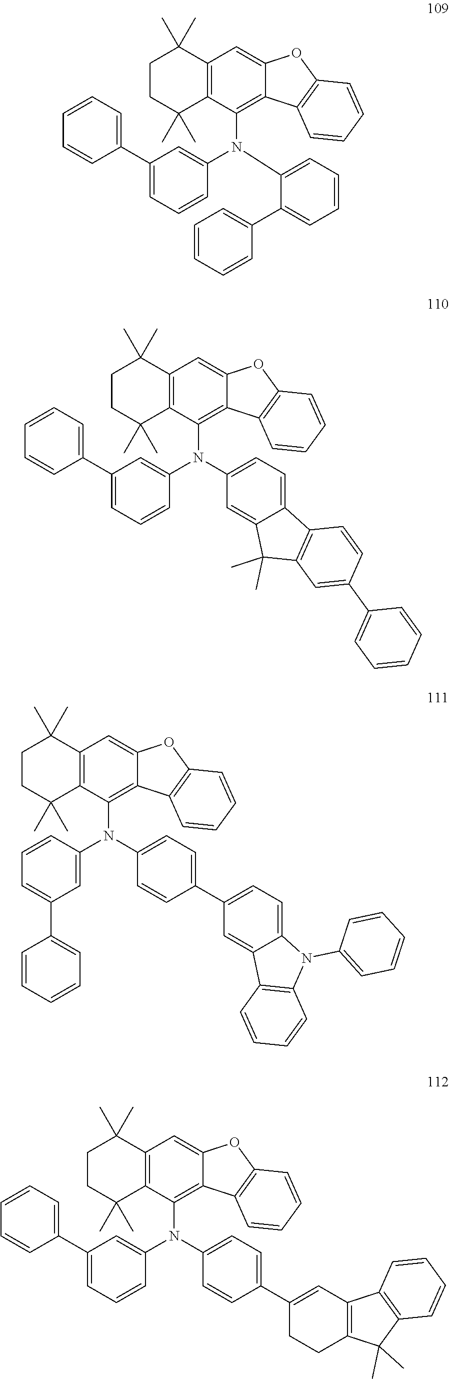 [SMILES: CC1(C)CCC(C)(C)c2c1cc1oc3ccccc3c1c2N(c1cccc(-c2ccccc2)c1)c1ccccc1-c1ccccc1 ; CC1(C)CCC(C)(C)c2c1cc1oc3ccccc3c1c2N(c1cccc(-c2ccccc2)c1)c1ccc2c(c1)C(C)(C)c1cc(-c3ccccc3)ccc1-2 ; CC1(C)CCC(C)(C)c2c1cc1oc3ccccc3c1c2N(c1ccc(-c2ccc3c(c2)c2ccccc2n3-c2ccccc2)cc1)c1cccc(-c2ccccc2)c1 ; CC1(C)CCC(C)(C)c2c1cc1oc3ccccc3c1c2N(c1ccc(C2=CC3=C(CC2)C(C)(C)c2ccccc23)cc1)c1cccc(-c2ccccc2)c1]

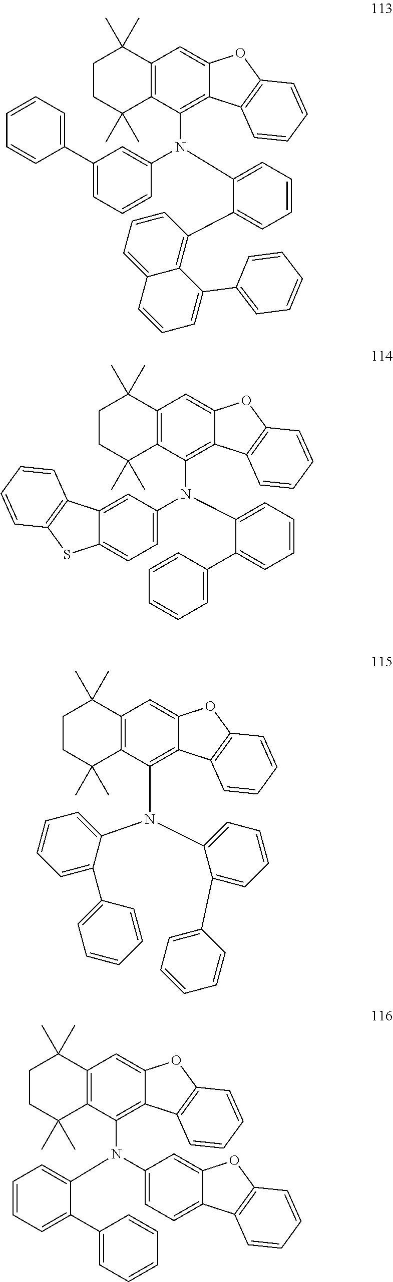

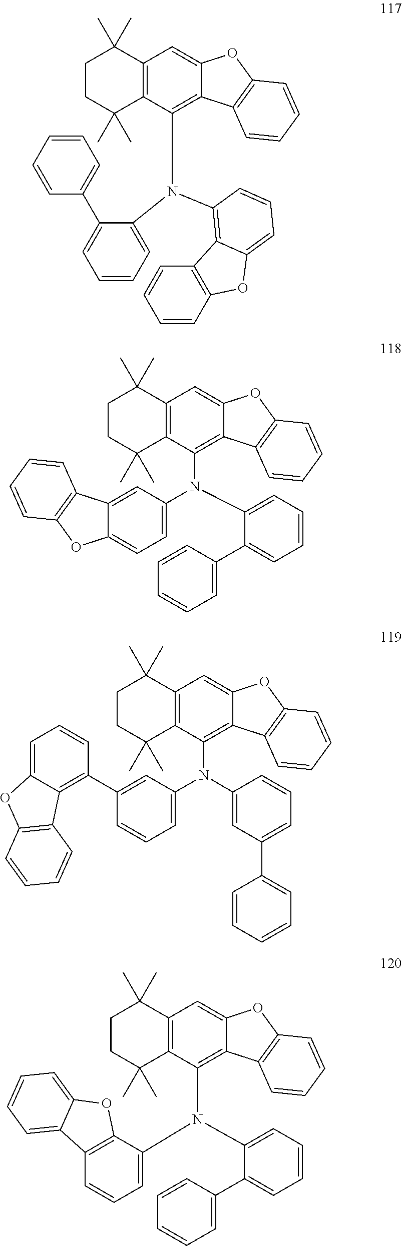

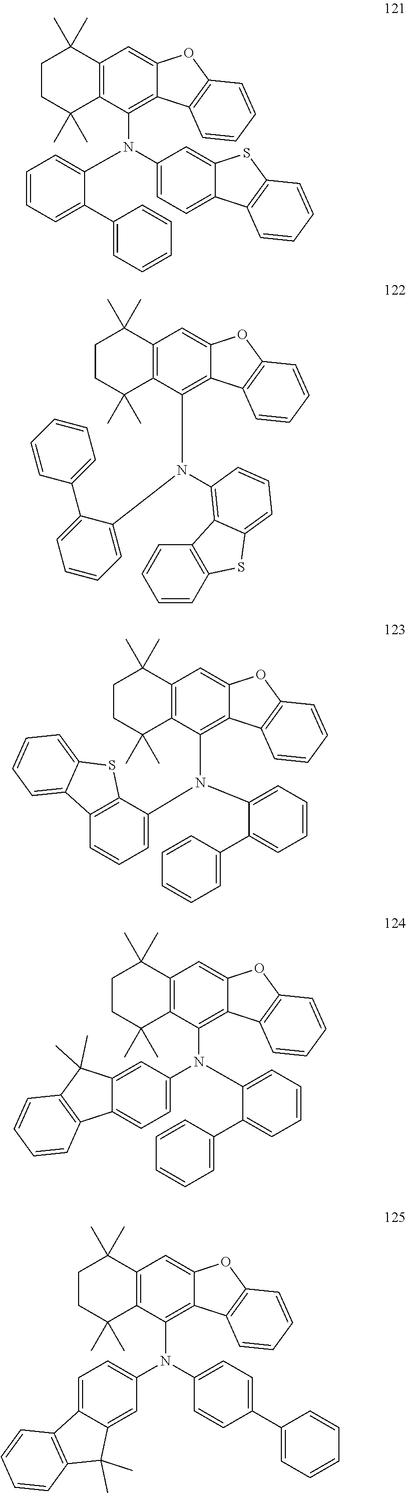

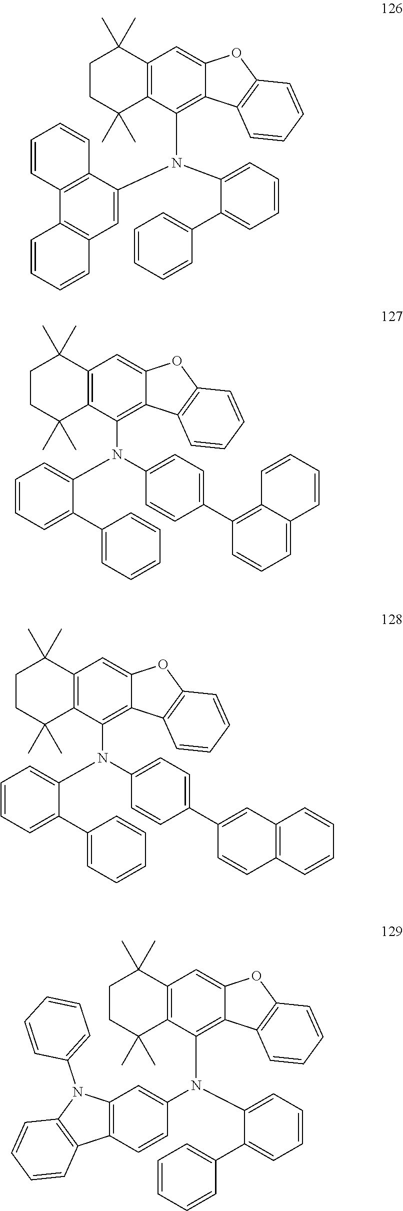

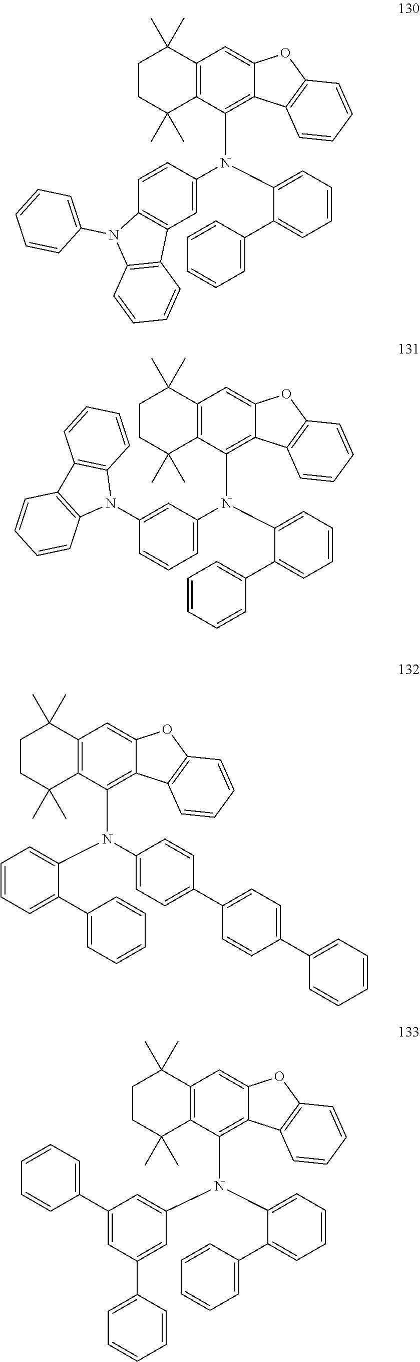

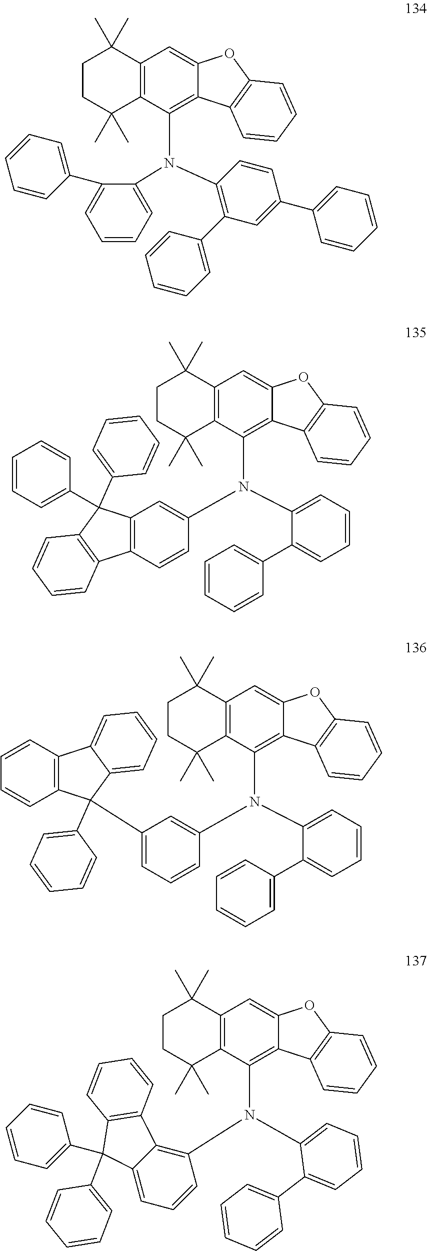 [SMILES: CC1(C)CCC(C)(C)c2c1cc1oc3ccccc3c1c2N(c1ccccc1-c1ccccc1)c1ccc(-c2ccccc2)cc1-c1ccccc1 ; CC1(C)CCC(C)(C)c2c1cc1oc3ccccc3c1c2N(c1ccc2c(c1)C(c1ccccc1)(c1ccccc1)c1ccccc1-2)c1ccccc1-c1ccccc1 ; CC1(C)CCC(C)(C)c2c1cc1oc3ccccc3c1c2N(c1cccc(C2(c3ccccc3)c3ccccc3-c3ccccc32)c1)c1ccccc1-c1ccccc1 ; CC1(C)CCC(C)(C)c2c1cc1oc3ccccc3c1c2N(c1ccccc1-c1ccccc1)c1cccc2c1-c1ccccc1C2(c1ccccc1)c1ccccc1]

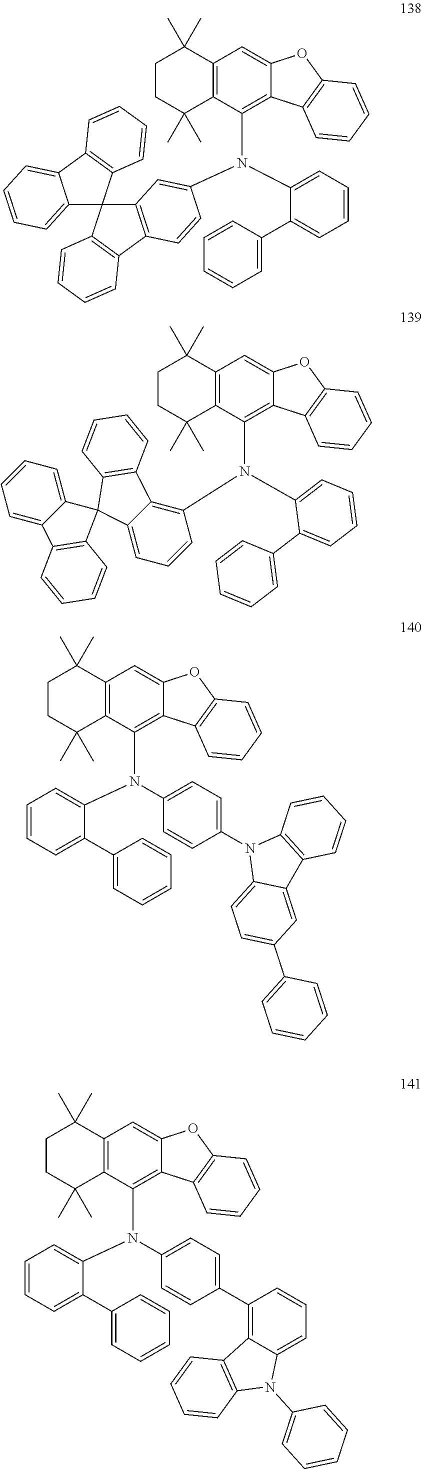

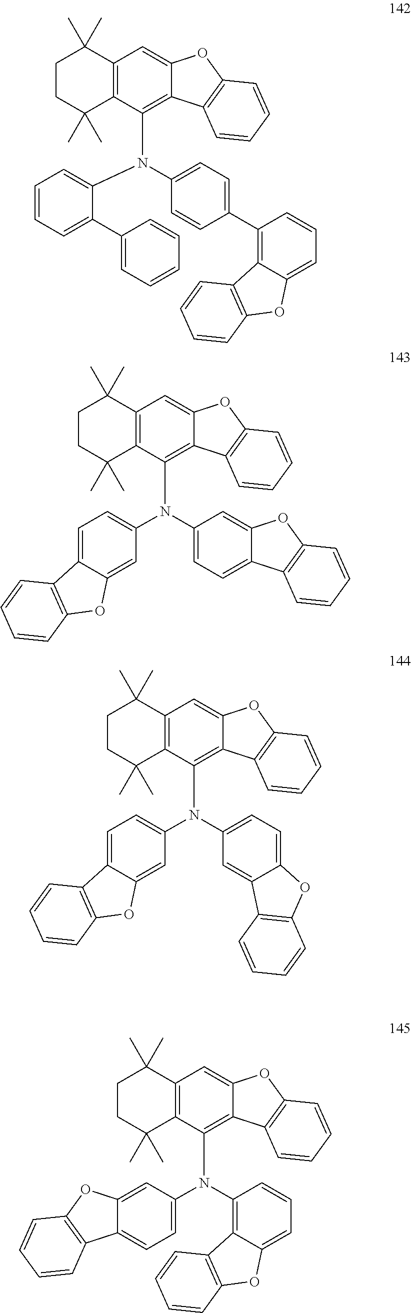 [SMILES: CC1(C)CCC(C)(C)c2c1cc1oc3ccccc3c1c2N(c1ccc(-c2cccc3oc4ccccc4c23)cc1)c1ccccc1-c1ccccc1 ; CC1(C)CCC(C)(C)c2c1cc1oc3ccccc3c1c2N(c1ccc2c(c1)oc1ccccc12)c1ccc2c(c1)oc1ccccc12 ; CC1(C)CCC(C)(C)c2c1cc1oc3ccccc3c1c2N(c1ccc2c(c1)oc1ccccc12)c1ccc2oc3ccccc3c2c1 ; CC1(C)CCC(C)(C)c2c1cc1oc3ccccc3c1c2N(c1ccc2c(c1)oc1ccccc12)c1cccc2oc3ccccc3c12]

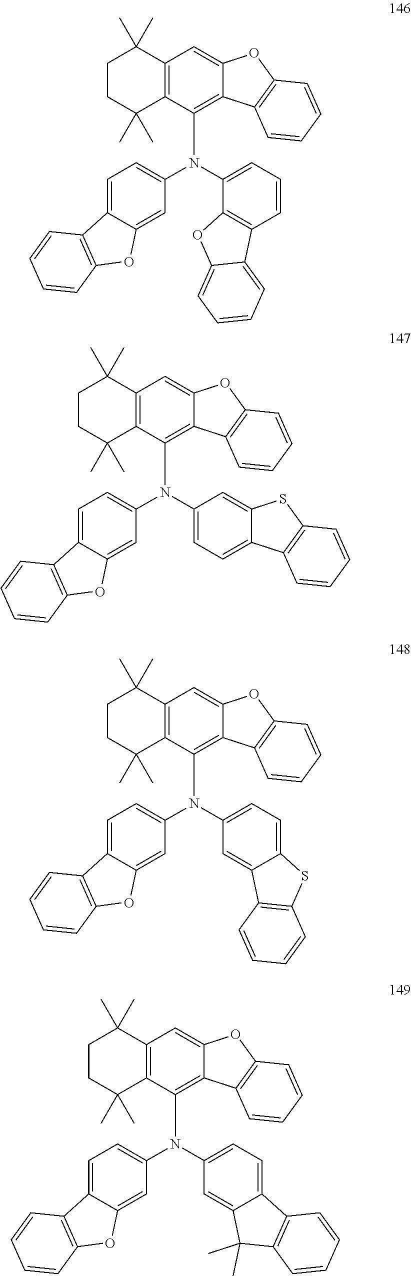 [SMILES: CC1(C)CCC(C)(C)c2c1cc1oc3ccccc3c1c2N(c1ccc2c(c1)oc1ccccc12)c1cccc2c1oc1ccccc12 ; CC1(C)CCC(C)(C)c2c1cc1oc3ccccc3c1c2N(c1ccc2c(c1)oc1ccccc12)c1ccc2c(c1)sc1ccccc12 ; CC1(C)CCC(C)(C)c2c1cc1oc3ccccc3c1c2N(c1ccc2c(c1)oc1ccccc12)c1ccc2sc3ccccc3c2c1 ; CC1(C)CCC(C)(C)c2c1cc1oc3ccccc3c1c2N(c1ccc2c(c1)C(C)(C)c1ccccc1-2)c1ccc2c(c1)oc1ccccc12]

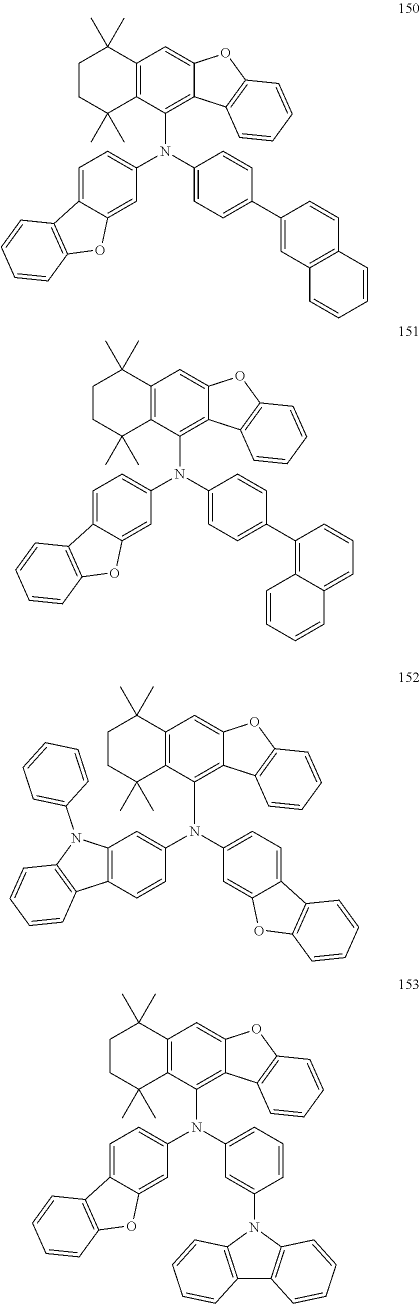 [SMILES: CC1(C)CCC(C)(C)c2c1cc1oc3ccccc3c1c2N(c1ccc(-c2ccc3ccccc3c2)cc1)c1ccc2c(c1)oc1ccccc12 ; CC1(C)CCC(C)(C)c2c1cc1oc3ccccc3c1c2N(c1ccc(-c2cccc3ccccc23)cc1)c1ccc2c(c1)oc1ccccc12 ; CC1(C)CCC(C)(C)c2c1cc1oc3ccccc3c1c2N(c1ccc2c(c1)oc1ccccc12)c1ccc2c3ccccc3n(-c3ccccc3)c2c1 ; CC1(C)CCC(C)(C)c2c1cc1oc3ccccc3c1c2N(c1cccc(-n2c3ccccc3c3ccccc32)c1)c1ccc2c(c1)oc1ccccc12]

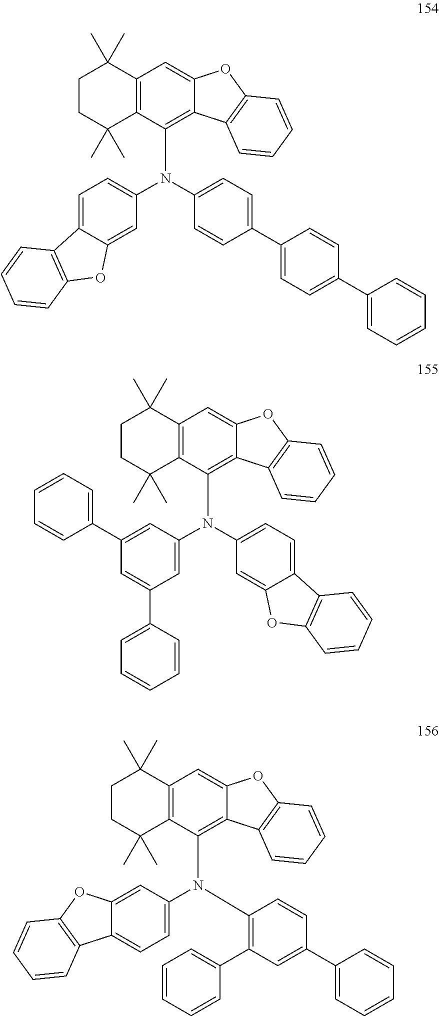 [SMILES: CC1(C)CCC(C)(C)c2c1cc1oc3ccccc3c1c2N(c1ccc(-c2ccc(-c3ccccc3)cc2)cc1)c1ccc2c(c1)oc1ccccc12 ; CC1(C)CCC(C)(C)c2c1cc1oc3ccccc3c1c2N(c1cc(-c2ccccc2)cc(-c2ccccc2)c1)c1ccc2c(c1)oc1ccccc12 ; CC1(C)CCC(C)(C)c2c1cc1oc3ccccc3c1c2N(c1ccc2c(c1)oc1ccccc12)c1ccc(-c2ccccc2)cc1-c1ccccc1]

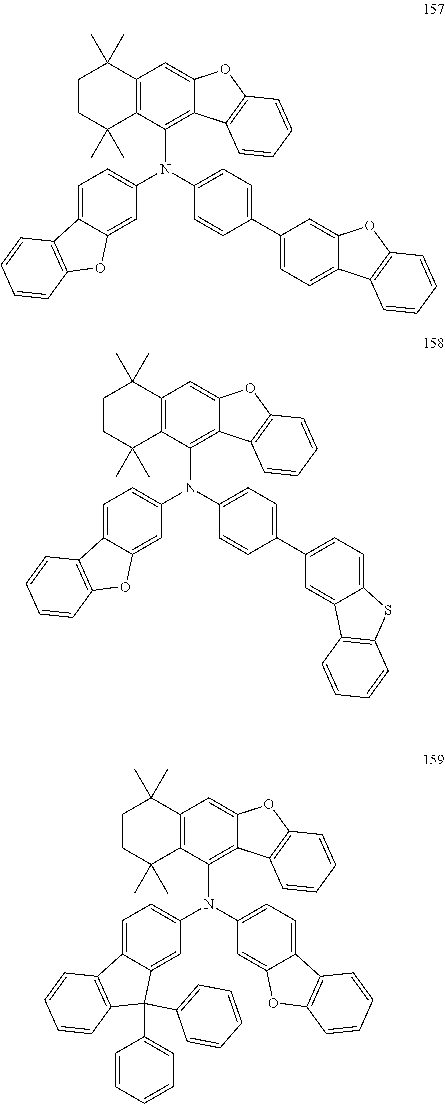 [SMILES: CC1(C)CCC(C)(C)c2c1cc1oc3ccccc3c1c2N(c1ccc(-c2ccc3c(c2)oc2ccccc23)cc1)c1ccc2c(c1)oc1ccccc12 ; CC1(C)CCC(C)(C)c2c1cc1oc3ccccc3c1c2N(c1ccc(-c2ccc3sc4ccccc4c3c2)cc1)c1ccc2c(c1)oc1ccccc12 ; CC1(C)CCC(C)(C)c2c1cc1oc3ccccc3c1c2N(c1ccc2c(c1)C(c1ccccc1)(c1ccccc1)c1ccccc1-2)c1ccc2c(c1)oc1ccccc12]

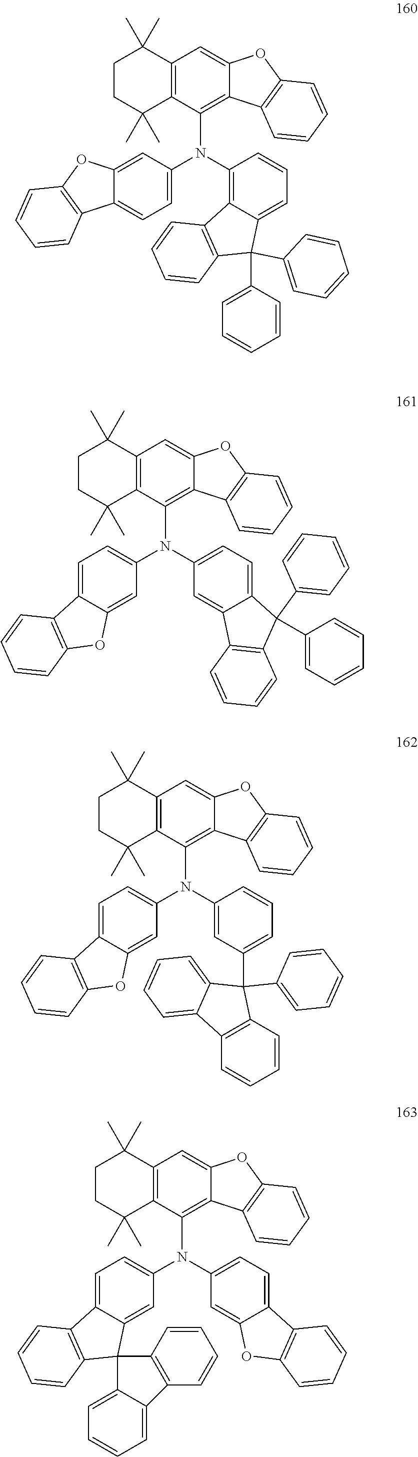 [SMILES: CC1(C)CCC(C)(C)c2c1cc1oc3ccccc3c1c2N(c1ccc2c(c1)oc1ccccc12)c1cccc2c1-c1ccccc1C2(c1ccccc1)c1ccccc1 ; CC1(C)CCC(C)(C)c2c1cc1oc3ccccc3c1c2N(c1ccc2c(c1)-c1ccccc1C2(c1ccccc1)c1ccccc1)c1ccc2c(c1)oc1ccccc12 ; CC1(C)CCC(C)(C)c2c1cc1oc3ccccc3c1c2N(c1cccc(C2(c3ccccc3)c3ccccc3-c3ccccc32)c1)c1ccc2c(c1)oc1ccccc12 ; CC1(C)CCC(C)(C)c2c1cc1oc3ccccc3c1c2N(c1ccc2c(c1)C1(c3ccccc3-c3ccccc31)c1ccccc1-2)c1ccc2c(c1)oc1ccccc12]

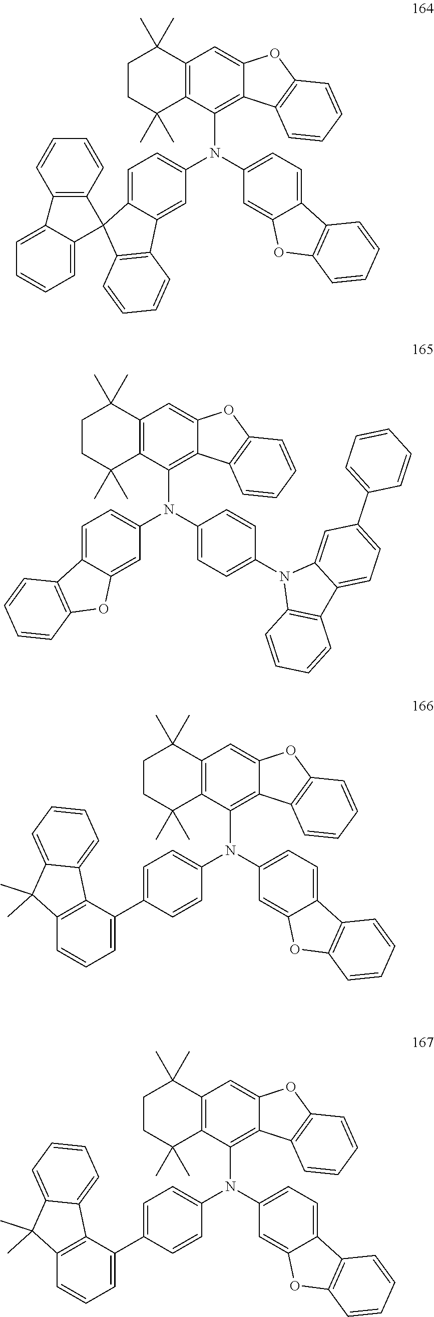 [SMILES: CC1(C)CCC(C)(C)c2c1cc1oc3ccccc3c1c2N(c1ccc2c(c1)-c1ccccc1C21c2ccccc2-c2ccccc21)c1ccc2c(c1)oc1ccccc12 ; CC1(C)CCC(C)(C)c2c1cc1oc3ccccc3c1c2N(c1ccc(-n2c3ccccc3c3ccc(-c4ccccc4)cc32)cc1)c1ccc2c(c1)oc1ccccc12 ; CC1(C)CCC(C)(C)c2c1cc1oc3ccccc3c1c2N(c1ccc(-c2cccc3c2-c2ccccc2C3(C)C)cc1)c1ccc2c(c1)oc1ccccc12 ; CC1(C)CCC(C)(C)c2c1cc1oc3ccccc3c1c2N(c1ccc(-c2cccc3c2-c2ccccc2C3(C)C)cc1)c1ccc2c(c1)oc1ccccc12]

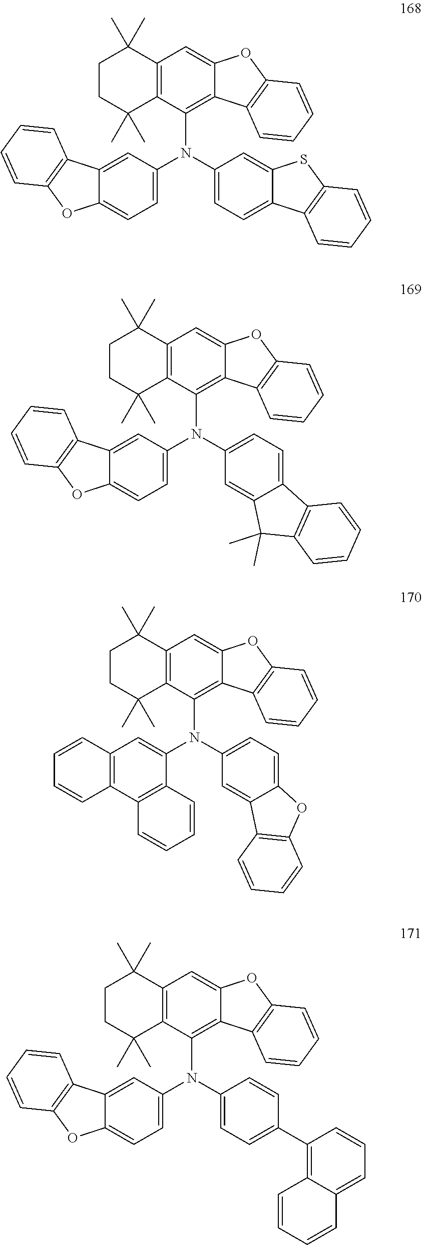 [SMILES: CC1(C)CCC(C)(C)c2c1cc1oc3ccccc3c1c2N(c1ccc2c(c1)sc1ccccc12)c1ccc2oc3ccccc3c2c1 ; CC1(C)CCC(C)(C)c2c1cc1oc3ccccc3c1c2N(c1ccc2c(c1)C(C)(C)c1ccccc1-2)c1ccc2oc3ccccc3c2c1 ; CC1(C)CCC(C)(C)c2c1cc1oc3ccccc3c1c2N(c1ccc2oc3ccccc3c2c1)c1cc2ccccc2c2ccccc12 ; CC1(C)CCC(C)(C)c2c1cc1oc3ccccc3c1c2N(c1ccc(-c2cccc3ccccc23)cc1)c1ccc2oc3ccccc3c2c1]

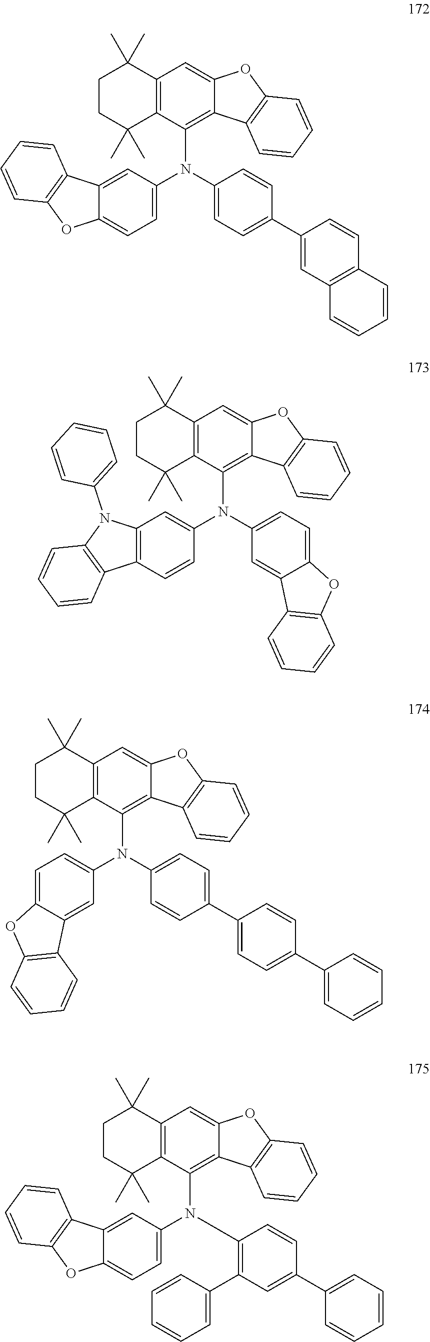 [SMILES: CC1(C)CCC(C)(C)c2c1cc1oc3ccccc3c1c2N(c1ccc(-c2ccc3ccccc3c2)cc1)c1ccc2oc3ccccc3c2c1 ; CC1(C)CCC(C)(C)c2c1cc1oc3ccccc3c1c2N(c1ccc2oc3ccccc3c2c1)c1ccc2c3ccccc3n(-c3ccccc3)c2c1 ; CC1(C)CCC(C)(C)c2c1cc1oc3ccccc3c1c2N(c1ccc(-c2ccc(-c3ccccc3)cc2)cc1)c1ccc2oc3ccccc3c2c1 ; CC1(C)CCC(C)(C)c2c1cc1oc3ccccc3c1c2N(c1ccc2oc3ccccc3c2c1)c1ccc(-c2ccccc2)cc1-c1ccccc1]

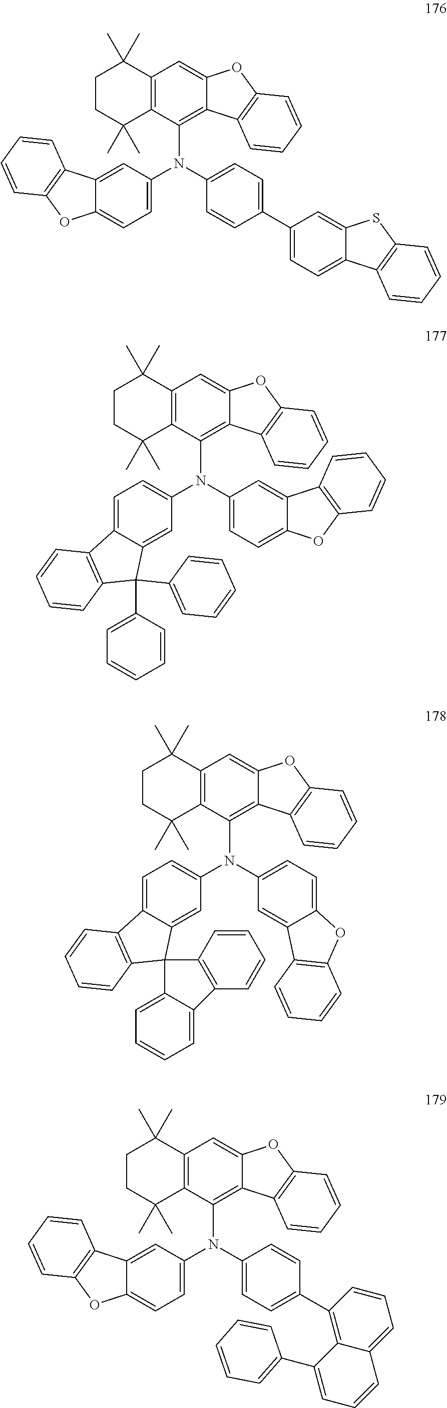 [SMILES: CC1(C)CCC(C)(C)c2c1cc1oc3ccccc3c1c2N(c1ccc(-c2ccc3c(c2)sc2ccccc23)cc1)c1ccc2oc3ccccc3c2c1 ; CC1(C)CCC(C)(C)c2c1cc1oc3ccccc3c1c2N(c1ccc2c(c1)C(c1ccccc1)(c1ccccc1)c1ccccc1-2)c1ccc2oc3ccccc3c2c1 ; CC1(C)CCC(C)(C)c2c1cc1oc3ccccc3c1c2N(c1ccc2c(c1)C1(c3ccccc3-c3ccccc31)c1ccccc1-2)c1ccc2oc3ccccc3c2c1 ; CC1(C)CCC(C)(C)c2c1cc1oc3ccccc3c1c2N(c1ccc(-c2cccc3cccc(-c4ccccc4)c23)cc1)c1ccc2oc3ccccc3c2c1]

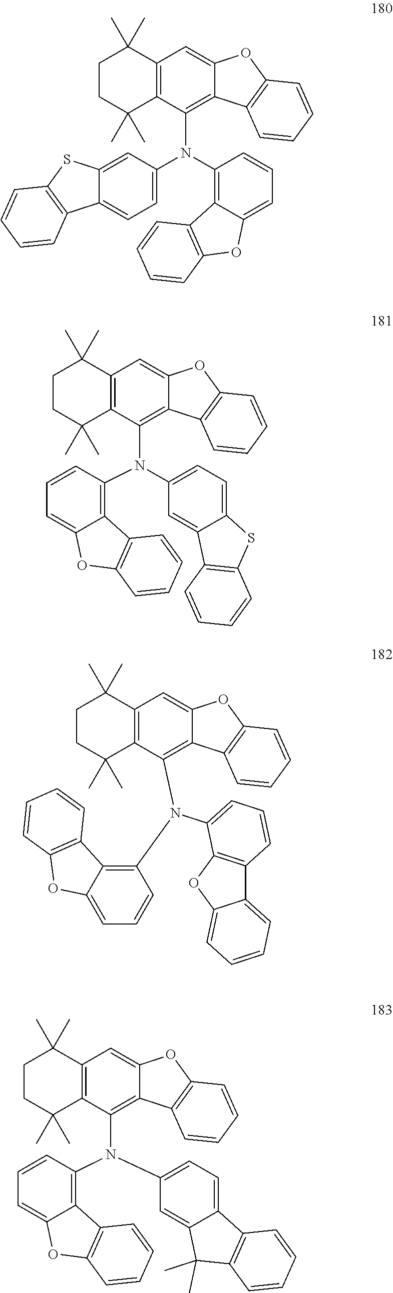 [SMILES: CC1(C)CCC(C)(C)c2c1cc1oc3ccccc3c1c2N(c1ccc2c(c1)sc1ccccc12)c1cccc2oc3ccccc3c12 ; CC1(C)CCC(C)(C)c2c1cc1oc3ccccc3c1c2N(c1ccc2sc3ccccc3c2c1)c1cccc2oc3ccccc3c12 ; CC1(C)CCC(C)(C)c2c1cc1oc3ccccc3c1c2N(c1cccc2c1oc1ccccc12)c1cccc2oc3ccccc3c12 ; CC1(C)CCC(C)(C)c2c1cc1oc3ccccc3c1c2N(c1ccc2c(c1)C(C)(C)c1ccccc1-2)c1cccc2oc3ccccc3c12]

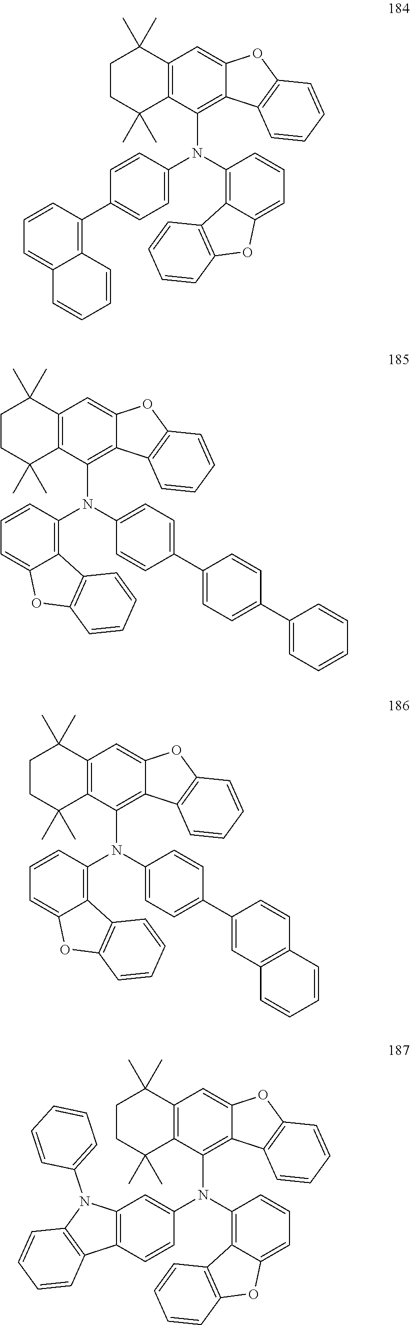 [SMILES: CC1(C)CCC(C)(C)c2c1cc1oc3ccccc3c1c2N(c1ccc(-c2cccc3ccccc23)cc1)c1cccc2oc3ccccc3c12 ; CC1(C)CCC(C)(C)c2c1cc1oc3ccccc3c1c2N(c1ccc(-c2ccc(-c3ccccc3)cc2)cc1)c1cccc2oc3ccccc3c12 ; CC1(C)CCC(C)(C)c2c1cc1oc3ccccc3c1c2N(c1ccc(-c2ccc3ccccc3c2)cc1)c1cccc2oc3ccccc3c12 ; CC1(C)CCC(C)(C)c2c1cc1oc3ccccc3c1c2N(c1ccc2c3ccccc3n(-c3ccccc3)c2c1)c1cccc2oc3ccccc3c12]

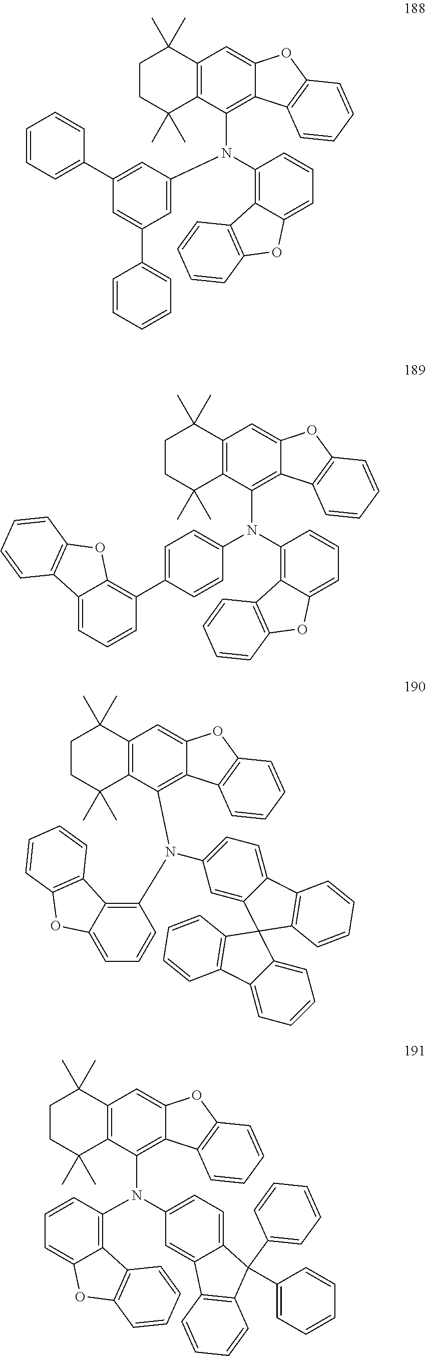 [SMILES: CC1(C)CCC(C)(C)c2c1cc1oc3ccccc3c1c2N(c1cc(-c2ccccc2)cc(-c2ccccc2)c1)c1cccc2oc3ccccc3c12 ; CC1(C)CCC(C)(C)c2c1cc1oc3ccccc3c1c2N(c1ccc(-c2cccc3c2oc2ccccc23)cc1)c1cccc2oc3ccccc3c12 ; CC1(C)CCC(C)(C)c2c1cc1oc3ccccc3c1c2N(c1ccc2c(c1)C1(c3ccccc3-c3ccccc31)c1ccccc1-2)c1cccc2oc3ccccc3c12 ; CC1(C)CCC(C)(C)c2c1cc1oc3ccccc3c1c2N(c1ccc2c(c1)-c1ccccc1C2(c1ccccc1)c1ccccc1)c1cccc2oc3ccccc3c12]

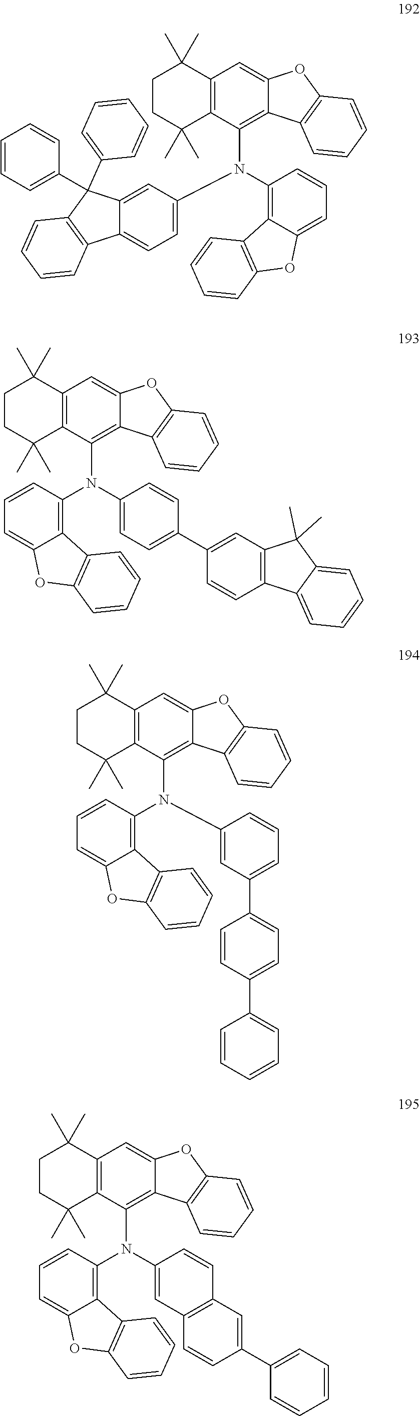 [SMILES: CC1(C)CCC(C)(C)c2c1cc1oc3ccccc3c1c2N(c1ccc2c(c1)C(c1ccccc1)(c1ccccc1)c1ccccc1-2)c1cccc2oc3ccccc3c12 ; CC1(C)CCC(C)(C)c2c1cc1oc3ccccc3c1c2N(c1ccc(-c2ccc3c(c2)C(C)(C)c2ccccc2-3)cc1)c1cccc2oc3ccccc3c12 ; CC1(C)CCC(C)(C)c2c1cc1oc3ccccc3c1c2N(c1cccc(-c2ccc(-c3ccccc3)cc2)c1)c1cccc2oc3ccccc3c12 ; CC1(C)CCC(C)(C)c2c1cc1oc3ccccc3c1c2N(c1ccc2cc(-c3ccccc3)ccc2c1)c1cccc2oc3ccccc3c12]

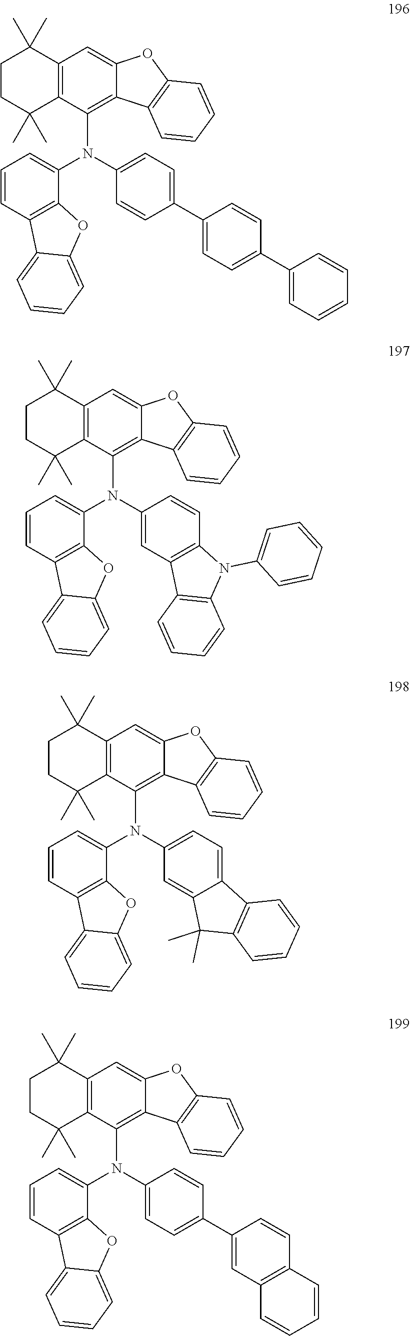

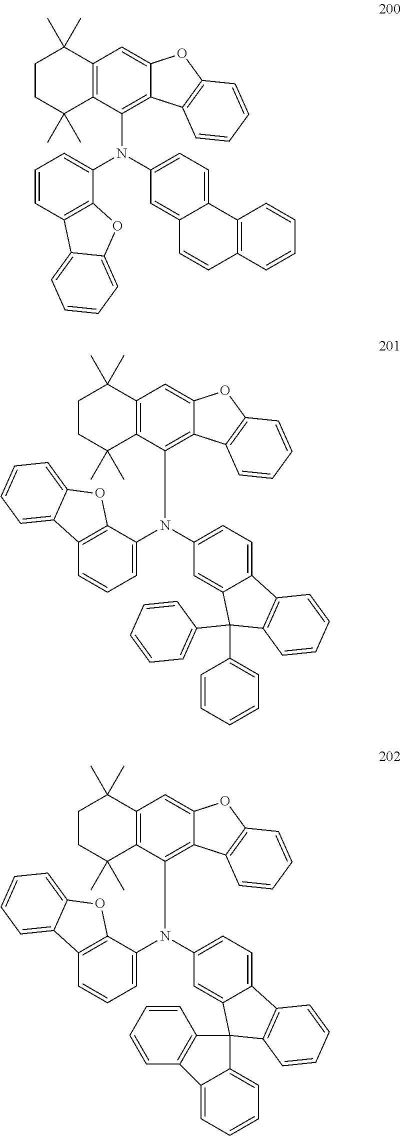

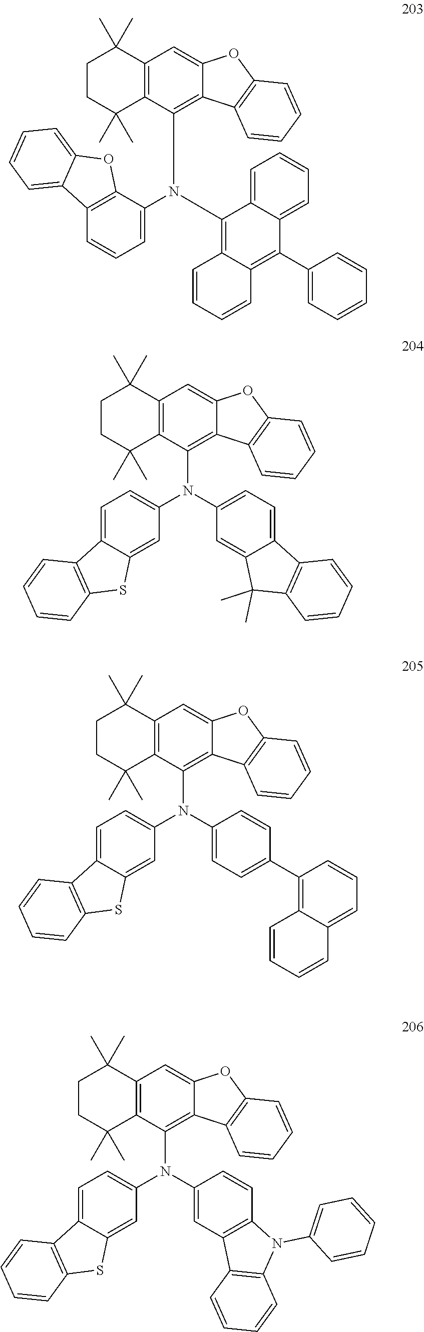 [SMILES: CC1(C)CCC(C)(C)c2c1cc1oc3ccccc3c1c2N(c1c2ccccc2c(-c2ccccc2)c2ccccc12)c1cccc2c1oc1ccccc12 ; CC1(C)CCC(C)(C)c2c1cc1oc3ccccc3c1c2N(c1ccc2c(c1)C(C)(C)c1ccccc1-2)c1ccc2c(c1)sc1ccccc12 ; CC1(C)CCC(C)(C)c2c1cc1oc3ccccc3c1c2N(c1ccc(-c2cccc3ccccc23)cc1)c1ccc2c(c1)sc1ccccc12 ; CC1(C)CCC(C)(C)c2c1cc1oc3ccccc3c1c2N(c1ccc2c(c1)sc1ccccc12)c1ccc2c(c1)c1ccccc1n2-c1ccccc1]

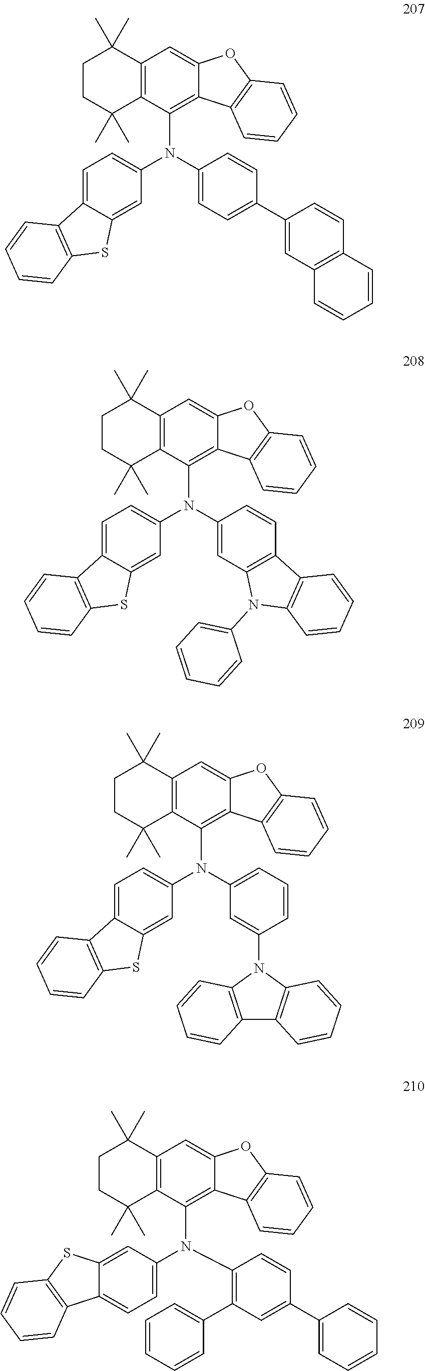 [SMILES: CC1(C)CCC(C)(C)c2c1cc1oc3ccccc3c1c2N(c1ccc(-c2ccc3ccccc3c2)cc1)c1ccc2c(c1)sc1ccccc12 ; CC1(C)CCC(C)(C)c2c1cc1oc3ccccc3c1c2N(c1ccc2c(c1)sc1ccccc12)c1ccc2c3ccccc3n(-c3ccccc3)c2c1 ; CC1(C)CCC(C)(C)c2c1cc1oc3ccccc3c1c2N(c1cccc(-n2c3ccccc3c3ccccc32)c1)c1ccc2c(c1)sc1ccccc12 ; CC1(C)CCC(C)(C)c2c1cc1oc3ccccc3c1c2N(c1ccc2c(c1)sc1ccccc12)c1ccc(-c2ccccc2)cc1-c1ccccc1]

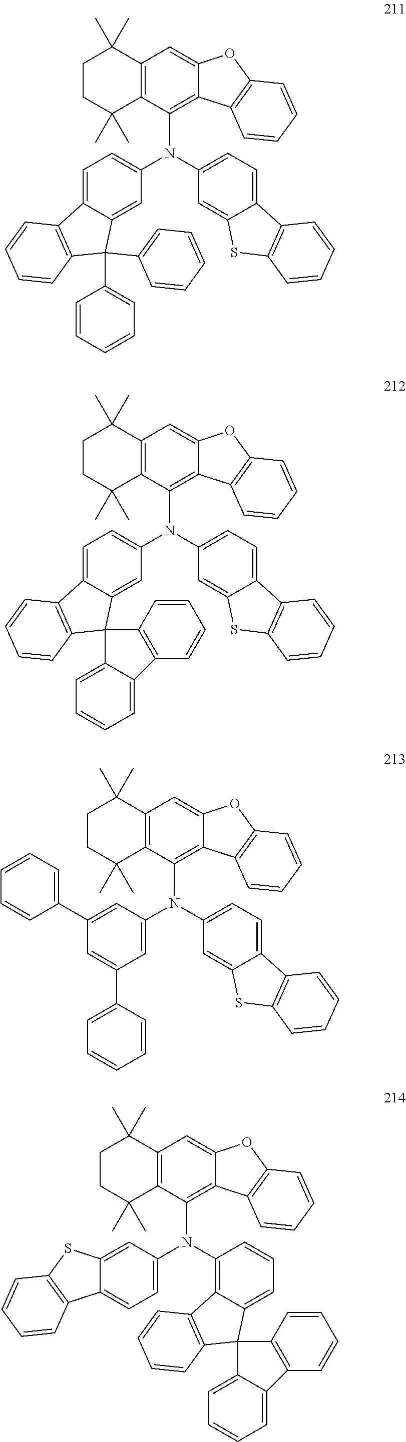

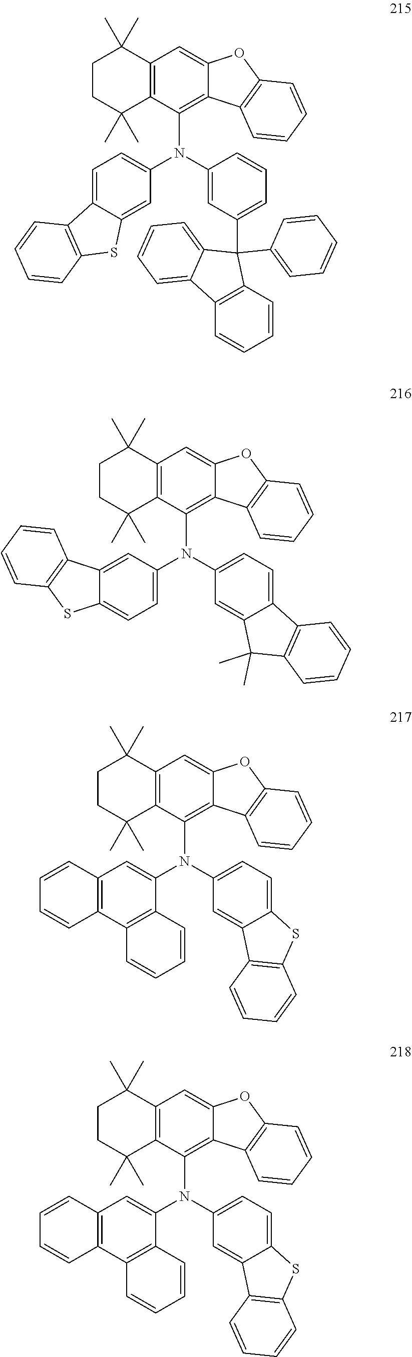 [SMILES: CC1(C)CCC(C)(C)c2c1cc1oc3ccccc3c1c2N(c1cccc(C2(c3ccccc3)c3ccccc3-c3ccccc32)c1)c1ccc2c(c1)sc1ccccc12 ; CC1(C)CCC(C)(C)c2c1cc1oc3ccccc3c1c2N(c1ccc2c(c1)C(C)(C)c1ccccc1-2)c1ccc2sc3ccccc3c2c1 ; CC1(C)CCC(C)(C)c2c1cc1oc3ccccc3c1c2N(c1ccc2sc3ccccc3c2c1)c1cc2ccccc2c2ccccc12 ; CC1(C)CCC(C)(C)c2c1cc1oc3ccccc3c1c2N(c1ccc2sc3ccccc3c2c1)c1cc2ccccc2c2ccccc12]

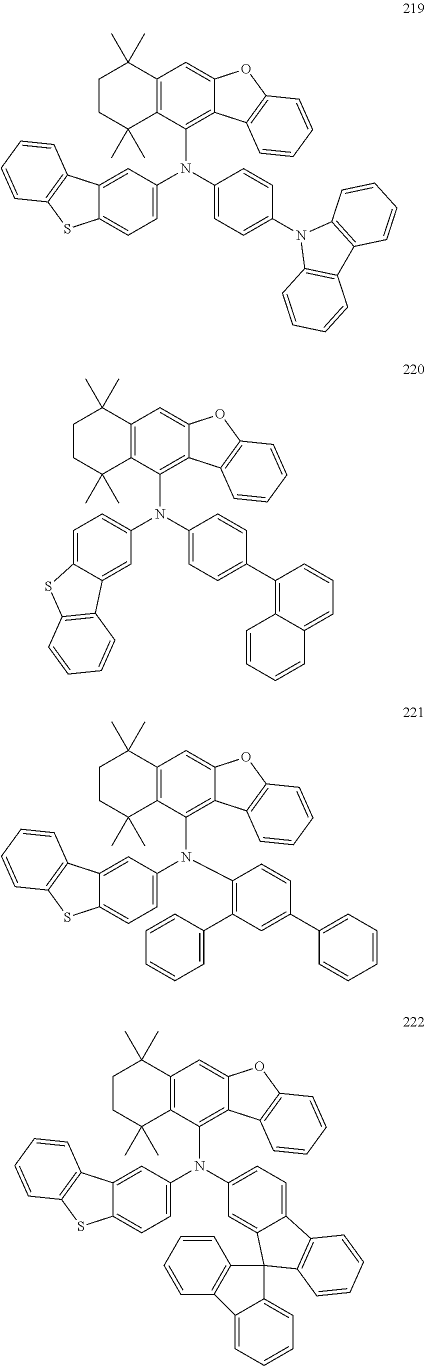 [SMILES: CC1(C)CCC(C)(C)c2c1cc1oc3ccccc3c1c2N(c1ccc(-n2c3ccccc3c3ccccc32)cc1)c1ccc2sc3ccccc3c2c1 ; CC1(C)CCC(C)(C)c2c1cc1oc3ccccc3c1c2N(c1ccc(-c2cccc3ccccc23)cc1)c1ccc2sc3ccccc3c2c1 ; CC1(C)CCC(C)(C)c2c1cc1oc3ccccc3c1c2N(c1ccc2sc3ccccc3c2c1)c1ccc(-c2ccccc2)cc1-c1ccccc1 ; CC1(C)CCC(C)(C)c2c1cc1oc3ccccc3c1c2N(c1ccc2c(c1)C1(c3ccccc3-c3ccccc31)c1ccccc1-2)c1ccc2sc3ccccc3c2c1]

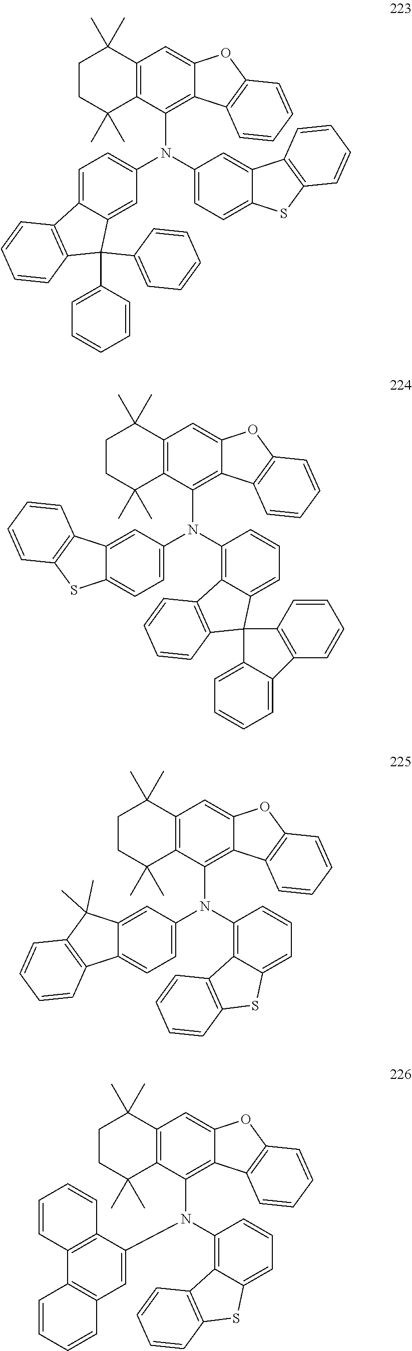

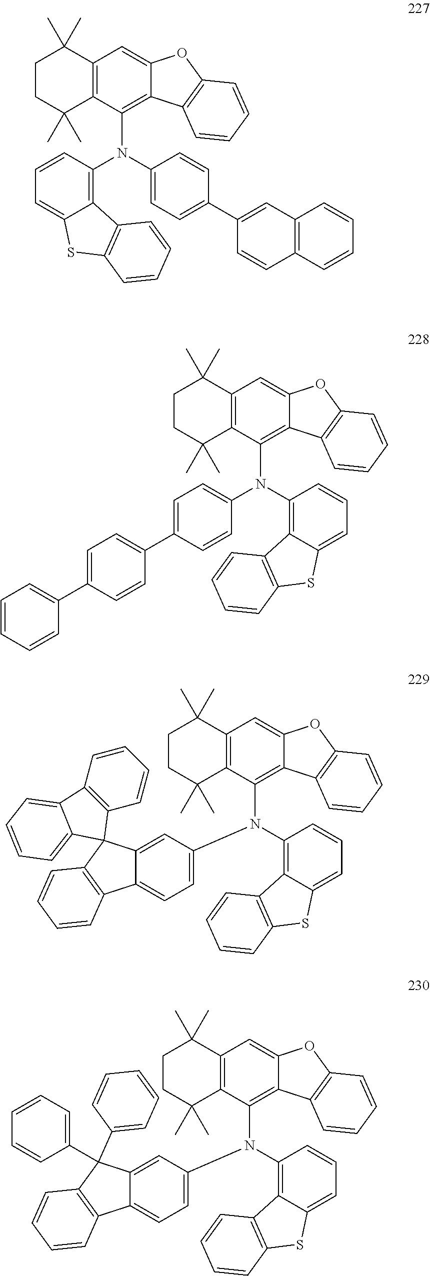 [SMILES: CC1(C)CCC(C)(C)c2c1cc1oc3ccccc3c1c2N(c1ccc(-c2ccc3ccccc3c2)cc1)c1cccc2sc3ccccc3c12 ; CC1(C)CCC(C)(C)c2c1cc1oc3ccccc3c1c2N(c1ccc(-c2ccc(-c3ccccc3)cc2)cc1)c1cccc2sc3ccccc3c12 ; CC1(C)CCC(C)(C)c2c1cc1oc3ccccc3c1c2N(c1ccc2c(c1)C1(c3ccccc3-c3ccccc31)c1ccccc1-2)c1cccc2sc3ccccc3c12 ; CC1(C)CCC(C)(C)c2c1cc1oc3ccccc3c1c2N(c1ccc2c(c1)C(c1ccccc1)(c1ccccc1)c1ccccc1-2)c1cccc2sc3ccccc3c12]

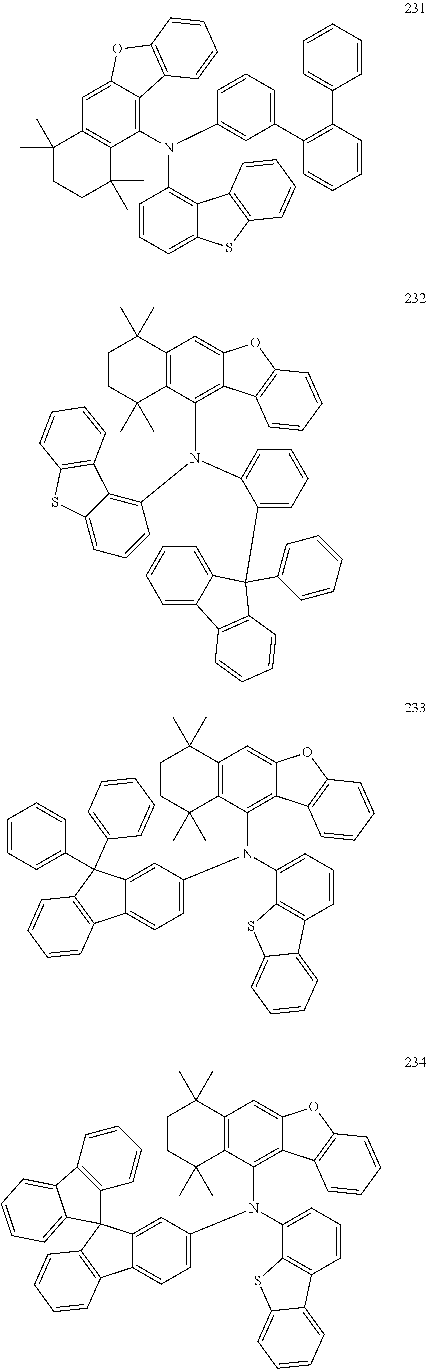 [SMILES: CC1(C)CCC(C)(C)c2c1cc1oc3ccccc3c1c2N(c1cccc(-c2ccccc2-c2ccccc2)c1)c1cccc2sc3ccccc3c12 ; CC1(C)CCC(C)(C)c2c1cc1oc3ccccc3c1c2N(c1ccccc1C1(c2ccccc2)c2ccccc2-c2ccccc21)c1cccc2sc3ccccc3c12 ; CC1(C)CCC(C)(C)c2c1cc1oc3ccccc3c1c2N(c1ccc2c(c1)C(c1ccccc1)(c1ccccc1)c1ccccc1-2)c1cccc2c1sc1ccccc12 ; CC1(C)CCC(C)(C)c2c1cc1oc3ccccc3c1c2N(c1ccc2c(c1)C1(c3ccccc3-c3ccccc31)c1ccccc1-2)c1cccc2c1sc1ccccc12]

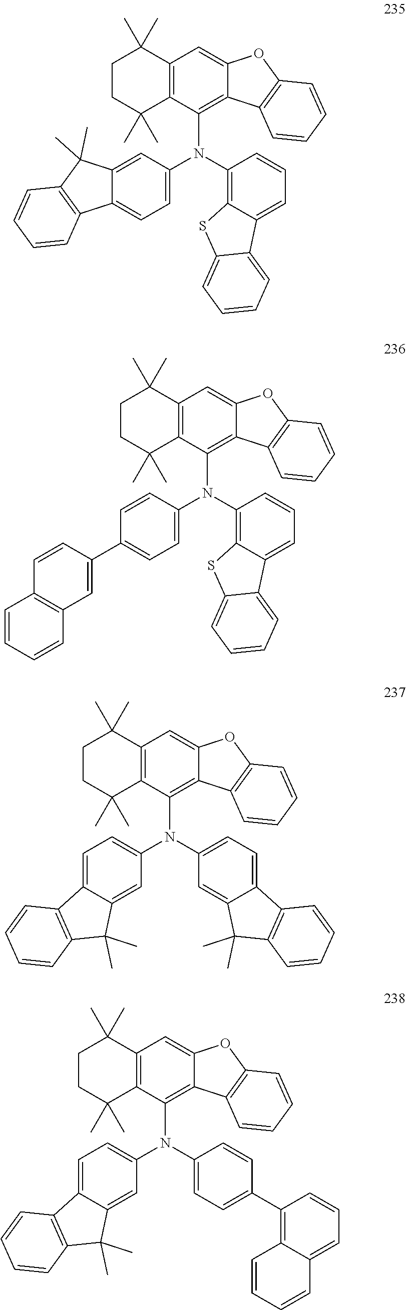

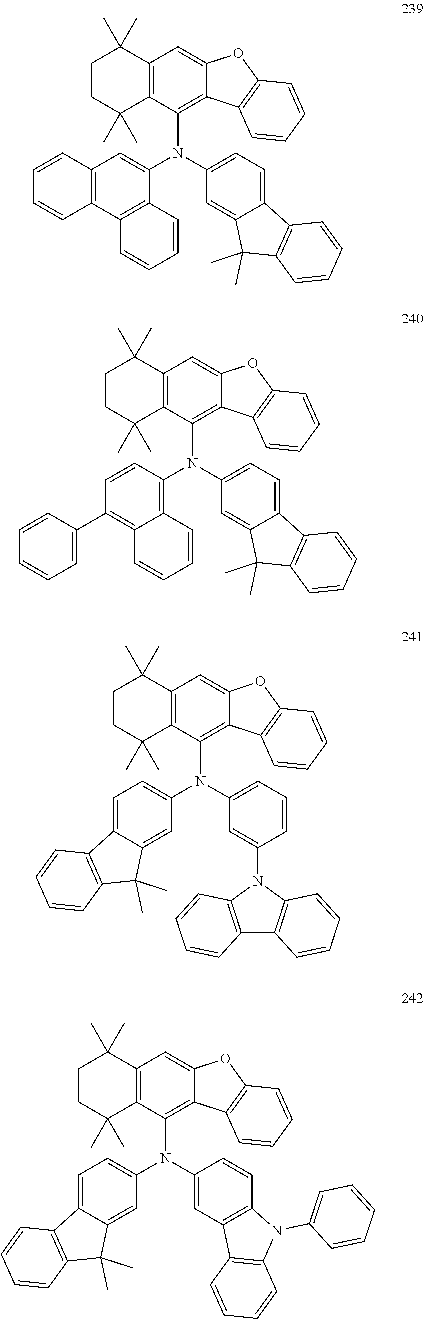 [SMILES: CC1(C)CCC(C)(C)c2c1cc1oc3ccccc3c1c2N(c1ccc2c(c1)C(C)(C)c1ccccc1-2)c1cc2ccccc2c2ccccc12 ; CC1(C)CCC(C)(C)c2c1cc1oc3ccccc3c1c2N(c1ccc2c(c1)C(C)(C)c1ccccc1-2)c1ccc(-c2ccccc2)c2ccccc12 ; CC1(C)CCC(C)(C)c2c1cc1oc3ccccc3c1c2N(c1cccc(-n2c3ccccc3c3ccccc32)c1)c1ccc2c(c1)C(C)(C)c1ccccc1-2 ; CC1(C)CCC(C)(C)c2c1cc1oc3ccccc3c1c2N(c1ccc2c(c1)C(C)(C)c1ccccc1-2)c1ccc2c(c1)c1ccccc1n2-c1ccccc1]

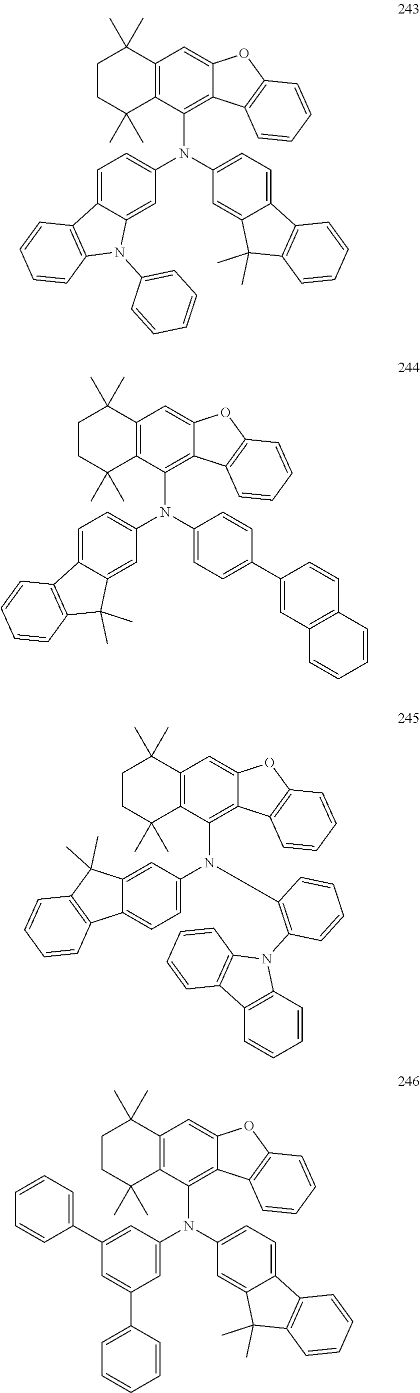 [SMILES: CC1(C)CCC(C)(C)c2c1cc1oc3ccccc3c1c2N(c1ccc2c(c1)C(C)(C)c1ccccc1-2)c1ccc2c3ccccc3n(-c3ccccc3)c2c1 ; CC1(C)CCC(C)(C)c2c1cc1oc3ccccc3c1c2N(c1ccc(-c2ccc3ccccc3c2)cc1)c1ccc2c(c1)C(C)(C)c1ccccc1-2 ; CC1(C)CCC(C)(C)c2c1cc1oc3ccccc3c1c2N(c1ccc2c(c1)C(C)(C)c1ccccc1-2)c1ccccc1-n1c2ccccc2c2ccccc21 ; CC1(C)CCC(C)(C)c2c1cc1oc3ccccc3c1c2N(c1cc(-c2ccccc2)cc(-c2ccccc2)c1)c1ccc2c(c1)C(C)(C)c1ccccc1-2]

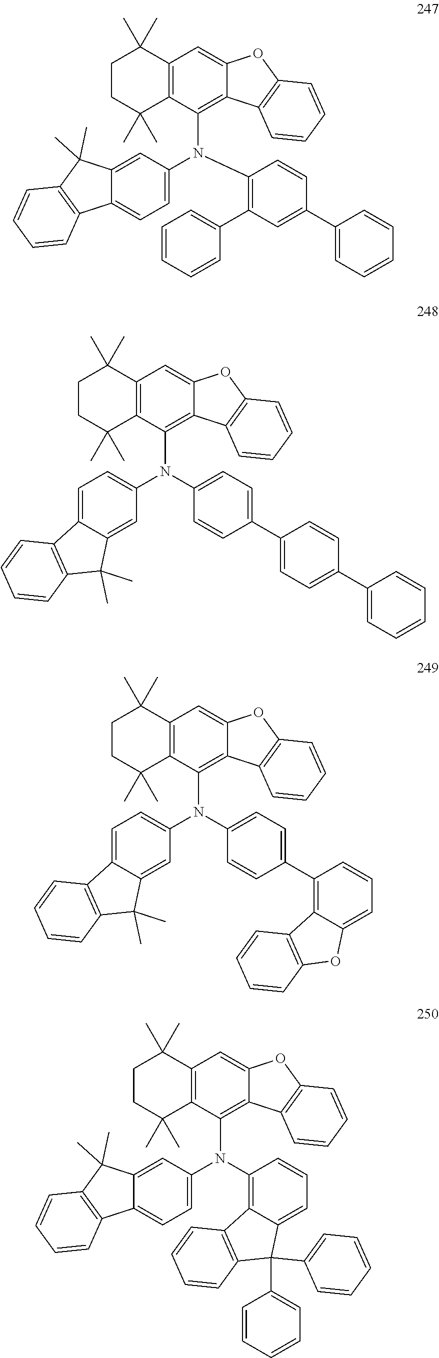 [SMILES: CC1(C)CCC(C)(C)c2c1cc1oc3ccccc3c1c2N(c1ccc2c(c1)C(C)(C)c1ccccc1-2)c1ccc(-c2ccccc2)cc1-c1ccccc1 ; CC1(C)CCC(C)(C)c2c1cc1oc3ccccc3c1c2N(c1ccc(-c2ccc(-c3ccccc3)cc2)cc1)c1ccc2c(c1)C(C)(C)c1ccccc1-2 ; CC1(C)CCC(C)(C)c2c1cc1oc3ccccc3c1c2N(c1ccc(-c2cccc3oc4ccccc4c23)cc1)c1ccc2c(c1)C(C)(C)c1ccccc1-2 ; CC1(C)CCC(C)(C)c2c1cc1oc3ccccc3c1c2N(c1ccc2c(c1)C(C)(C)c1ccccc1-2)c1cccc2c1-c1ccccc1C2(c1ccccc1)c1ccccc1]

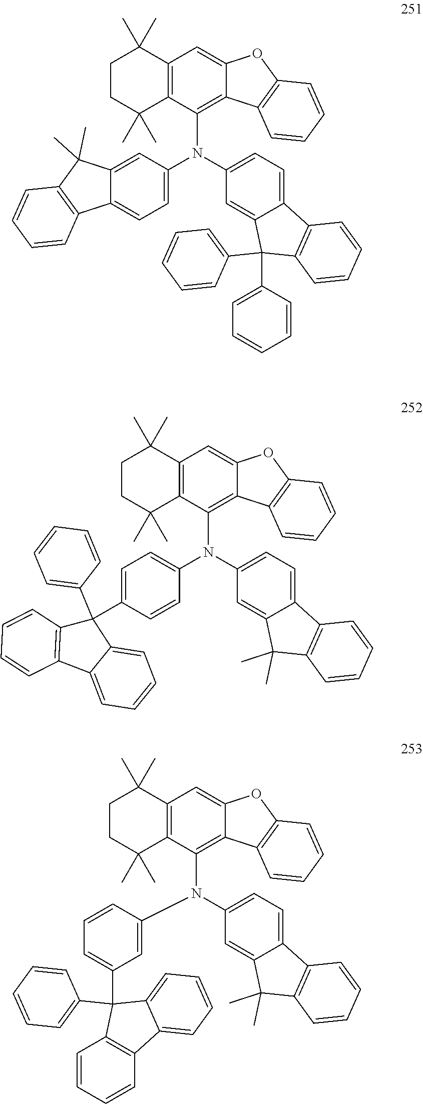 [SMILES: CC1(C)CCC(C)(C)c2c1cc1oc3ccccc3c1c2N(c1ccc2c(c1)C(C)(C)c1ccccc1-2)c1ccc2c(c1)C(c1ccccc1)(c1ccccc1)c1ccccc1-2 ; CC1(C)CCC(C)(C)c2c1cc1oc3ccccc3c1c2N(c1ccc(C2(c3ccccc3)c3ccccc3-c3ccccc32)cc1)c1ccc2c(c1)C(C)(C)c1ccccc1-2 ; CC1(C)CCC(C)(C)c2c1cc1oc3ccccc3c1c2N(c1cccc(C2(c3ccccc3)c3ccccc3-c3ccccc32)c1)c1ccc2c(c1)C(C)(C)c1ccccc1-2]

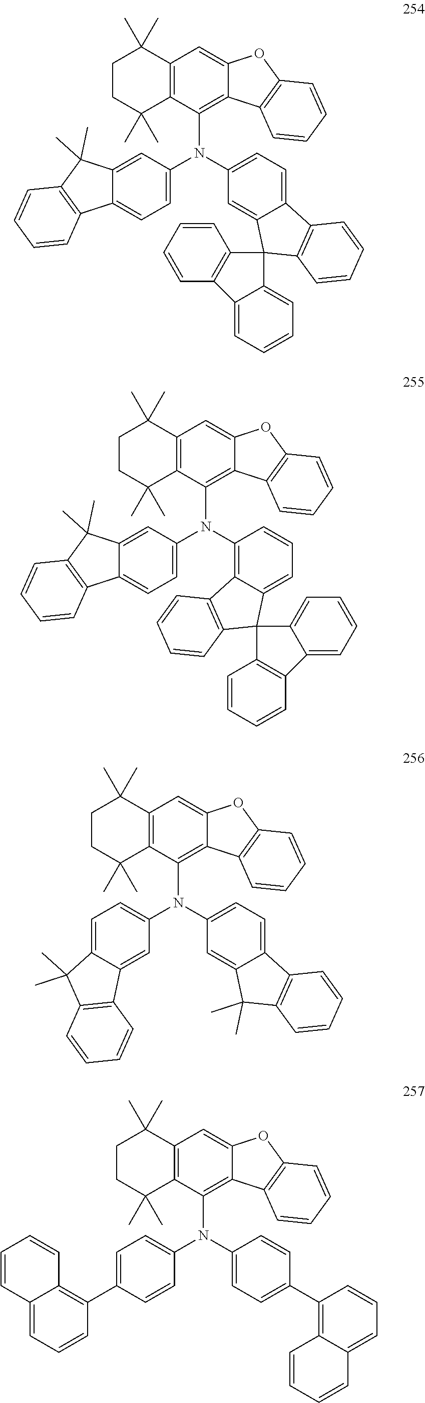

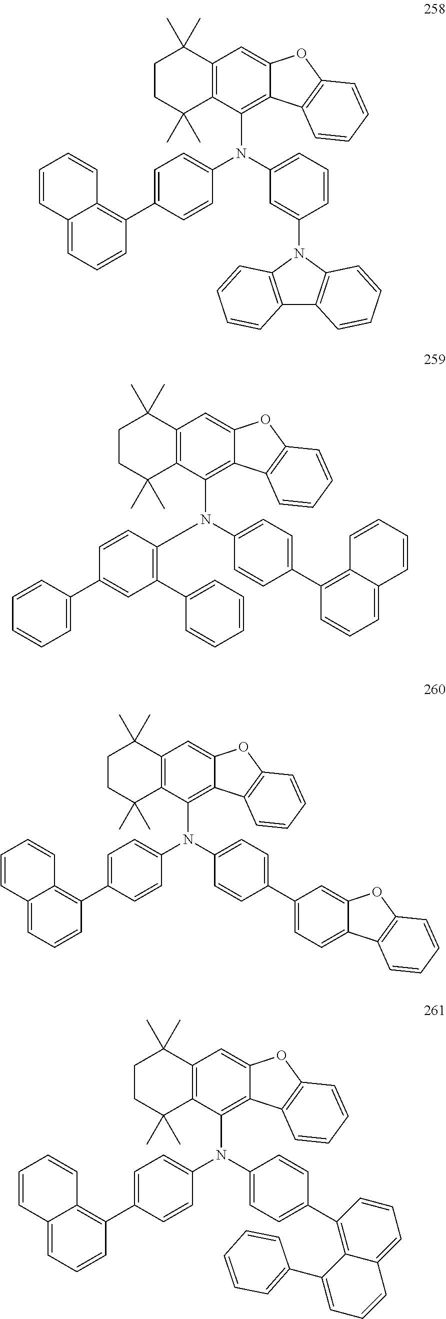 [SMILES: CC1(C)CCC(C)(C)c2c1cc1oc3ccccc3c1c2N(c1ccc(-c2cccc3ccccc23)cc1)c1cccc(-n2c3ccccc3c3ccccc32)c1 ; CC1(C)CCC(C)(C)c2c1cc1oc3ccccc3c1c2N(c1ccc(-c2cccc3ccccc23)cc1)c1ccc(-c2ccccc2)cc1-c1ccccc1 ; CC1(C)CCC(C)(C)c2c1cc1oc3ccccc3c1c2N(c1ccc(-c2ccc3c(c2)oc2ccccc23)cc1)c1ccc(-c2cccc3ccccc23)cc1 ; CC1(C)CCC(C)(C)c2c1cc1oc3ccccc3c1c2N(c1ccc(-c2cccc3ccccc23)cc1)c1ccc(-c2cccc3cccc(-c4ccccc4)c23)cc1]

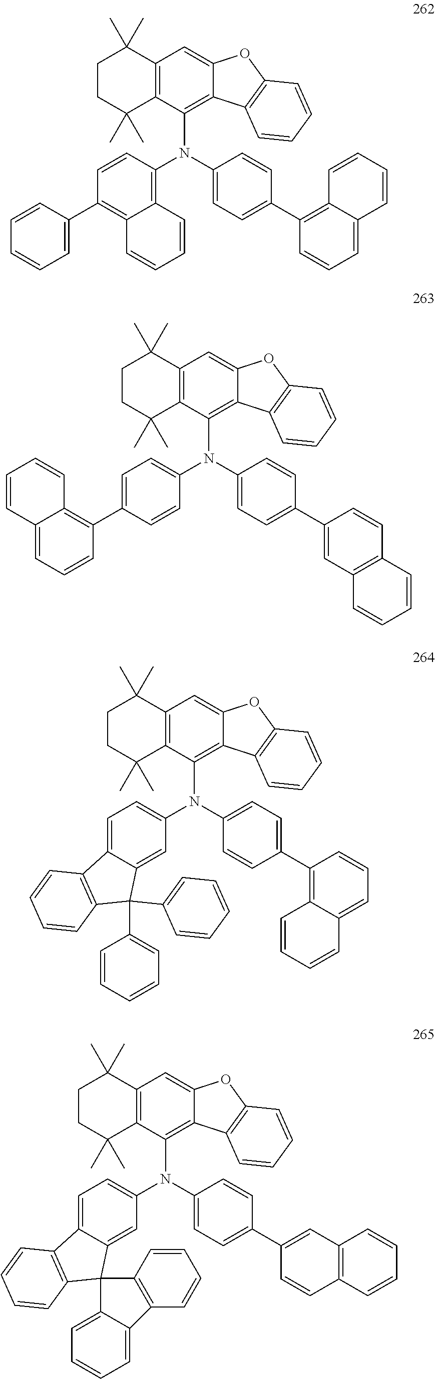

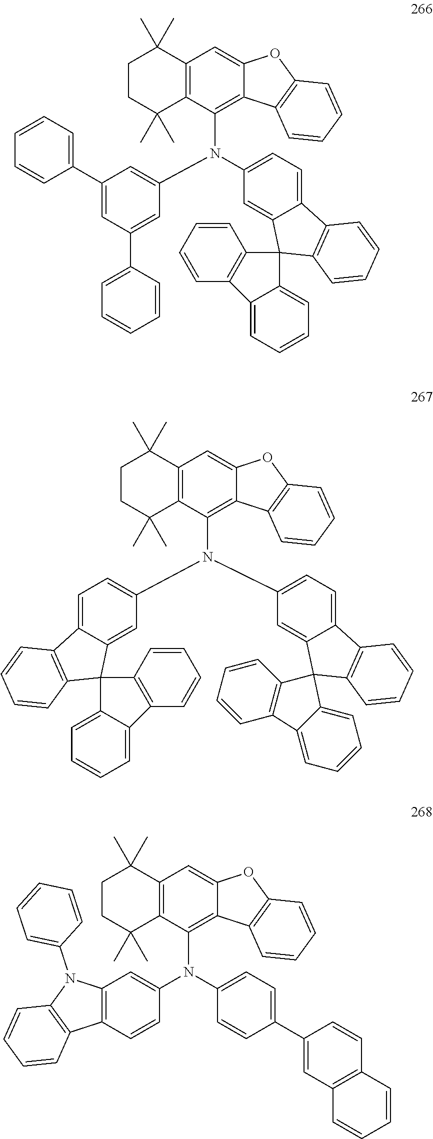 [SMILES: CC1(C)CCC(C)(C)c2c1cc1oc3ccccc3c1c2N(c1cc(-c2ccccc2)cc(-c2ccccc2)c1)c1ccc2c(c1)C1(c3ccccc3-c3ccccc31)c1ccccc1-2 ; CC1(C)CCC(C)(C)c2c1cc1oc3ccccc3c1c2N(c1ccc2c(c1)C1(c3ccccc3-c3ccccc31)c1ccccc1-2)c1ccc2c(c1)C1(c3ccccc3-c3ccccc31)c1ccccc1-2 ; CC1(C)CCC(C)(C)c2c1cc1oc3ccccc3c1c2N(c1ccc(-c2ccc3ccccc3c2)cc1)c1ccc2c3ccccc3n(-c3ccccc3)c2c1]

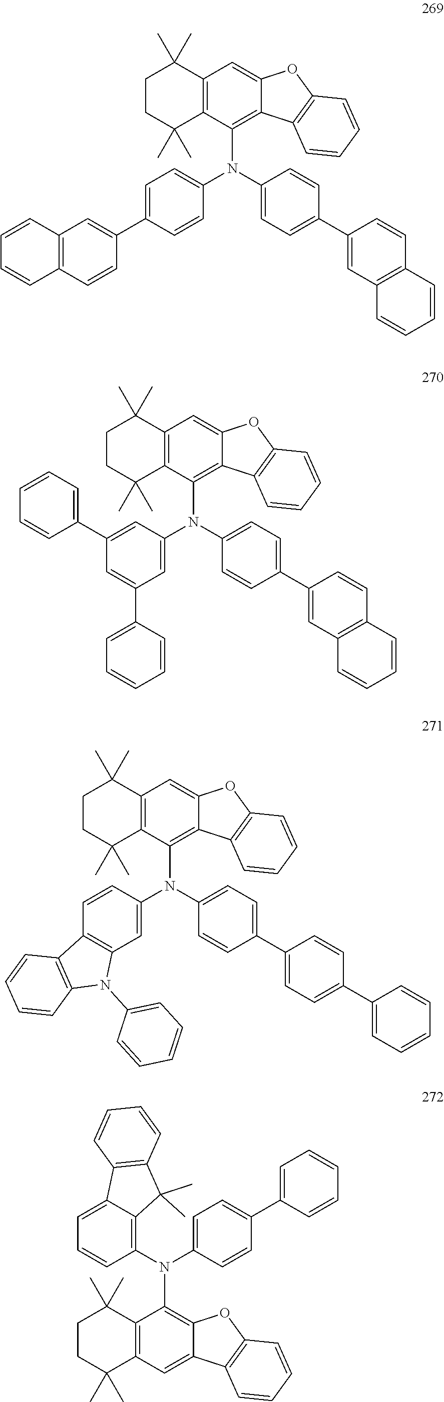

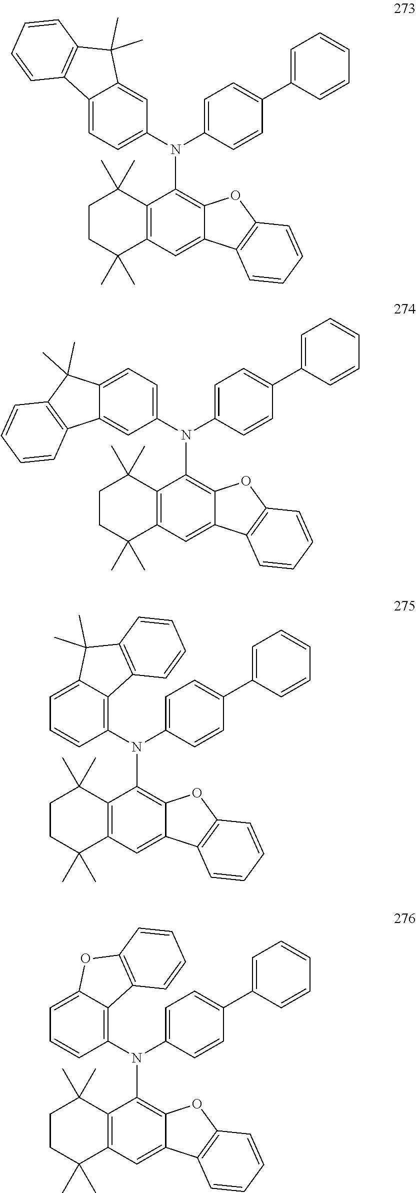 [SMILES: CC1(C)CCC(C)(C)c2c1cc1c(oc3ccccc31)c2N(c1ccc(-c2ccccc2)cc1)c1ccc2c(c1)C(C)(C)c1ccccc1-2 ; CC1(C)CCC(C)(C)c2c1cc1c(oc3ccccc31)c2N(c1ccc(-c2ccccc2)cc1)c1ccc2c(c1)-c1ccccc1C2(C)C ; CC1(C)CCC(C)(C)c2c1cc1c(oc3ccccc31)c2N(c1ccc(-c2ccccc2)cc1)c1cccc2c1-c1ccccc1C2(C)C ; CC1(C)CCC(C)(C)c2c1cc1c(oc3ccccc31)c2N(c1ccc(-c2ccccc2)cc1)c1cccc2oc3ccccc3c12]

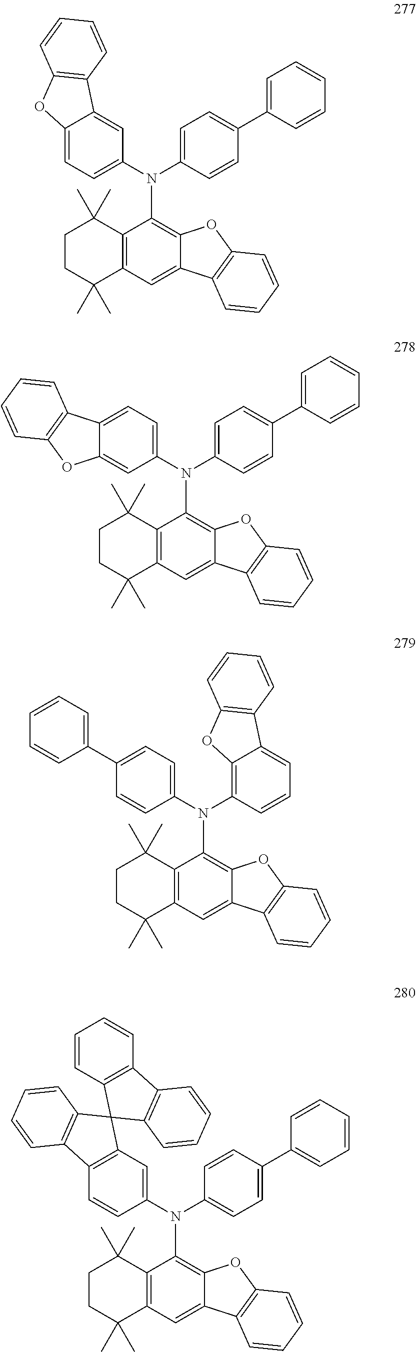 [SMILES: CC1(C)CCC(C)(C)c2c1cc1c(oc3ccccc31)c2N(c1ccc(-c2ccccc2)cc1)c1ccc2oc3ccccc3c2c1 ; CC1(C)CCC(C)(C)c2c1cc1c(oc3ccccc31)c2N(c1ccc(-c2ccccc2)cc1)c1ccc2c(c1)oc1ccccc12 ; CC1(C)CCC(C)(C)c2c1cc1c(oc3ccccc31)c2N(c1ccc(-c2ccccc2)cc1)c1cccc2c1oc1ccccc12 ; CC1(C)CCC(C)(C)c2c1cc1c(oc3ccccc31)c2N(c1ccc(-c2ccccc2)cc1)c1ccc2c(c1)C1(c3ccccc3-c3ccccc31)c1ccccc1-2]

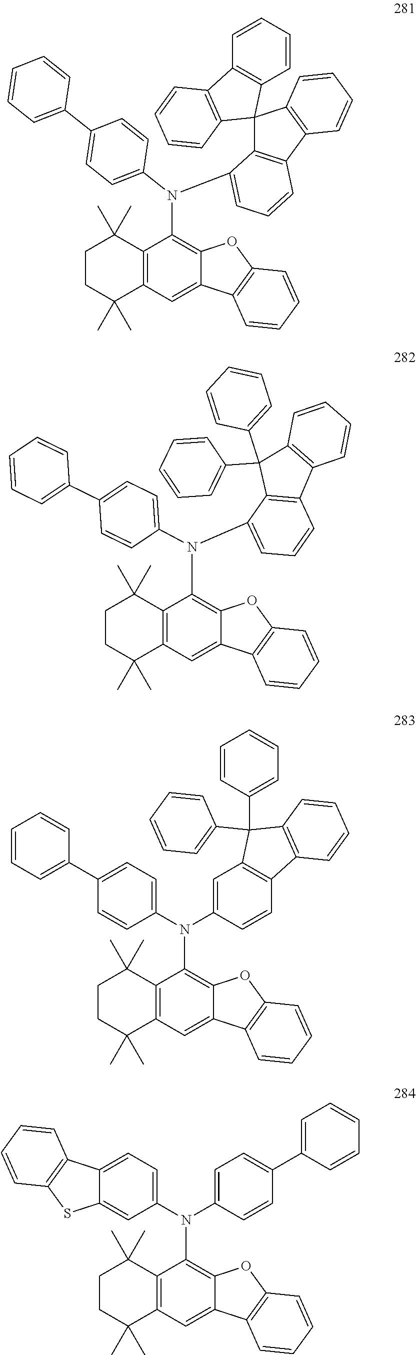

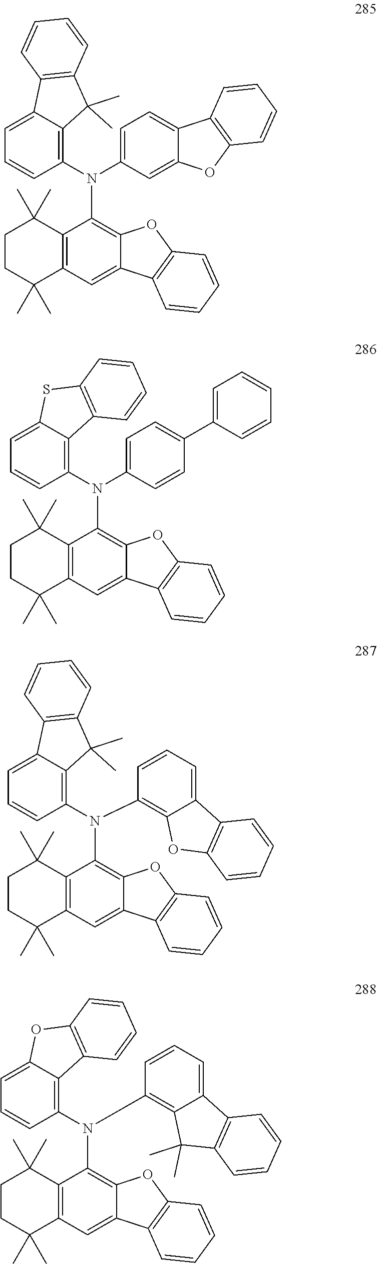

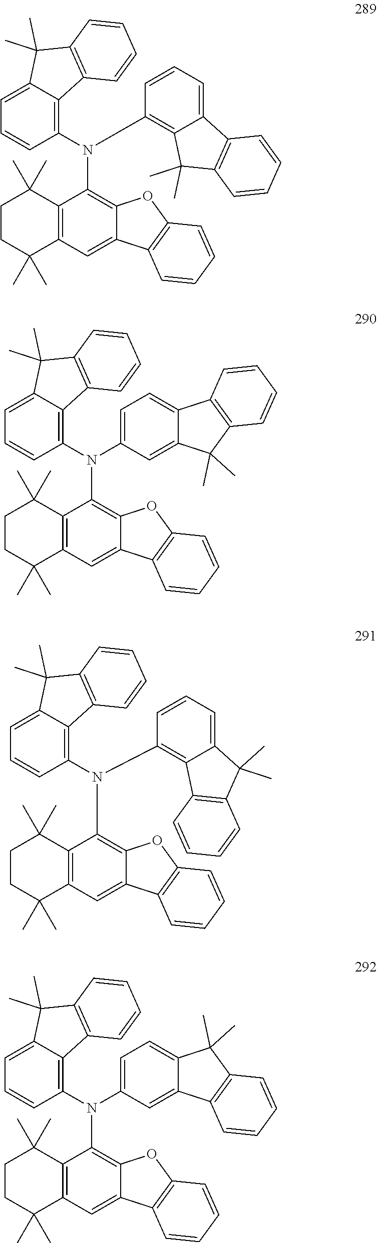

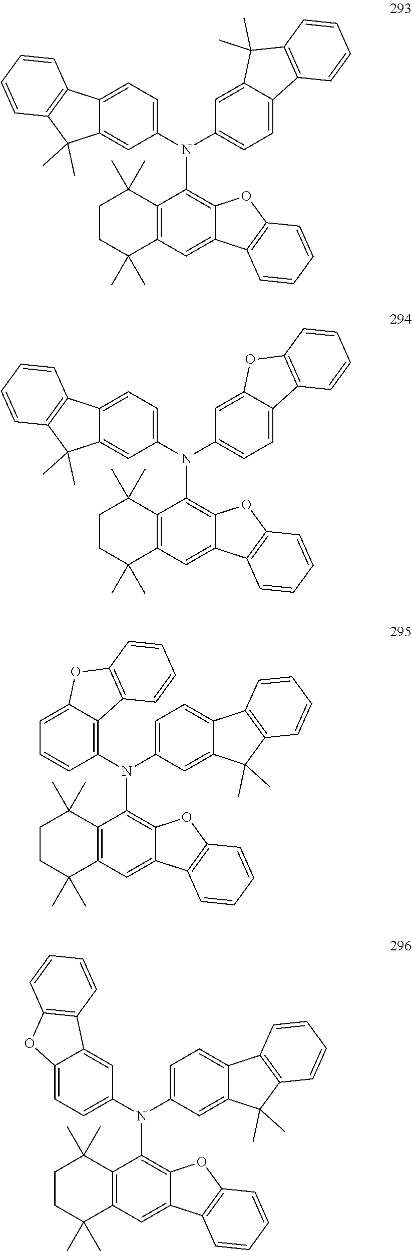

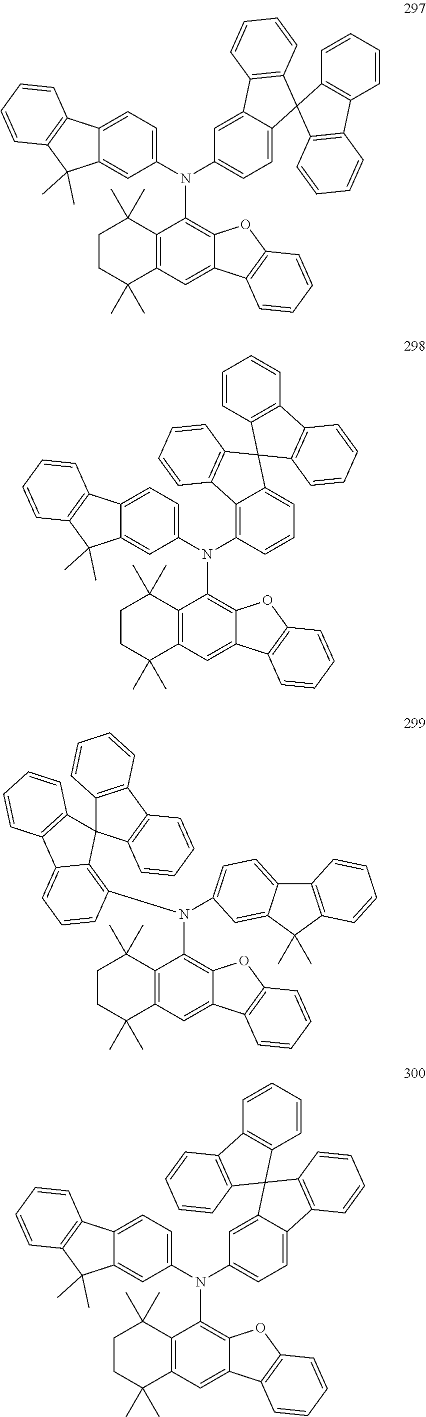

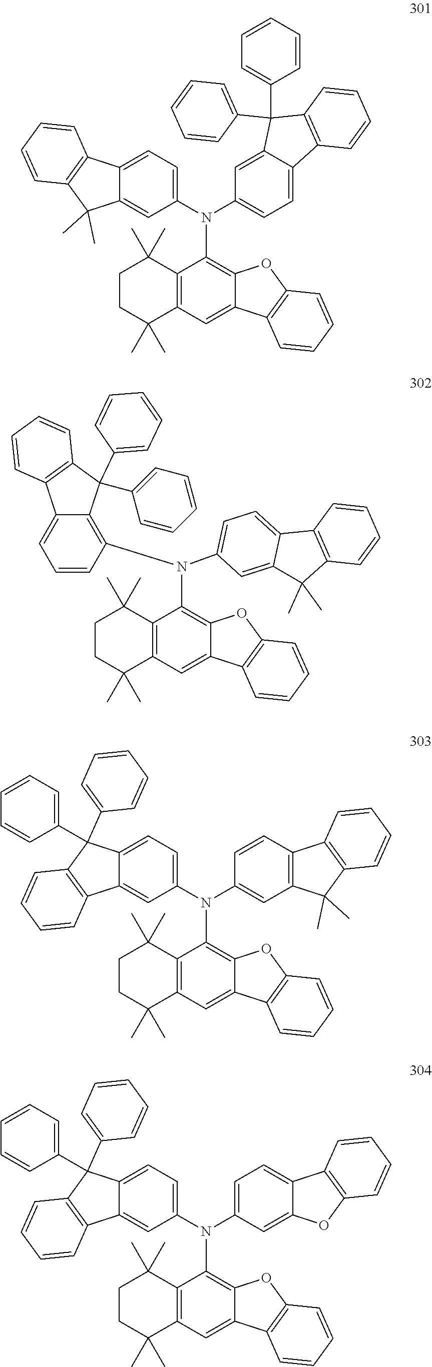

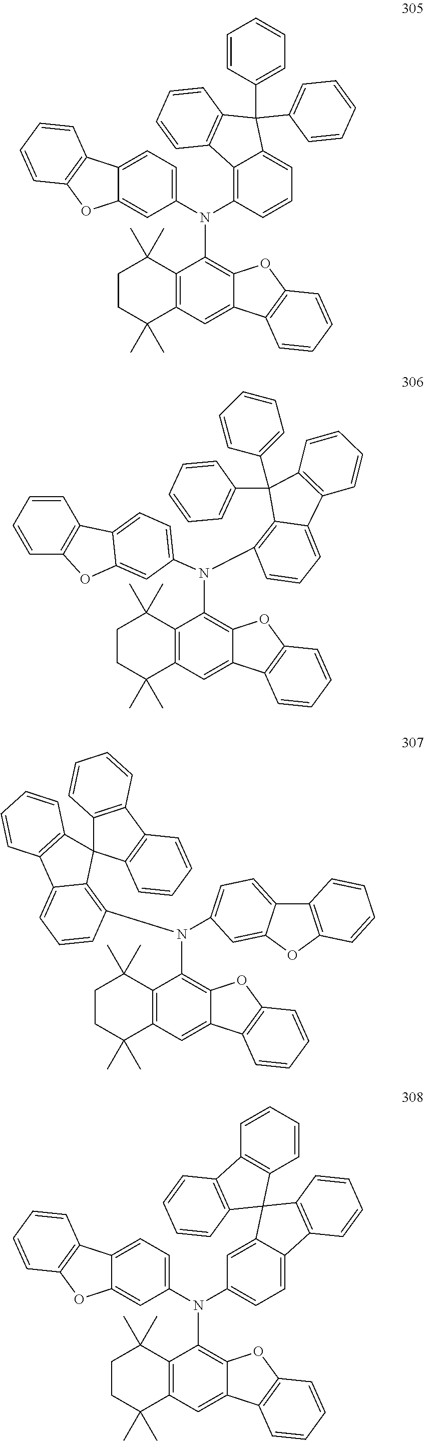

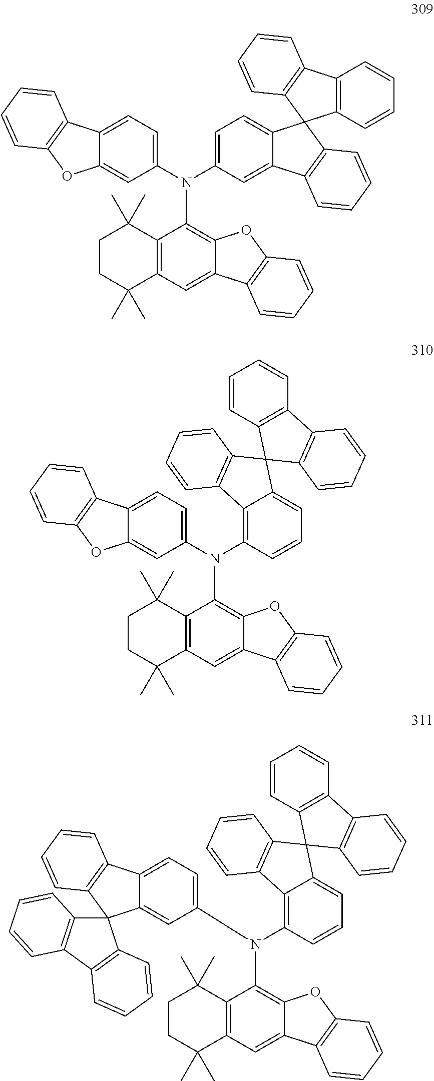

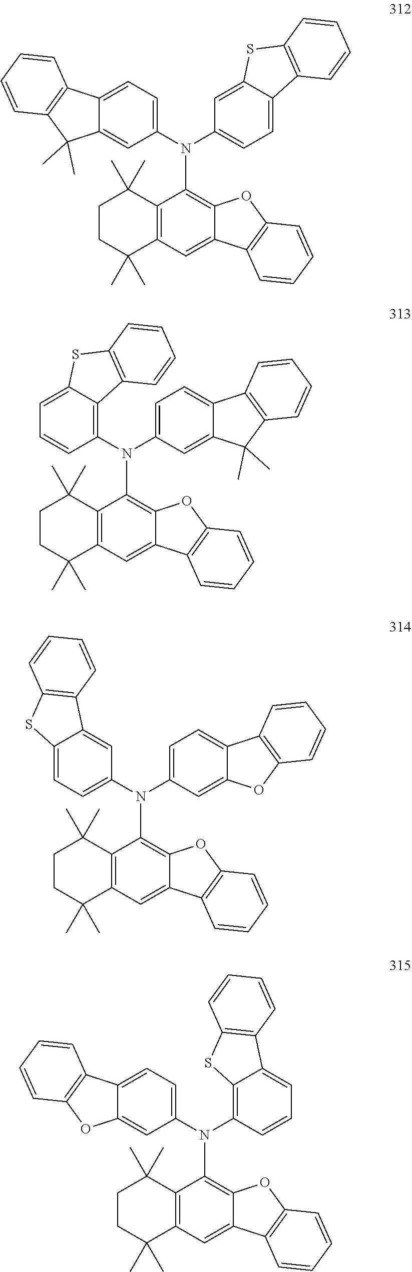

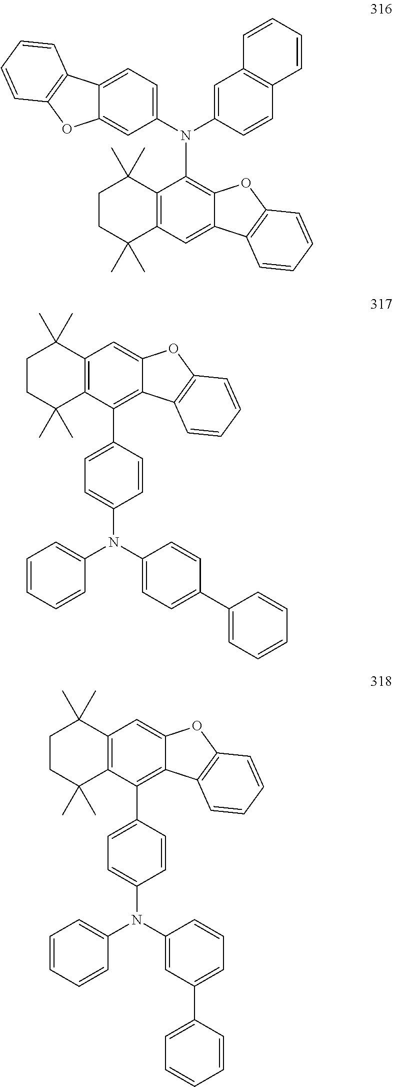 [SMILES: CC1(C)CCC(C)(C)c2c1cc1c(oc3ccccc31)c2N(c1ccc2ccccc2c1)c1ccc2c(c1)oc1ccccc12 ; CC1(C)CCC(C)(C)c2c1cc1oc3ccccc3c1c2-c1ccc(N(c2ccccc2)c2ccc(-c3ccccc3)cc2)cc1 ; CC1(C)CCC(C)(C)c2c1cc1oc3ccccc3c1c2-c1ccc(N(c2ccccc2)c2cccc(-c3ccccc3)c2)cc1]

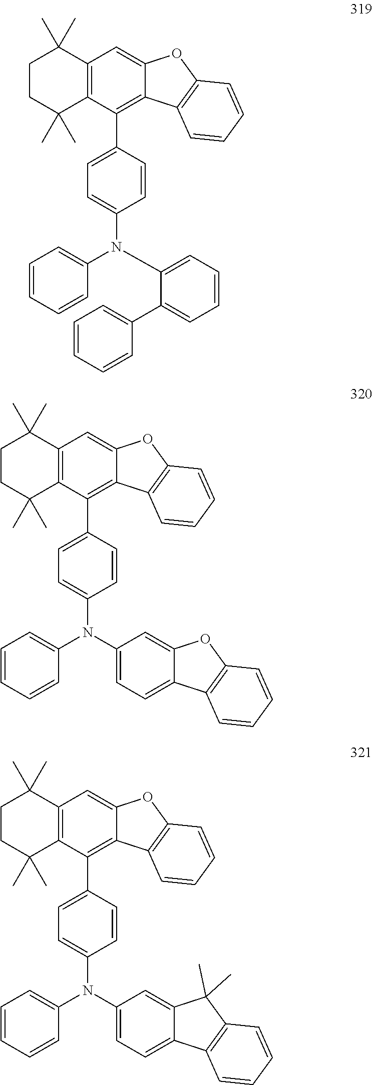 [SMILES: CC1(C)CCC(C)(C)c2c1cc1oc3ccccc3c1c2-c1ccc(N(c2ccccc2)c2ccccc2-c2ccccc2)cc1 ; CC1(C)CCC(C)(C)c2c1cc1oc3ccccc3c1c2-c1ccc(N(c2ccccc2)c2ccc3c(c2)oc2ccccc23)cc1 ; CC1(C)CCC(C)(C)c2c1cc1oc3ccccc3c1c2-c1ccc(N(c2ccccc2)c2ccc3c(c2)C(C)(C)c2ccccc2-3)cc1]

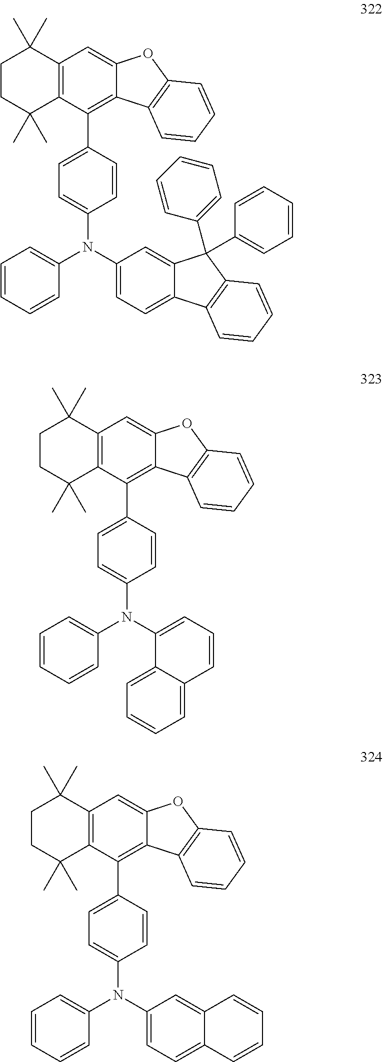 [SMILES: CC1(C)CCC(C)(C)c2c1cc1oc3ccccc3c1c2-c1ccc(N(c2ccccc2)c2ccc3c(c2)C(c2ccccc2)(c2ccccc2)c2ccccc2-3)cc1 ; CC1(C)CCC(C)(C)c2c1cc1oc3ccccc3c1c2-c1ccc(N(c2ccccc2)c2cccc3ccccc23)cc1 ; CC1(C)CCC(C)(C)c2c1cc1oc3ccccc3c1c2-c1ccc(N(c2ccccc2)c2ccc3ccccc3c2)cc1]

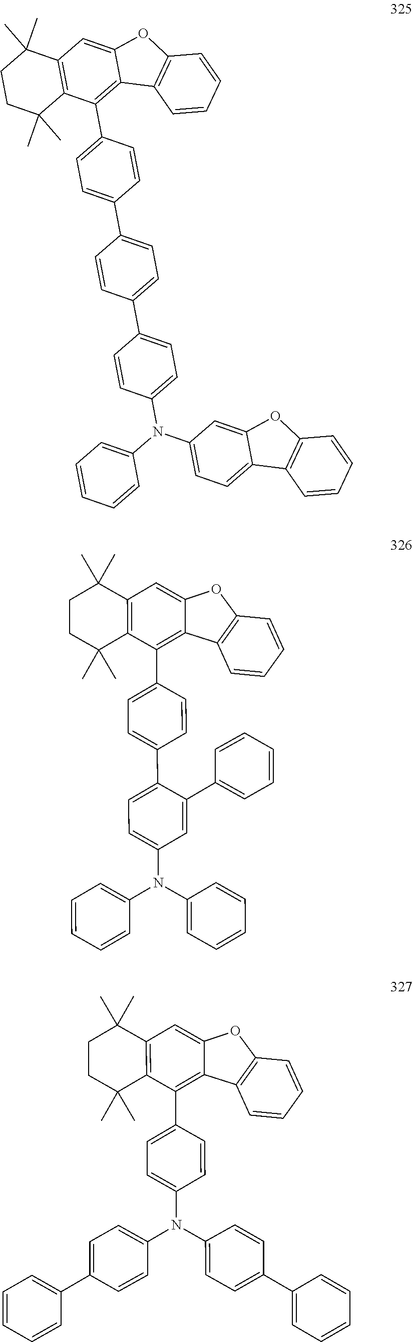

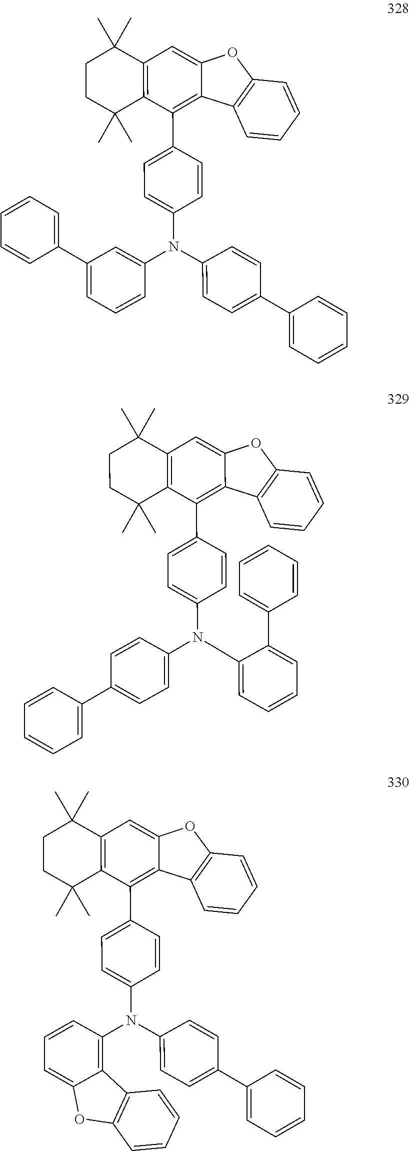

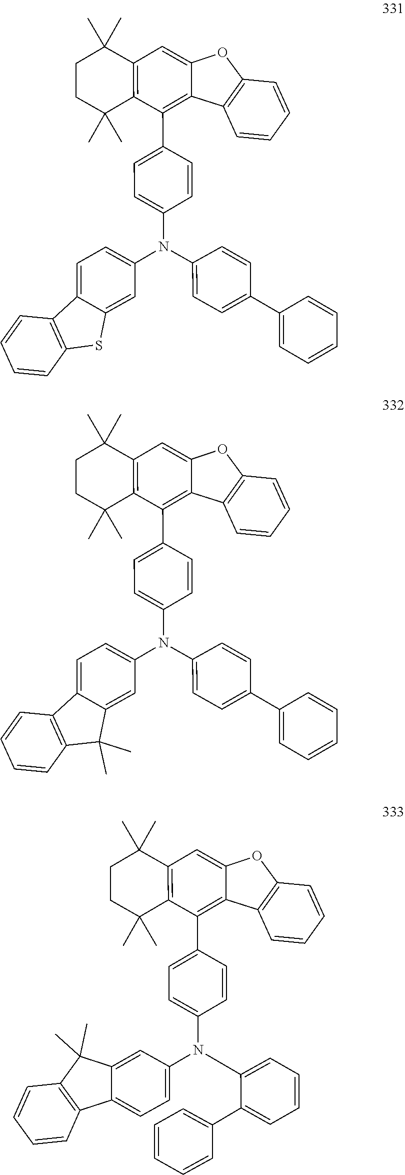 [SMILES: CC1(C)CCC(C)(C)c2c1cc1oc3ccccc3c1c2-c1ccc(N(c2ccc(-c3ccccc3)cc2)c2ccc3c(c2)sc2ccccc23)cc1 ; CC1(C)CCC(C)(C)c2c1cc1oc3ccccc3c1c2-c1ccc(N(c2ccc(-c3ccccc3)cc2)c2ccc3c(c2)C(C)(C)c2ccccc2-3)cc1 ; CC1(C)CCC(C)(C)c2c1cc1oc3ccccc3c1c2-c1ccc(N(c2ccc3c(c2)C(C)(C)c2ccccc2-3)c2ccccc2-c2ccccc2)cc1]

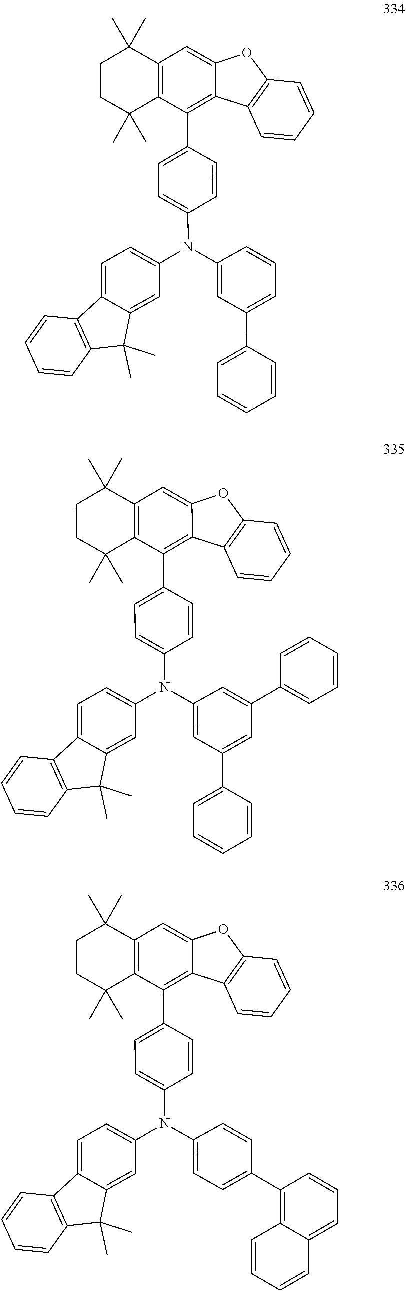 [SMILES: CC1(C)CCC(C)(C)c2c1cc1oc3ccccc3c1c2-c1ccc(N(c2cccc(-c3ccccc3)c2)c2ccc3c(c2)C(C)(C)c2ccccc2-3)cc1 ; CC1(C)CCC(C)(C)c2c1cc1oc3ccccc3c1c2-c1ccc(N(c2cc(-c3ccccc3)cc(-c3ccccc3)c2)c2ccc3c(c2)C(C)(C)c2ccccc2-3)cc1 ; CC1(C)CCC(C)(C)c2c1cc1oc3ccccc3c1c2-c1ccc(N(c2ccc(-c3cccc4ccccc34)cc2)c2ccc3c(c2)C(C)(C)c2ccccc2-3)cc1]

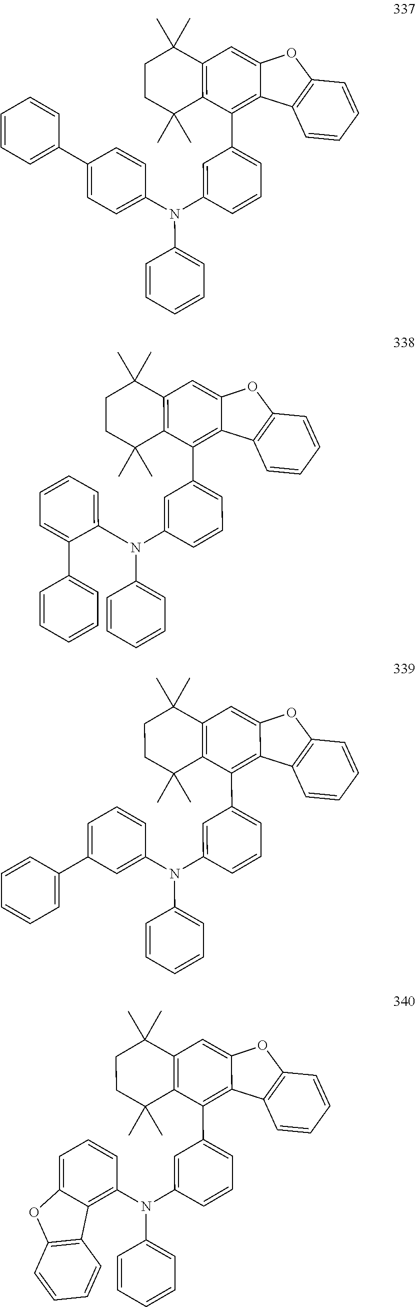

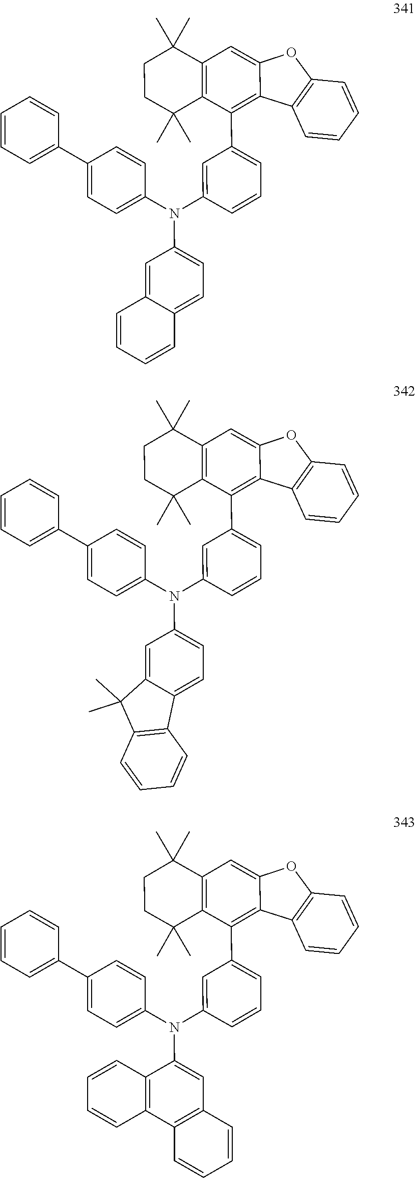

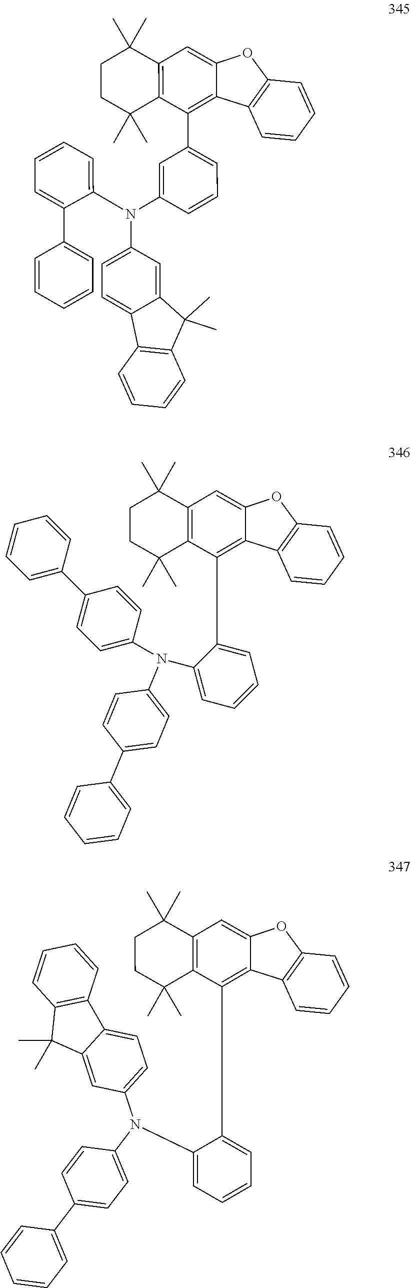 [SMILES: CC1(C)CCC(C)(C)c2c1cc1oc3ccccc3c1c2-c1cccc(N(c2ccc3c(c2)C(C)(C)c2ccccc2-3)c2ccccc2-c2ccccc2)c1 ; CC1(C)CCC(C)(C)c2c1cc1oc3ccccc3c1c2-c1ccccc1N(c1ccc(-c2ccccc2)cc1)c1ccc(-c2ccccc2)cc1 ; CC1(C)CCC(C)(C)c2c1cc1oc3ccccc3c1c2-c1ccccc1N(c1ccc(-c2ccccc2)cc1)c1ccc2c(c1)C(C)(C)c1ccccc1-2]

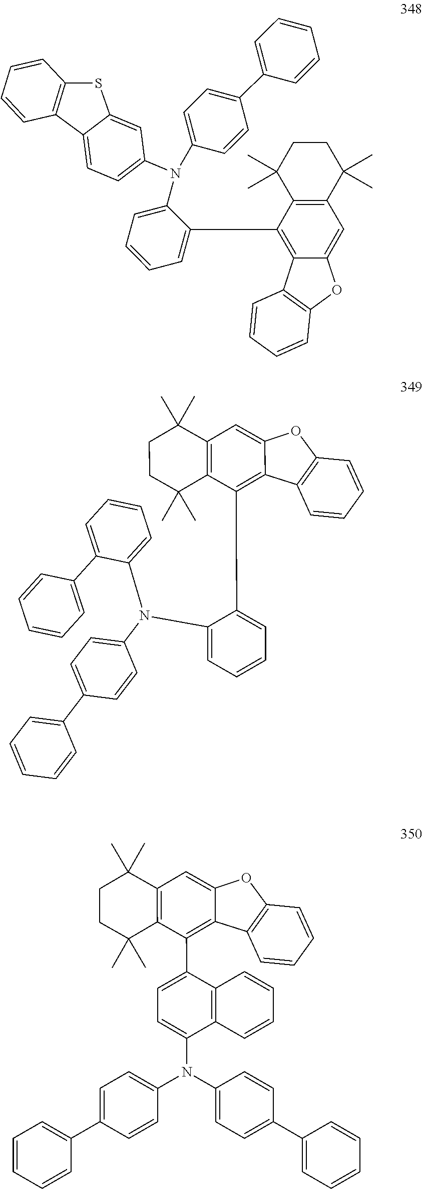 [SMILES: CC1(C)CCC(C)(C)c2c1cc1oc3ccccc3c1c2-c1ccccc1N(c1ccc(-c2ccccc2)cc1)c1ccc2c(c1)sc1ccccc12 ; CC1(C)CCC(C)(C)c2c1cc1oc3ccccc3c1c2-c1ccccc1N(c1ccc(-c2ccccc2)cc1)c1ccccc1-c1ccccc1 ; CC1(C)CCC(C)(C)c2c1cc1oc3ccccc3c1c2-c1ccc(N(c2ccc(-c3ccccc3)cc2)c2ccc(-c3ccccc3)cc2)c2ccccc12]

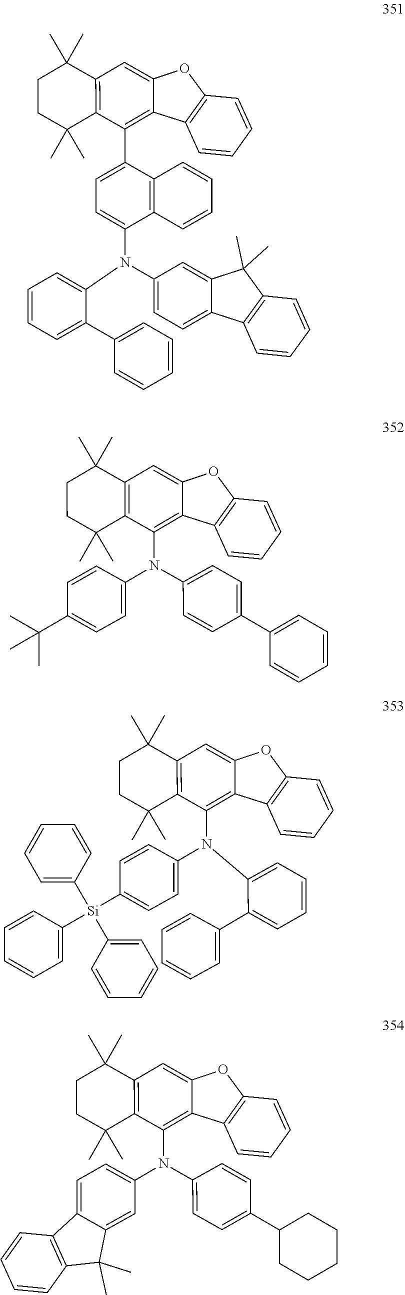

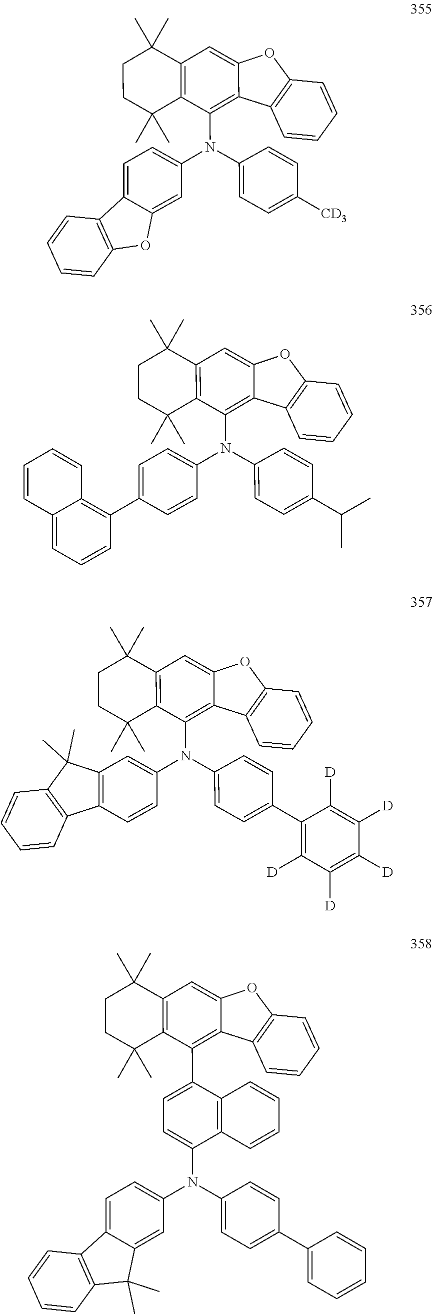 [SMILES: [2H]C([2H])([2H])c1ccc(N(c2ccc3c(c2)oc2ccccc23)c2c3c(cc4oc5ccccc5c24)C(C)(C)CCC3(C)C)cc1 ; CC(C)c1ccc(N(c2ccc(-c3cccc4ccccc34)cc2)c2c3c(cc4oc5ccccc5c24)C(C)(C)CCC3(C)C)cc1 ; [2H]c1c([2H])c([2H])c(-c2ccc(N(c3ccc4c(c3)C(C)(C)c3ccccc3-4)c3c4c(cc5oc6ccccc6c35)C(C)(C)CCC4(C)C)cc2)c([2H])c1[2H] ; CC1(C)CCC(C)(C)c2c1cc1oc3ccccc3c1c2-c1ccc(N(c2ccc(-c3ccccc3)cc2)c2ccc3c(c2)C(C)(C)c2ccccc2-3)c2ccccc12]

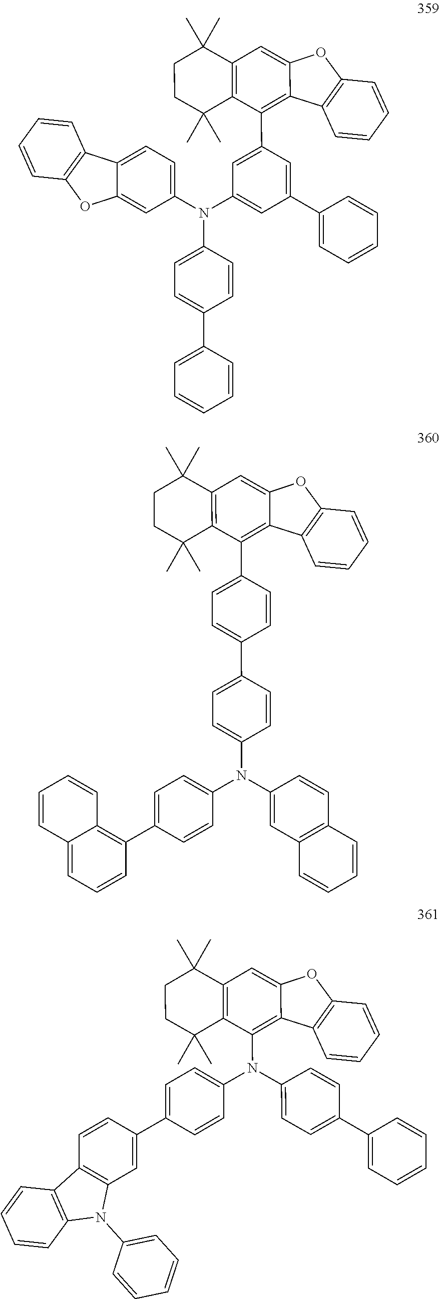

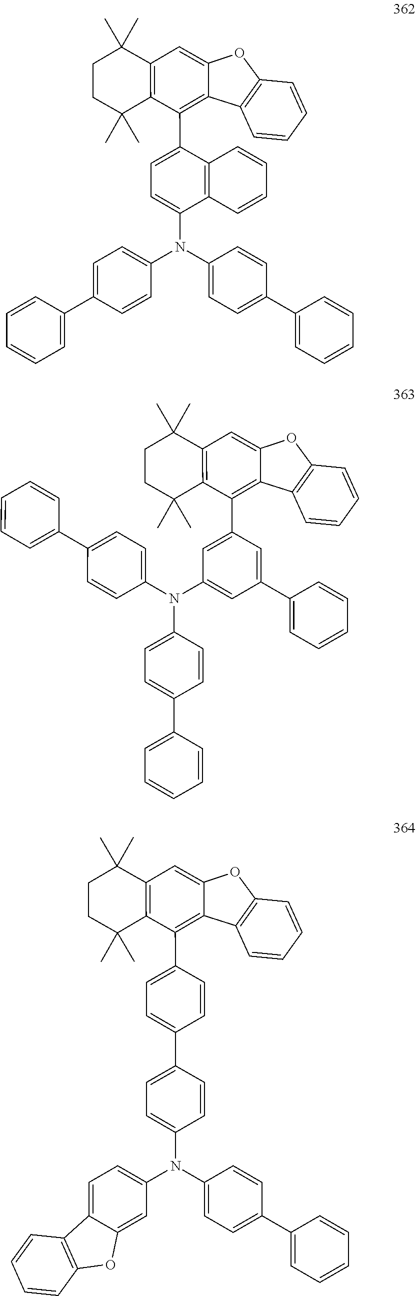 [SMILES: CC1(C)CCC(C)(C)c2c1cc1oc3ccccc3c1c2-c1ccc(N(c2ccc(-c3ccccc3)cc2)c2ccc(-c3ccccc3)cc2)c2ccccc12 ; CC1(C)CCC(C)(C)c2c1cc1oc3ccccc3c1c2-c1cc(-c2ccccc2)cc(N(c2ccc(-c3ccccc3)cc2)c2ccc(-c3ccccc3)cc2)c1 ; CC1(C)CCC(C)(C)c2c1cc1oc3ccccc3c1c2-c1ccc(-c2ccc(N(c3ccc(-c4ccccc4)cc3)c3ccc4c(c3)oc3ccccc34)cc2)cc1]

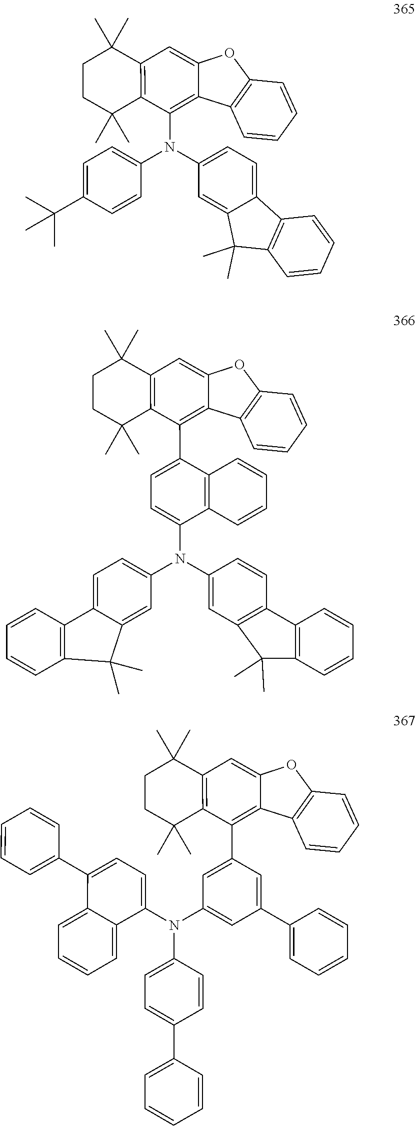 [SMILES: CC(C)(C)c1ccc(N(c2ccc3c(c2)C(C)(C)c2ccccc2-3)c2c3c(cc4oc5ccccc5c24)C(C)(C)CCC3(C)C)cc1 ; CC1(C)CCC(C)(C)c2c1cc1oc3ccccc3c1c2-c1ccc(N(c2ccc3c(c2)C(C)(C)c2ccccc2-3)c2ccc3c(c2)C(C)(C)c2ccccc2-3)c2ccccc12 ; CC1(C)CCC(C)(C)c2c1cc1oc3ccccc3c1c2-c1cc(-c2ccccc2)cc(N(c2ccc(-c3ccccc3)cc2)c2ccc(-c3ccccc3)c3ccccc23)c1]

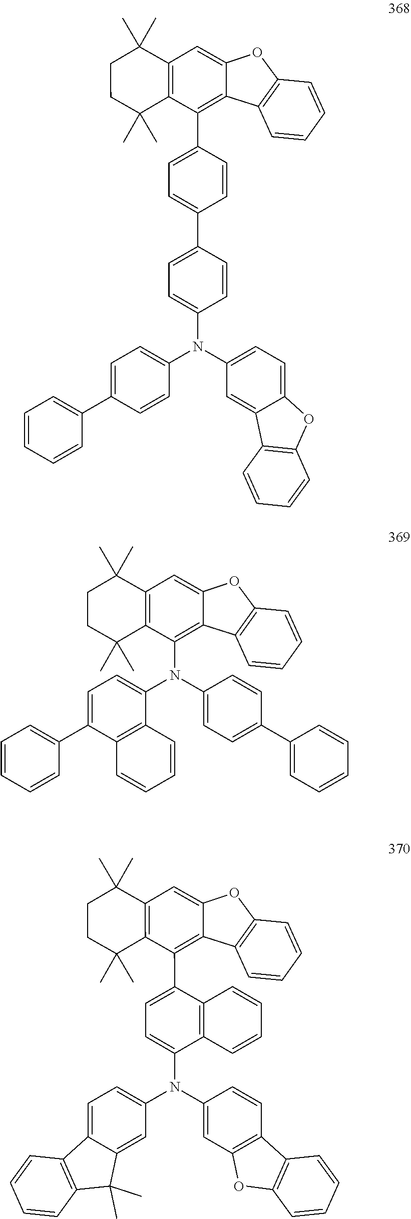 [SMILES: CC1(C)CCC(C)(C)c2c1cc1oc3ccccc3c1c2-c1ccc(-c2ccc(N(c3ccc(-c4ccccc4)cc3)c3ccc4oc5ccccc5c4c3)cc2)cc1 ; CC1(C)CCC(C)(C)c2c1cc1oc3ccccc3c1c2N(c1ccc(-c2ccccc2)cc1)c1ccc(-c2ccccc2)c2ccccc12 ; CC1(C)CCC(C)(C)c2c1cc1oc3ccccc3c1c2-c1ccc(N(c2ccc3c(c2)C(C)(C)c2ccccc2-3)c2ccc3c(c2)oc2ccccc23)c2ccccc12]

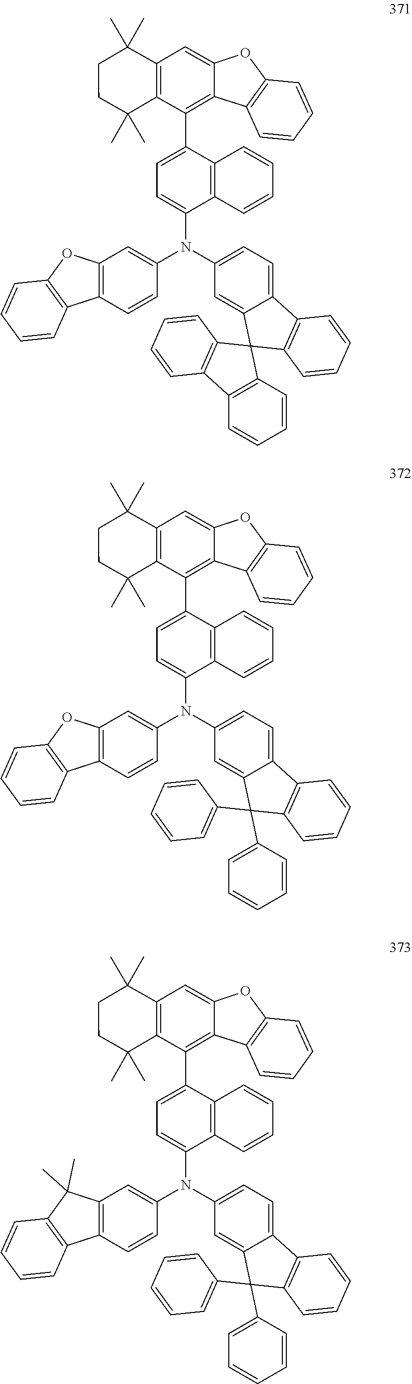 [SMILES: CC1(C)CCC(C)(C)c2c1cc1oc3ccccc3c1c2-c1ccc(N(c2ccc3c(c2)C2(c4ccccc4-c4ccccc42)c2ccccc2-3)c2ccc3c(c2)oc2ccccc23)c2ccccc12 ; CC1(C)CCC(C)(C)c2c1cc1oc3ccccc3c1c2-c1ccc(N(c2ccc3c(c2)C(c2ccccc2)(c2ccccc2)c2ccccc2-3)c2ccc3c(c2)oc2ccccc23)c2ccccc12 ; CC1(C)CCC(C)(C)c2c1cc1oc3ccccc3c1c2-c1ccc(N(c2ccc3c(c2)C(C)(C)c2ccccc2-3)c2ccc3c(c2)C(c2ccccc2)(c2ccccc2)c2ccccc2-3)c2ccccc12]

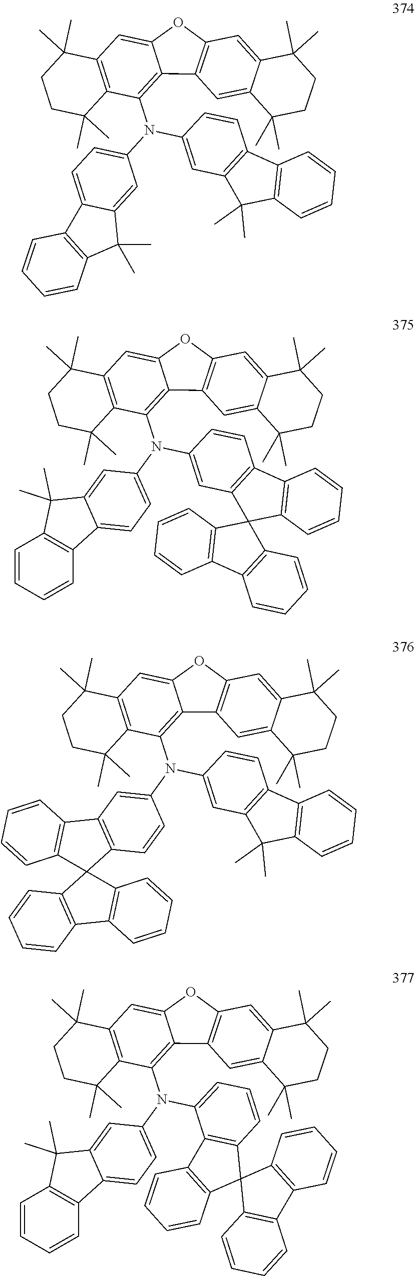

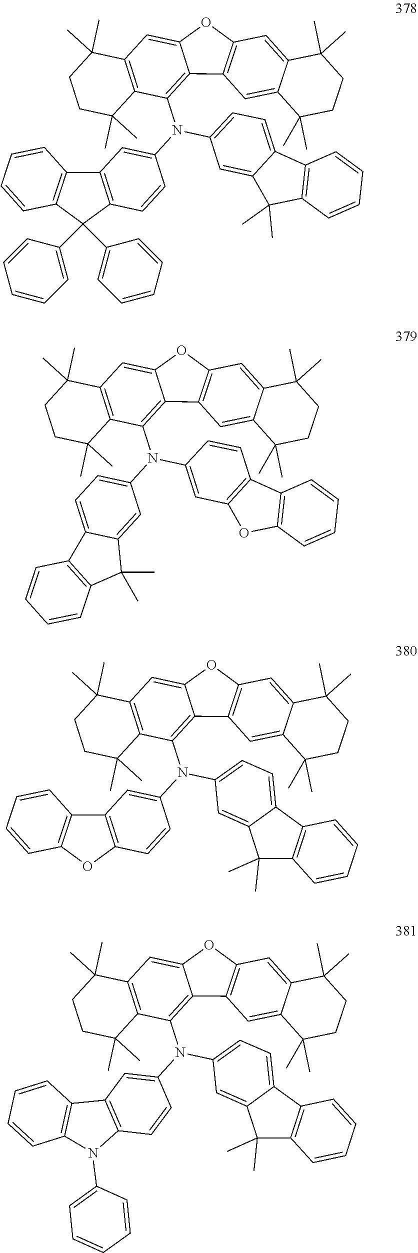 [SMILES: CC1(C)CCC(C)(C)c2cc3c(cc21)oc1cc2c(c(N(c4ccc5c(c4)-c4ccccc4C5(c4ccccc4)c4ccccc4)c4ccc5c(c4)C(C)(C)c4ccccc4-5)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5c(c4)oc4ccccc45)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5oc6ccccc6c5c4)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5c(c4)c4ccccc4n5-c4ccccc4)c13)C(C)(C)CCC2(C)C]

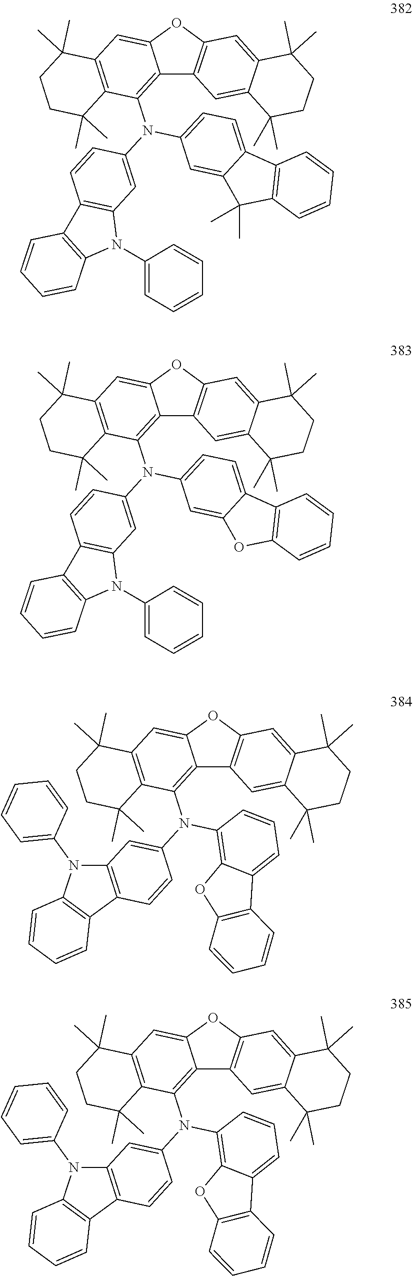 [SMILES: CC1(C)CCC(C)(C)c2cc3c(cc21)oc1cc2c(c(N(c4ccc5c(c4)C(C)(C)c4ccccc4-5)c4ccc5c6ccccc6n(-c6ccccc6)c5c4)c13)C(C)(C)CCC2(C)C ; CC1(C)CCC(C)(C)c2cc3c(cc21)oc1cc2c(c(N(c4ccc5c(c4)oc4ccccc45)c4ccc5c6ccccc6n(-c6ccccc6)c5c4)c13)C(C)(C)CCC2(C)C ; CC1(C)CCC(C)(C)c2cc3c(cc21)oc1cc2c(c(N(c4ccc5c6ccccc6n(-c6ccccc6)c5c4)c4cccc5c4oc4ccccc45)c13)C(C)(C)CCC2(C)C ; CC1(C)CCC(C)(C)c2cc3c(cc21)oc1cc2c(c(N(c4ccc5c6ccccc6n(-c6ccccc6)c5c4)c4cccc5c4oc4ccccc45)c13)C(C)(C)CCC2(C)C]

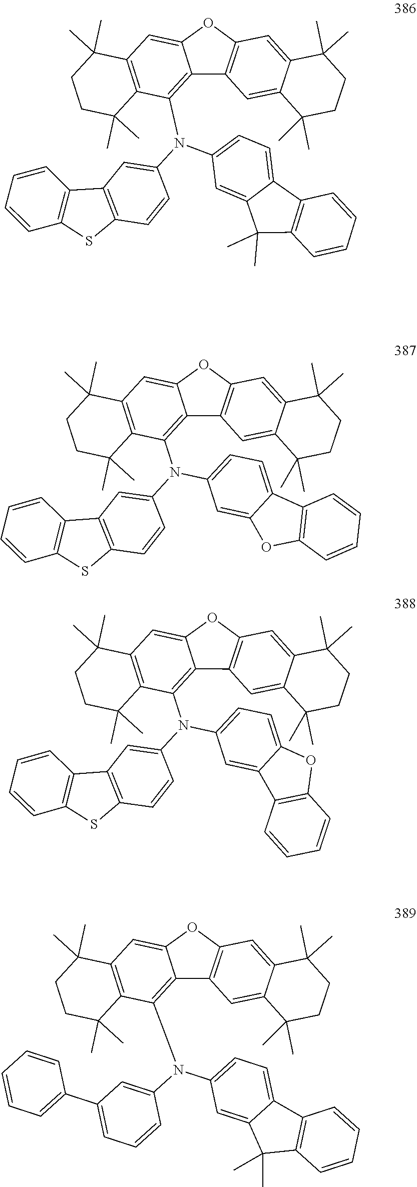 [SMILES: CC1(C)CCC(C)(C)c2cc3c(cc21)oc1cc2c(c(N(c4ccc5c(c4)C(C)(C)c4ccccc4-5)c4ccc5sc6ccccc6c5c4)c13)C(C)(C)CCC2(C)C ; CC1(C)CCC(C)(C)c2cc3c(cc21)oc1cc2c(c(N(c4ccc5c(c4)oc4ccccc45)c4ccc5sc6ccccc6c5c4)c13)C(C)(C)CCC2(C)C ; CC1(C)CCC(C)(C)c2cc3c(cc21)oc1cc2c(c(N(c4ccc5oc6ccccc6c5c4)c4ccc5sc6ccccc6c5c4)c13)C(C)(C)CCC2(C)C ; CC1(C)CCC(C)(C)c2cc3c(cc21)oc1cc2c(c(N(c4cccc(-c5ccccc5)c4)c4ccc5c(c4)C(C)(C)c4ccccc4-5)c13)C(C)(C)CCC2(C)C]

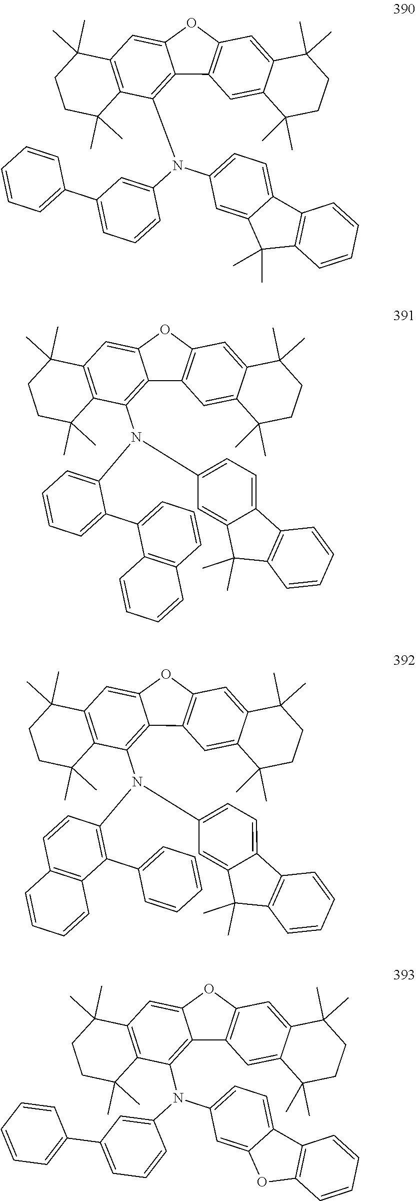

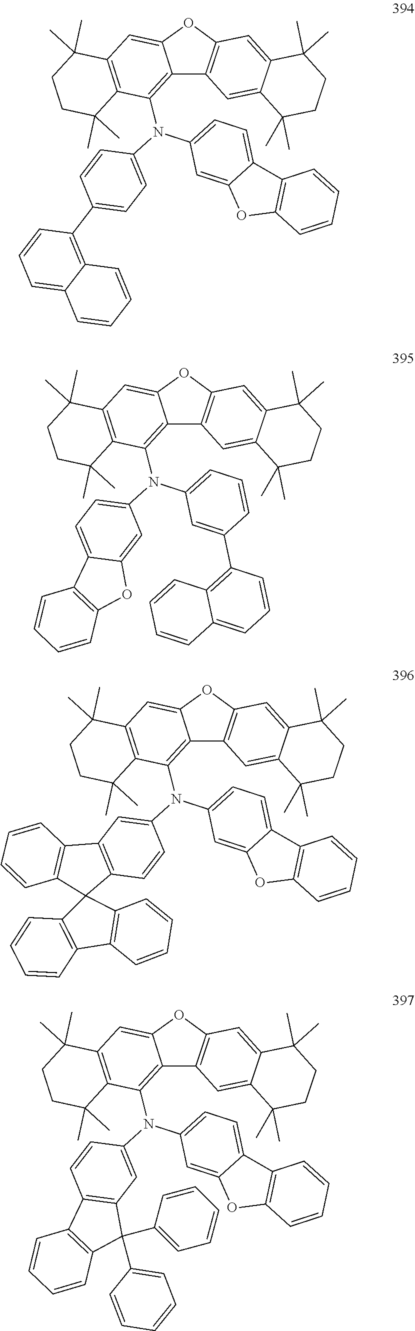

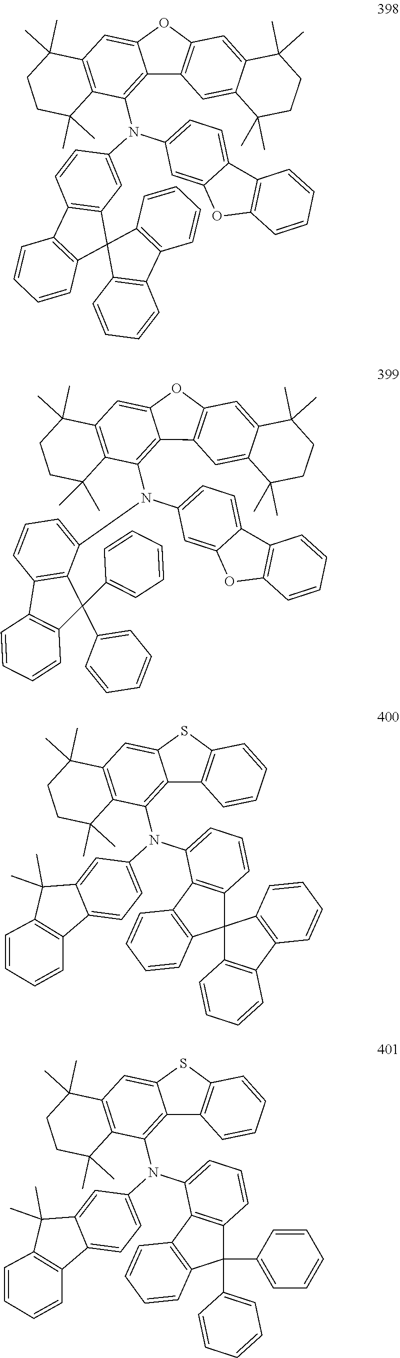

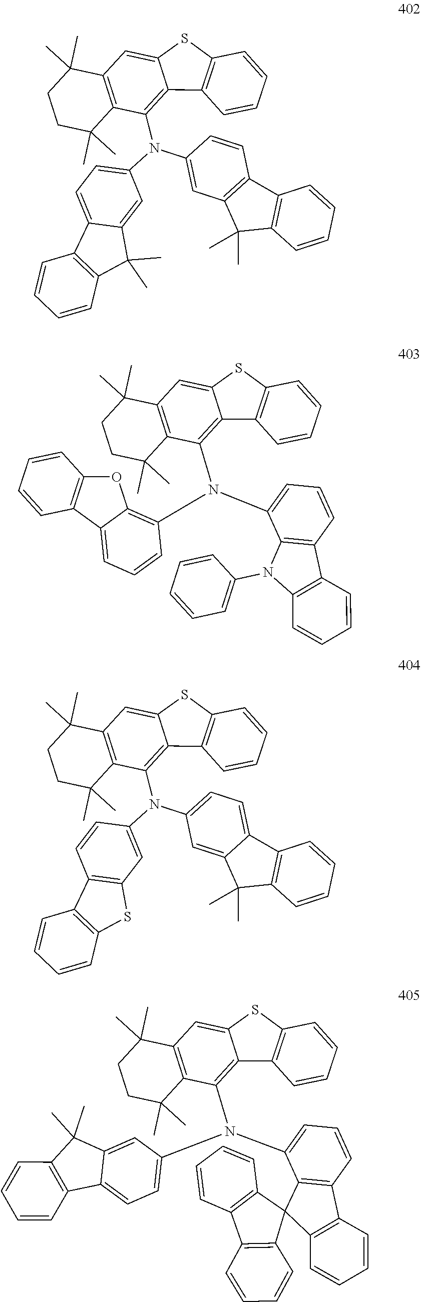 [SMILES: CC1(C)CCC(C)(C)c2c1cc1sc3ccccc3c1c2N(c1ccc2c(c1)C(C)(C)c1ccccc1-2)c1ccc2c(c1)C(C)(C)c1ccccc1-2 ; CC1(C)CCC(C)(C)c2c1cc1sc3ccccc3c1c2N(c1cccc2c1oc1ccccc12)c1cccc2c3ccccc3n(-c3ccccc3)c12 ; CC1(C)CCC(C)(C)c2c1cc1sc3ccccc3c1c2N(c1ccc2c(c1)C(C)(C)c1ccccc1-2)c1ccc2c(c1)sc1ccccc12 ; CC1(C)CCC(C)(C)c2c1cc1sc3ccccc3c1c2N(c1ccc2c(c1)C(C)(C)c1ccccc1-2)c1cccc2c1C1(c3ccccc3-c3ccccc31)c1ccccc1-2]

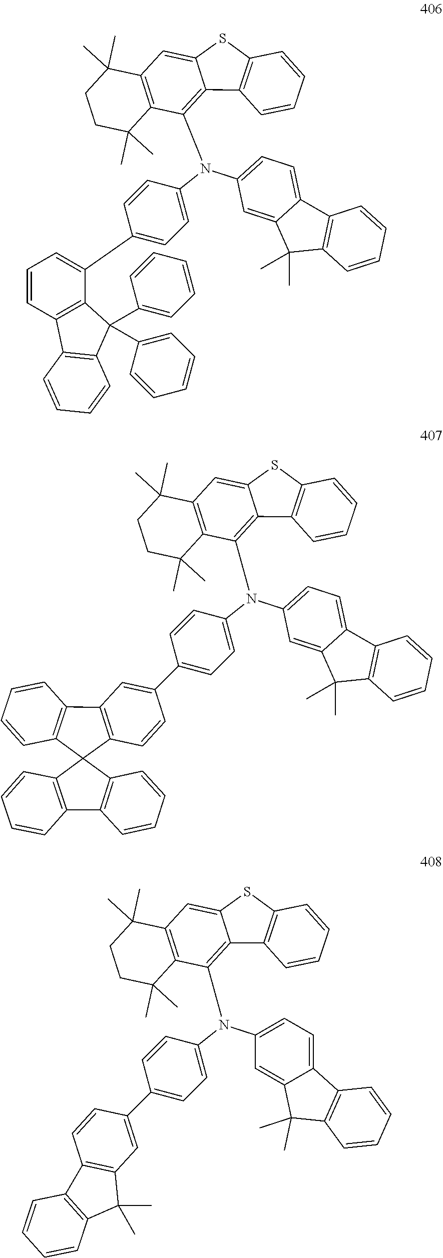 [SMILES: CC1(C)CCC(C)(C)c2c1cc1sc3ccccc3c1c2N(c1ccc(-c2cccc3c2C(c2ccccc2)(c2ccccc2)c2ccccc2-3)cc1)c1ccc2c(c1)C(C)(C)c1ccccc1-2 ; CC1(C)CCC(C)(C)c2c1cc1sc3ccccc3c1c2N(c1ccc(-c2ccc3c(c2)-c2ccccc2C32c3ccccc3-c3ccccc32)cc1)c1ccc2c(c1)C(C)(C)c1ccccc1-2 ; CC1(C)CCC(C)(C)c2c1cc1sc3ccccc3c1c2N(c1ccc(-c2ccc3c(c2)C(C)(C)c2ccccc2-3)cc1)c1ccc2c(c1)C(C)(C)c1ccccc1-2]

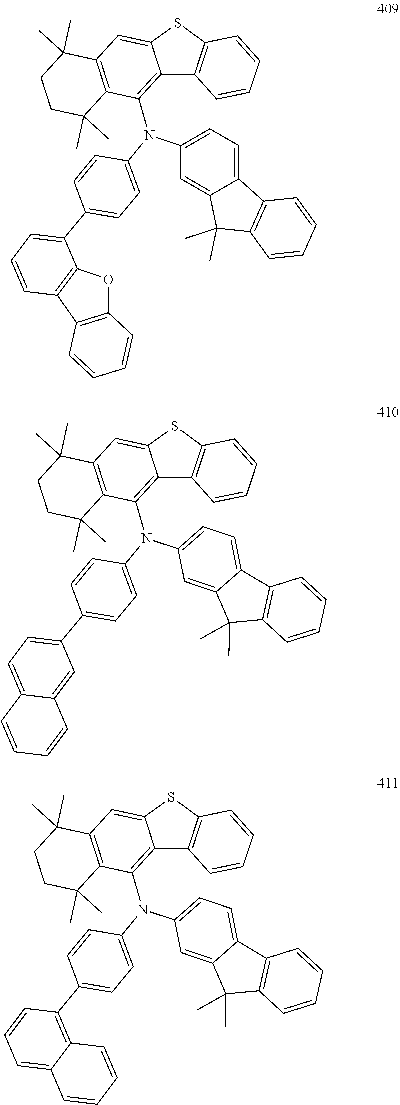

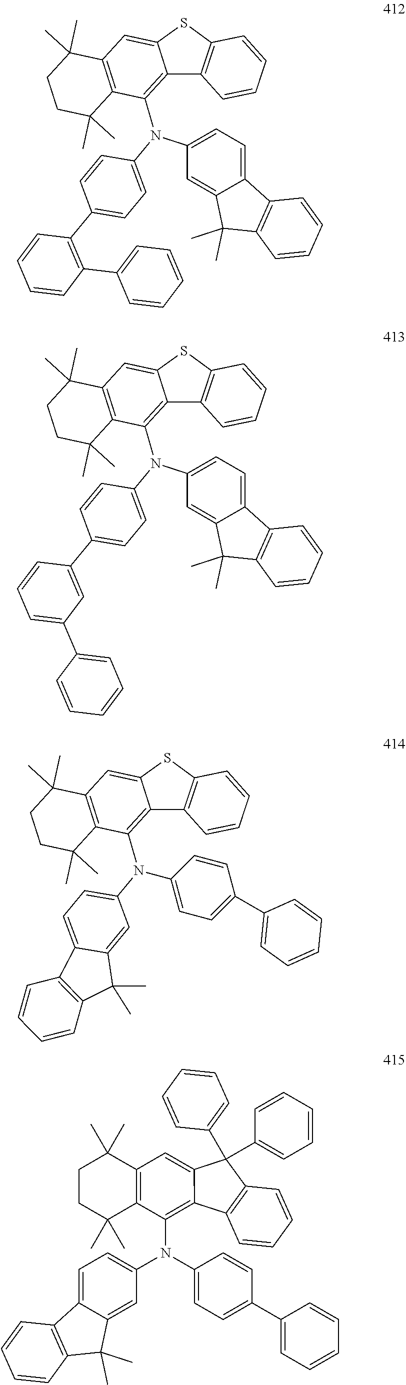

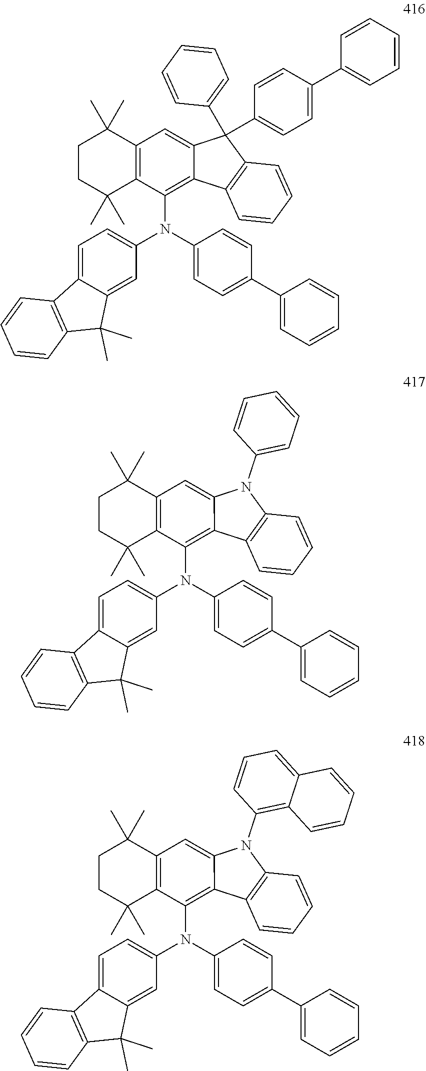 [SMILES: CC1(C)CCC(C)(C)c2c1cc1c(c2N(c2ccc(-c3ccccc3)cc2)c2ccc3c(c2)C(C)(C)c2ccccc2-3)-c2ccccc2C1(c1ccccc1)c1ccc(-c2ccccc2)cc1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1ccccc1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1cccc2ccccc12]

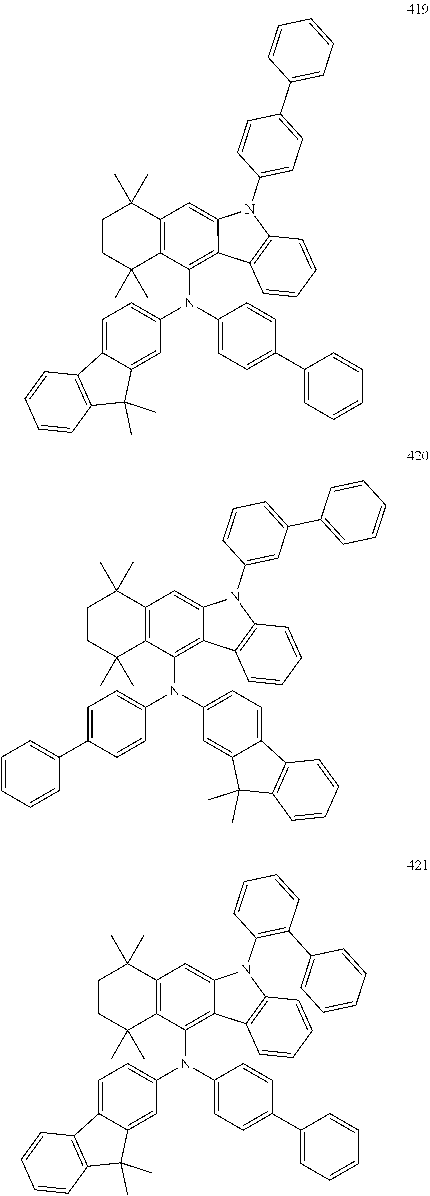 [SMILES: CC1(C)CCC(C)(C)c2c1cc1c(c2N(c2ccc(-c3ccccc3)cc2)c2ccc3c(c2)C(C)(C)c2ccccc2-3)c2ccccc2n1-c1ccc(-c2ccccc2)cc1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1cccc(-c2ccccc2)c1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1ccccc1-c1ccccc1]

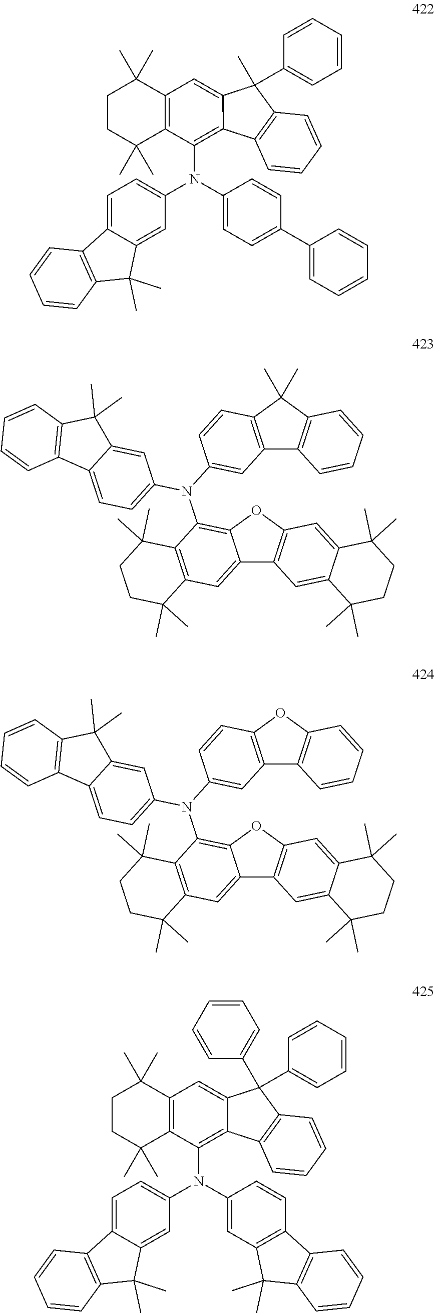 [SMILES: CC1(C)CCC(C)(C)c2c1cc1c(c2N(c2ccc(-c3ccccc3)cc2)c2ccc3c(c2)C(C)(C)c2ccccc2-3)-c2ccccc2C1(C)c1ccccc1 ; CC1(C)CCC(C)(C)c2cc3c(cc21)oc1c(N(c2ccc4c(c2)-c2ccccc2C4(C)C)c2ccc4c(c2)C(C)(C)c2ccccc2-4)c2c(cc13)C(C)(C)CCC2(C)C ; CC1(C)CCC(C)(C)c2cc3c(cc21)oc1c(N(c2ccc4c(c2)C(C)(C)c2ccccc2-4)c2ccc4oc5ccccc5c4c2)c2c(cc13)C(C)(C)CCC2(C)C ; CC1(C)CCC(C)(C)c2c1cc1c(c2N(c2ccc3c(c2)C(C)(C)c2ccccc2-3)c2ccc3c(c2)C(C)(C)c2ccccc2-3)-c2ccccc2C1(c1ccccc1)c1ccccc1]

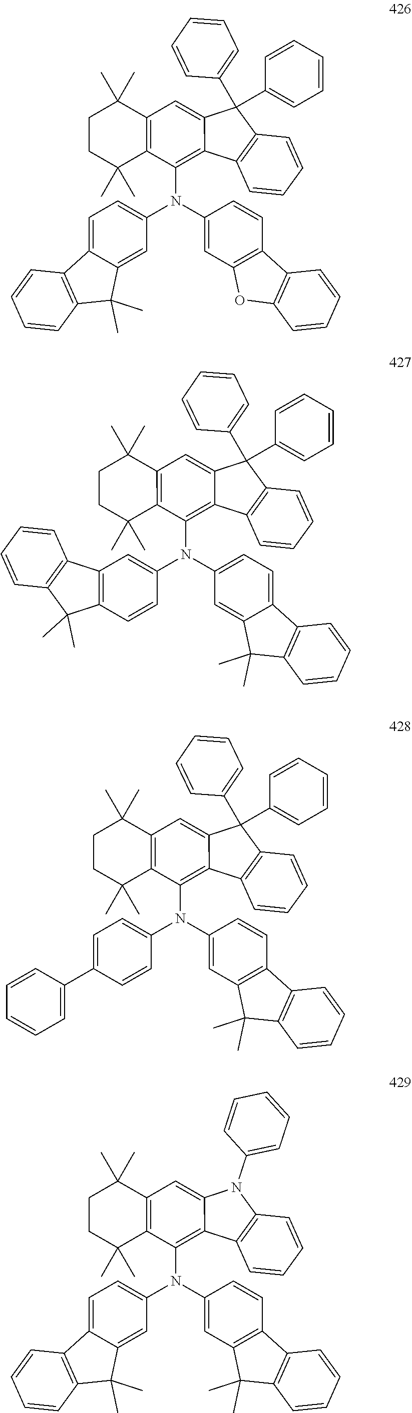 [SMILES: CC1(C)CCC(C)(C)c2c1cc1c(c2N(c2ccc3c(c2)C(C)(C)c2ccccc2-3)c2ccc3c(c2)oc2ccccc23)-c2ccccc2C1(c1ccccc1)c1ccccc1 ; CC1(C)CCC(C)(C)c2c1cc1c(c2N(c2ccc3c(c2)-c2ccccc2C3(C)C)c2ccc3c(c2)C(C)(C)c2ccccc2-3)-c2ccccc2C1(c1ccccc1)c1ccccc1 ; CC1(C)CCC(C)(C)c2c1cc1c(c2N(c2ccc(-c3ccccc3)cc2)c2ccc3c(c2)C(C)(C)c2ccccc2-3)-c2ccccc2C1(c1ccccc1)c1ccccc1 ; CC1(C)CCC(C)(C)c2c1cc1c(c2N(c2ccc3c(c2)C(C)(C)c2ccccc2-3)c2ccc3c(c2)C(C)(C)c2ccccc2-3)c2ccccc2n1-c1ccccc1]

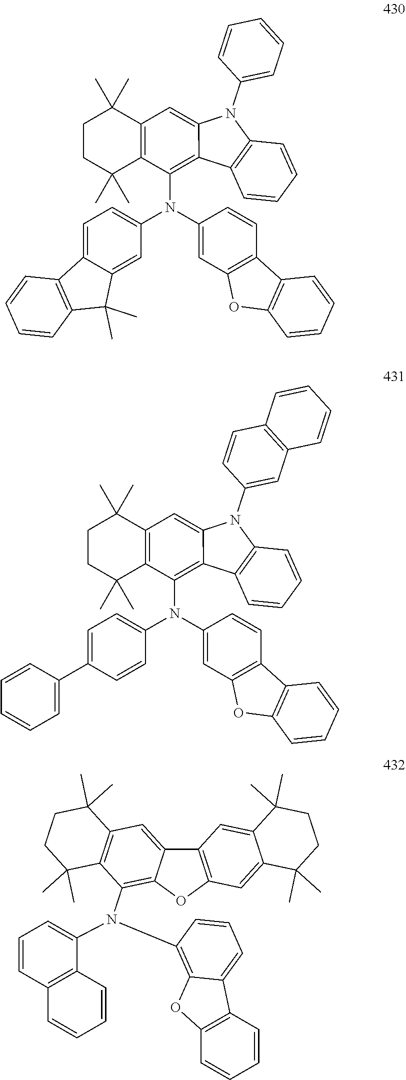

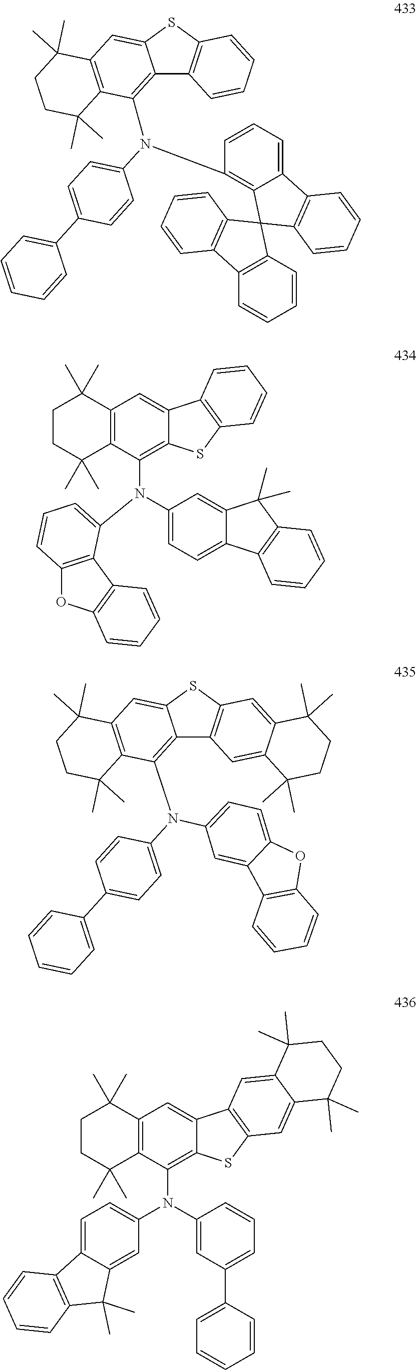

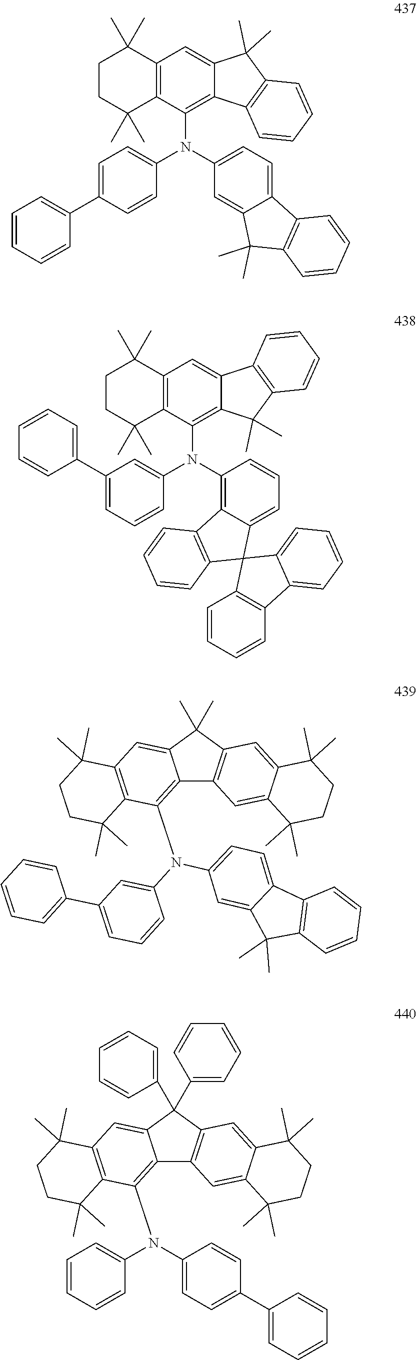 [SMILES: CC1(C)CCC(C)(C)c2c1cc1c(c2N(c2ccc(-c3ccccc3)cc2)c2ccc3c(c2)C(C)(C)c2ccccc2-3)-c2ccccc2C1(C)C ; CC1(C)CCC(C)(C)c2c1cc1c(c2N(c2cccc(-c3ccccc3)c2)c2cccc3c2-c2ccccc2C32c3ccccc3-c3ccccc32)C(C)(C)c2ccccc2-1 ; CC1(C)CCC(C)(C)c2cc3c(cc21)-c1c(cc2c(c1N(c1cccc(-c4ccccc4)c1)c1ccc4c(c1)C(C)(C)c1ccccc1-4)C(C)(C)CCC2(C)C)C3(C)C ; CC1(C)CCC(C)(C)c2cc3c(cc21)-c1c(cc2c(c1N(c1ccccc1)c1ccc(-c4ccccc4)cc1)C(C)(C)CCC2(C)C)C3(c1ccccc1)c1ccccc1]

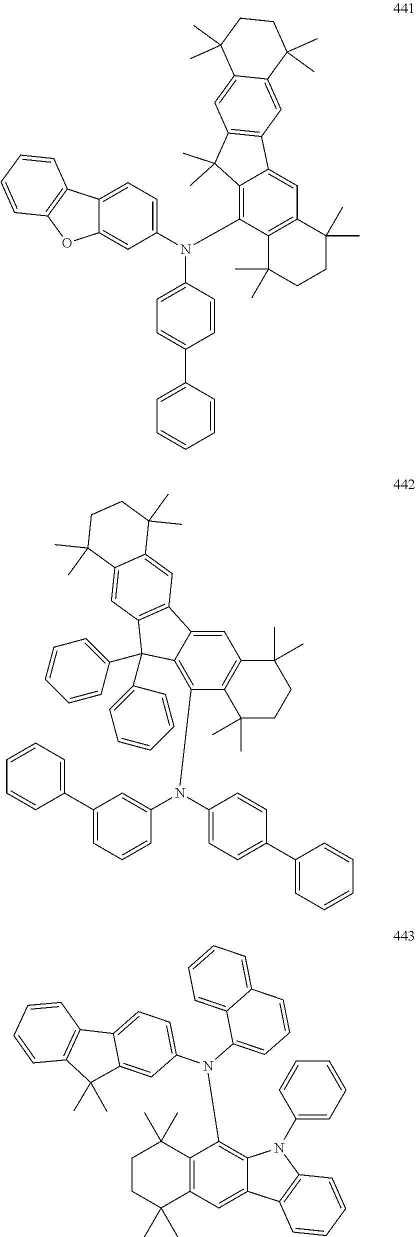 [SMILES: CC1(C)CCC(C)(C)c2cc3c(cc21)-c1cc2c(c(N(c4ccc(-c5ccccc5)cc4)c4ccc5c(c4)oc4ccccc45)c1C3(C)C)C(C)(C)CCC2(C)C ; CC1(C)CCC(C)(C)c2cc3c(cc21)-c1cc2c(c(N(c4ccc(-c5ccccc5)cc4)c4cccc(-c5ccccc5)c4)c1C3(c1ccccc1)c1ccccc1)C(C)(C)CCC2(C)C ; CC1(C)CCC(C)(C)c2c1cc1c3ccccc3n(-c3ccccc3)c1c2N(c1ccc2c(c1)C(C)(C)c1ccccc1-2)c1cccc2ccccc12]

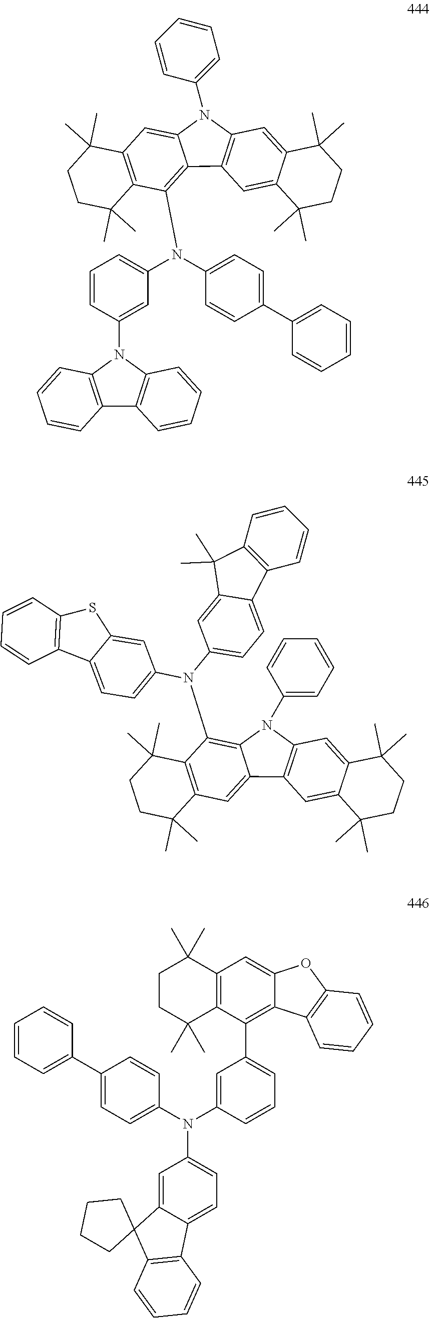 [SMILES: CC1(C)CCC(C)(C)c2cc3c(cc21)c1c(N(c2ccc(-c4ccccc4)cc2)c2cccc(-n4c5ccccc5c5ccccc54)c2)c2c(cc1n3-c1ccccc1)C(C)(C)CCC2(C)C ; CC1(C)CCC(C)(C)c2cc3c(cc21)c1cc2c(c(N(c4ccc5c(c4)C(C)(C)c4ccccc4-5)c4ccc5c(c4)sc4ccccc45)c1n3-c1ccccc1)C(C)(C)CCC2(C)C ; CC1(C)CCC(C)(C)c2c1cc1oc3ccccc3c1c2-c1cccc(N(c2ccc(-c3ccccc3)cc2)c2ccc3c(c2)C2(CCCC2)c2ccccc2-3)c1]

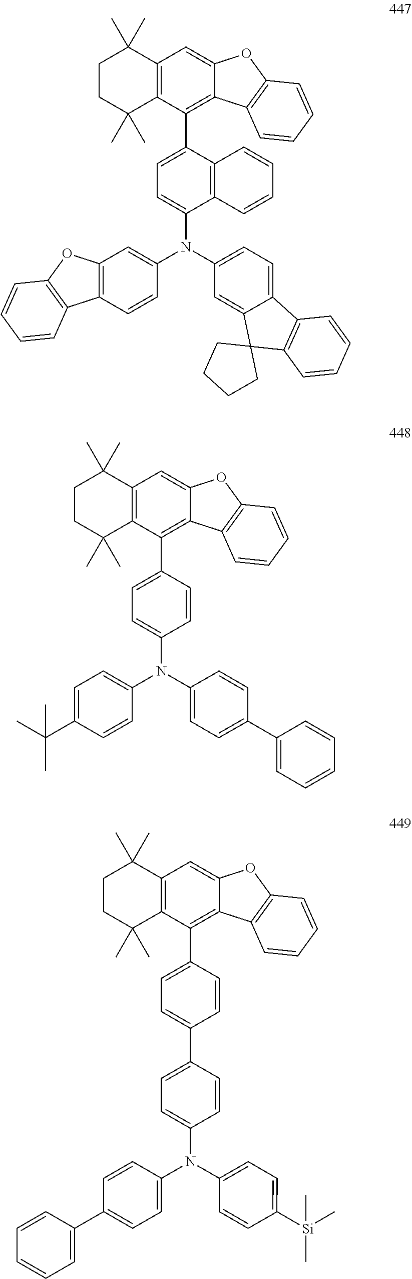 [SMILES: CC1(C)CCC(C)(C)c2c1cc1oc3ccccc3c1c2-c1ccc(N(c2ccc3c(c2)C2(CCCC2)c2ccccc2-3)c2ccc3c(c2)oc2ccccc23)c2ccccc12 ; CC(C)(C)c1ccc(N(c2ccc(-c3ccccc3)cc2)c2ccc(-c3c4c(cc5oc6ccccc6c35)C(C)(C)CCC4(C)C)cc2)cc1 ; CC1(C)CCC(C)(C)c2c1cc1oc3ccccc3c1c2-c1ccc(-c2ccc(N(c3ccc(-c4ccccc4)cc3)c3ccc([Si](C)(C)C)cc3)cc2)cc1]

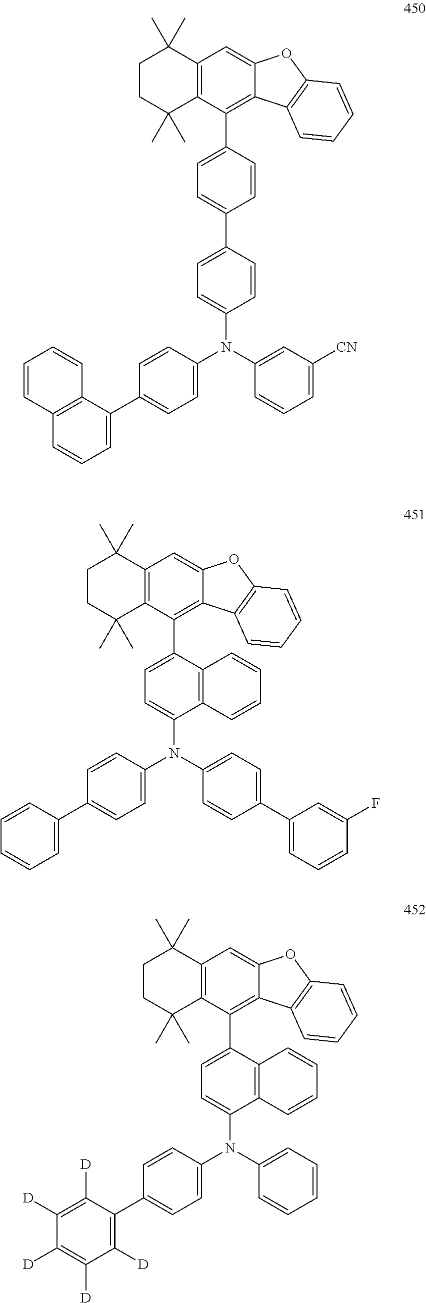 [SMILES: CC1(C)CCC(C)(C)c2c1cc1oc3ccccc3c1c2-c1ccc(-c2ccc(N(c3ccc(-c4cccc5ccccc45)cc3)c3cccc(C#N)c3)cc2)cc1 ; CC1(C)CCC(C)(C)c2c1cc1oc3ccccc3c1c2-c1ccc(N(c2ccc(-c3ccccc3)cc2)c2ccc(-c3cccc(F)c3)cc2)c2ccccc12 ; [2H]c1c([2H])c([2H])c(-c2ccc(N(c3ccccc3)c3ccc(-c4c5c(cc6oc7ccccc7c46)C(C)(C)CCC5(C)C)c4ccccc34)cc2)c([2H])c1[2H]]

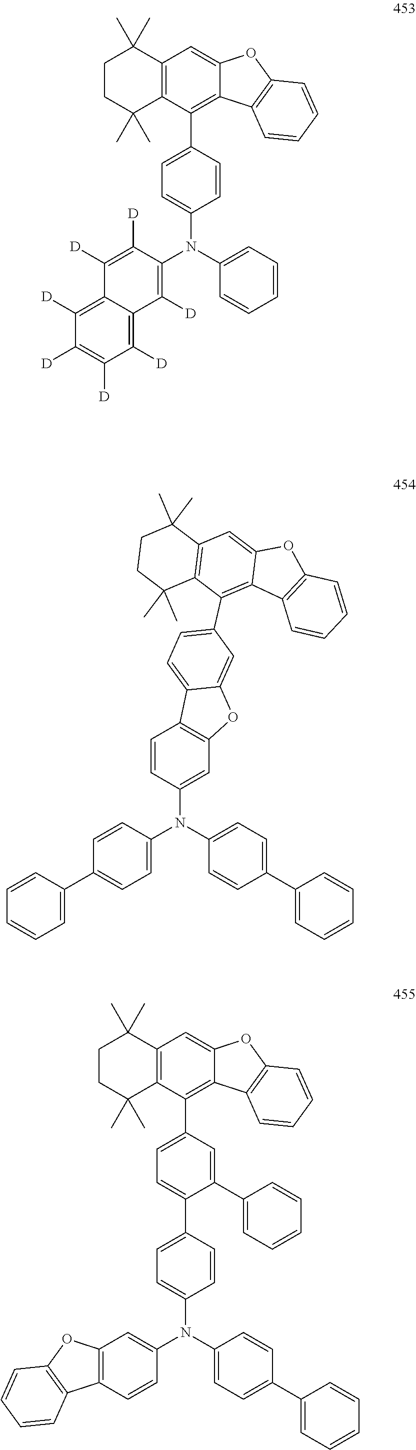 [SMILES: [2H]c1c([2H])c([2H])c2c([2H])c(N(c3ccccc3)c3ccc(-c4c5c(cc6oc7ccccc7c46)C(C)(C)CCC5(C)C)cc3)c([2H])c([2H])c2c1[2H] ; CC1(C)CCC(C)(C)c2c1cc1oc3ccccc3c1c2-c1ccc2c(c1)oc1cc(N(c3ccc(-c4ccccc4)cc3)c3ccc(-c4ccccc4)cc3)ccc12 ; CC1(C)CCC(C)(C)c2c1cc1oc3ccccc3c1c2-c1ccc(-c2ccc(N(c3ccc(-c4ccccc4)cc3)c3ccc4c(c3)oc3ccccc34)cc2)c(-c2ccccc2)c1]

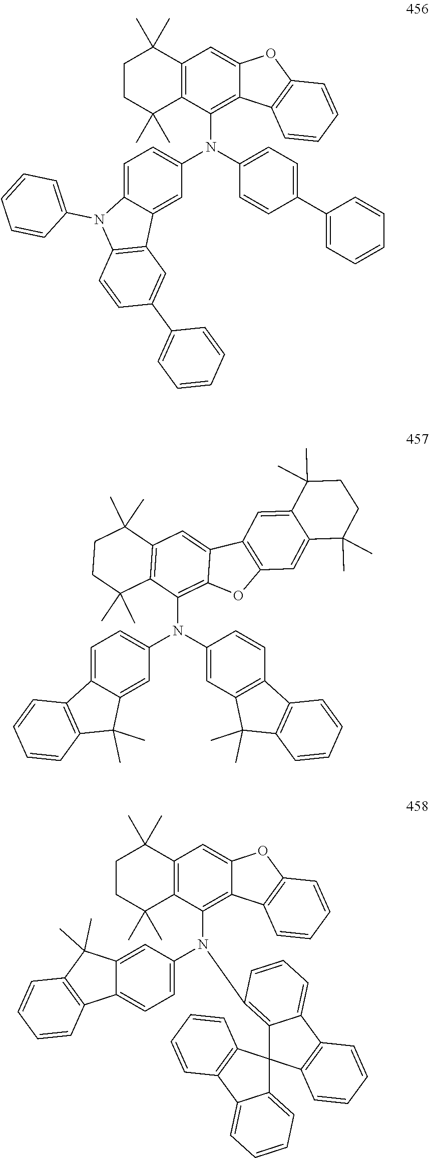 [SMILES: CC1(C)CCC(C)(C)c2c1cc1oc3ccccc3c1c2N(c1ccc(-c2ccccc2)cc1)c1ccc2c(c1)c1cc(-c3ccccc3)ccc1n2-c1ccccc1 ; CC1(C)CCC(C)(C)c2cc3c(cc21)oc1c(N(c2ccc4c(c2)C(C)(C)c2ccccc2-4)c2ccc4c(c2)C(C)(C)c2ccccc2-4)c2c(cc13)C(C)(C)CCC2(C)C ; CC1(C)CCC(C)(C)c2c1cc1oc3ccccc3c1c2N(c1ccc2c(c1)C(C)(C)c1ccccc1-2)c1cccc2c1C1(c3ccccc3-c3ccccc31)c1ccccc1-2]

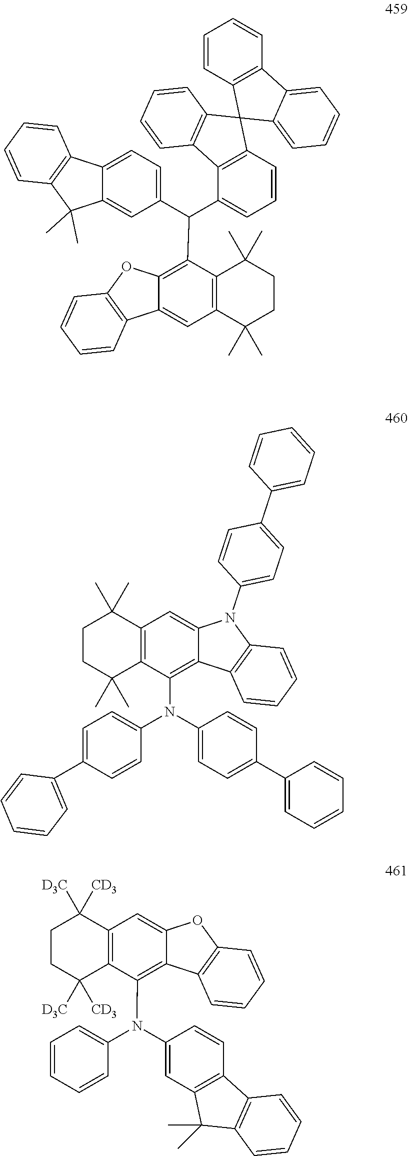 [SMILES: CC1(C)CCC(C)(C)c2c1cc1c(oc3ccccc31)c2C(c1ccc2c(c1)C(C)(C)c1ccccc1-2)c1cccc2c1-c1ccccc1C21c2ccccc2-c2ccccc21 ; CC1(C)CCC(C)(C)c2c1cc1c(c2N(c2ccc(-c3ccccc3)cc2)c2ccc(-c3ccccc3)cc2)c2ccccc2n1-c1ccc(-c2ccccc2)cc1 ; [2H]C([2H])([2H])C1(C([2H])([2H])[2H])CCC(C([2H])([2H])[2H])(C([2H])([2H])[2H])c2c1cc1oc3ccccc3c1c2N(c1ccccc1)c1ccc2c(c1)C(C)(C)c1ccccc1-2]

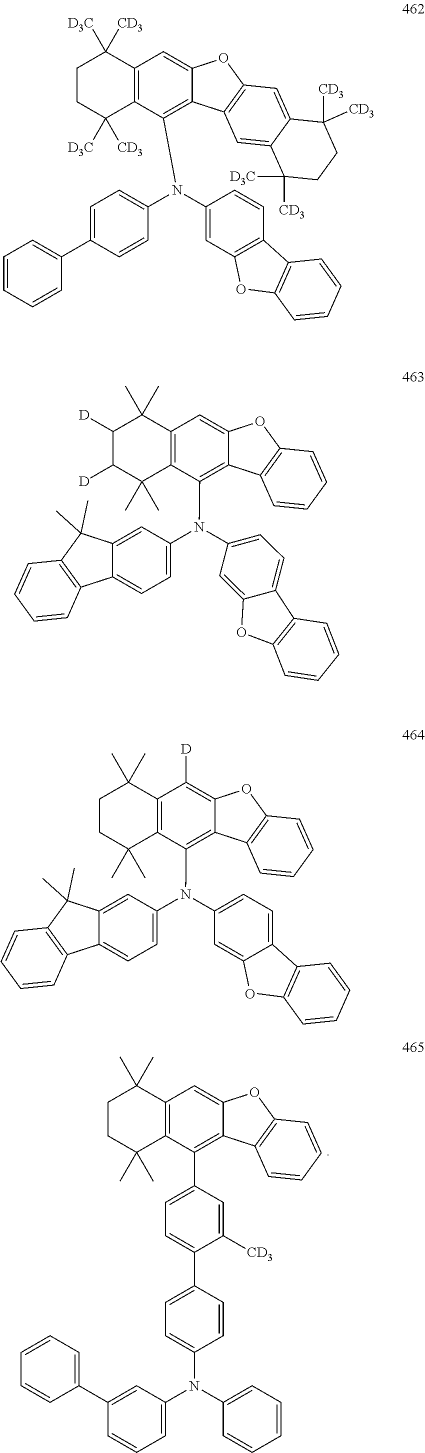

In a second aspect, the present disclosure provides an organic electroluminescent device comprising an anode, a cathode, and a functional layer disposed between the anode and the cathode; wherein the functional layer comprises the organic compound described in the first aspect of the present disclosure.

The compound provided in the present disclosure can be used to form at least one organic film layer in the functional layer, to improve characteristics such as luminous efficiency and lifetime of the organic electroluminescent device.

Optionally, the functional layer comprises a hole auxiliary layer, which contains the compound.

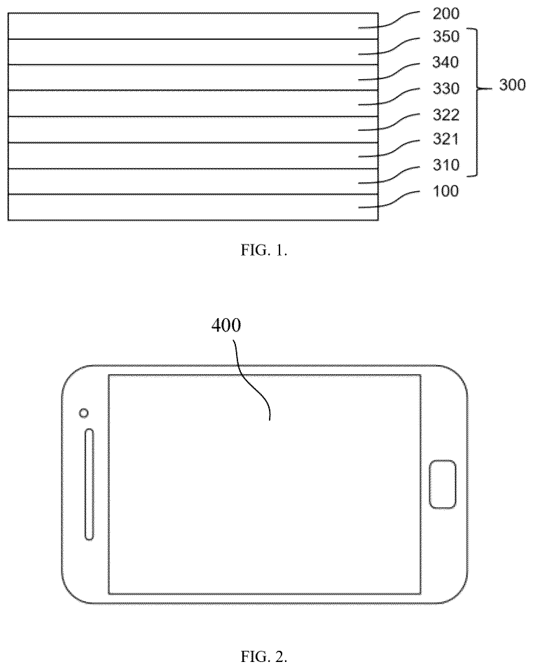

According to a specific embodiment, the organic electroluminescent device, as shown in FIG. 1, may include an anode 100, a hole injection layer 310, a first hole transport layer 321, a hole auxiliary layer 322, an organic light-emitting layer 330, an electron transport layer 340, an electron injection layer 350, and a cathode 200 sequentially stacked.

In the present disclosure, the anode 100 comprises an anode material, which is preferably a high-work function material contributing to injection of holes into the functional layer. Specific examples of the anode material include: metals such as nickel, platinum, vanadium, chromium, copper, zinc and gold, or alloys thereof; metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy)thiophene] (PEDT), polypyrrole, and polyaniline, but are not limited thereto. Preferably, a transparent electrode comprising indium tin oxide (ITO) is included as the anode.

In the present disclosure, the hole transport layer may include one or more hole transport material(s). The hole transport materials may be selected from carbazole multimers, carbazole-linked triarylamine compounds, and other types of compounds, and specifically may be selected from the following compound or any combination thereof:

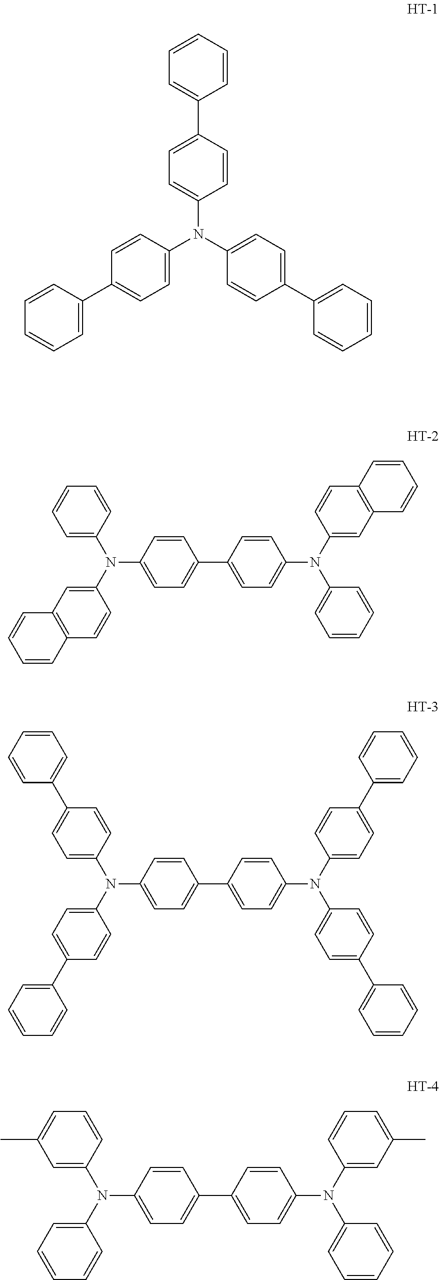

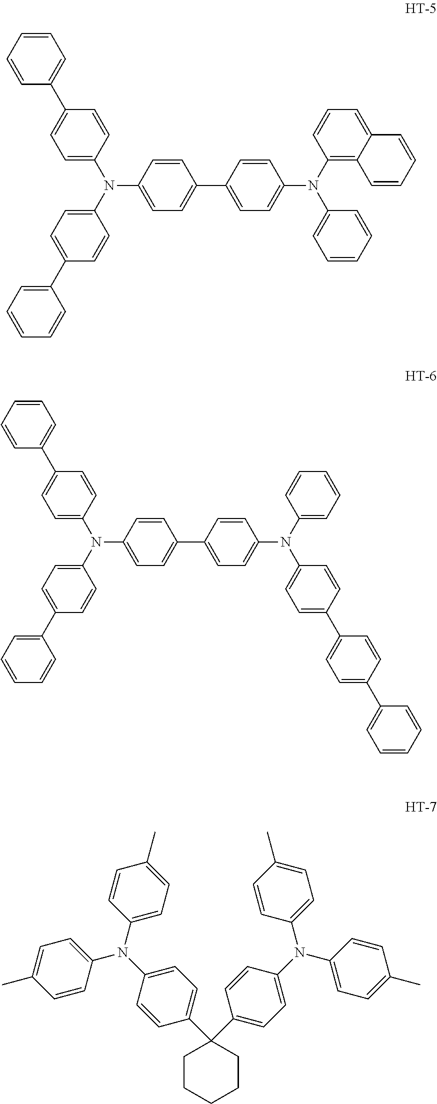

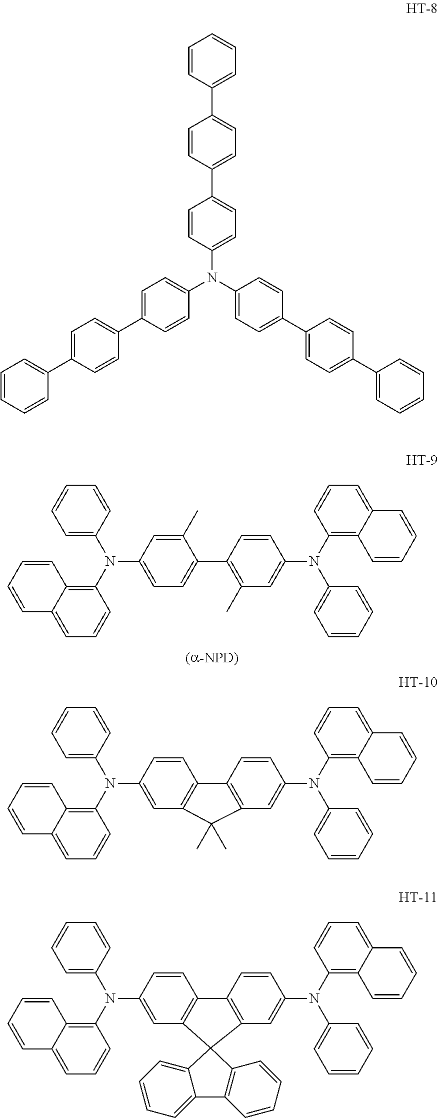 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; Cc1cc(N(c2ccccc2)c2cccc3ccccc23)ccc1-c1ccc(N(c2ccccc2)c2cccc3ccccc23)cc1C ; CC1(C)c2cc(N(c3ccccc3)c3cccc4ccccc34)ccc2-c2ccc(N(c3ccccc3)c3cccc4ccccc34)cc21 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

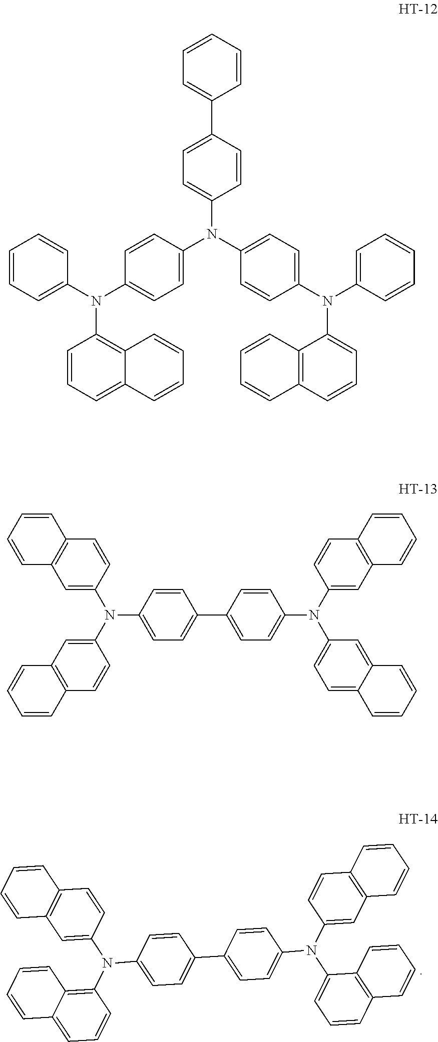 [SMILES: c1ccc(-c2ccc(N(c3ccc(N(c4ccccc4)c4cccc5ccccc45)cc3)c3ccc(N(c4ccccc4)c4cccc5ccccc45)cc3)cc2)cc1 ; c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1]

Those skilled in the art may make choices with reference to the prior art, and it is not particularly limited in the present disclosure.

In an embodiment, the first hole transport layer 321 is composed of HT-12.

In an embodiment, the hole auxiliary layer 322 is the compound of the present disclosure.

Optionally, a hole injection layer 310 may be further provided between the anode 100 and the first hole transport layer 321 to enhance the ability to inject holes into the first hole transport layer 321. The hole injection layer 310 may choose to use a benzidine derivative, a starburst arylamine-based compound, a phthalocyanine derivative or other materials. It is not particularly limited in the present disclosure. The material of the hole injection layer 310 is, for example, selected from the following compounds or any combination thereof;

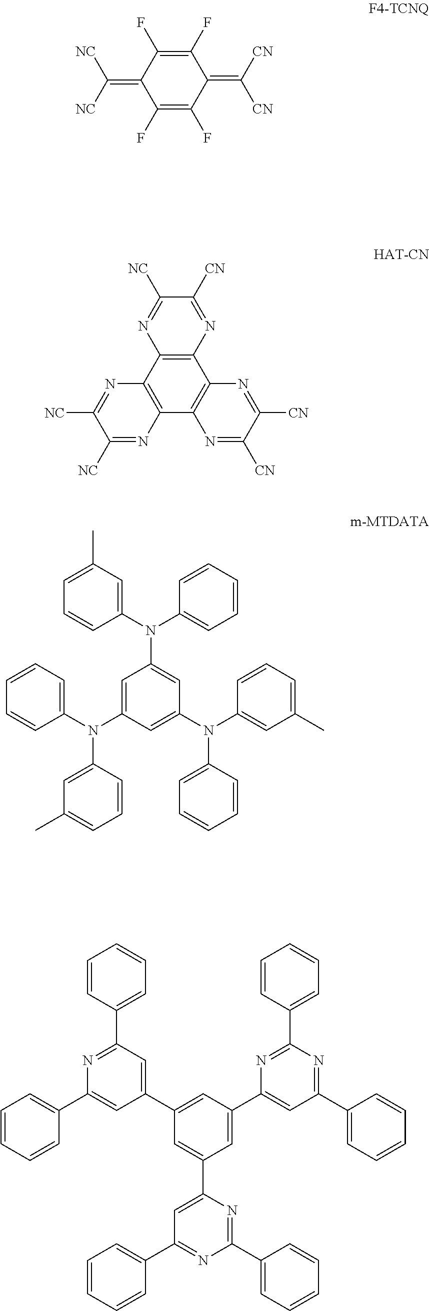 [SMILES: N#CC(C#N)=c1c(F)c(F)c(=C(C#N)C#N)c(F)c1F ; N#Cc1nc2c3nc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

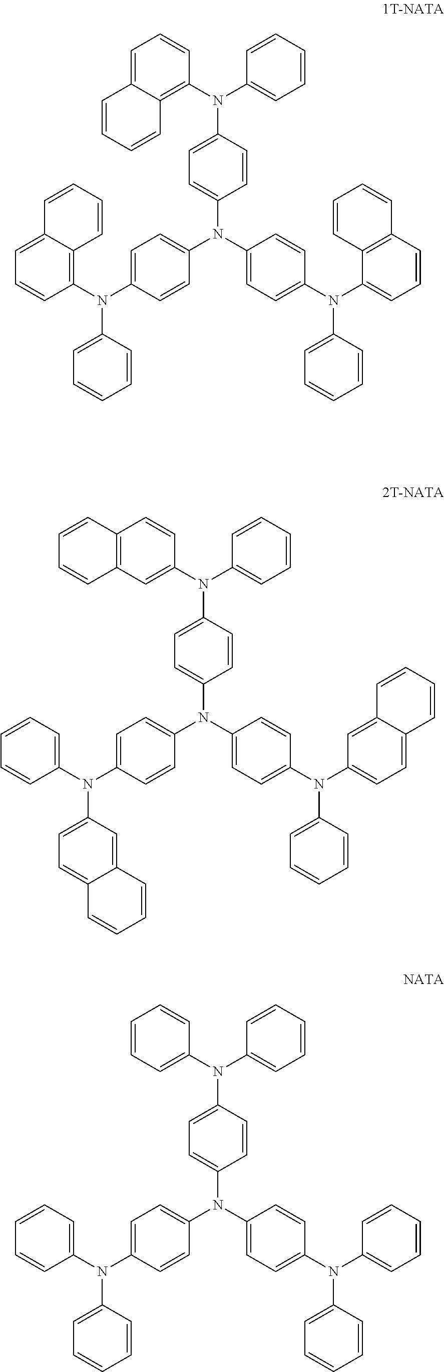 [SMILES: c1ccc(N(c2ccc(N(c3ccc(N(c4ccccc4)c4cccc5ccccc45)cc3)c3ccc(N(c4ccccc4)c4cccc5ccccc45)cc3)cc2)c2cccc3ccccc23)cc1 ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccccc2)c2ccc(N(c3ccc(N(c4ccccc4)c4ccccc4)cc3)c3ccc(N(c4ccccc4)c4ccccc4)cc3)cc2)cc1]

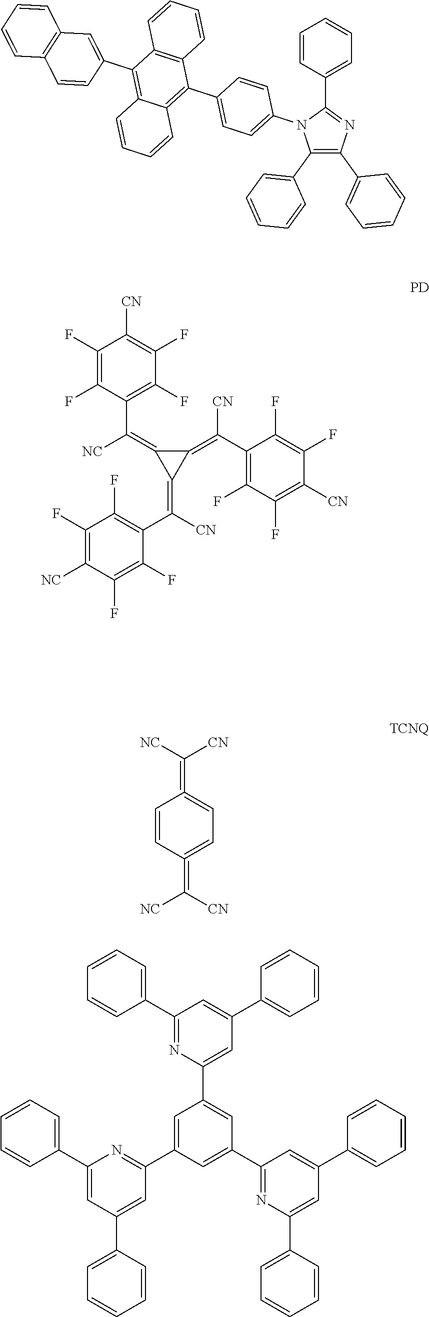

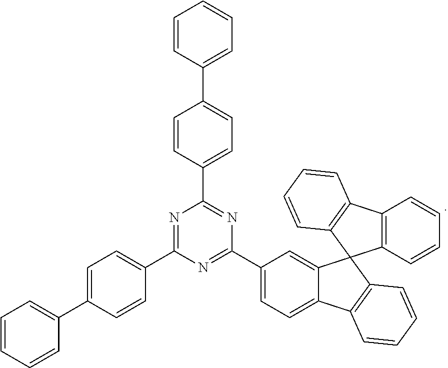 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In an embodiment, the hole injection layer 310 is composed of PD and HT-12.

In the present disclosure, the organic light-emitting layer 330 may be composed of a single light-emitting material or may include a host material and a dopant material. Optionally, the organic light-emitting layer 330 is composed of a host material and a dopant material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the dopant material, thereby enabling the dopant material to emit light.

The host material of the organic light-emitting layer 330 may contain a metal chelating compound, a stilbene-based derivative, an aromatic amine derivative, a dibenzofuran derivative, or other types of materials.

In some embodiments of the present disclosure, the host material of the organic light-emitting layer 330 is GH-01 and Compound GH-02.

The dopant material of the organic light-emitting layer 330 may be a compound having a condensed aryl ring or its derivative, a compound having a heteroaryl ring or its derivative, an aromatic amine derivative, or other materials. It is not particularly limited in the present disclosure. The dopant material is also known as a doping material or a dopant. Based on the type of luminescence, the dopant can be classified as fluorescent dopants and phosphorescent dopants. The specific examples of the phosphorescent dopant include but are not limited to,

[chemical structure]

In some embodiments of the present disclosure, the host material of the organic light-emitting layer 330 is GH-01 and Compound GH-02, and the dopant material is GD-01.

The electron transport layer 340 may be a single-layer structure or a multi-layer structure, which may comprise one or more electron transport material(s). The electron transport materials may be selected from but are not limited to, BTB, LiQ, benzimidazole derivatives, oxadiazole derivatives, quinoxaline derivatives, and other electron transport materials, and it is not particularly limited in the present disclosure. The material of the electron transport layer 340 includes but is not limited to the following compounds:

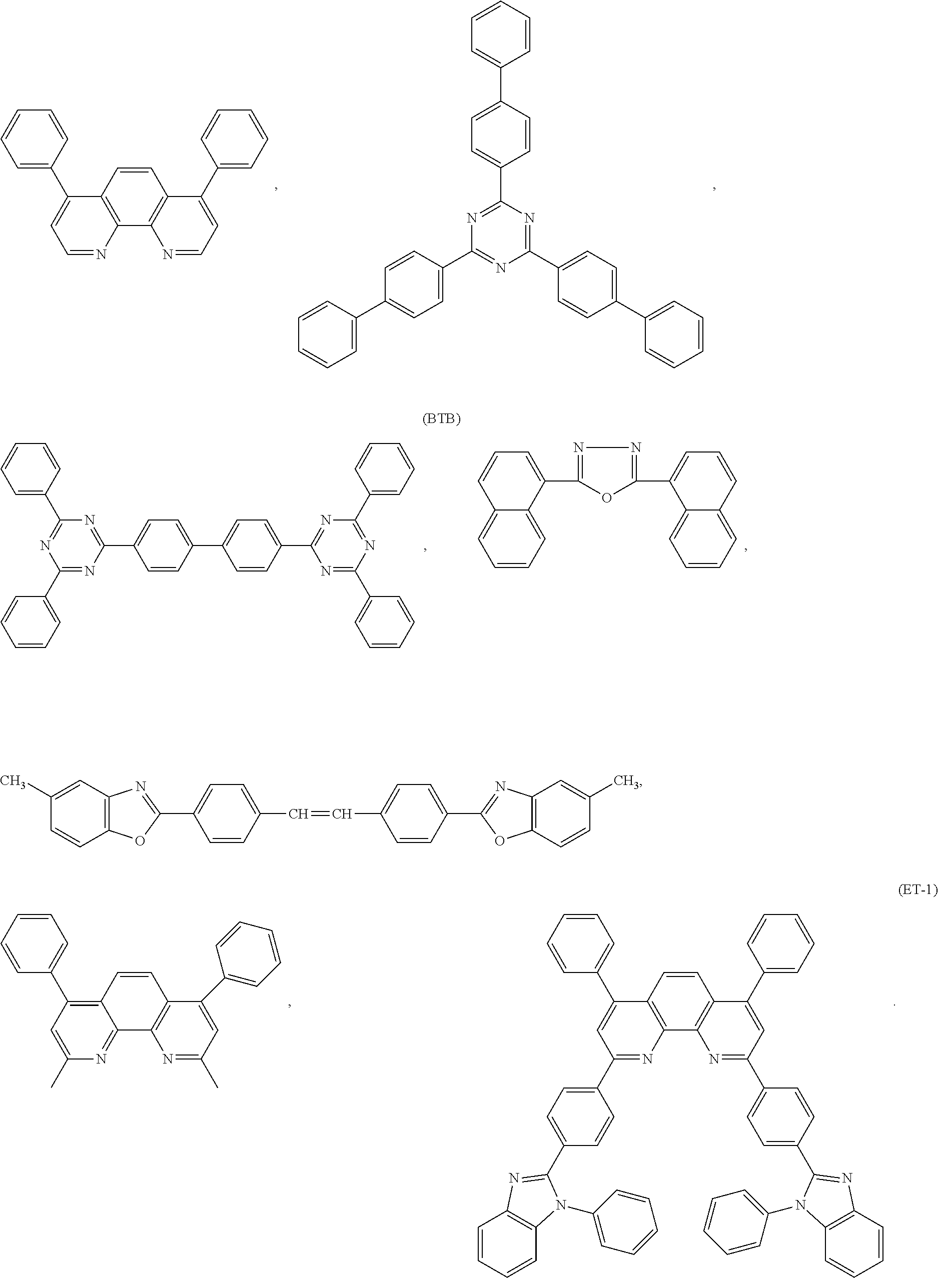

In an embodiment of the present disclosure, the electron transport layer 340 may be composed of BTB and LiQ, or be composed of ET-1 and LiQ.

In the present disclosure, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al, and BaF₂/Ca. Optionally, a metal electrode comprising magnesium and silver as the cathode is included.

Optionally, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide and an alkali metal halide or may comprise a complex of an alkali metal and an organic compound. In an embodiment of the present disclosure, the electron injection layer 350 comprises ytterbium (Yb).

In a third aspect, the present disclosure provides an electronic apparatus, comprising the organic electroluminescent device described in the second aspect of the present disclosure.

According to an embodiment, as shown in FIG. 2, the electronic apparatus provided is an electronic apparatus 400 comprising the above-described organic electroluminescent device. The electronic apparatus 400 may be a display apparatus, a lighting apparatus, an optical communication apparatus, or other type of electronic apparatus, examples of which may include, but are not limited to, computer screens, mobile phone screens, televisions, electronic paper, emergency lamps, optical modules, etc.

The synthesis method of the organic compound in the present disclosure will be demonstrated in detail with the following synthesis examples, but the present disclosure is not limited in any way by this.

### Synthesis Example

Professionals in their field should realize that the chemical reactions described in this present disclosure can be used to properly prepare many of the organic compounds in this present disclosure, and other methods used to prepare the compounds in this present disclosure are considered to be within the scope of this present disclosure. For example, according to the present disclosure, the synthesis of those non-exemplified compounds can be successfully completed by the technicians in the field through modification methods, such as appropriate protection of interfering groups, by using other known reagents in addition to the ones described in the present disclosure, or by making some conventional modifications to the reaction conditions. Compounds for which synthesis methods are not mentioned in the present disclosure can be obtained through commercial sources.

### Preparation of Intermediate

### 1. Preparation of Intermediate a

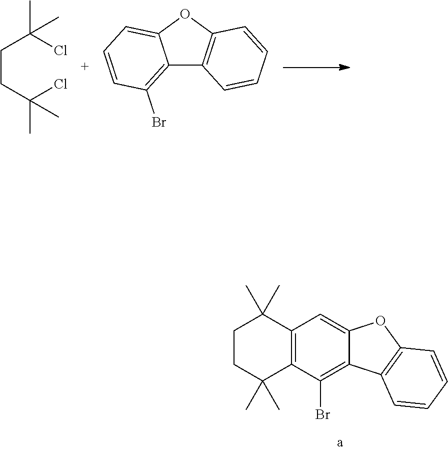

2,5-dichloro-2,5-dimethylhexane (70.0 g, 382.26 mmol), 1-bromodibenzofuran (57.03 g, 230.81 mmol), and 560 mL of dichloromethane (DCM) were added into a three-necked round-bottomed flask. The mixture was stirred and cooled to −60° C. under a nitrogen atmosphere. Anhydrous aluminum chloride (AlCl₃, 75.49 g, 498.78 mmol) was added in small amount over multiple times in batches. The reaction was immediately ceased upon completion of the addition of anhydrous aluminum chloride, and the reaction mixture was slowly poured into ice water to quench the reaction. Following extraction with DCM, the organic phase was separated after washing with water and was subsequently dried over anhydrous magnesium sulfate, followed by the removal of the solvent under reduced pressure. The crude product was subjected to recrystallization using petroleum ether, to obtain a solid Intermediate a (28.7 g, yield: 35%).

Following the same method as for Intermediate a, Intermediates al to a9 were synthesized respectively by replacing 1-bromodibenzofuran with corresponding reactant A in Table 1.

TABLE 1

| Intermediate | Reactant A | Intermediate Structure | Yield (%) |
|---|---|---|---|
| Intermediate a1 | 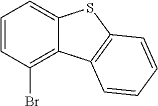 [SMILES: Brc1cccc2sc3ccccc3c12] | 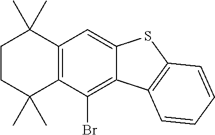 [SMILES: CC1(C)CCC(C)(C)c2c1cc1sc3ccccc3c1c2Br] | 40 |
| Intermediate a2 | 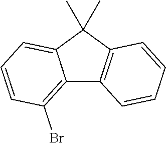 [SMILES: CC1(C)c2ccccc2-c2c(Br)cccc21] | 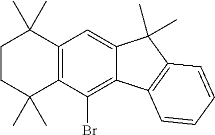 [SMILES: CC1(C)CCC(C)(C)c2c1cc1c(c2Br)-c2ccccc2C1(C)C] | 35 |
| Intermediate a3 | 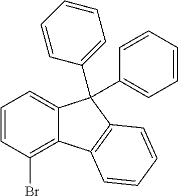 [SMILES: Brc1cccc2c1-c1ccccc1C2(c1ccccc1)c1ccccc1] | 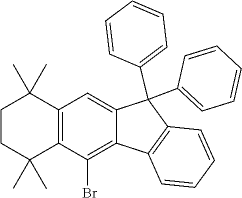 [SMILES: CC1(C)CCC(C)(C)c2c1cc1c(c2Br)-c2ccccc2C1(c1ccccc1)c1ccccc1] | 41 |
| Intermediate a4 | 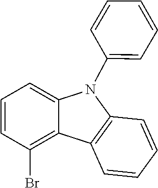 [SMILES: Brc1cccc2c1c1ccccc1n2-c1ccccc1] | 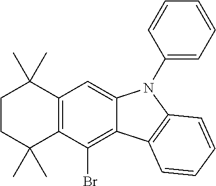 [SMILES: CC1(C)CCC(C)(C)c2c1cc1c(c2Br)c2ccccc2n1-c1ccccc1] | 36 |
| Intermediate a5 | 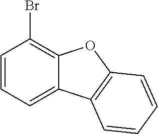 [SMILES: Brc1cccc2c1oc1ccccc12] | 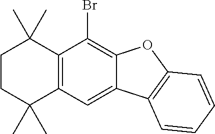 [SMILES: CC1(C)CCC(C)(C)c2c1cc1c(oc3ccccc31)c2Br] | 38 |
| Intermediate a6 | 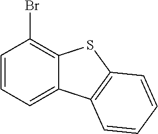 [SMILES: Brc1cccc2c1sc1ccccc12] | 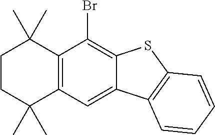 [SMILES: CC1(C)CCC(C)(C)c2c1cc1c(sc3ccccc31)c2Br] | 50 |
| Intermediate a7 | 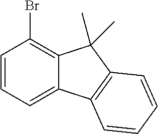 [SMILES: CC1(C)c2ccccc2-c2cccc(Br)c21] | 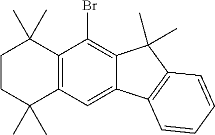 [SMILES: CC1(C)CCC(C)(C)c2c1cc1c(c2Br)C(C)(C)c2ccccc2-1] | 67 |
| Intermediate a8 | 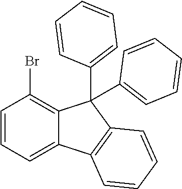 [SMILES: Brc1cccc2c1C(c1ccccc1)(c1ccccc1)c1ccccc1-2] | 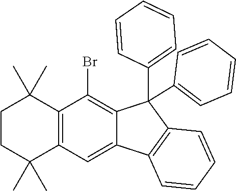 [SMILES: CC1(C)CCC(C)(C)c2c1cc1c(c2Br)C(c2ccccc2)(c2ccccc2)c2ccccc2-1] | 38 |
| Intermediate a9 | 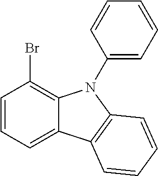 [SMILES: Brc1cccc2c3ccccc3n(-c3ccccc3)c12] | 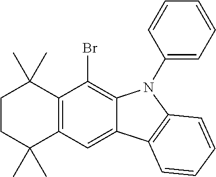 [SMILES: CC1(C)CCC(C)(C)c2c1cc1c3ccccc3n(-c3ccccc3)c1c2Br] | 49 |

### Synthesis of Intermediate b

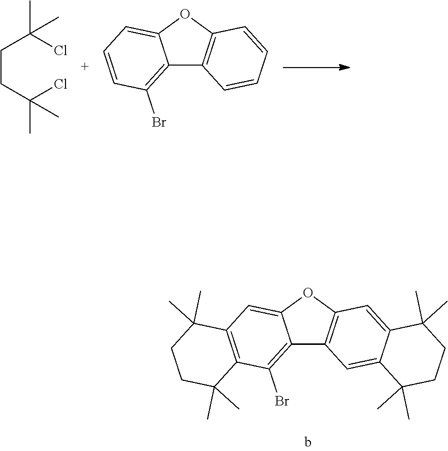

2,5-dichloro-2,5-dimethylhexane (70.0 g, 382.26 mmol), 1-bromodibenzofuran (46.67 g, 188.86 mmol), and 560 mL of dichloromethane (DCM) were added into a three-necked round-bottomed flask. The mixture was stirred and cooled to −60° C. under a nitrogen atmosphere. Anhydrous aluminum chloride (100.73 g, 755.44 mmol) was added in small amount over multiple times in batches. The reaction was immediately ceased upon completion of the addition of anhydrous aluminum chloride, and the reaction mixture was slowly poured into ice water to quench the reaction. The reaction mixture was extracted with DCM and washed with water, the organic phase was separated and was dried over anhydrous magnesium sulfate, followed by the removal of the solvent under reduced pressure; the crude product was recrystallized using petroleum ether, to obtain a solid Intermediate b (56 g, yield: 63%).

Following the same method as for Intermediate b, Intermediates b1 to b9 were synthesized respectively by replacing 1-bromodibenzofuran with corresponding reactant B in Table 2.

TABLE 2

| Intermediate | Reactant B | Intermediate Structure | Yield (%) |
|---|---|---|---|
| Intermediate b1 | 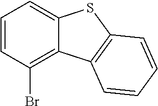 [SMILES: Brc1cccc2sc3ccccc3c12] | 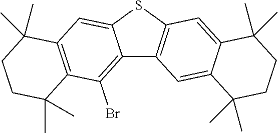 | 46 |
| Intermediate b2 | 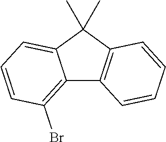 [SMILES: CC1(C)c2ccccc2-c2c(Br)cccc21] | 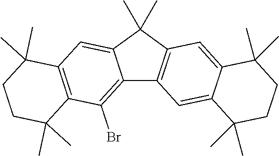 | 52 |
| Intermediate b3 | 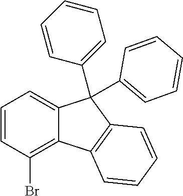 [SMILES: Brc1cccc2c1-c1ccccc1C2(c1ccccc1)c1ccccc1] | 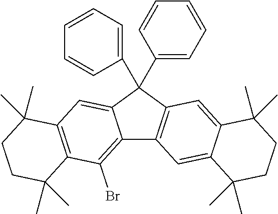 | 49 |
| Intermediate b4 | 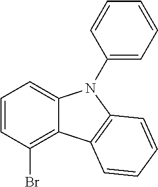 [SMILES: Brc1cccc2c1c1ccccc1n2-c1ccccc1] | 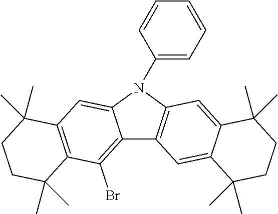 | 47 |
| Intermediate b5 | 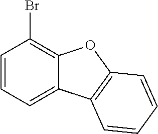 [SMILES: Brc1cccc2c1oc1ccccc12] | 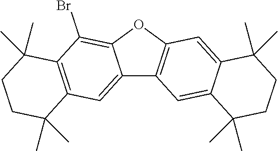 | 36 |
| Intermediate b6 | 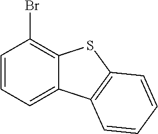 [SMILES: Brc1cccc2c1sc1ccccc12] | 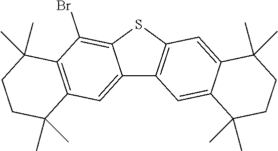 | 51 |
| Intermediate b7 | 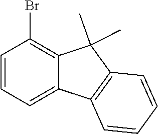 [SMILES: CC1(C)c2ccccc2-c2cccc(Br)c21] | 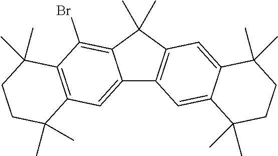 | 39 |
| Intermediate b8 | 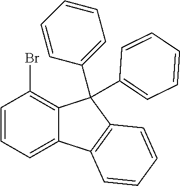 [SMILES: Brc1cccc2c1C(c1ccccc1)(c1ccccc1)c1ccccc1-2] | 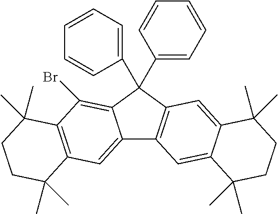 | 45 |
| Intermediate b9 | 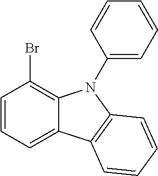 [SMILES: Brc1cccc2c3ccccc3n(-c3ccccc3)c12] | 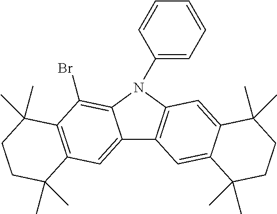 | 40 |

### Synthesis of Intermediate c

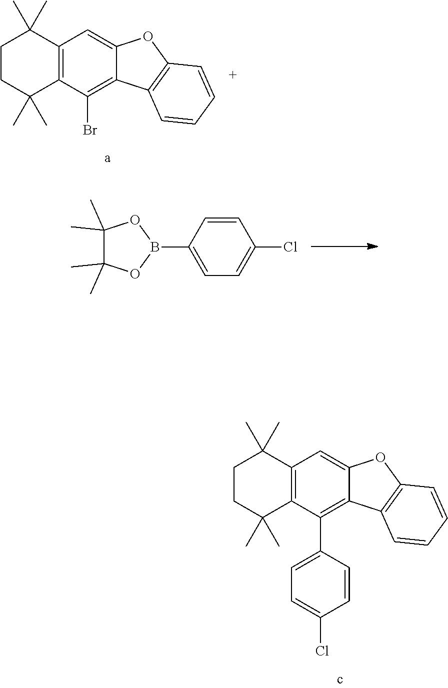 [SMILES: CC1(C)CCC(C)(C)c2c1cc1oc3ccccc3c1c2Br ; CC1(C)OB(c2ccc(Cl)cc2)OC1(C)C ; CC1(C)CCC(C)(C)c2c1cc1oc3ccccc3c1c2-c1ccc(Cl)cc1]

The Intermediate a (10.0 g, 27.99 mmol), p-chlorophenylboronic acid (6.81 g, 28.55 mmol), tetrakistriphenylphosphine palladium (Pd(PPh₃)₄, 0.6 g, 0.5 mmol), potassium carbonate (K₂CO₃, 7.0 g, 50.4 mmol), tetrabutylammonium bromide (TBAB, 1.6 g, 50 mmol), toluene (PhMe, 80 mL), ethanol (EtOH, 20 mL), and deionized water (H₂O, 20 mL) were added into a round-bottomed flask. The mixture was stirred and heated to 75° C. to 80° C. under a nitrogen atmosphere for 12 hours of reaction. The reaction mixture was cooled to room temperature, washed with water, and the organic phase was separated and dried over anhydrous magnesium sulfate, and the solvent was removed under reduced pressure. The crude product was recrystallized using dichloromethane/n-heptane to obtain Intermediate c as a white solid (7.8 g, yield: 72%).

Following the same method as that for Intermediate c, Intermediates c1 to c4 were synthesized respectively by replacing p-chlorophenylboronic acid with corresponding reactants C in Table 3.

TABLE 3

| Intermediate | Reactant C | Intermediate Structure | Yield (%) |
|---|---|---|---|
| Intermediate c1 | 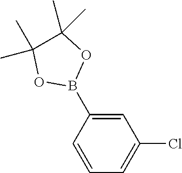 [SMILES: CC1(C)OB(c2cccc(Cl)c2)OC1(C)C] | 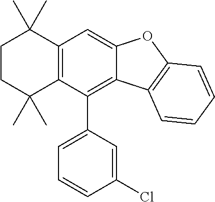 [SMILES: CC1(C)CCC(C)(C)c2c1cc1oc3ccccc3c1c2-c1cccc(Cl)c1] | 68 |
| Intermediate c2 | 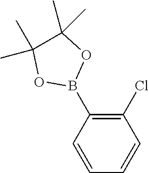 [SMILES: CC1(C)OB(c2ccccc2Cl)OC1(C)C] | 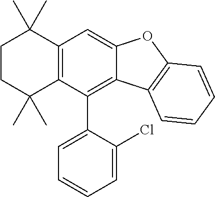 [SMILES: CC1(C)CCC(C)(C)c2c1cc1oc3ccccc3c1c2-c1ccccc1Cl] | 59 |
| Intermediate c3 | 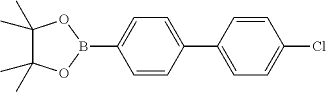 [SMILES: CC1(C)OB(c2ccc(-c3ccc(Cl)cc3)cc2)OC1(C)C] | 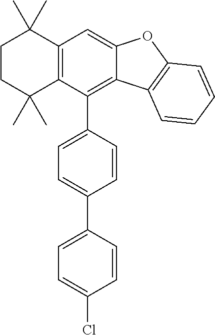 [SMILES: CC1(C)CCC(C)(C)c2c1cc1oc3ccccc3c1c2-c1ccc(-c2ccc(Cl)cc2)cc1] | 57 |
| Intermediate c4 | 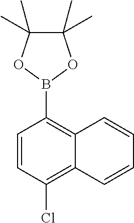 [SMILES: CC1(C)OB(c2ccc(Cl)c3ccccc23)OC1(C)C] | 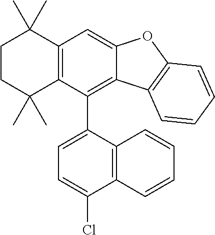 [SMILES: CC1(C)CCC(C)(C)c2c1cc1oc3ccccc3c1c2-c1ccc(Cl)c2ccccc12] | 42 |

### Synthesis of Compound 2

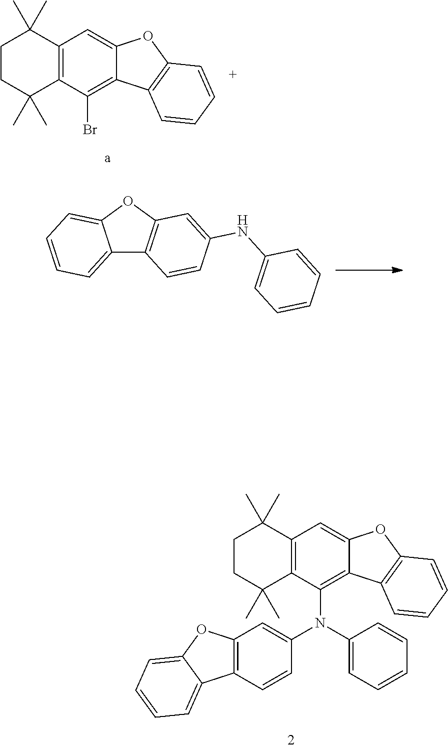 [SMILES: CC1(C)CCC(C)(C)c2c1cc1oc3ccccc3c1c2Br ; c1ccc(Nc2ccc3c(c2)oc2ccccc23)cc1 ; CC1(C)CCC(C)(C)c2c1cc1oc3ccccc3c1c2N(c1ccccc1)c1ccc2c(c1)oc1ccccc12]

The Intermediate a (4.0 g, 11.2 mmol), N-phenyl-3-dibenzofuran-2-amine (2.9 g, 11.2 mmol) (CAS RN: 406488-21-9), tris(dibenzylidene acetone) dipalladium (Pd₂(dba)₃, 0.2 g, 0.3 mmol), 2-dicyclohexylphosphino-2,6-dimethoxybiphenyl (SPhos, 0.2 g, 0.5 mmol), sodium tert-butoxide (tBuONa, 1.9 g, 19.7 mmol), and toluene (PhMe, 50 mL) were added into a nitrogen-protected round-bottomed flask. The mixture was heated to 105° C. to 110° C. under stirring and reacted for 16 hours; the reaction solution was cooled down to room temperature and the organic phase was separated after washing with water and dried over anhydrous magnesium sulfate, followed by the removal of the solvent under reduced pressure; the resulting crude product was purified by silica gel column chromatography using dichloromethane/n-heptane, followed by recrystallization using dichloromethane/n-heptane, to obtain Compound 2 as a white solid (3.6 g, yield: 60%). Mass Spectra m/z=536.25 ([M+H]⁺).

Following the synthesis method of Compound 2, the corresponding Intermediate X in Table 4 was used instead of Intermediate a, and the corresponding Reactant D in Table 4 was used instead of N-phenyl-3-dibenzofuran-2-amine, to respectively synthesize the compounds of the present disclosure listed in Table 4.

TABLE 4

| | | | | | Mass |
|---|---|---|---|---|---|
| | | | | | Spectra |
| | | | | Yield | m/z |
| Compound | Intermediate X | Reactant D | Compound structure | (%) | ([M + H]⁺) |
| 8 | 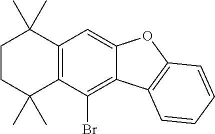 [SMILES: CC1(C)CCC(C)(C)c2c1cc1oc3ccccc3c1c2Br] | 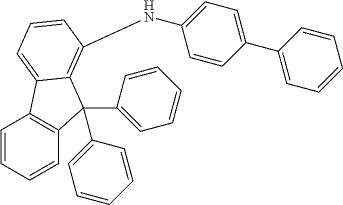 [SMILES: c1ccc(-c2ccc(Nc3cccc4c3C(c3ccccc3)(c3ccccc3)c3ccccc3-4)cc2)cc1] | 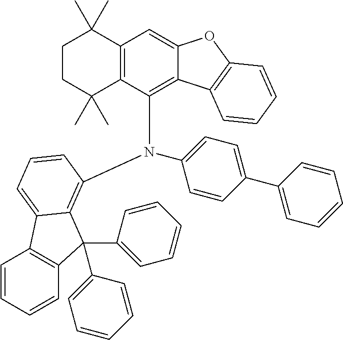 [SMILES: CC1(C)CCC(C)(C)c2c1cc1oc3ccccc3c1c2N(c1ccc(-c2ccccc2)cc1)c1cccc2c1C(c1ccccc1)(c1ccccc1)c1ccccc1-2] | 59 | 762.37 |
| 16 | 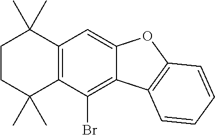 [SMILES: CC1(C)CCC(C)(C)c2c1cc1oc3ccccc3c1c2Br] | 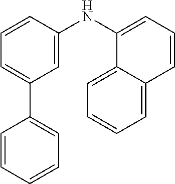 [SMILES: c1ccc(-c2cccc(Nc3cccc4ccccc34)c2)cc1] | 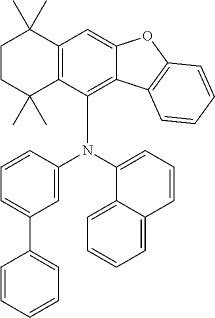 [SMILES: CC1(C)CCC(C)(C)c2c1cc1oc3ccccc3c1c2N(c1cccc(-c2ccccc2)c1)c1cccc2ccccc12] | 48 | 572.29 |
| 17 | 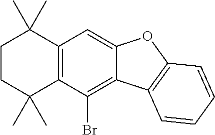 [SMILES: CC1(C)CCC(C)(C)c2c1cc1oc3ccccc3c1c2Br] | 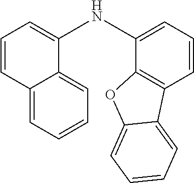 [SMILES: c1ccc2c(Nc3cccc4c3oc3ccccc34)cccc2c1] | 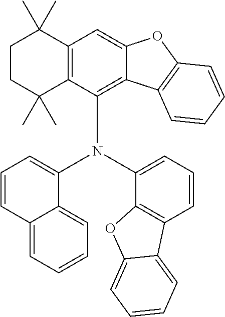 [SMILES: CC1(C)CCC(C)(C)c2c1cc1oc3ccccc3c1c2N(c1cccc2ccccc12)c1cccc2c1oc1ccccc12] | 67 | 586.27 |
| 21 | 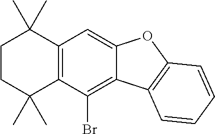 [SMILES: CC1(C)CCC(C)(C)c2c1cc1oc3ccccc3c1c2Br] | 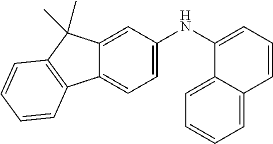 [SMILES: CC1(C)c2ccccc2-c2ccc(Nc3cccc4ccccc34)cc21] | 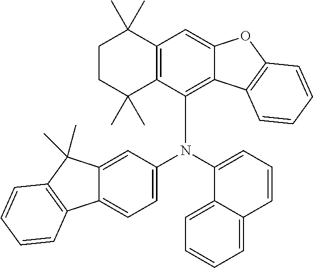 [SMILES: CC1(C)CCC(C)(C)c2c1cc1oc3ccccc3c1c2N(c1ccc2c(c1)C(C)(C)c1ccccc1-2)c1cccc2ccccc12] | 53 | 612.32 |
| 24 | 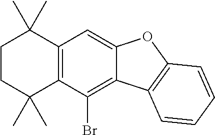 [SMILES: CC1(C)CCC(C)(C)c2c1cc1oc3ccccc3c1c2Br] | 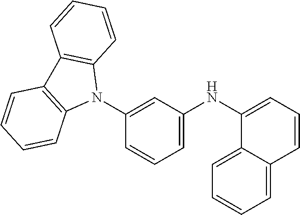 [SMILES: c1cc(Nc2cccc3ccccc23)cc(-n2c3ccccc3c3ccccc32)c1] | 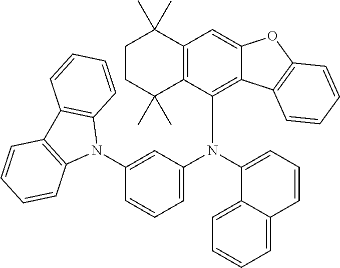 [SMILES: CC1(C)CCC(C)(C)c2c1cc1oc3ccccc3c1c2N(c1cccc(-n2c3ccccc3c3ccccc32)c1)c1cccc2ccccc12] | 46 | 661.32 |
| 35 | 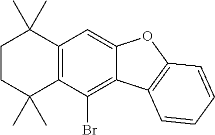 [SMILES: CC1(C)CCC(C)(C)c2c1cc1oc3ccccc3c1c2Br] | 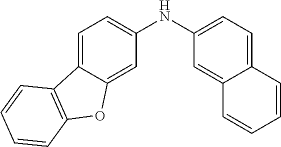 [SMILES: c1ccc2cc(Nc3ccc4c(c3)oc3ccccc34)ccc2c1] | 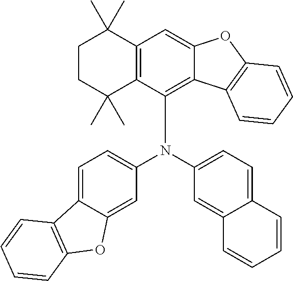 [SMILES: CC1(C)CCC(C)(C)c2c1cc1oc3ccccc3c1c2N(c1ccc2ccccc2c1)c1ccc2c(c1)oc1ccccc12] | 58 | 586.27 |
| 48 | 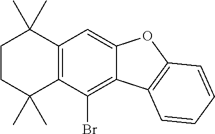 [SMILES: CC1(C)CCC(C)(C)c2c1cc1oc3ccccc3c1c2Br] | 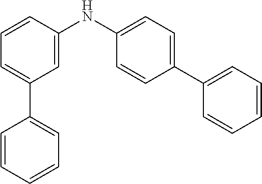 [SMILES: c1ccc(-c2ccc(Nc3cccc(-c4ccccc4)c3)cc2)cc1] | 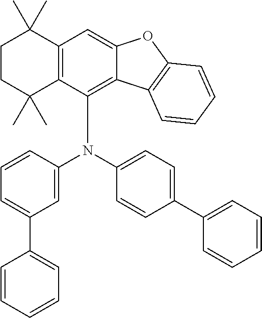 [SMILES: CC1(C)CCC(C)(C)c2c1cc1oc3ccccc3c1c2N(c1ccc(-c2ccccc2)cc1)c1cccc(-c2ccccc2)c1] | 59 | 598.31 |
| 52 | 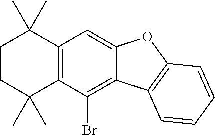 [SMILES: CC1(C)CCC(C)(C)c2c1cc1oc3ccccc3c1c2Br] | 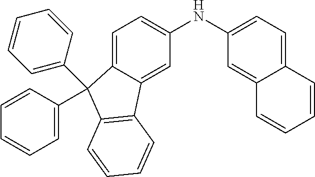 [SMILES: c1ccc(C2(c3ccccc3)c3ccccc3-c3cc(Nc4ccc5ccccc5c4)ccc32)cc1] | 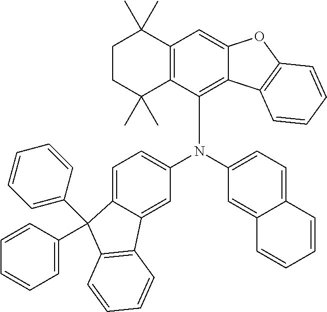 [SMILES: CC1(C)CCC(C)(C)c2c1cc1oc3ccccc3c1c2N(c1ccc2c(c1)-c1ccccc1C2(c1ccccc1)c1ccccc1)c1ccc2ccccc2c1] | 60 | 736.35 |
| 54 | 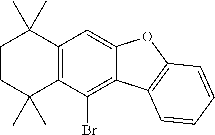 [SMILES: CC1(C)CCC(C)(C)c2c1cc1oc3ccccc3c1c2Br] | 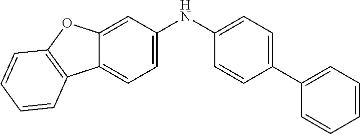 [SMILES: c1ccc(-c2ccc(Nc3ccc4c(c3)oc3ccccc34)cc2)cc1] | 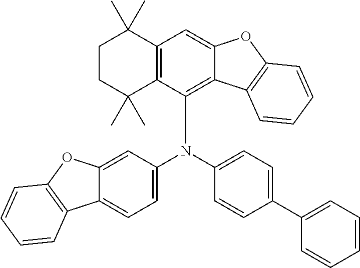 [SMILES: CC1(C)CCC(C)(C)c2c1cc1oc3ccccc3c1c2N(c1ccc(-c2ccccc2)cc1)c1ccc2c(c1)oc1ccccc12] | 63 | 612.29 |
| 62 | 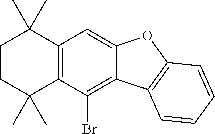 [SMILES: CC1(C)CCC(C)(C)c2c1cc1oc3ccccc3c1c2Br] | 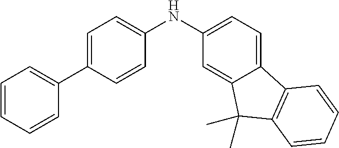 [SMILES: CC1(C)c2ccccc2-c2ccc(Nc3ccc(-c4ccccc4)cc3)cc21] | 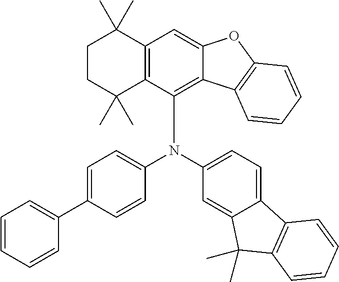 [SMILES: CC1(C)CCC(C)(C)c2c1cc1oc3ccccc3c1c2N(c1ccc(-c2ccccc2)cc1)c1ccc2c(c1)C(C)(C)c1ccccc1-2] | 59 | 638.34 |
| 71 | 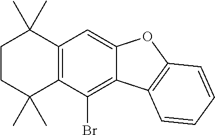 [SMILES: CC1(C)CCC(C)(C)c2c1cc1oc3ccccc3c1c2Br] | 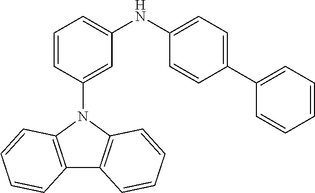 [SMILES: c1ccc(-c2ccc(Nc3cccc(-n4c5ccccc5c5ccccc54)c3)cc2)cc1] | 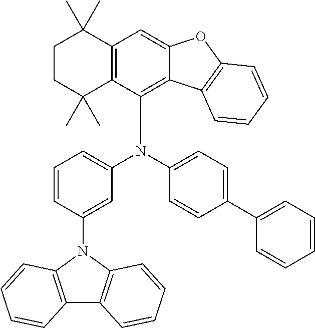 [SMILES: CC1(C)CCC(C)(C)c2c1cc1oc3ccccc3c1c2N(c1ccc(-c2ccccc2)cc1)c1cccc(-n2c3ccccc3c3ccccc32)c1] | 46 | 687.33 |
| 80 | 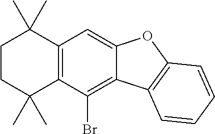 [SMILES: CC1(C)CCC(C)(C)c2c1cc1oc3ccccc3c1c2Br] | 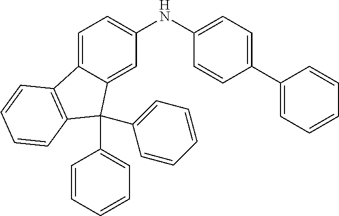 [SMILES: c1ccc(-c2ccc(Nc3ccc4c(c3)C(c3ccccc3)(c3ccccc3)c3ccccc3-4)cc2)cc1] | 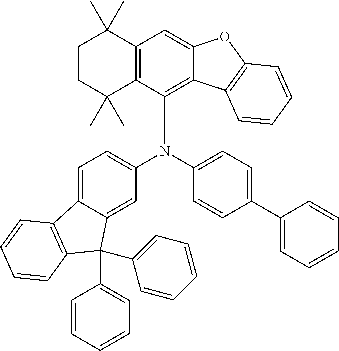 [SMILES: CC1(C)CCC(C)(C)c2c1cc1oc3ccccc3c1c2N(c1ccc(-c2ccccc2)cc1)c1ccc2c(c1)C(c1ccccc1)(c1ccccc1)c1ccccc1-2] | 43 | 762.37 |
| 108 | 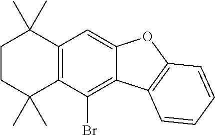 [SMILES: CC1(C)CCC(C)(C)c2c1cc1oc3ccccc3c1c2Br] | 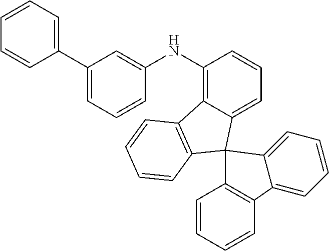 [SMILES: c1ccc(-c2cccc(Nc3cccc4c3-c3ccccc3C43c4ccccc4-c4ccccc43)c2)cc1] | 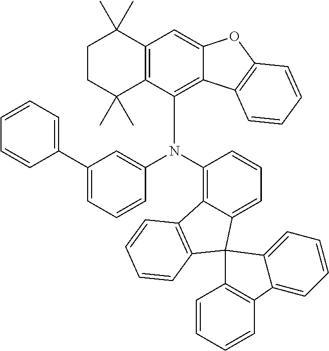 [SMILES: CC1(C)CCC(C)(C)c2c1cc1oc3ccccc3c1c2N(c1cccc(-c2ccccc2)c1)c1cccc2c1-c1ccccc1C21c2ccccc2-c2ccccc21] | 46 | 760.35 |
| 125 | 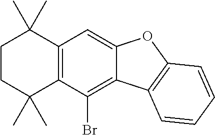 [SMILES: CC1(C)CCC(C)(C)c2c1cc1oc3ccccc3c1c2Br] | 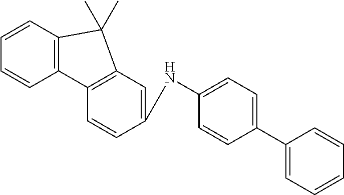 [SMILES: CC1(C)c2ccccc2-c2ccc(Nc3ccc(-c4ccccc4)cc3)cc21] | 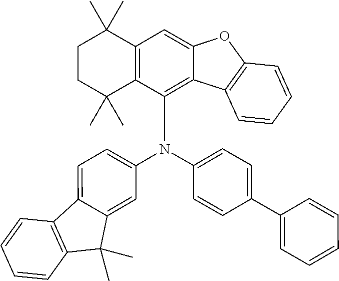 [SMILES: CC1(C)CCC(C)(C)c2c1cc1oc3ccccc3c1c2N(c1ccc(-c2ccccc2)cc1)c1ccc2c(c1)C(C)(C)c1ccccc1-2] | 53 | 638.34 |
| 145 | 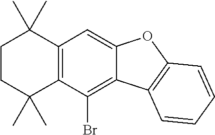 [SMILES: CC1(C)CCC(C)(C)c2c1cc1oc3ccccc3c1c2Br] | 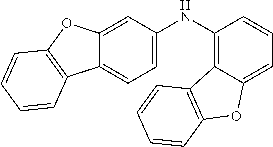 [SMILES: c1ccc2c(c1)oc1cc(Nc3cccc4oc5ccccc5c34)ccc12] | 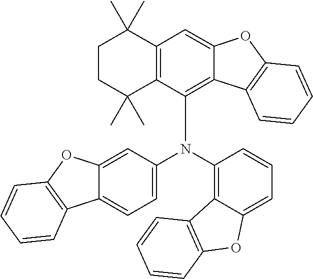 [SMILES: CC1(C)CCC(C)(C)c2c1cc1oc3ccccc3c1c2N(c1ccc2c(c1)oc1ccccc12)c1cccc2oc3ccccc3c12] | 55 | 626.27 |
| 152 | 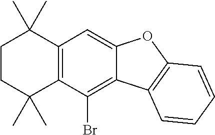 [SMILES: CC1(C)CCC(C)(C)c2c1cc1oc3ccccc3c1c2Br] | 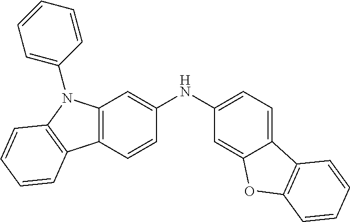 [SMILES: c1ccc(-n2c3ccccc3c3ccc(Nc4ccc5c(c4)oc4ccccc45)cc32)cc1] | 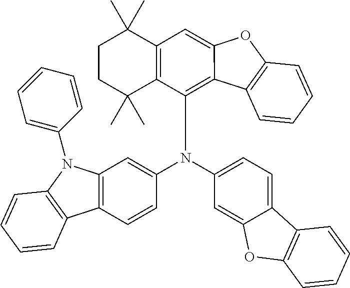 [SMILES: CC1(C)CCC(C)(C)c2c1cc1oc3ccccc3c1c2N(c1ccc2c(c1)oc1ccccc12)c1ccc2c3ccccc3n(-c3ccccc3)c2c1] | 61 | 701.31 |
| 161 | 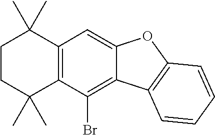 [SMILES: CC1(C)CCC(C)(C)c2c1cc1oc3ccccc3c1c2Br] | 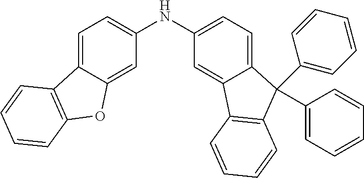 [SMILES: c1ccc(C2(c3ccccc3)c3ccccc3-c3cc(Nc4ccc5c(c4)oc4ccccc45)ccc32)cc1] | 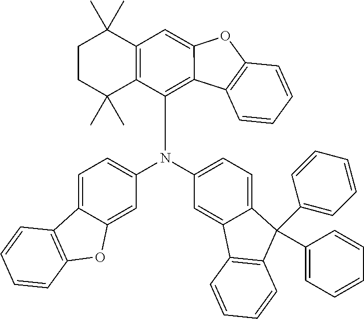 [SMILES: CC1(C)CCC(C)(C)c2c1cc1oc3ccccc3c1c2N(c1ccc2c(c1)-c1ccccc1C2(c1ccccc1)c1ccccc1)c1ccc2c(c1)oc1ccccc12] | 42 | 776.35 |
| 169 | 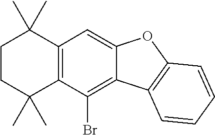 [SMILES: CC1(C)CCC(C)(C)c2c1cc1oc3ccccc3c1c2Br] | 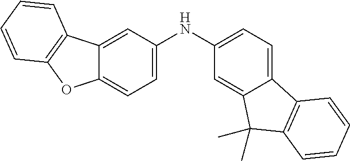 [SMILES: CC1(C)c2ccccc2-c2ccc(Nc3ccc4oc5ccccc5c4c3)cc21] | 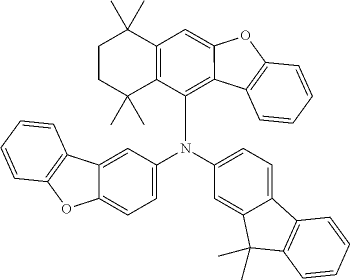 [SMILES: CC1(C)CCC(C)(C)c2c1cc1oc3ccccc3c1c2N(c1ccc2c(c1)C(C)(C)c1ccccc1-2)c1ccc2oc3ccccc3c2c1] | 56 | 652.32 |
| 178 | 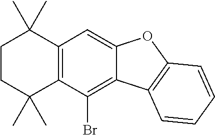 [SMILES: CC1(C)CCC(C)(C)c2c1cc1oc3ccccc3c1c2Br] | 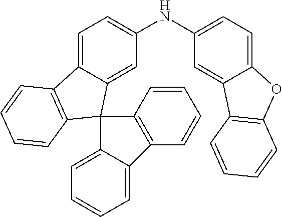 [SMILES: c1ccc2c(c1)-c1ccccc1C21c2ccccc2-c2ccc(Nc3ccc4oc5ccccc5c4c3)cc21] | 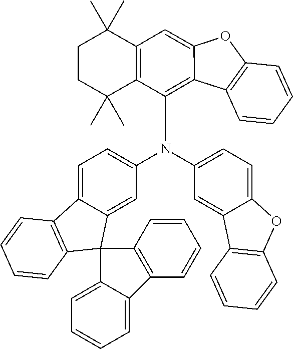 [SMILES: CC1(C)CCC(C)(C)c2c1cc1oc3ccccc3c1c2N(c1ccc2c(c1)C1(c3ccccc3-c3ccccc31)c1ccccc1-2)c1ccc2oc3ccccc3c2c1] | 59 | 774.33 |
| 193 | 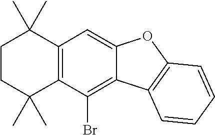 [SMILES: CC1(C)CCC(C)(C)c2c1cc1oc3ccccc3c1c2Br] | 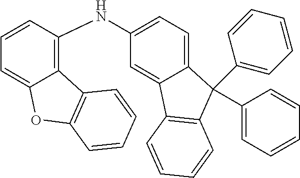 [SMILES: c1ccc(C2(c3ccccc3)c3ccccc3-c3cc(Nc4cccc5oc6ccccc6c45)ccc32)cc1] | 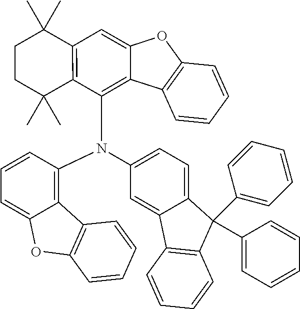 [SMILES: CC1(C)CCC(C)(C)c2c1cc1oc3ccccc3c1c2N(c1ccc2c(c1)-c1ccccc1C2(c1ccccc1)c1ccccc1)c1cccc2oc3ccccc3c12] | 43 | 776.35 |
| 198 | 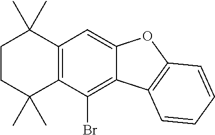 [SMILES: CC1(C)CCC(C)(C)c2c1cc1oc3ccccc3c1c2Br] | 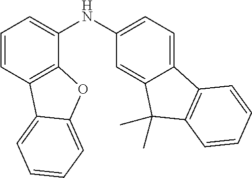 [SMILES: CC1(C)c2ccccc2-c2ccc(Nc3cccc4c3oc3ccccc34)cc21] | 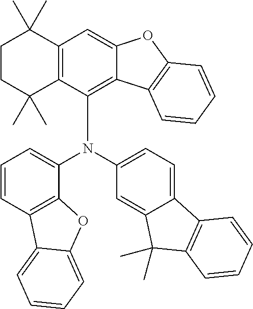 [SMILES: CC1(C)CCC(C)(C)c2c1cc1oc3ccccc3c1c2N(c1ccc2c(c1)C(C)(C)c1ccccc1-2)c1cccc2c1oc1ccccc12] | 66 | 652.32 |
| 204 | 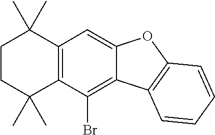 [SMILES: CC1(C)CCC(C)(C)c2c1cc1oc3ccccc3c1c2Br] | 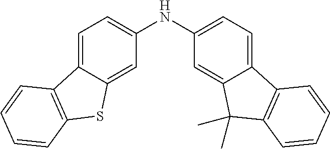 [SMILES: CC1(C)c2ccccc2-c2ccc(Nc3ccc4c(c3)sc3ccccc34)cc21] | 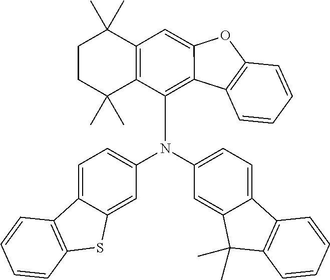 [SMILES: CC1(C)CCC(C)(C)c2c1cc1oc3ccccc3c1c2N(c1ccc2c(c1)C(C)(C)c1ccccc1-2)c1ccc2c(c1)sc1ccccc12] | 40 | 668.29 |
| 208 | 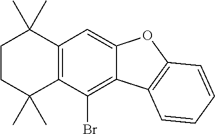 [SMILES: CC1(C)CCC(C)(C)c2c1cc1oc3ccccc3c1c2Br] | 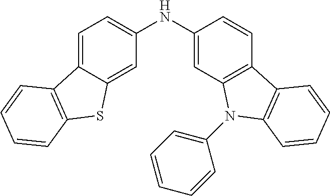 [SMILES: c1ccc(-n2c3ccccc3c3ccc(Nc4ccc5c(c4)sc4ccccc45)cc32)cc1] | 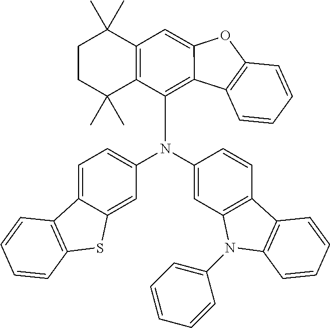 [SMILES: CC1(C)CCC(C)(C)c2c1cc1oc3ccccc3c1c2N(c1ccc2c(c1)sc1ccccc12)c1ccc2c3ccccc3n(-c3ccccc3)c2c1] | 49 | 717.29 |
| 237 | 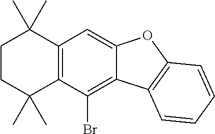 [SMILES: CC1(C)CCC(C)(C)c2c1cc1oc3ccccc3c1c2Br] | 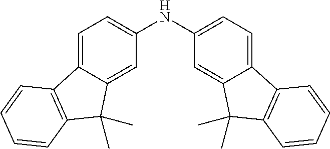 [SMILES: CC1(C)c2ccccc2-c2ccc(Nc3ccc4c(c3)C(C)(C)c3ccccc3-4)cc21] | 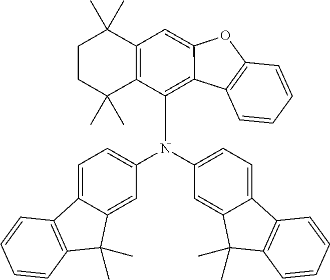 [SMILES: CC1(C)CCC(C)(C)c2c1cc1oc3ccccc3c1c2N(c1ccc2c(c1)C(C)(C)c1ccccc1-2)c1ccc2c(c1)C(C)(C)c1ccccc1-2] | 69 | 678.37 |
| 242 | 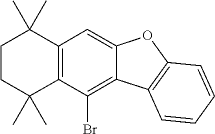 [SMILES: CC1(C)CCC(C)(C)c2c1cc1oc3ccccc3c1c2Br] | 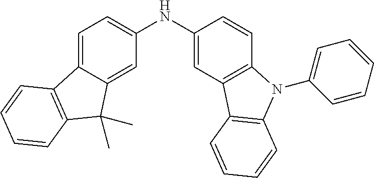 [SMILES: CC1(C)c2ccccc2-c2ccc(Nc3ccc4c(c3)c3ccccc3n4-c3ccccc3)cc21] | 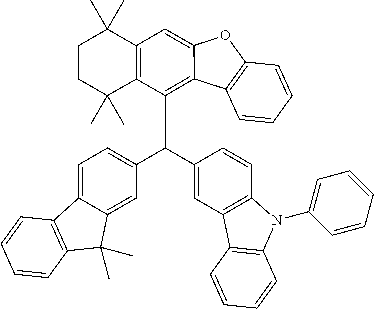 [SMILES: CC1(C)CCC(C)(C)c2c1cc1oc3ccccc3c1c2C(c1ccc2c(c1)C(C)(C)c1ccccc1-2)c1ccc2c(c1)c1ccccc1n2-c1ccccc1] | 62 | 727.36 |
| 251 | 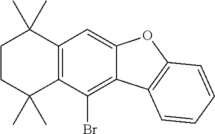 [SMILES: CC1(C)CCC(C)(C)c2c1cc1oc3ccccc3c1c2Br] | 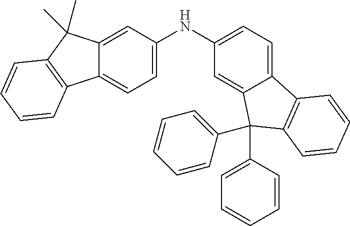 [SMILES: CC1(C)c2ccccc2-c2ccc(Nc3ccc4c(c3)C(c3ccccc3)(c3ccccc3)c3ccccc3-4)cc21] | 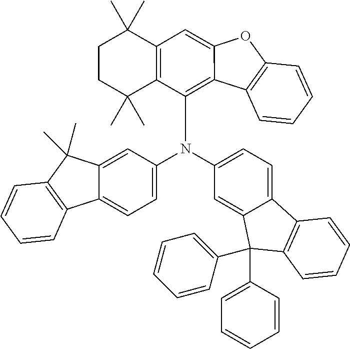 [SMILES: CC1(C)CCC(C)(C)c2c1cc1oc3ccccc3c1c2N(c1ccc2c(c1)C(C)(C)c1ccccc1-2)c1ccc2c(c1)C(c1ccccc1)(c1ccccc1)c1ccccc1-2] | 46 | 802.40 |
| 254 | 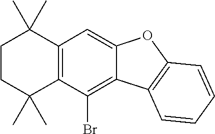 [SMILES: CC1(C)CCC(C)(C)c2c1cc1oc3ccccc3c1c2Br] | 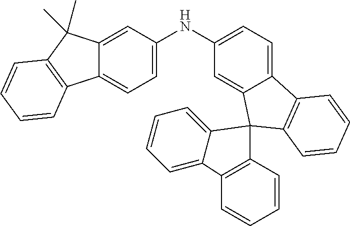 [SMILES: CC1(C)c2ccccc2-c2ccc(Nc3ccc4c(c3)C3(c5ccccc5-c5ccccc53)c3ccccc3-4)cc21] | 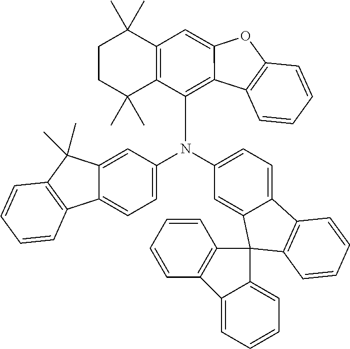 [SMILES: CC1(C)CCC(C)(C)c2c1cc1oc3ccccc3c1c2N(c1ccc2c(c1)C(C)(C)c1ccccc1-2)c1ccc2c(c1)C1(c3ccccc3-c3ccccc31)c1ccccc1-2] | 63 | 800.38 |
| 255 | 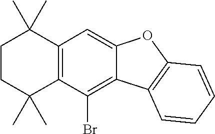 [SMILES: CC1(C)CCC(C)(C)c2c1cc1oc3ccccc3c1c2Br] | 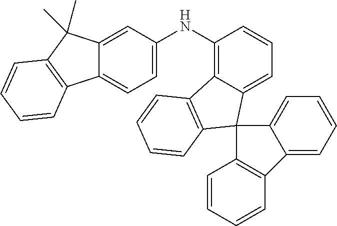 [SMILES: CC1(C)c2ccccc2-c2ccc(Nc3cccc4c3-c3ccccc3C43c4ccccc4-c4ccccc43)cc21] | 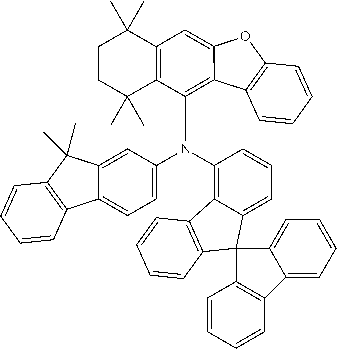 [SMILES: CC1(C)CCC(C)(C)c2c1cc1oc3ccccc3c1c2N(c1ccc2c(c1)C(C)(C)c1ccccc1-2)c1cccc2c1-c1ccccc1C21c2ccccc2-c2ccccc21] | 49 | 800.38 |
| 458 | 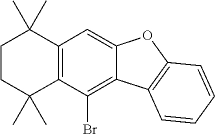 [SMILES: CC1(C)CCC(C)(C)c2c1cc1oc3ccccc3c1c2Br] | 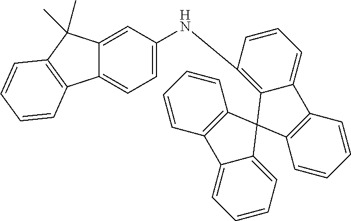 [SMILES: CC1(C)c2ccccc2-c2ccc(Nc3cccc4c3C3(c5ccccc5-c5ccccc53)c3ccccc3-4)cc21] | 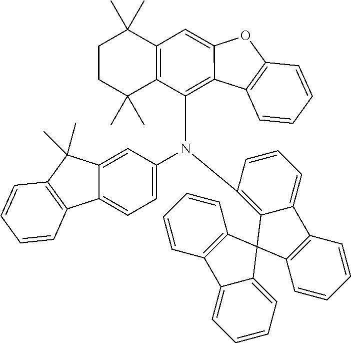 [SMILES: CC1(C)CCC(C)(C)c2c1cc1oc3ccccc3c1c2N(c1ccc2c(c1)C(C)(C)c1ccccc1-2)c1cccc2c1C1(c3ccccc3-c3ccccc31)c1ccccc1-2] | 37 | 800.38 |
| 256 | 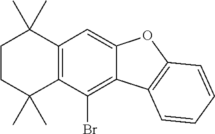 [SMILES: CC1(C)CCC(C)(C)c2c1cc1oc3ccccc3c1c2Br] | 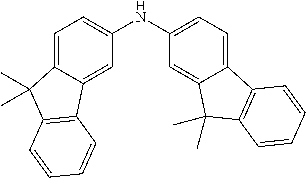 [SMILES: CC1(C)c2ccccc2-c2cc(Nc3ccc4c(c3)C(C)(C)c3ccccc3-4)ccc21] | 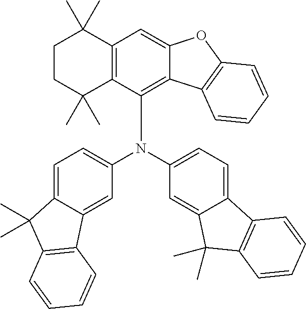 [SMILES: CC1(C)CCC(C)(C)c2c1cc1oc3ccccc3c1c2N(c1ccc2c(c1)-c1ccccc1C2(C)C)c1ccc2c(c1)C(C)(C)c1ccccc1-2] | 57 | 678.37 |
| 277 | 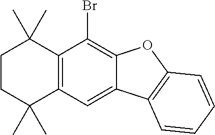 [SMILES: CC1(C)CCC(C)(C)c2c1cc1c(oc3ccccc31)c2Br] | 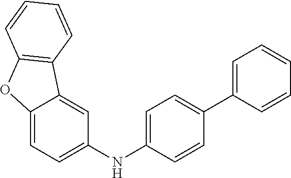 [SMILES: c1ccc(-c2ccc(Nc3ccc4oc5ccccc5c4c3)cc2)cc1] | 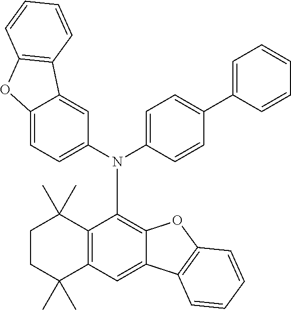 [SMILES: CC1(C)CCC(C)(C)c2c1cc1c(oc3ccccc31)c2N(c1ccc(-c2ccccc2)cc1)c1ccc2oc3ccccc3c2c1] | 68 | 612.29 |
| 281 | 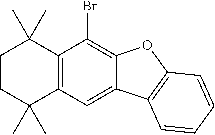 [SMILES: CC1(C)CCC(C)(C)c2c1cc1c(oc3ccccc31)c2Br] | 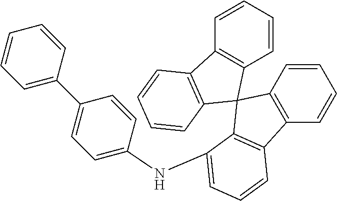 [SMILES: c1ccc(-c2ccc(Nc3cccc4c3C3(c5ccccc5-c5ccccc53)c3ccccc3-4)cc2)cc1] | 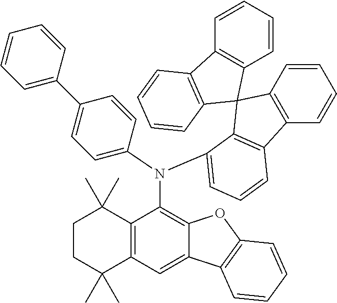 [SMILES: CC1(C)CCC(C)(C)c2c1cc1c(oc3ccccc31)c2N(c1ccc(-c2ccccc2)cc1)c1cccc2c1C1(c3ccccc3-c3ccccc31)c1ccccc1-2] | 56 | 760.35 |
| 283 | 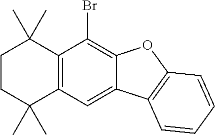 [SMILES: CC1(C)CCC(C)(C)c2c1cc1c(oc3ccccc31)c2Br] | 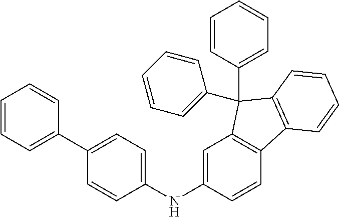 [SMILES: c1ccc(-c2ccc(Nc3ccc4c(c3)C(c3ccccc3)(c3ccccc3)c3ccccc3-4)cc2)cc1] | 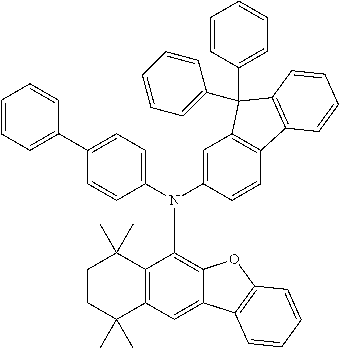 [SMILES: CC1(C)CCC(C)(C)c2c1cc1c(oc3ccccc31)c2N(c1ccc(-c2ccccc2)cc1)c1ccc2c(c1)C(c1ccccc1)(c1ccccc1)c1ccccc1-2] | 62 | 762.37 |
| 293 | 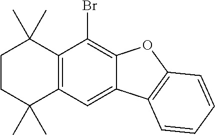 [SMILES: CC1(C)CCC(C)(C)c2c1cc1c(oc3ccccc31)c2Br] | 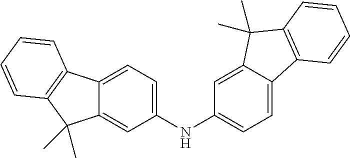 [SMILES: CC1(C)c2ccccc2-c2ccc(Nc3ccc4c(c3)C(C)(C)c3ccccc3-4)cc21] | 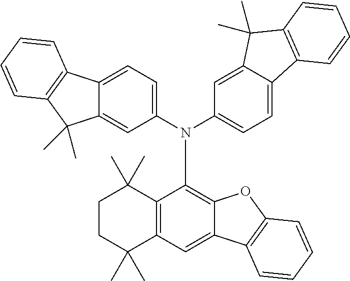 [SMILES: CC1(C)CCC(C)(C)c2c1cc1c(oc3ccccc31)c2N(c1ccc2c(c1)C(C)(C)c1ccccc1-2)c1ccc2c(c1)C(C)(C)c1ccccc1-2] | 52 | 678.37 |
| 295 | 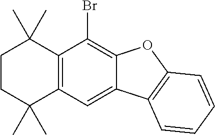 [SMILES: CC1(C)CCC(C)(C)c2c1cc1c(oc3ccccc31)c2Br] | 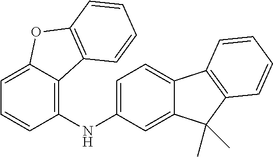 [SMILES: CC1(C)c2ccccc2-c2ccc(Nc3cccc4oc5ccccc5c34)cc21] | 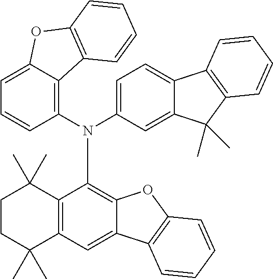 [SMILES: CC1(C)CCC(C)(C)c2c1cc1c(oc3ccccc31)c2N(c1ccc2c(c1)C(C)(C)c1ccccc1-2)c1cccc2oc3ccccc3c12] | 40 | 652.32 |
| 302 | 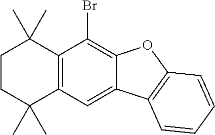 [SMILES: CC1(C)CCC(C)(C)c2c1cc1c(oc3ccccc31)c2Br] | 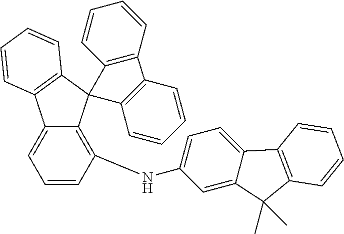 [SMILES: CC1(C)c2ccccc2-c2ccc(Nc3cccc4c3C3(c5ccccc5-c5ccccc53)c3ccccc3-4)cc21] | 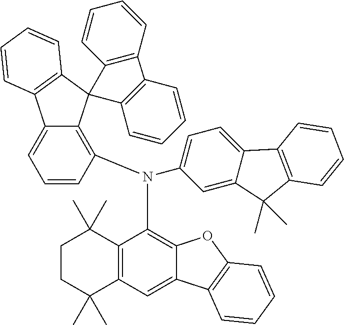 [SMILES: CC1(C)CCC(C)(C)c2c1cc1c(oc3ccccc31)c2N(c1ccc2c(c1)C(C)(C)c1ccccc1-2)c1cccc2c1C1(c3ccccc3-c3ccccc31)c1ccccc1-2] | 42 | 800.38 |
| 459 | 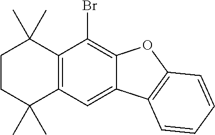 [SMILES: CC1(C)CCC(C)(C)c2c1cc1c(oc3ccccc31)c2Br] | 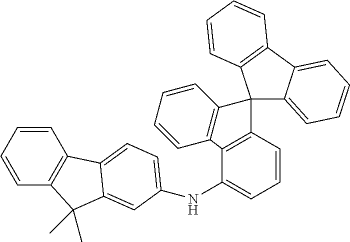 [SMILES: CC1(C)c2ccccc2-c2ccc(Nc3cccc4c3-c3ccccc3C43c4ccccc4-c4ccccc43)cc21] | 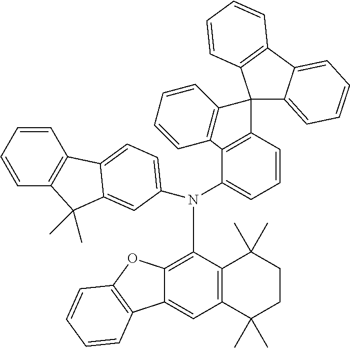 [SMILES: CC1(C)CCC(C)(C)c2c1cc1c(oc3ccccc31)c2N(c1ccc2c(c1)C(C)(C)c1ccccc1-2)c1cccc2c1-c1ccccc1C21c2ccccc2-c2ccccc21] | 51 | 800.38 |
| 308 | 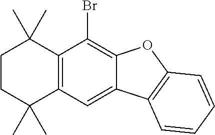 [SMILES: CC1(C)CCC(C)(C)c2c1cc1c(oc3ccccc31)c2Br] | 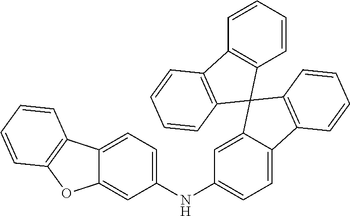 [SMILES: c1ccc2c(c1)-c1ccccc1C21c2ccccc2-c2ccc(Nc3ccc4c(c3)oc3ccccc34)cc21] | 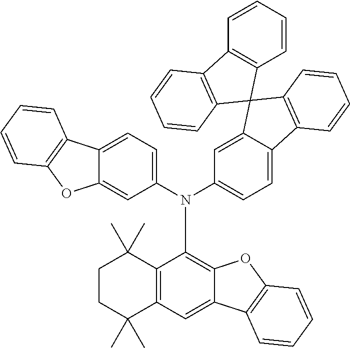 [SMILES: CC1(C)CCC(C)(C)c2c1cc1c(oc3ccccc31)c2N(c1ccc2c(c1)C1(c3ccccc3-c3ccccc31)c1ccccc1-2)c1ccc2c(c1)oc1ccccc12] | 63 | 774.33 |
| 375 | 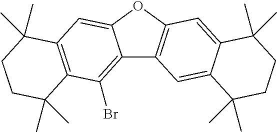 | 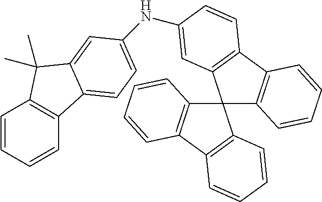 [SMILES: CC1(C)c2ccccc2-c2ccc(Nc3ccc4c(c3)C3(c5ccccc5-c5ccccc53)c3ccccc3-4)cc21] | 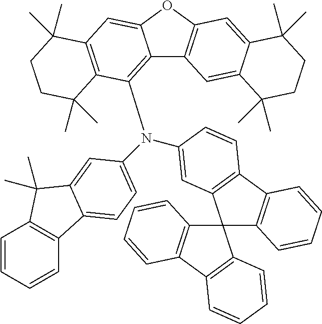 [SMILES: CC1(C)CCC(C)(C)c2cc3c(cc21)oc1cc2c(c(N(c4ccc5c(c4)C(C)(C)c4ccccc4-5)c4ccc5c(c4)C4(c6ccccc6-c6ccccc64)c4ccccc4-5)c13)C(C)(C)CCC2(C)C] | 47 | 910.49 |
| 376 | 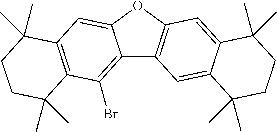 | 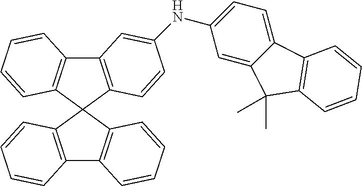 [SMILES: CC1(C)c2ccccc2-c2ccc(Nc3ccc4c(c3)-c3ccccc3C43c4ccccc4-c4ccccc43)cc21] | 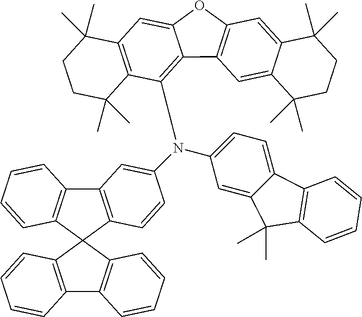 [SMILES: CC1(C)CCC(C)(C)c2cc3c(cc21)oc1cc2c(c(N(c4ccc5c(c4)-c4ccccc4C54c5ccccc5-c5ccccc54)c4ccc5c(c4)C(C)(C)c4ccccc4-5)c13)C(C)(C)CCC2(C)C] | 40 | 910.49 |
| 377 | 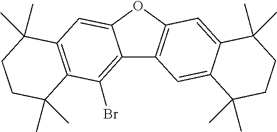 | 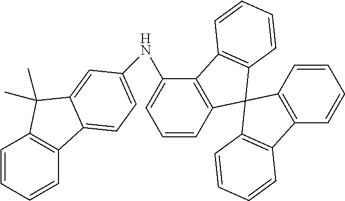 [SMILES: CC1(C)c2ccccc2-c2ccc(Nc3cccc4c3-c3ccccc3C43c4ccccc4-c4ccccc43)cc21] | 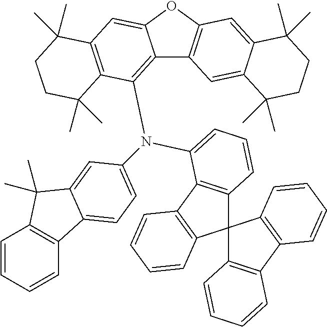 [SMILES: CC1(C)CCC(C)(C)c2cc3c(cc21)oc1cc2c(c(N(c4ccc5c(c4)C(C)(C)c4ccccc4-5)c4cccc5c4-c4ccccc4C54c5ccccc5-c5ccccc54)c13)C(C)(C)CCC2(C)C] | 36 | 910.49 |
| 382 | 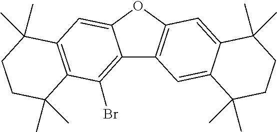 | 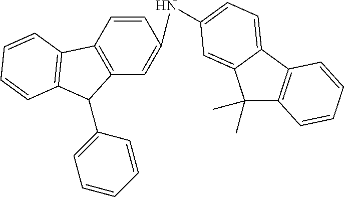 [SMILES: CC1(C)c2ccccc2-c2ccc(Nc3ccc4c(c3)C(c3ccccc3)c3ccccc3-4)cc21] | 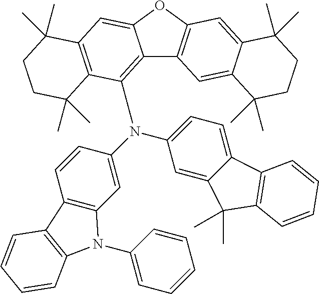 [SMILES: CC1(C)CCC(C)(C)c2cc3c(cc21)oc1cc2c(c(N(c4ccc5c(c4)C(C)(C)c4ccccc4-5)c4ccc5c6ccccc6n(-c6ccccc6)c5c4)c13)C(C)(C)CCC2(C)C] | 45 | 837.47 |
| 383 | 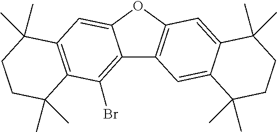 | 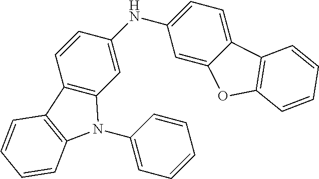 [SMILES: c1ccc(-n2c3ccccc3c3ccc(Nc4ccc5c(c4)oc4ccccc45)cc32)cc1] | 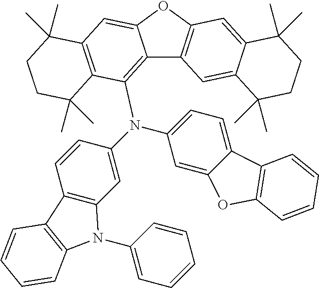 [SMILES: CC1(C)CCC(C)(C)c2cc3c(cc21)oc1cc2c(c(N(c4ccc5c(c4)oc4ccccc45)c4ccc5c6ccccc6n(-c6ccccc6)c5c4)c13)C(C)(C)CCC2(C)C] | 46 | 811.42 |
| 390 | 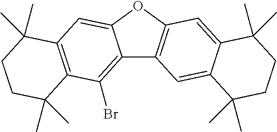 | 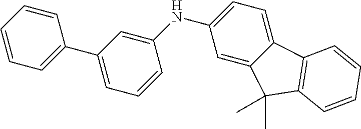 [SMILES: CC1(C)c2ccccc2-c2ccc(Nc3cccc(-c4ccccc4)c3)cc21] | 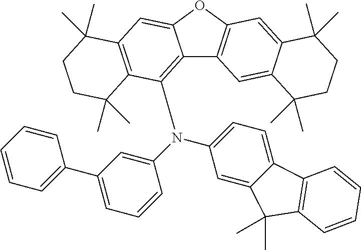 [SMILES: CC1(C)CCC(C)(C)c2cc3c(cc21)oc1cc2c(c(N(c4cccc(-c5ccccc5)c4)c4ccc5c(c4)C(C)(C)c4ccccc4-5)c13)C(C)(C)CCC2(C)C] | 56 | 748.45 |
| 432 | 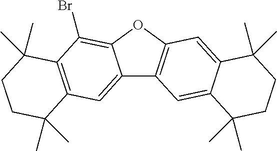 | 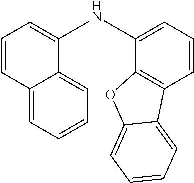 [SMILES: c1ccc2c(Nc3cccc4c3oc3ccccc34)cccc2c1] | 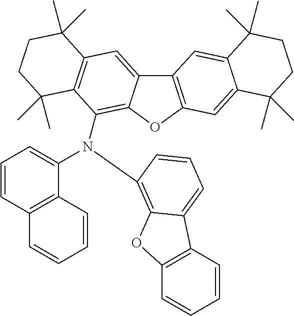 [SMILES: CC1(C)CCC(C)(C)c2cc3c(cc21)oc1c(N(c2cccc4ccccc24)c2cccc4c2oc2ccccc24)c2c(cc13)C(C)(C)CCC2(C)C] | 48 | 696.38 |
| 457 | 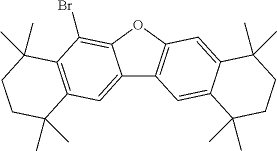 | 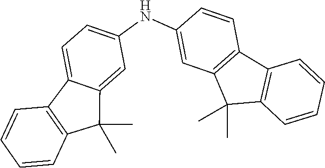 [SMILES: CC1(C)c2ccccc2-c2ccc(Nc3ccc4c(c3)C(C)(C)c3ccccc3-4)cc21] | 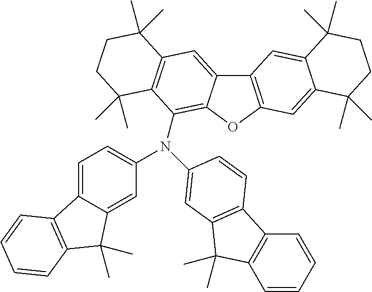 [SMILES: CC1(C)CCC(C)(C)c2cc3c(cc21)oc1c(N(c2ccc4c(c2)C(C)(C)c2ccccc2-4)c2ccc4c(c2)C(C)(C)c2ccccc2-4)c2c(cc13)C(C)(C)CCC2(C)C] | 61 | 788.48 |
| 402 | 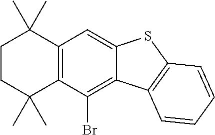 [SMILES: CC1(C)CCC(C)(C)c2c1cc1sc3ccccc3c1c2Br] | 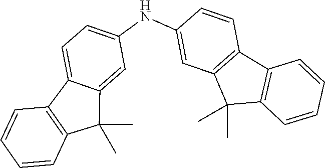 [SMILES: CC1(C)c2ccccc2-c2ccc(Nc3ccc4c(c3)C(C)(C)c3ccccc3-4)cc21] | 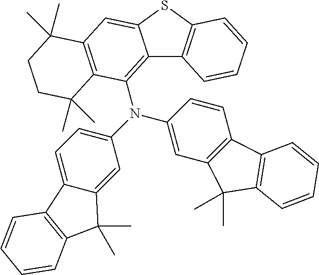 [SMILES: CC1(C)CCC(C)(C)c2c1cc1sc3ccccc3c1c2N(c1ccc2c(c1)C(C)(C)c1ccccc1-2)c1ccc2c(c1)C(C)(C)c1ccccc1-2] | 65 | 694.35 |
| 412 | 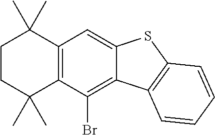 [SMILES: CC1(C)CCC(C)(C)c2c1cc1sc3ccccc3c1c2Br] | 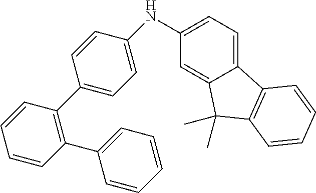 [SMILES: CC1(C)c2ccccc2-c2ccc(Nc3ccc(-c4ccccc4-c4ccccc4)cc3)cc21] | 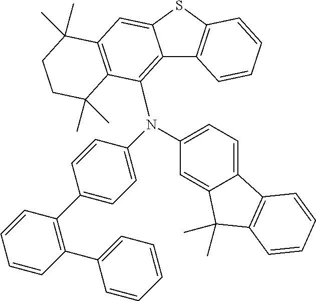 [SMILES: CC1(C)CCC(C)(C)c2c1cc1sc3ccccc3c1c2N(c1ccc(-c2ccccc2-c2ccccc2)cc1)c1ccc2c(c1)C(C)(C)c1ccccc1-2] | 68 | 730.35 |
| 433 | 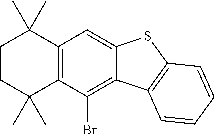 [SMILES: CC1(C)CCC(C)(C)c2c1cc1sc3ccccc3c1c2Br] | 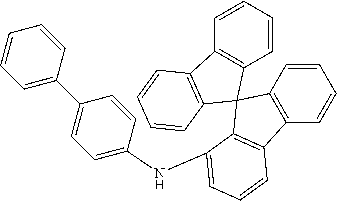 [SMILES: c1ccc(-c2ccc(Nc3cccc4c3C3(c5ccccc5-c5ccccc53)c3ccccc3-4)cc2)cc1] | 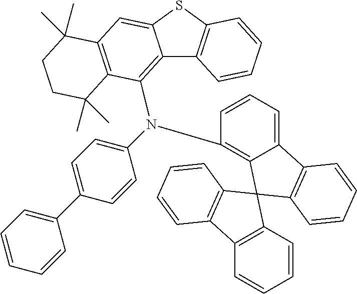 [SMILES: CC1(C)CCC(C)(C)c2c1cc1sc3ccccc3c1c2N(c1ccc(-c2ccccc2)cc1)c1cccc2c1C1(c3ccccc3-c3ccccc31)c1ccccc1-2] | 46 | 776.33 |
| 434 | 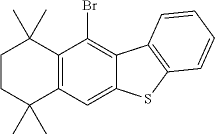 [SMILES: CC1(C)CCC(C)(C)c2c1cc1sc3ccccc3c1c2Br] | 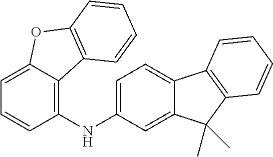 [SMILES: CC1(C)c2ccccc2-c2ccc(Nc3cccc4oc5ccccc5c34)cc21] | 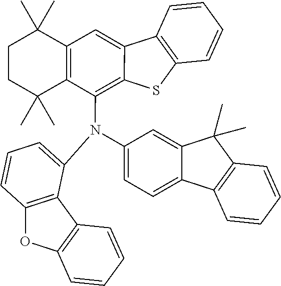 [SMILES: CC1(C)CCC(C)(C)c2c1cc1c(sc3ccccc31)c2N(c1ccc2c(c1)C(C)(C)c1ccccc1-2)c1cccc2oc3ccccc3c12] | 52 | 668.29 |
| 435 | 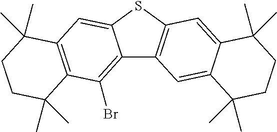 | 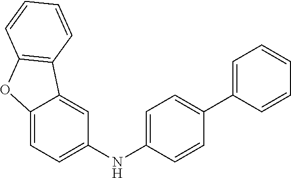 [SMILES: c1ccc(-c2ccc(Nc3ccc4oc5ccccc5c4c3)cc2)cc1] | 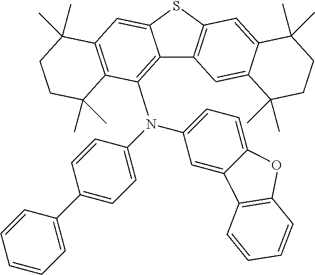 [SMILES: CC1(C)CCC(C)(C)c2cc3c(cc21)sc1cc2c(c(N(c4ccc(-c5ccccc5)cc4)c4ccc5oc6ccccc6c5c4)c13)C(C)(C)CCC2(C)C] | 58 | 738.37 |
| 436 | 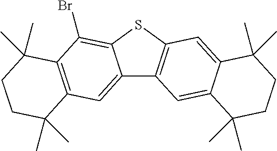 | 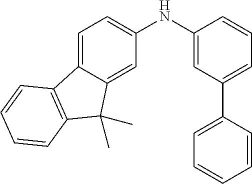 [SMILES: CC1(C)c2ccccc2-c2ccc(Nc3cccc(-c4ccccc4)c3)cc21] | 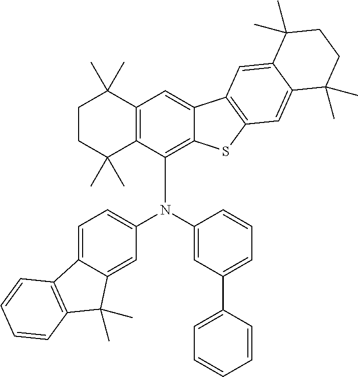 [SMILES: CC1(C)CCC(C)(C)c2cc3c(cc21)sc1c(N(c2cccc(-c4ccccc4)c2)c2ccc4c(c2)C(C)(C)c2ccccc2-4)c2c(cc13)C(C)(C)CCC2(C)C] | 56 | 764.42 |
| 426 | 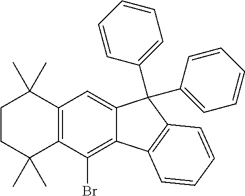 [SMILES: CC1(C)CCC(C)(C)c2c1cc1c(c2Br)-c2ccccc2C1(c1ccccc1)c1ccccc1] | 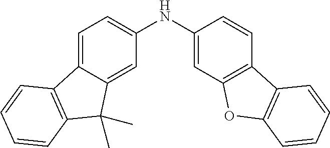 [SMILES: CC1(C)c2ccccc2-c2ccc(Nc3ccc4c(c3)oc3ccccc34)cc21] | 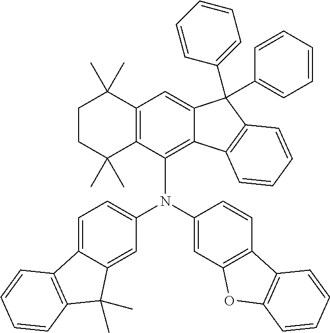 [SMILES: CC1(C)CCC(C)(C)c2c1cc1c(c2N(c2ccc3c(c2)C(C)(C)c2ccccc2-3)c2ccc3c(c2)oc2ccccc23)-c2ccccc2C1(c1ccccc1)c1ccccc1] | 42 | 802.40 |
| 427 | 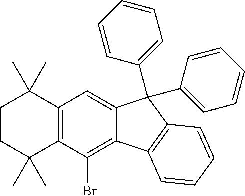 [SMILES: CC1(C)CCC(C)(C)c2c1cc1c(c2Br)-c2ccccc2C1(c1ccccc1)c1ccccc1] | 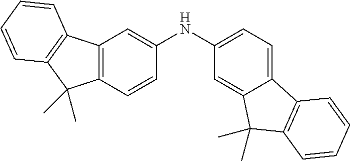 [SMILES: CC1(C)c2ccccc2-c2cc(Nc3ccc4c(c3)C(C)(C)c3ccccc3-4)ccc21] | 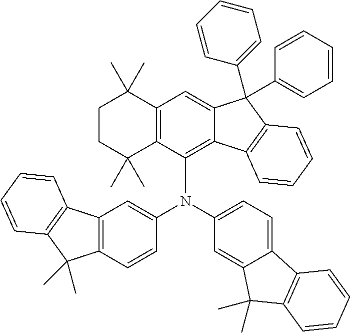 [SMILES: CC1(C)CCC(C)(C)c2c1cc1c(c2N(c2ccc3c(c2)-c2ccccc2C3(C)C)c2ccc3c(c2)C(C)(C)c2ccccc2-3)-c2ccccc2C1(c1ccccc1)c1ccccc1] | 53 | 828.45 |
| 437 | 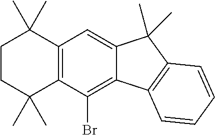 [SMILES: CC1(C)CCC(C)(C)c2c1cc1c(c2Br)-c2ccccc2C1(C)C] | 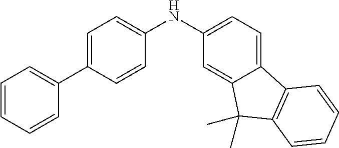 [SMILES: CC1(C)c2ccccc2-c2ccc(Nc3ccc(-c4ccccc4)cc3)cc21] | 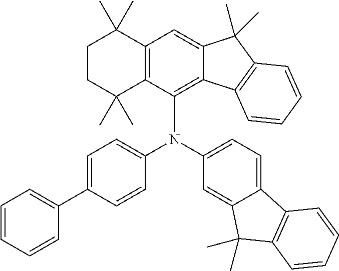 [SMILES: CC1(C)CCC(C)(C)c2c1cc1c(c2N(c2ccc(-c3ccccc3)cc2)c2ccc3c(c2)C(C)(C)c2ccccc2-3)-c2ccccc2C1(C)C] | 62 | 664.39 |
| 438 | 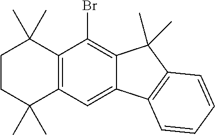 [SMILES: CC1(C)CCC(C)(C)c2c1cc1c(c2Br)C(C)(C)c2ccccc2-1] | 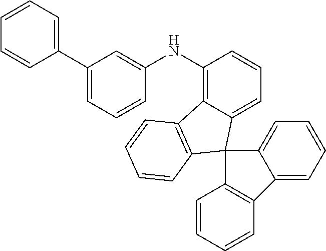 [SMILES: c1ccc(-c2cccc(Nc3cccc4c3-c3ccccc3C43c4ccccc4-c4ccccc43)c2)cc1] | 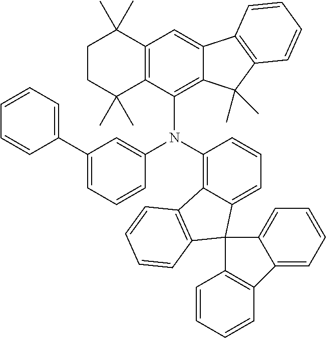 [SMILES: CC1(C)CCC(C)(C)c2c1cc1c(c2N(c2cccc(-c3ccccc3)c2)c2cccc3c2-c2ccccc2C32c3ccccc3-c3ccccc32)C(C)(C)c2ccccc2-1] | 69 | 786.41 |
| 439 | 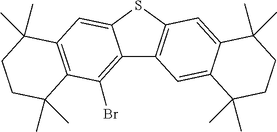 | 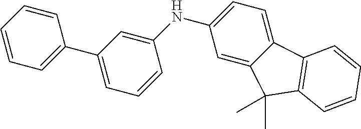 [SMILES: CC1(C)c2ccccc2-c2ccc(Nc3cccc(-c4ccccc4)c3)cc21] | 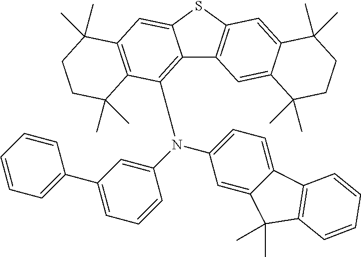 [SMILES: CC1(C)CCC(C)(C)c2cc3c(cc21)sc1cc2c(c(N(c4cccc(-c5ccccc5)c4)c4ccc5c(c4)C(C)(C)c4ccccc4-5)c13)C(C)(C)CCC2(C)C] | 55 | 774.50 |
| 440 | 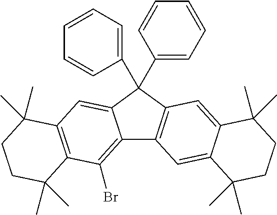 | 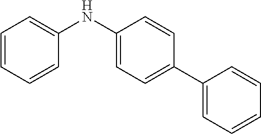 [SMILES: c1ccc(Nc2ccc(-c3ccccc3)cc2)cc1] | 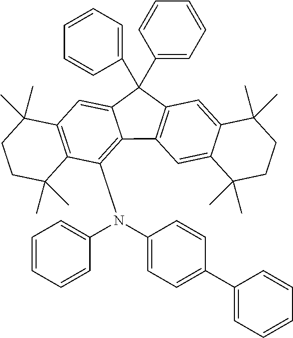 [SMILES: CC1(C)CCC(C)(C)c2cc3c(cc21)-c1c(cc2c(c1N(c1ccccc1)c1ccc(-c4ccccc4)cc1)C(C)(C)CCC2(C)C)C3(c1ccccc1)c1ccccc1] | 63 | 782.47 |
| 441 | 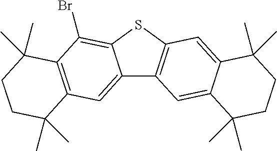 | 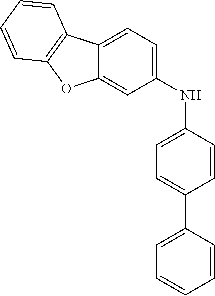 [SMILES: c1ccc(-c2ccc(Nc3ccc4c(c3)oc3ccccc34)cc2)cc1] | 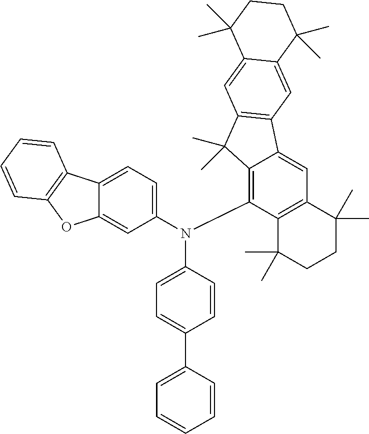 [SMILES: CC1(C)CCC(C)(C)c2cc3c(cc21)-c1cc2c(c(N(c4ccc(-c5ccccc5)cc4)c4ccc5c(c4)oc4ccccc45)c1C3(C)C)C(C)(C)CCC2(C)C] | 46 | 748.45 |
| 442 | 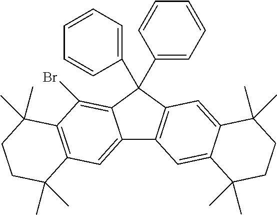 | 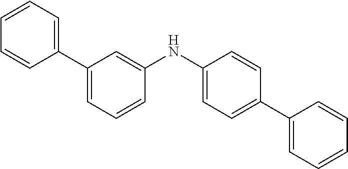 [SMILES: c1ccc(-c2ccc(Nc3cccc(-c4ccccc4)c3)cc2)cc1] | 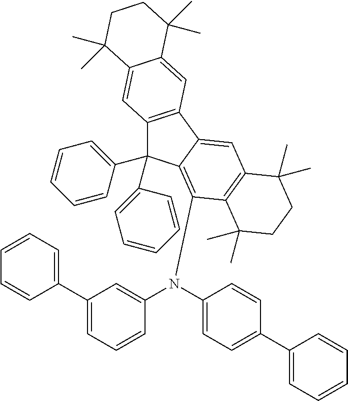 [SMILES: CC1(C)CCC(C)(C)c2cc3c(cc21)-c1cc2c(c(N(c4ccc(-c5ccccc5)cc4)c4cccc(-c5ccccc5)c4)c1C3(c1ccccc1)c1ccccc1)C(C)(C)CCC2(C)C] | 49 | 858.50 |
| 430 | 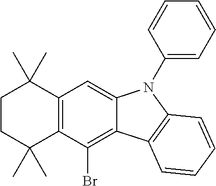 [SMILES: CC1(C)CCC(C)(C)c2c1cc1c(c2Br)c2ccccc2n1-c1ccccc1] | 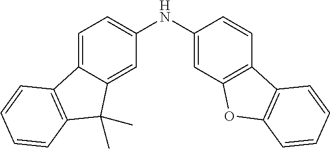 [SMILES: CC1(C)c2ccccc2-c2ccc(Nc3ccc4c(c3)oc3ccccc34)cc21] | 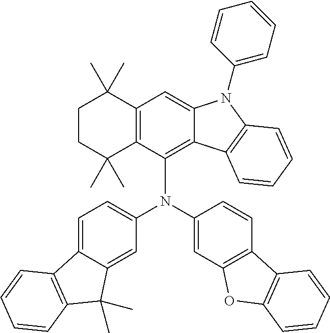 [SMILES: CC1(C)CCC(C)(C)c2c1cc1c(c2N(c2ccc3c(c2)C(C)(C)c2ccccc2-3)c2ccc3c(c2)oc2ccccc23)c2ccccc2n1-c1ccccc1] | 47 | 727.36 |
| 443 | 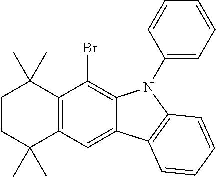 [SMILES: CC1(C)CCC(C)(C)c2c1cc1c3ccccc3n(-c3ccccc3)c1c2Br] | 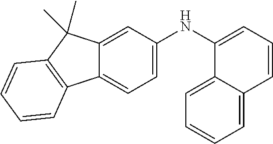 [SMILES: CC1(C)c2ccccc2-c2ccc(Nc3cccc4ccccc34)cc21] | 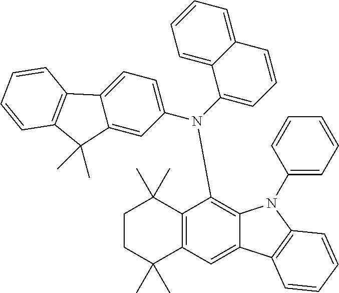 [SMILES: CC1(C)CCC(C)(C)c2c1cc1c3ccccc3n(-c3ccccc3)c1c2N(c1ccc2c(c1)C(C)(C)c1ccccc1-2)c1cccc2ccccc12] | 59 | 701.39 |
| 444 | 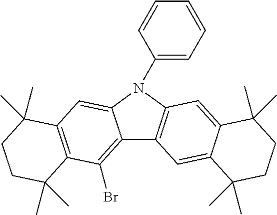 | 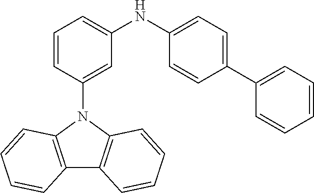 [SMILES: c1ccc(-c2ccc(Nc3cccc(-n4c5ccccc5c5ccccc54)c3)cc2)cc1] | 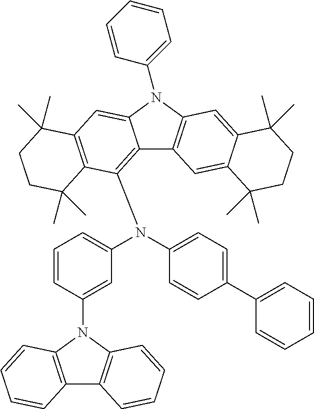 [SMILES: CC1(C)CCC(C)(C)c2cc3c(cc21)c1c(N(c2ccc(-c4ccccc4)cc2)c2cccc(-n4c5ccccc5c5ccccc54)c2)c2c(cc1n3-c1ccccc1)C(C)(C)CCC2(C)C] | 52 | 872.49 |
| 445 | 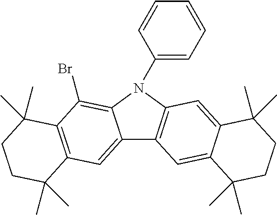 | 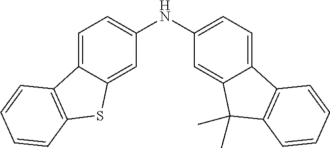 [SMILES: CC1(C)c2ccccc2-c2ccc(Nc3ccc4c(c3)sc3ccccc34)cc21] | 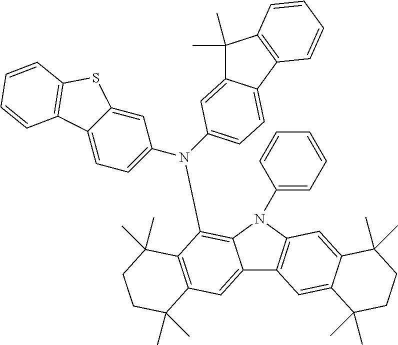 [SMILES: CC1(C)CCC(C)(C)c2cc3c(cc21)c1cc2c(c(N(c4ccc5c(c4)C(C)(C)c4ccccc4-5)c4ccc5c(c4)sc4ccccc45)c1n3-c1ccccc1)C(C)(C)CCC2(C)C] | 56 | 853.45 |

Following the synthesis method of Compound 2, the corresponding Intermediate X in Table 5 was used instead of Intermediate a, and Reactant F was used instead of N-phenyl-1-naphthalene-2-amine, to respectively synthesize the compounds of the present disclosure listed in Table 5.

TABLE 5

| | | | | | Mass |
|---|---|---|---|---|---|
| | Intermediate | | | Yield | Spectra |
| Compound | X | Reactant F | Structure | (%) | ([M + H]⁺) |
| 317 | 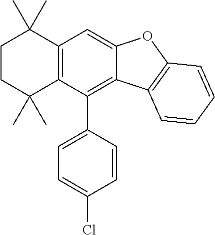 [SMILES: CC1(C)CCC(C)(C)c2c1cc1oc3ccccc3c1c2-c1ccc(Cl)cc1] | 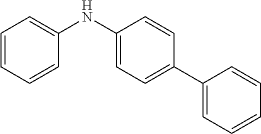 [SMILES: c1ccc(Nc2ccc(-c3ccccc3)cc2)cc1] | 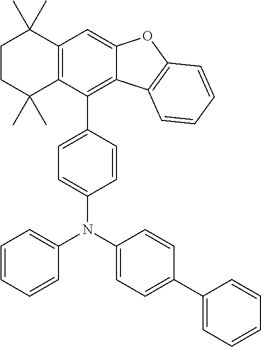 [SMILES: CC1(C)CCC(C)(C)c2c1cc1oc3ccccc3c1c2-c1ccc(N(c2ccccc2)c2ccc(-c3ccccc3)cc2)cc1] | 53 | 598.31 |
| 328 | 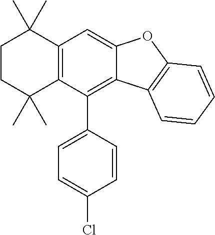 [SMILES: CC1(C)CCC(C)(C)c2c1cc1oc3ccccc3c1c2-c1ccc(Cl)cc1] | 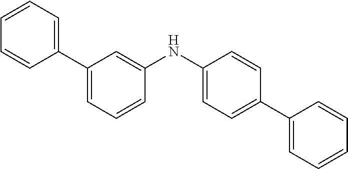 [SMILES: c1ccc(-c2ccc(Nc3cccc(-c4ccccc4)c3)cc2)cc1] | 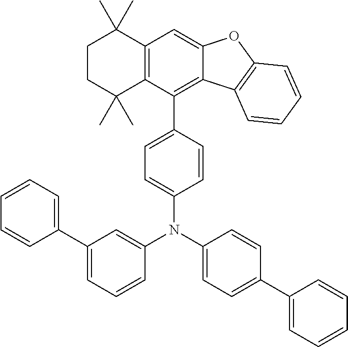 [SMILES: CC1(C)CCC(C)(C)c2c1cc1oc3ccccc3c1c2-c1ccc(N(c2ccc(-c3ccccc3)cc2)c2cccc(-c3ccccc3)c2)cc1] | 65 | 674.34 |
| 334 | 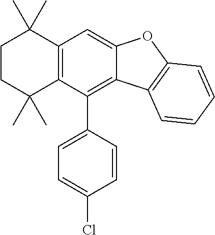 [SMILES: CC1(C)CCC(C)(C)c2c1cc1oc3ccccc3c1c2-c1ccc(Cl)cc1] | 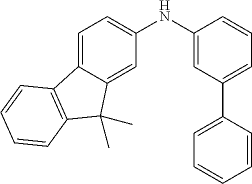 [SMILES: CC1(C)c2ccccc2-c2ccc(Nc3cccc(-c4ccccc4)c3)cc21] | 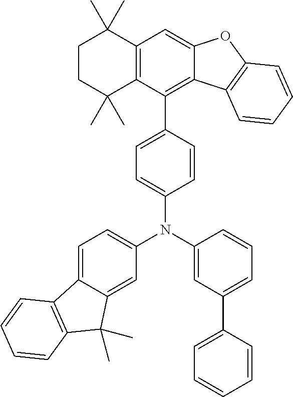 [SMILES: CC1(C)CCC(C)(C)c2c1cc1oc3ccccc3c1c2-c1ccc(N(c2cccc(-c3ccccc3)c2)c2ccc3c(c2)C(C)(C)c2ccccc2-3)cc1] | 49 | 714.37 |
| 448 | 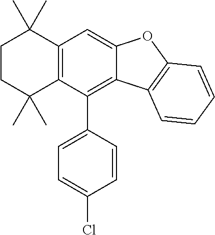 [SMILES: CC1(C)CCC(C)(C)c2c1cc1oc3ccccc3c1c2-c1ccc(Cl)cc1] | 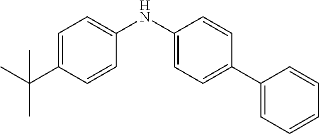 [SMILES: CC(C)(C)c1ccc(Nc2ccc(-c3ccccc3)cc2)cc1] | 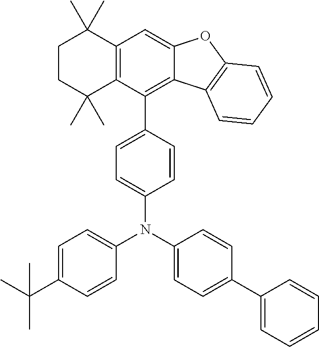 [SMILES: CC(C)(C)c1ccc(N(c2ccc(-c3ccccc3)cc2)c2ccc(-c3c4c(cc5oc6ccccc6c35)C(C)(C)CCC4(C)C)cc2)cc1] | 66 | 654.37 |
| 342 | 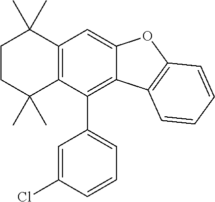 [SMILES: CC1(C)CCC(C)(C)c2c1cc1oc3ccccc3c1c2-c1cccc(Cl)c1] | 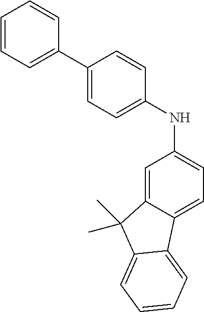 [SMILES: CC1(C)c2ccccc2-c2ccc(Nc3ccc(-c4ccccc4)cc3)cc21] | 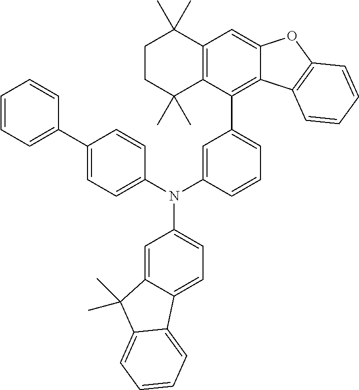 [SMILES: CC1(C)CCC(C)(C)c2c1cc1oc3ccccc3c1c2-c1cccc(N(c2ccc(-c3ccccc3)cc2)c2ccc3c(c2)C(C)(C)c2ccccc2-3)c1] | 58 | 714.37 |
| 359 | 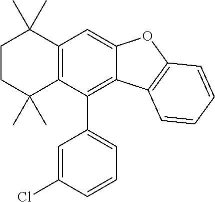 [SMILES: CC1(C)CCC(C)(C)c2c1cc1oc3ccccc3c1c2-c1cccc(Cl)c1] | 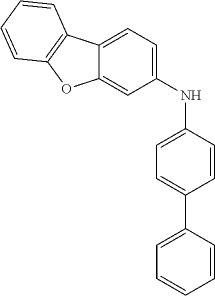 [SMILES: c1ccc(-c2ccc(Nc3ccc4c(c3)oc3ccccc34)cc2)cc1] | 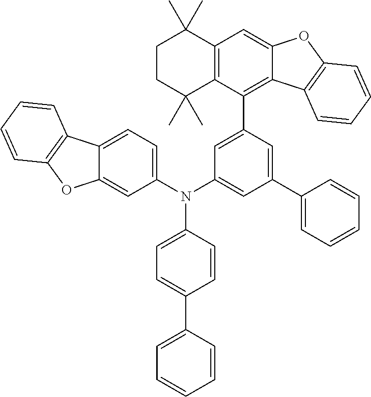 [SMILES: CC1(C)CCC(C)(C)c2c1cc1oc3ccccc3c1c2-c1cc(-c2ccccc2)cc(N(c2ccc(-c3ccccc3)cc2)c2ccc3c(c2)oc2ccccc23)c1] | 63 | 764.35 |
| 368 | 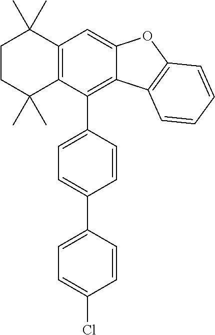 [SMILES: CC1(C)CCC(C)(C)c2c1cc1oc3ccccc3c1c2-c1ccc(-c2ccc(Cl)cc2)cc1] | 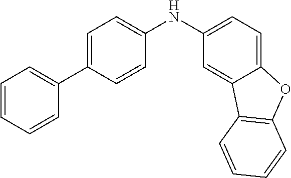 [SMILES: c1ccc(-c2ccc(Nc3ccc4oc5ccccc5c4c3)cc2)cc1] | 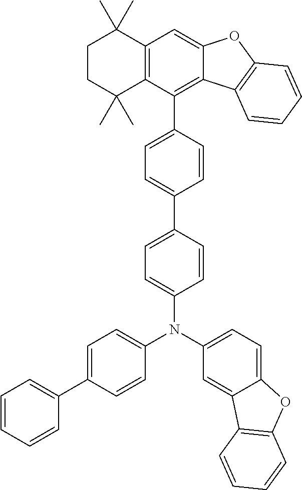 [SMILES: CC1(C)CCC(C)(C)c2c1cc1oc3ccccc3c1c2-c1ccc(-c2ccc(N(c3ccc(-c4ccccc4)cc3)c3ccc4oc5ccccc5c4c3)cc2)cc1] | 52 | 764.35 |
| 449 | 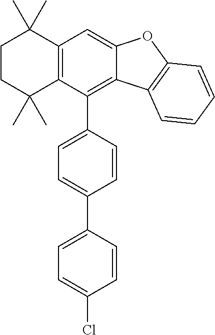 [SMILES: CC1(C)CCC(C)(C)c2c1cc1oc3ccccc3c1c2-c1ccc(-c2ccc(Cl)cc2)cc1] | 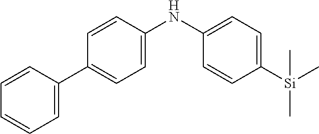 [SMILES: C[Si](C)(C)c1ccc(Nc2ccc(-c3ccccc3)cc2)cc1] | 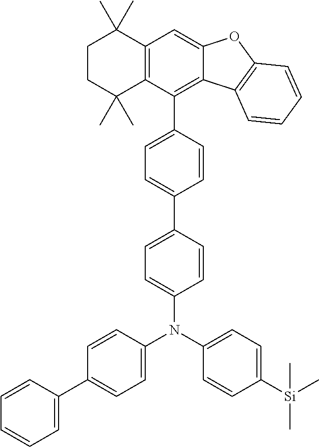 [SMILES: CC1(C)CCC(C)(C)c2c1cc1oc3ccccc3c1c2-c1ccc(-c2ccc(N(c3ccc(-c4ccccc4)cc3)c3ccc([Si](C)(C)C)cc3)cc2)cc1] | 64 | 746.38 |
| 450 | 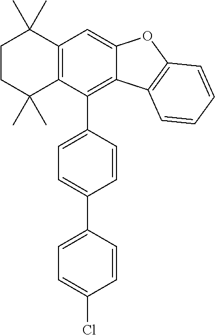 [SMILES: CC1(C)CCC(C)(C)c2c1cc1oc3ccccc3c1c2-c1ccc(-c2ccc(Cl)cc2)cc1] | 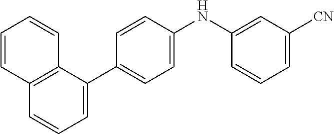 [SMILES: N#Cc1cccc(Nc2ccc(-c3cccc4ccccc34)cc2)c1] | 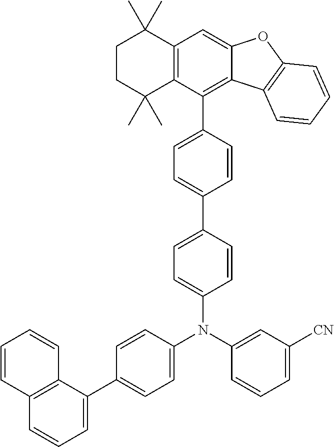 [SMILES: CC1(C)CCC(C)(C)c2c1cc1oc3ccccc3c1c2-c1ccc(-c2ccc(N(c3ccc(-c4cccc5ccccc45)cc3)c3cccc(C#N)c3)cc2)cc1] | 49 | 749.35 |
| 372 | 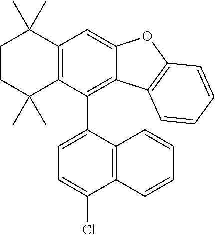 [SMILES: CC1(C)CCC(C)(C)c2c1cc1oc3ccccc3c1c2-c1ccc(Cl)c2ccccc12] | 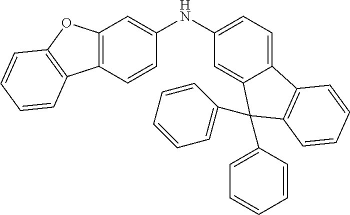 [SMILES: c1ccc(C2(c3ccccc3)c3ccccc3-c3ccc(Nc4ccc5c(c4)oc4ccccc45)cc32)cc1] | 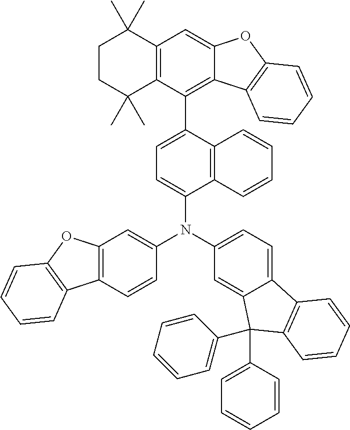 [SMILES: CC1(C)CCC(C)(C)c2c1cc1oc3ccccc3c1c2-c1ccc(N(c2ccc3c(c2)C(c2ccccc2)(c2ccccc2)c2ccccc2-3)c2ccc3c(c2)oc2ccccc23)c2ccccc12] | 47 | 902.40 |
| 451 | 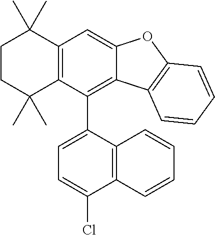 [SMILES: CC1(C)CCC(C)(C)c2c1cc1oc3ccccc3c1c2-c1ccc(Cl)c2ccccc12] | 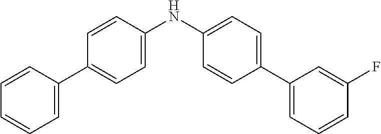 [SMILES: Fc1cccc(-c2ccc(Nc3ccc(-c4ccccc4)cc3)cc2)c1] | 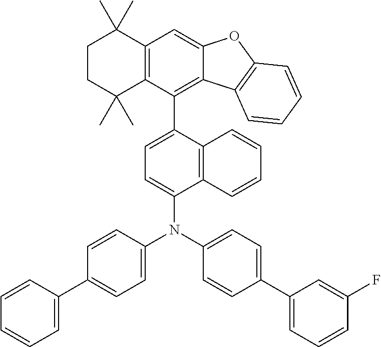 [SMILES: CC1(C)CCC(C)(C)c2c1cc1oc3ccccc3c1c2-c1ccc(N(c2ccc(-c3ccccc3)cc2)c2ccc(-c3cccc(F)c3)cc2)c2ccccc12] | 58 | 742.34 |
| 452 | 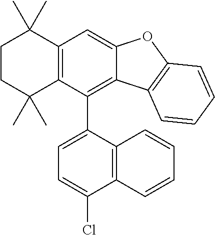 [SMILES: CC1(C)CCC(C)(C)c2c1cc1oc3ccccc3c1c2-c1ccc(Cl)c2ccccc12] | 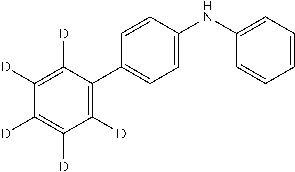 [SMILES: [2H]c1c([2H])c([2H])c(-c2ccc(Nc3ccccc3)cc2)c([2H])c1[2H]] | 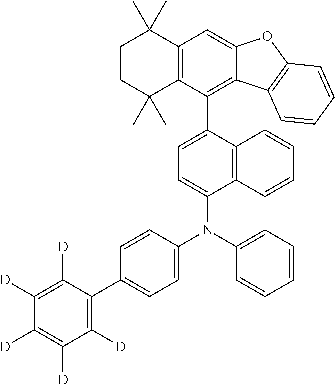 [SMILES: [2H]c1c([2H])c([2H])c(-c2ccc(N(c3ccccc3)c3ccc(-c4c5c(cc6oc7ccccc7c46)C(C)(C)CCC5(C)C)c4ccccc34)cc2)c([2H])c1[2H]] | 46 | 653.35 |

NMR data of some Intermediates and Compounds are shown in Table 6 below:

TABLE 6

| Compound | NMR data |
|---|---|
| Intermediate a | ¹H NMR (CD₂Cl₂, 400 MHz): δ ppm 7.55-8.01 (m, 4H), 7.36 (t, 1H), |
| | 1.50 (d, 2H), 1.42 (d, 2H), 0.85-0.90 (m, 12H). |
| Compound 237 | ¹H NMR (CD₂Cl₂, 400 MHz): δ ppm 7.60 (d, 2H), 7.52 (d, 2H), 7.36- |
| | 7.50 (m, 6H), 7.21-7.29 (m, 4H), 7.05-7.10 (m, 2H), 6.89-6.92 (m, 3H), |
| | 1.71-1.76 (m, 2H), 1.63-1.66 (m, 2H), 1.50-1.56(m, 6H), 1.34-1.39(m, |
| | 12H), 1.33-1.71(m, 6H). |

### Example 1: Green Organic Electroluminescent Device

First, an anode pretreatment was performed by the following processes: the surface of ITO/Ag/ITO substrate with a thickness of 100 Å, 1000 Å, and 100 Å in sequence was treated using ultraviolet ozone and O₂:N₂ plasma to increase the work function of the anode. The surface of the ITO substrate can also be cleaned with an organic solvent to remove impurities and oil thereon.

On the test substrate (anode), PD and HT-12 were co-vapor deposited at a vapor-deposition rate ratio of 2%:98%, to form a hole injection layer (HIL) with a thickness of 110 Å. Subsequently, HT-12 was vacuum-vapor deposited on the hole injection layer to form a hole transport layer with a thickness of 1025 Å.

Compound 8 was vacuum vapor deposited on the hole transport layer to form a hole auxiliary layer with a thickness of 700 Å.

Compounds GH-01, GH-02, and GD-01 were co-vapor deposited on the hole auxiliary layer at a film thickness ratio of 50%:40%: 10% to form an organic light-emitting layer (EML) with a thickness of 430 Å.

ET-1 and LiQ were vapor deposited at a film thickness ratio of 1:1 to form an electron transport layer (ETL) with a thickness of 350 Å. Yb was vapor deposited on the electron transport layer to form an electron injection layer (EIL) with a thickness of 20 Å. Subsequently, magnesium (Mg) and silver (Ag) were vacuum vapor deposited on the electron injection layer at a film thickness ratio of 1:10 to form a cathode with a thickness of 150 Å.

In addition, CP-5 was vacuum vapor deposited on the above cathode to form an organic cover layer (CPL) with a thickness of 630 Å, thus completing the fabrication of the organic electroluminescent device.

### Examples 2 to 76

Organic electroluminescent devices were fabricated using the same method as in Example 1, except that Compound 8 was replaced with the compounds as indicated in Table 8 during the formation of the hole auxiliary layer.

### Comparative Example 1

Organic electroluminescent device was fabricated using the same method as in Example 1, except that Compound 8 was replaced with Compound A listed in Table 8 during the formation of the hole auxiliary layer.

### Comparative Example 2

Organic electroluminescent device was fabricated using the same method as in Example 1, except that Compound 8 was replaced with Compound B listed in Table 8 during the formation of the hole auxiliary layer.

### Comparative Example 3

Organic electroluminescent device was fabricated using the same method as in Example 1, except that Compound 8 was replaced with Compound C listed in Table 8 during the formation of the hole auxiliary layer.

### Comparative Example 4

Organic electroluminescent device was fabricated using the same method as in Example 1, except that Compound 8 was replaced with Compound D listed in Table 8 during the formation of the hole auxiliary layer.

The material structures used in the above examples and comparative examples are shown in Table 7 below:

TABLE 7 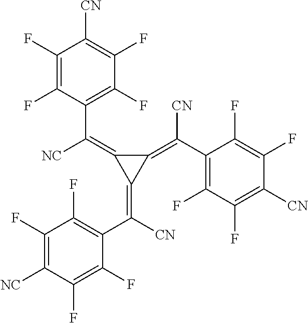 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]
PD 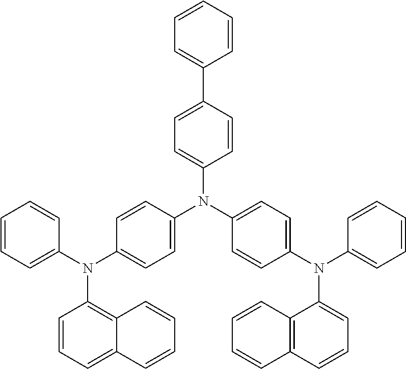 [SMILES: c1ccc(-c2ccc(N(c3ccc(N(c4ccccc4)c4cccc5ccccc45)cc3)c3ccc(N(c4ccccc4)c4cccc5ccccc45)cc3)cc2)cc1]
HT-12 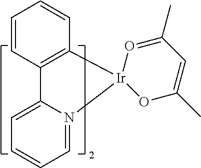
GD-01 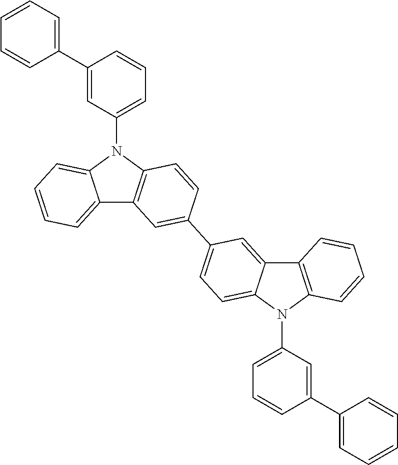 [SMILES: c1ccc(-c2cccc(-n3c4ccccc4c4cc(-c5ccc6c(c5)c5ccccc5n6-c5cccc(-c6ccccc6)c5)ccc43)c2)cc1]
GH-01 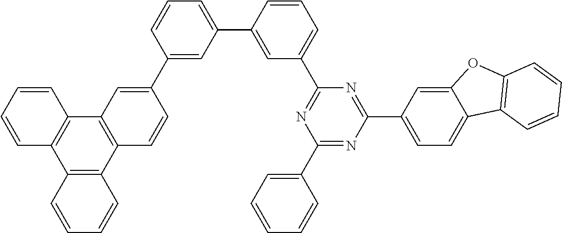 [SMILES: c1ccc(-c2nc(-c3cccc(-c4cccc(-c5ccc6c7ccccc7c7ccccc7c6c5)c4)c3)nc(-c3ccc4c(c3)oc3ccccc34)n2)cc1]
GH-02 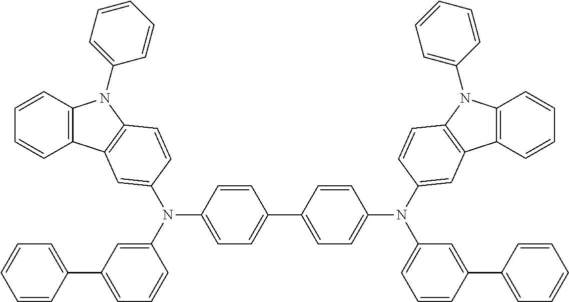 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]
CP-5 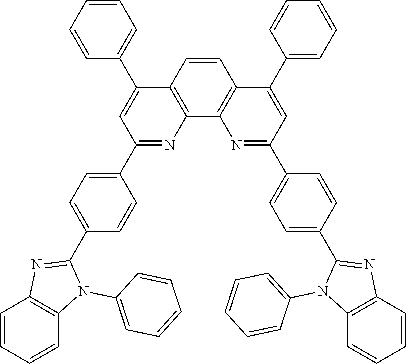 [SMILES: c1ccc(-c2cc(-c3ccc(-c4nc5ccccc5n4-c4ccccc4)cc3)nc3c2ccc2c(-c4ccccc4)cc(-c4ccc(-c5nc6ccccc6n5-c5ccccc5)cc4)nc23)cc1]
ET-1 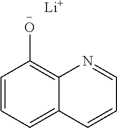 [SMILES: [Li+] ; Oc1cccc2cccnc12]
LiQ 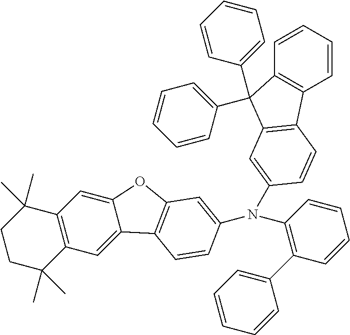 [SMILES: CC1(C)CCC(C)(C)c2cc3c(cc21)oc1cc(N(c2ccc4c(c2)C(c2ccccc2)(c2ccccc2)c2ccccc2-4)c2ccccc2-c2ccccc2)ccc13]
Compound A 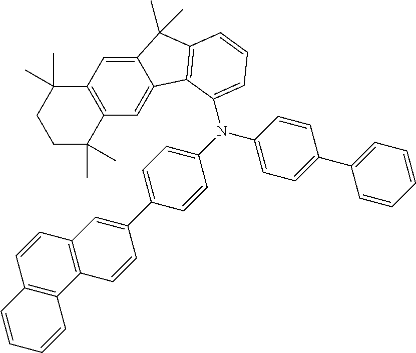 [SMILES: CC1(C)CCC(C)(C)c2cc3c(cc21)-c1c(N(c2ccc(-c4ccccc4)cc2)c2ccc(-c4ccc5c(ccc6ccccc65)c4)cc2)cccc1C3(C)C]
Compound B 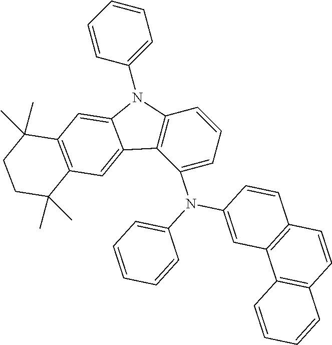 [SMILES: CC1(C)CCC(C)(C)c2cc3c(cc21)c1c(N(c2ccccc2)c2ccc4ccc5ccccc5c4c2)cccc1n3-c1ccccc1]
Compound C 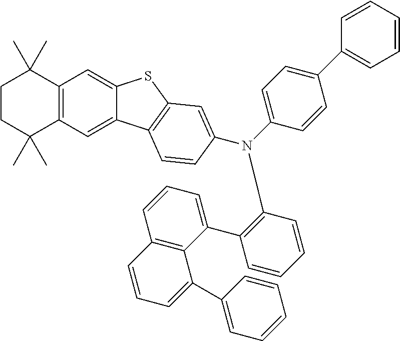 [SMILES: CC1(C)CCC(C)(C)c2cc3c(cc21)sc1cc(N(c2ccc(-c4ccccc4)cc2)c2ccccc2-c2cccc4cccc(-c5ccccc5)c24)ccc13]
Compound D

The voltage efficiency performance of the organic electroluminescent devices fabricated as described above was tested under the condition of 10 mA/cm², and the lifetime performance of the devices was tested under the condition of 20 mA/cm². The results are presented in Table 8 below.

TABLE 8
Performance test results of organic electroluminescent devices

| | | Oper- | Current | | | |
|---|---|---|---|---|---|---|
| | Hole | ating | effi- | | | T₉₅(hrs) |
| Example | Auxiliary | voltage | ciency | CIE- | CIE- | @20 |
| No. | Layer | (V) | Cd/A | x | y | mA/cm² |
| Example1 | Compound 8 | 4.09 | 99.2 | 0.22 | 0.73 | 385 |
| Example2 | Compound 16 | 3.93 | 102.7 | 0.22 | 0.73 | 393 |
| Example3 | Compound 17 | 4.04 | 102.8 | 0.22 | 0.73 | 391 |
| Example4 | Compound 21 | 3.96 | 100.7 | 0.22 | 0.73 | 390 |
| Example5 | Compound 24 | 3.94 | 102.9 | 0.22 | 0.73 | 386 |
| Example6 | Compound 35 | 4.00 | 100.1 | 0.22 | 0.73 | 383 |
| Example7 | Compound 48 | 4.11 | 101.1 | 0.22 | 0.73 | 393 |
| Example8 | Compound 52 | 4.07 | 101.5 | 0.22 | 0.73 | 384 |
| Example9 | Compound 54 | 4.06 | 101.9 | 0.22 | 0.73 | 389 |
| Example10 | Compound 62 | 4.09 | 100.7 | 0.22 | 0.73 | 387 |
| Example11 | Compound 71 | 3.90 | 101.7 | 0.22 | 0.73 | 392 |
| Example12 | Compound 80 | 4.01 | 97.2 | 0.22 | 0.73 | 380 |
| Example13 | Compound 108 | 3.98 | 101.4 | 0.22 | 0.73 | 386 |
| Example14 | Compound 125 | 3.95 | 101.6 | 0.22 | 0.73 | 391 |
| Example15 | Compound 145 | 4.06 | 102.2 | 0.22 | 0.73 | 387 |
| Example16 | Compound 152 | 4.02 | 102.5 | 0.22 | 0.73 | 392 |
| Example17 | Compound 161 | 3.99 | 102.0 | 0.22 | 0.73 | 390 |
| Example18 | Compound 169 | 4.08 | 99.5 | 0.22 | 0.73 | 393 |
| Example19 | Compound 178 | 4.10 | 100.6 | 0.22 | 0.73 | 384 |
| Example20 | Compound 193 | 3.98 | 102.4 | 0.22 | 0.73 | 388 |
| Example21 | Compound 198 | 3.97 | 98.4 | 0.22 | 0.73 | 383 |
| Example22 | Compound 204 | 4.03 | 99.3 | 0.22 | 0.73 | 389 |
| Example23 | Compound 208 | 3.92 | 97.5 | 0.22 | 0.73 | 393 |
| Example24 | Compound 237 | 3.91 | 100.5 | 0.22 | 0.73 | 385 |
| Example25 | Compound 242 | 4.02 | 103.0 | 0.22 | 0.73 | 383 |
| Example26 | Compound 251 | 3.99 | 100.9 | 0.22 | 0.73 | 388 |
| Example27 | Compound 254 | 4.06 | 99.7 | 0.22 | 0.73 | 386 |
| Example28 | Compound 255 | 3.92 | 99.9 | 0.22 | 0.73 | 381 |
| Example29 | Compound 458 | 4.11 | 99.6 | 0.22 | 0.73 | 390 |
| Example30 | Compound 256 | 4.11 | 100.4 | 0.22 | 0.73 | 391 |
| Example31 | Compound 277 | 4.02 | 97.9 | 0.22 | 0.73 | 384 |
| Example32 | Compound 281 | 3.93 | 99.6 | 0.22 | 0.73 | 388 |
| Example33 | Compound 283 | 4.05 | 97.7 | 0.22 | 0.73 | 391 |
| Example34 | Compound 293 | 3.97 | 102.3 | 0.22 | 0.73 | 385 |
| Example35 | Compound 295 | 3.91 | 100.2 | 0.22 | 0.73 | 392 |
| Example36 | Compound 302 | 3.95 | 96.7 | 0.22 | 0.73 | 386 |
| Example37 | Compound 459 | 4.03 | 97.1 | 0.22 | 0.73 | 381 |
| Example38 | Compound 308 | 3.96 | 98.8 | 0.22 | 0.73 | 380 |
| Example39 | Compound 317 | 4.04 | 100.8 | 0.22 | 0.73 | 389 |
| Example40 | Compound 328 | 3.94 | 98.1 | 0.22 | 0.73 | 385 |
| Example41 | Compound 334 | 4.10 | 98.6 | 0.22 | 0.73 | 390 |
| Example42 | Compound 448 | 4.01 | 101.0 | 0.22 | 0.73 | 380 |
| Example43 | Compound 342 | 4.07 | 101.8 | 0.22 | 0.73 | 387 |
| Example44 | Compound 359 | 4.07 | 100.3 | 0.22 | 0.73 | 382 |
| Example45 | Compound 368 | 4.01 | 99.8 | 0.22 | 0.73 | 384 |
| Example46 | Compound 449 | 4.08 | 101.2 | 0.22 | 0.73 | 381 |
| Example47 | Compound 450 | 3.92 | 99.2 | 0.22 | 0.73 | 391 |
| Example48 | Compound 372 | 3.93 | 96.9 | 0.22 | 0.73 | 390 |
| Example49 | Compound 451 | 3.94 | 96.8 | 0.22 | 0.73 | 382 |
| Example50 | Compound 452 | 4.10 | 99.1 | 0.22 | 0.73 | 382 |
| Example51 | Compound 375 | 4.00 | 106.8 | 0.22 | 0.73 | 389 |
| Example52 | Compound 376 | 4.05 | 107.0 | 0.22 | 0.73 | 381 |
| Example53 | Compound 377 | 4.03 | 106.8 | 0.22 | 0.73 | 392 |
| Example54 | Compound 382 | 3.98 | 107.1 | 0.22 | 0.73 | 387 |
| Example55 | Compound 383 | 3.90 | 107.3 | 0.22 | 0.73 | 392 |
| Example56 | Compound 390 | 3.91 | 108.0 | 0.22 | 0.73 | 389 |
| Example57 | Compound 432 | 4.06 | 107.9 | 0.22 | 0.73 | 386 |
| Example58 | Compound 457 | 4.02 | 107.6 | 0.22 | 0.73 | 388 |
| Example 59 | Compound 426 | 4.04 | 96.7 | 0.22 | 0.73 | 363 |
| Example 60 | Compound 427 | 3.95 | 98.5 | 0.22 | 0.73 | 362 |
| Example 61 | Compound 437 | 4.00 | 102.0 | 0.22 | 0.73 | 363 |
| Example 62 | Compound 438 | 4.11 | 101.0 | 0.22 | 0.73 | 366 |
| Example 63 | Compound 439 | 3.99 | 107.3 | 0.22 | 0.73 | 360 |
| Example 64 | Compound 440 | 4.01 | 107.2 | 0.22 | 0.73 | 367 |
| Example 65 | Compound 441 | 4.08 | 106.8 | 0.22 | 0.73 | 359 |
| Example 66 | Compound 442 | 4.10 | 108.0 | 0.22 | 0.73 | 359 |
| Example 67 | Compound 430 | 3.92 | 97.4 | 0.22 | 0.73 | 366 |
| Example 68 | Compound 443 | 3.95 | 100.6 | 0.22 | 0.73 | 364 |
| Example 69 | Compound 444 | 3.97 | 106.9 | 0.22 | 0.73 | 361 |
| Example 70 | Compound 445 | 3.94 | 107.8 | 0.22 | 0.73 | 365 |
| Example 71 | Compound 402 | 3.99 | 97.6 | 0.22 | 0.73 | 340 |
| Example 72 | Compound 412 | 3.94 | 97.3 | 0.22 | 0.73 | 339 |
| Example 73 | Compound 433 | 4.08 | 99.3 | 0.22 | 0.73 | 341 |
| Example 74 | Compound186 | 4.03 | 102.0 | 0.22 | 0.73 | 342 |
| Example 75 | Compound 435 | 3.91 | 106.0 | 0.22 | 0.73 | 340 |
| Example 76 | Compound186 | 4.01 | 106.5 | 0.22 | 0.73 | 341 |
| Comparative | Compound A | 4.41 | 85.5 | 0.22 | 0.73 | 292 |
| Example 1 | | | | | | |
| Comparative | Compound B | 4.40 | 86.5 | 0.22 | 0.73 | 282 |
| Example 2 | | | | | | |
| Comparative | Compound C | 4.41 | 84.2 | 0.22 | 0.73 | 276 |
| Example 3 | | | | | | |
| Comparative | Compound D | 4.38 | 84.5 | 0.22 | 0.73 | 263 |
| Example 4 | | | | | | |

Therefore, when the novel compounds of the present disclosure was utilized in the fabrication of green organic electroluminescent devices, it ensure that the devices meet the requirement of a lower voltage while effectively enhancing the efficiency of the organic electroluminescent devices and maintaining a long lifetime for the devices. It can be known from the results presented in Table 8 that, compared to the organic electroluminescent devices of Comparative Examples 1 to 4, the current efficiency of the green organic electroluminescent devices of Examples 1 to 76 has been enhanced by at least 11.8%, and their lifetime has been extended by at least 16.1%. Therefore, the utilization of the organic compound proposed in the present disclosure as a hole-auxiliary layer in organic electroluminescent devices can significantly enhance the performance of the devices.

1. An organic compound, having the structure represented by Formula (1): 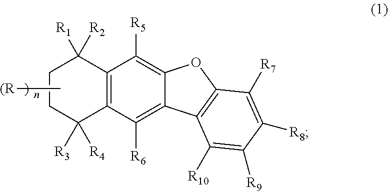
wherein, R₁, R₂, R₃, and R₄ are the same or different, and are each independently selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
one of R₅ and R₆ is a hydrogen, a deuterium, a halogen group, or a cyano, and the other is 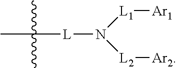
R₇, R₈, R₉, and R₁₀ are the same or different, and are each independently selected from a hydrogen, a deuterium, a halogen group, a cyano, an alkyl having 1 to 10 carbon atoms, an aryl having 6 to 20 carbon atoms, a heteroaryl having 3 to 20 carbon atoms, a deuterated alkyl having 1 to 10 carbon atoms, a deuterated aryl having 6 to 20 carbon atoms, and a deuterated heteroaryl having 3 to 20 carbon atoms;
optionally, R₇ and R₈, and/or R₈ and R₉, and/or R₉ and R₁₀, are interconnected to form a substituted or unsubstituted 5- to 13-membered ring;
substituent(s) on the 5- to 13-membered ring are the same or different, and are each independently selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
X is selected from C(R₁₁R₁₂), O, S, or N(R₁₃);
R₁₁ and R₁₂ are the same or different, and are each independently selected from a hydrogen, a deuterium, an alkyl having 1 to 10 carbon atoms, an aryl having 6 to 20 carbon atoms, a deuterated alkyl having 1 to 10 carbon atoms, and a deuterated aryl having 6 to 20 carbon atoms;
R₁₃ is selected from an alkyl having 1 to 10 carbon atoms, an aryl having 6 to 20 carbon atoms, a deuterated alkyl having 1 to 10 carbon atoms, and a deuterated aryl having 6 to 20 carbon atoms;
each R is independently selected from a deuterium, a halogen group, a cyano, an alkyl having 1 to 10 carbon atoms, an aryl having 6 to 20 carbon atoms, a deuterated alkyl having 1 to 10 carbon atoms, and a deuterated aryl having 6 to 20 carbon atoms;
n is the number of R, and n is selected from 0, 1, 2, 3, and 4;
L, L₁, and L₂ are the same or different, and are each independently selected from a single bond, a substituted or unsubstituted arylene having 6 to 30 carbon atoms, and a substituted or unsubstituted heteroarylene having 3 to 30 carbon atoms;
Ar₁ and Ar₂ are the same or different, and are each independently selected from a substituted or unsubstituted aryl having 6 to 40 carbon atoms, and a substituted or unsubstituted heteroaryl having 3 to 40 carbon atoms;
substituent(s) in Ar₁, Ar₂, L, L₁, and L₂ are the same or different, and are each independently selected from a deuterium, a cyano, a fluorine, an alkyl having 1 to 10 carbon atoms, a haloalkyl having 1 to 10 carbon atoms, a deuterated alkyl having 1 to 10 carbon atoms, an aryl having 6 to 20 carbon atoms, a haloaryl having 6 to 20 carbon atoms, a deuterated aryl having 6 to 20 carbon atoms, a heteroaryl having 3 to 20 carbon atoms, a haloheteroaryl having 3 to 20 carbon atoms, a deuterated heteroaryl having 3 to 20 carbon atoms, a cycloalkyl having 3 to 10 carbon atoms, a halocycloalkyl having 3 to 10 carbon atoms, a deuterated cycloalkyl having 3 to 10 carbon atoms, a triarylsilyl having 18 to 24 carbon atoms, and a trialkylsilyl having 3 to 12 carbon atoms;
optionally, any two adjacent substituents in Ar₁ and Ar₂ form a saturated or unsaturated 5- to 13-membered ring.

2. The organic compound according to claim 1, wherein Ar₁ and Ar₂ are the same or different, and are each independently selected from a substituted or unsubstituted aryl having 6 to 25 carbon atoms, and a substituted or unsubstituted heteroaryl having 12 to 24 carbon atoms;
optionally, substituent(s) in Ar₁ and Ar₂ are each independently selected from a deuterium, a cyano, a fluorine, an alkyl having 1 to 4 carbon atoms, a haloalkyl having 1 to 4 carbon atoms, a deuterated alkyl having 1 to 4 carbon atoms, a phenyl, a naphthyl, a heteroaryl having 5 to 12 carbon atoms, a cycloalkyl having 5 to 10 carbon atoms, a trimethylsilyl, and a triphenylsilyl;
optionally, any two adjacent substituents in Ar₁ and Ar₂ form a cyclopentyl, a cyclohexyl, a phenyl, or a fluorenyl.

3. The organic compound according to claim 1, wherein Ar₁ and Ar₂ are the same or different, and are each independently selected from a substituted or unsubstituted phenyl, a substituted or unsubstituted naphthyl, a substituted or unsubstituted biphenyl, a substituted or unsubstituted terphenyl, a substituted or unsubstituted phenanthryl, a substituted or unsubstituted anthracenyl, a substituted or unsubstituted triphenylene, a substituted or unsubstituted fluorenyl, a substituted or unsubstituted dibenzothienyl, a substituted or unsubstituted dibenzofuranyl, a substituted or unsubstituted carbazolyl, a substituted or unsubstituted spirobifluorenyl, a substituted or unsubstituted cyclopentane-spiro-fluorenyl, and a substituted or unsubstituted cyclohexane-spiro-fluorenyl;
optionally, substituent(s) in Ar₁ and Ar₂ are the same or different, and are each independently selected from a deuterium, a fluorine, a cyano, a methyl, an ethyl, an isopropyl, a tert-butyl, a trifluoromethyl, a trideuterated methyl, a phenyl, a naphthyl, a carbazolyl, a trimethylsilyl, a triphenylsilyl, a cyclopentyl, and a cyclohexyl.

4. The organic compound according to claim 1, wherein Ar₁ and Ar₂ are the same or different, and are each independently selected from the following groups:

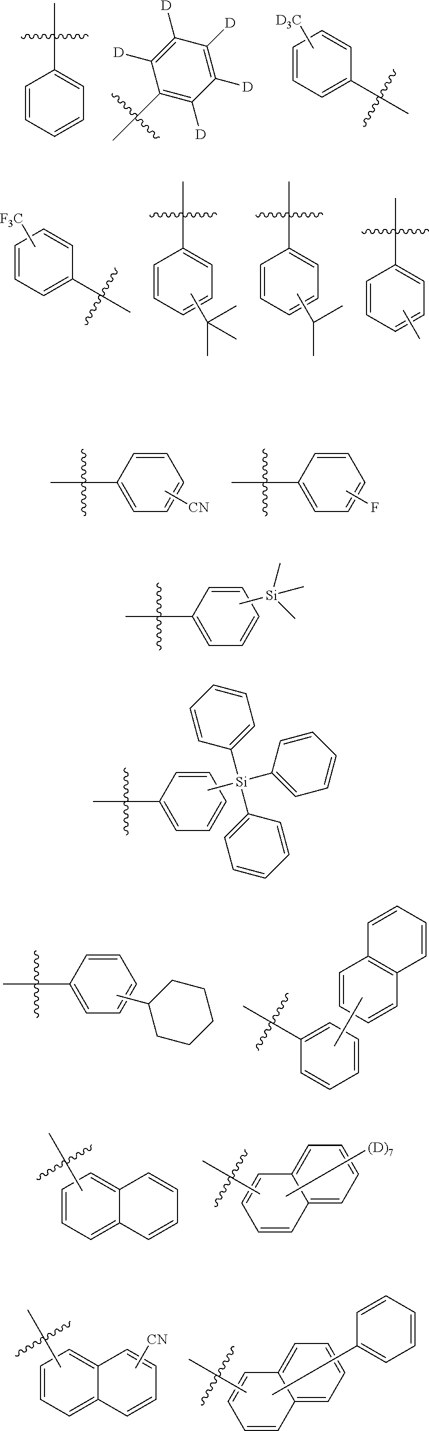

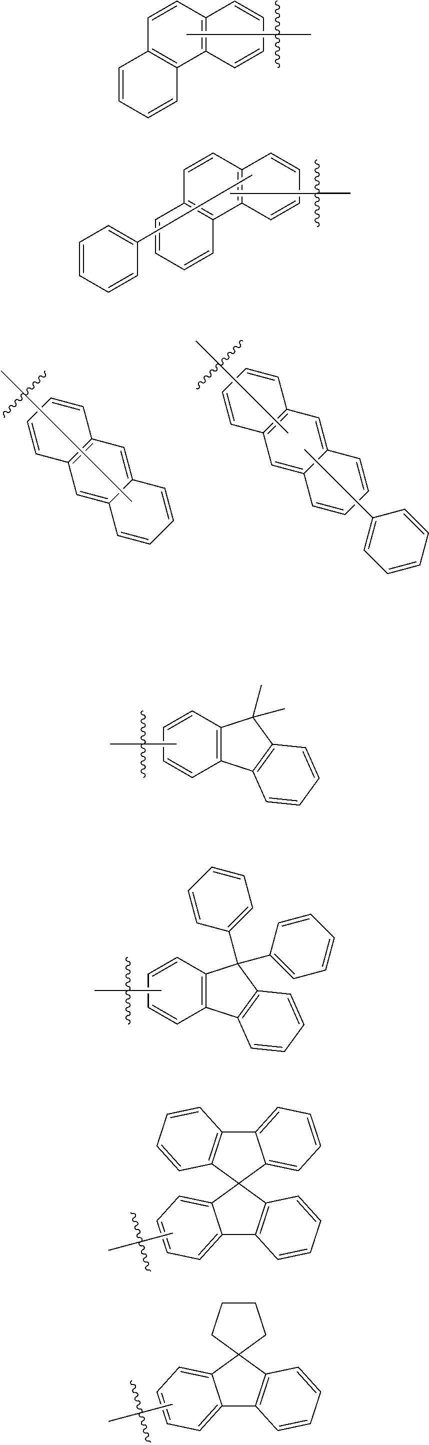

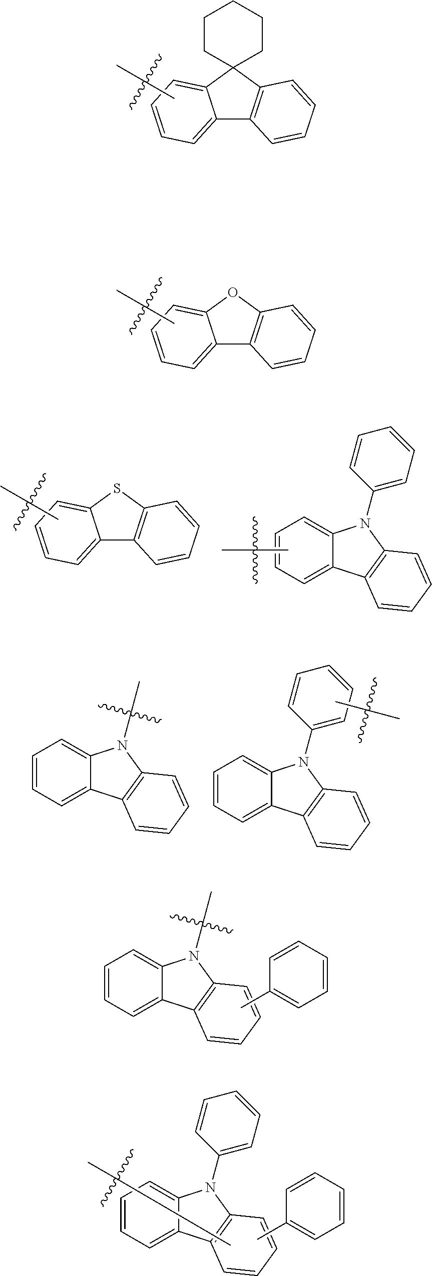

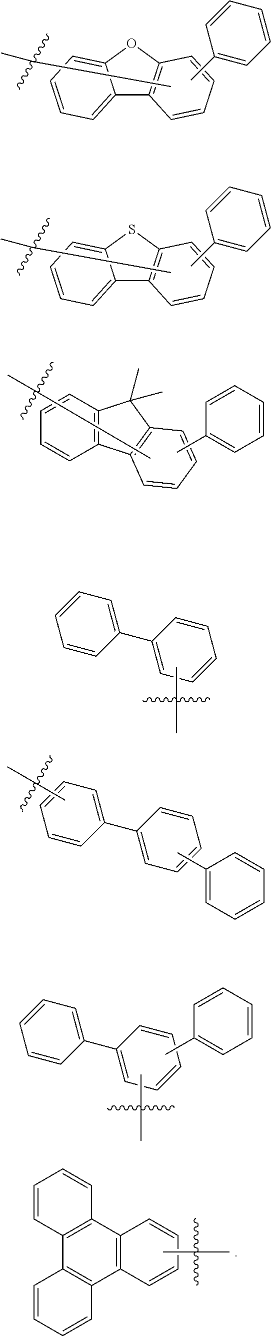

5. The organic compound according to claim 1, wherein L, L₁, and L₂ are the same or different, and are each independently selected from a single bond, a substituted or unsubstituted arylene having 6 to 18 carbon atoms, and a substituted or unsubstituted heteroarylene having 12 to 18 carbon atoms;
optionally, substituent(s) in L, L₁ and L₂ are the same or different, and are each independently selected from a deuterium, a cyano, a halogen group, an alkyl having 1 to 4 carbon atoms, a haloalkyl having 1 to 4 carbon atoms, a deuterated alkyl having 1 to 4 carbon atoms, an aryl having 6 to 12 carbon atoms, a haloaryl having 6 to 12 carbon atoms, a deuterated aryl having 6 to 12 carbon atoms, a heteroaryl having 5 to 12 carbon atoms, a haloheteroaryl having 5 to 12 carbon atoms, and a deuterated heteroaryl having 5 to 12 carbon atoms.

6. The organic compound according to claim 1, wherein L, L₁, and L₂ are the same or different, and are each independently selected from a single bond, a substituted or unsubstituted phenylene, a substituted or unsubstituted naphthylene, a substituted or unsubstituted biphenylene, a substituted or unsubstituted terphenylene, a substituted or unsubstituted phenanthrylene, a substituted or unsubstituted dibenzothienylene, a substituted or unsubstituted dibenzofuranylene, and a substituted or unsubstituted carbazolylene;
optionally, substituent(s) in L, L₁, and L₂ are the same or different, and are each independently selected from a deuterium, a fluorine, a cyano, a methyl, an ethyl, an isopropyl, a tert-butyl, a trifluoromethyl, a trideuterated methyl, a phenyl, and a naphthyl.

7. The organic compound according to claim 1, wherein L is selected from the group consisting of the following groups:

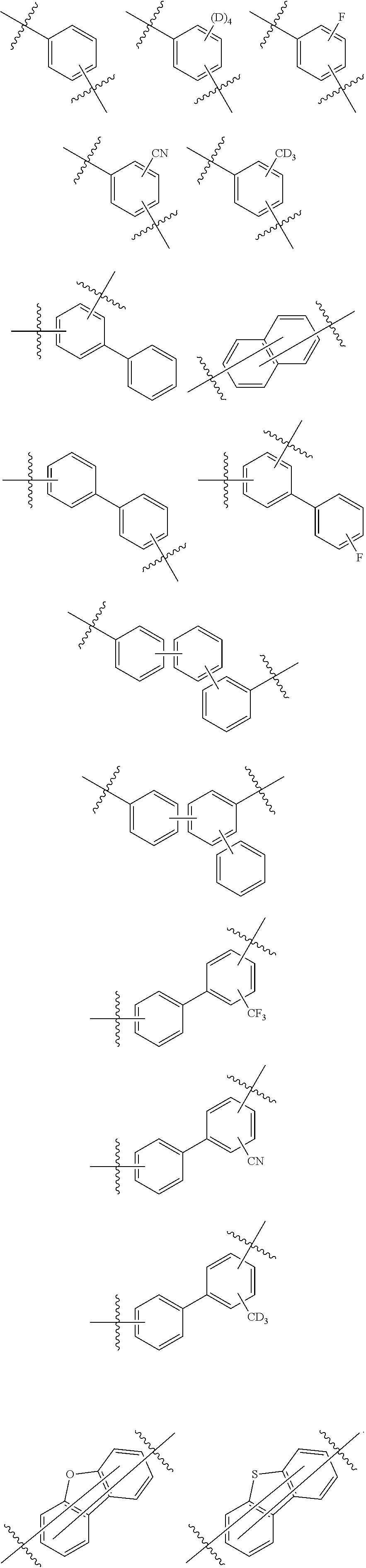

8. The organic compound according to claim 1, wherein L₁ and L₂ are selected from a single bond or the group consisting of the following groups:

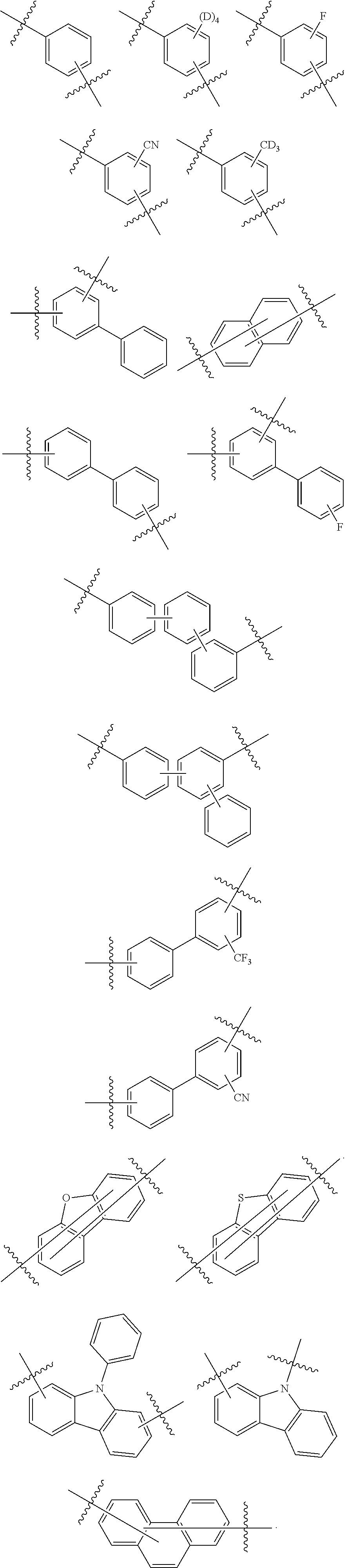

9. The organic compound according to claim 1, wherein 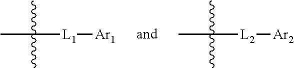
are each independently selected from the following groups:

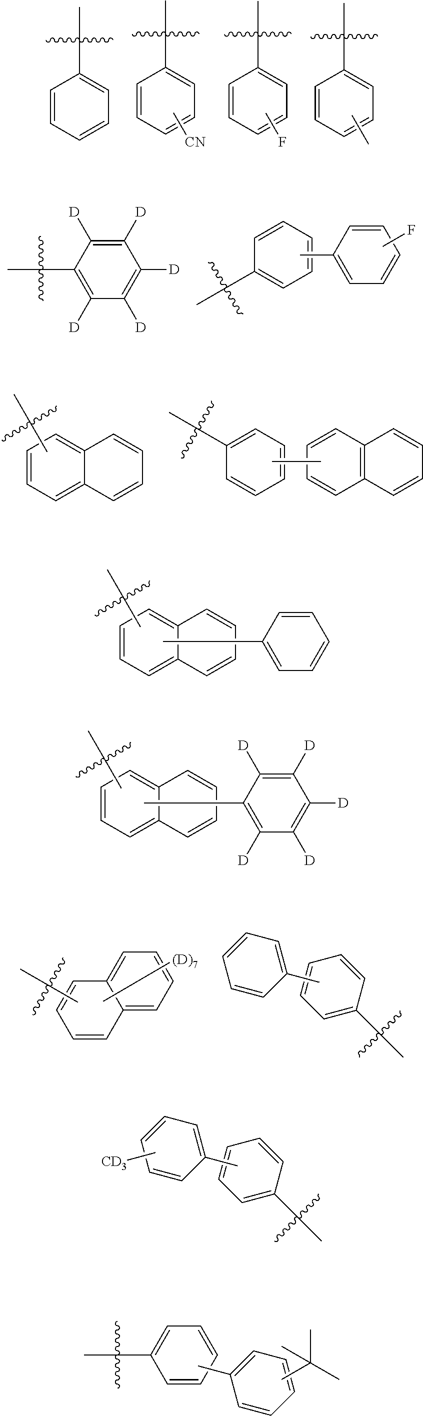

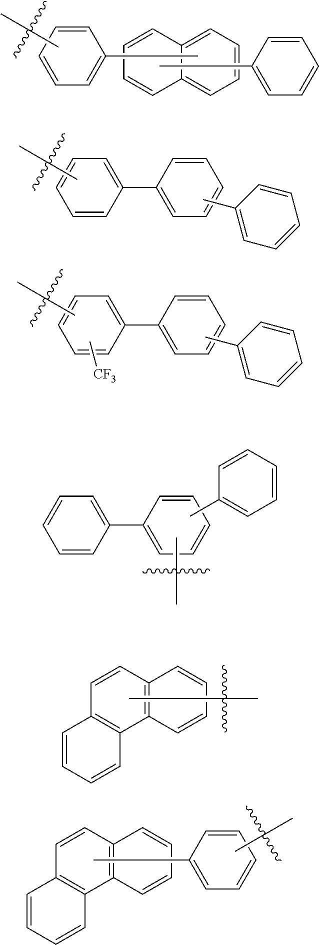

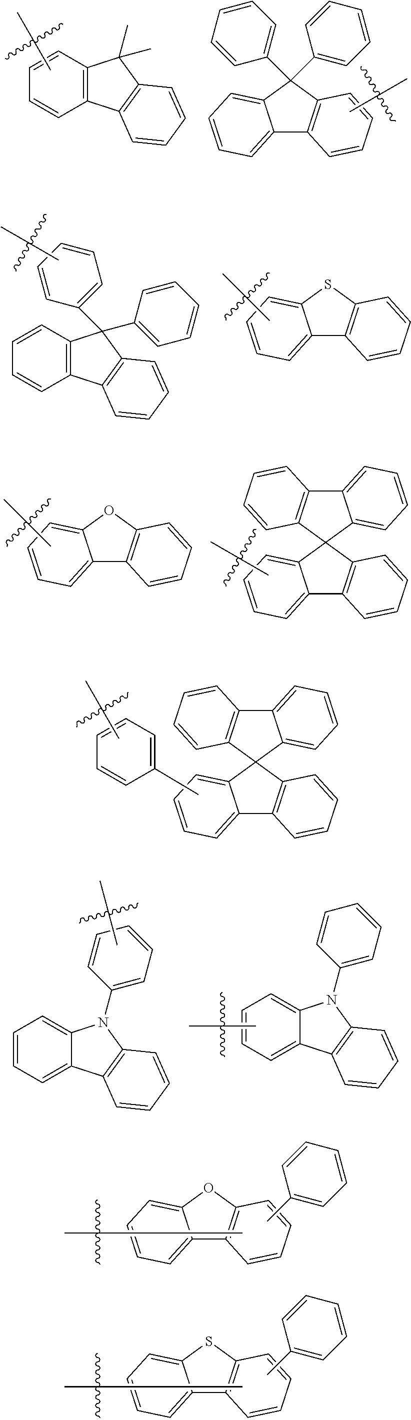

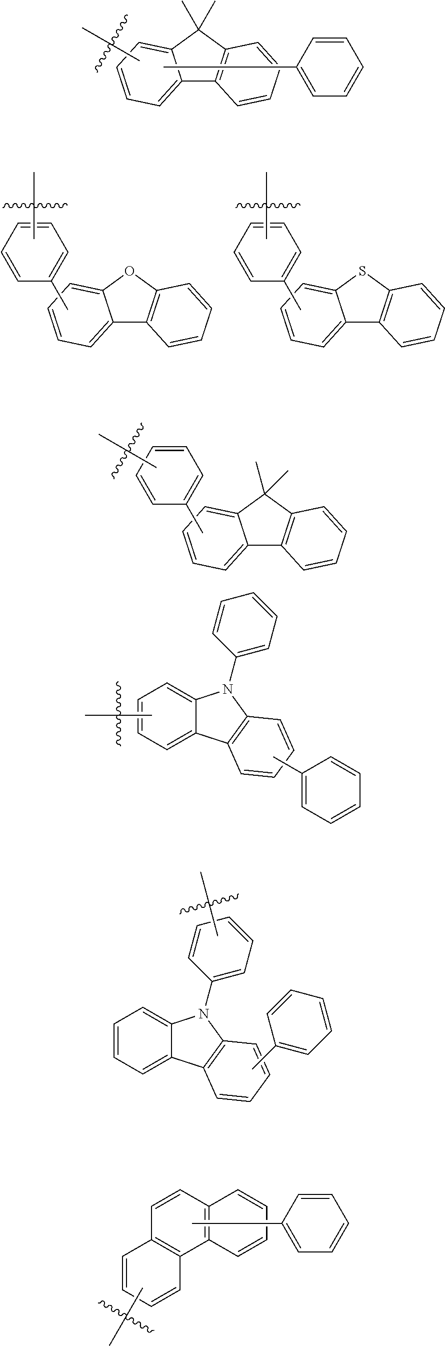

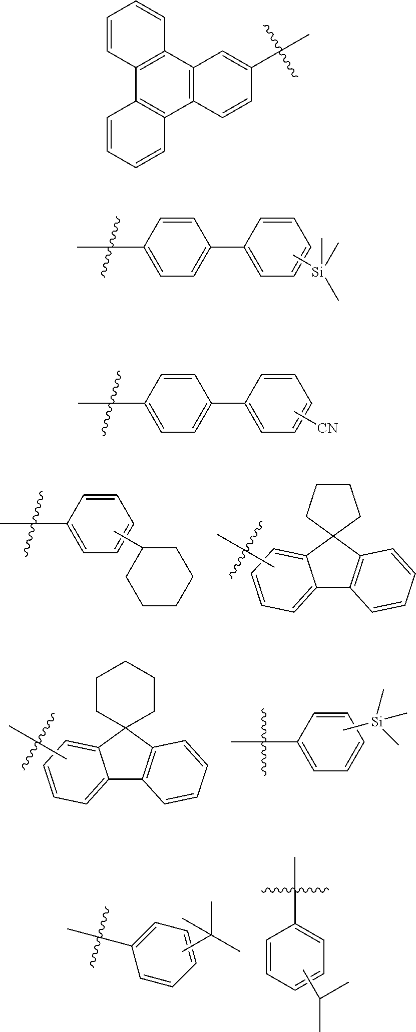

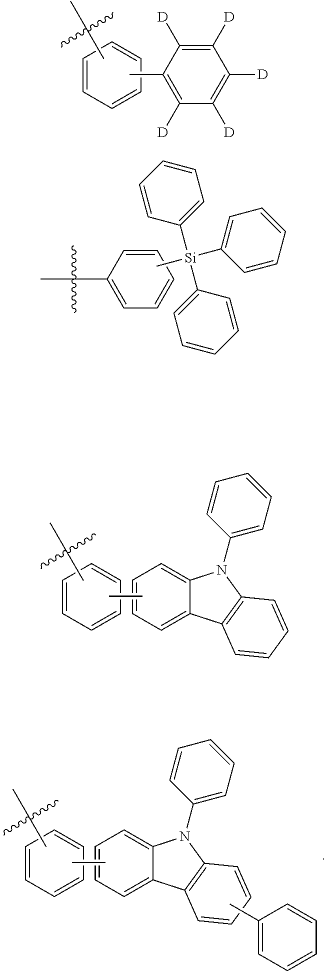

10. The organic compound according to claim 1, wherein R₁, R₂, R₃ and R₄ are the same or different, and are each independently selected from a methyl, a trideuterated methyl, a phenyl, and a penta-deuterated phenyl;
optionally, each R is independently selected from a deuterium, a fluorine, a cyano, a methyl, a tert-butyl, and a phenyl;
optionally, R₇, R₈, R₉ and R₁₀ are the same or different, and are each independently selected from a hydrogen, a deuterium, a methyl, an ethyl, an isopropyl, a tert-butyl, a phenyl, a deuterated methyl, a deuterated ethyl, a deuterated isopropyl, a deuterated tert-butyl, and a deuterated phenyl;
optionally, R₇ and R₈, and/or R₈ and R₉, and/or R₉ and R₁₀, are interconnected to form a substituted or unsubstituted cyclohexane;
substituent(s) in the cyclohexane are the same or different, and are each independently selected from a methyl, a trideuterated methyl, a phenyl, and a penta-deuterated phenyl.

11. The organic compound according to claim 1, wherein R₁₁ and R₁₂ are the same or different, and are each independently selected from the following groups:
optionally, R₁₃ is selected from a phenyl, a naphthyl, and a biphenyl.

12. The organic compound according to claim 1, wherein the compound is selected from the group consisting of the following compounds:

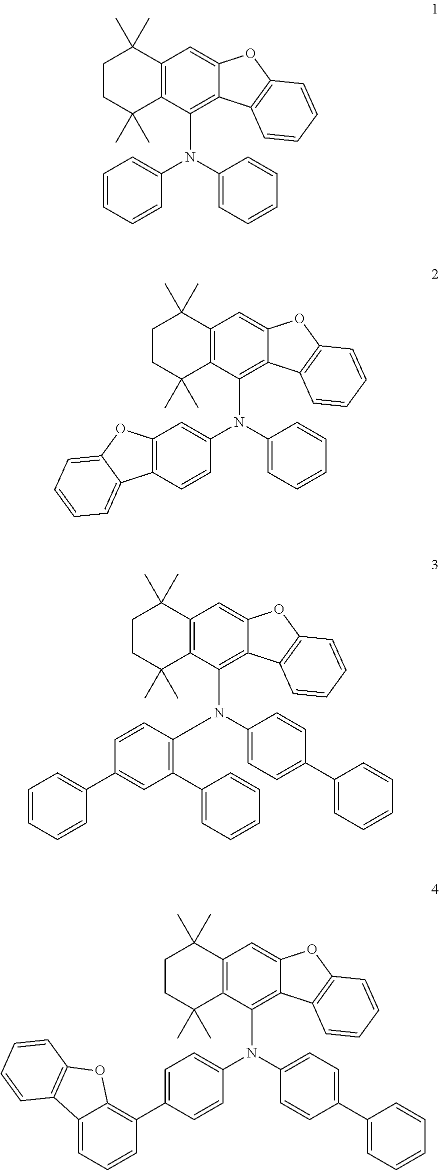 [SMILES: CC1(C)CCC(C)(C)c2c1cc1oc3ccccc3c1c2N(c1ccccc1)c1ccccc1 ; CC1(C)CCC(C)(C)c2c1cc1oc3ccccc3c1c2N(c1ccccc1)c1ccc2c(c1)oc1ccccc12 ; CC1(C)CCC(C)(C)c2c1cc1oc3ccccc3c1c2N(c1ccc(-c2ccccc2)cc1)c1ccc(-c2ccccc2)cc1-c1ccccc1 ; CC1(C)CCC(C)(C)c2c1cc1oc3ccccc3c1c2N(c1ccc(-c2ccccc2)cc1)c1ccc(-c2cccc3c2oc2ccccc23)cc1]

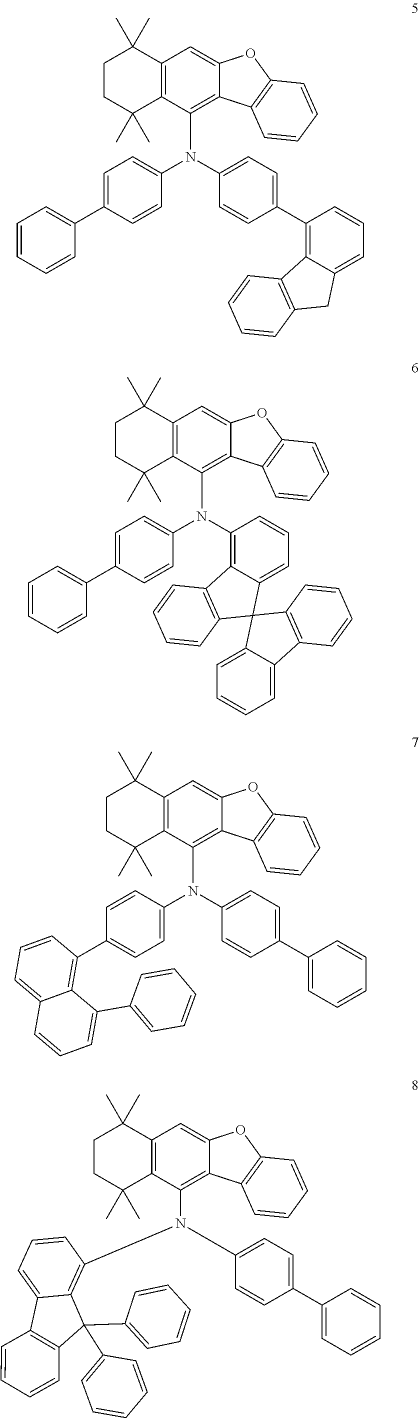 [SMILES: CC1(C)CCC(C)(C)c2c1cc1oc3ccccc3c1c2N(c1ccc(-c2ccccc2)cc1)c1ccc(-c2cccc3c2-c2ccccc2C3)cc1 ; CC1(C)CCC(C)(C)c2c1cc1oc3ccccc3c1c2N(c1ccc(-c2ccccc2)cc1)c1cccc2c1-c1ccccc1C21c2ccccc2-c2ccccc21 ; CC1(C)CCC(C)(C)c2c1cc1oc3ccccc3c1c2N(c1ccc(-c2ccccc2)cc1)c1ccc(-c2cccc3cccc(-c4ccccc4)c23)cc1 ; CC1(C)CCC(C)(C)c2c1cc1oc3ccccc3c1c2N(c1ccc(-c2ccccc2)cc1)c1cccc2c1C(c1ccccc1)(c1ccccc1)c1ccccc1-2]

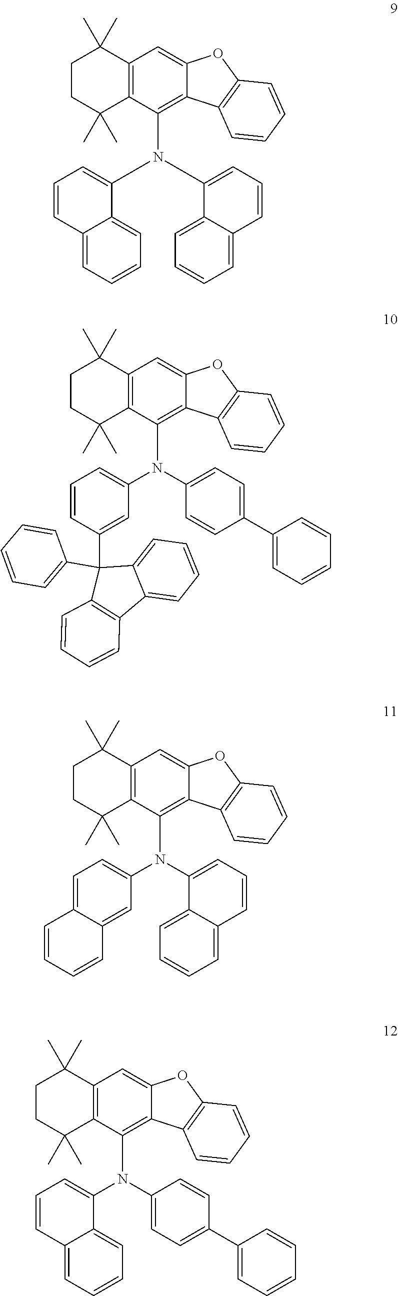

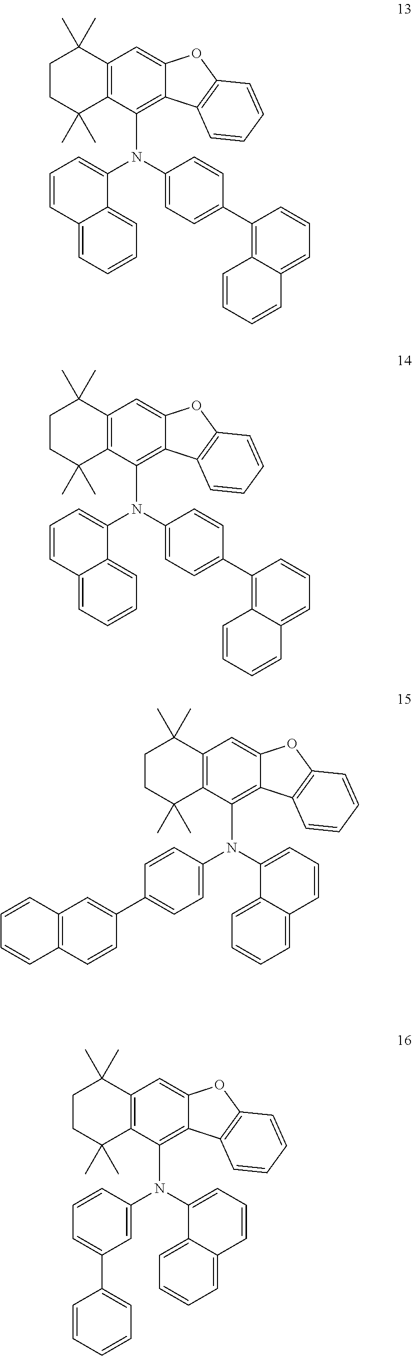 [SMILES: CC1(C)CCC(C)(C)c2c1cc1oc3ccccc3c1c2N(c1ccc(-c2cccc3ccccc23)cc1)c1cccc2ccccc12 ; CC1(C)CCC(C)(C)c2c1cc1oc3ccccc3c1c2N(c1ccc(-c2cccc3ccccc23)cc1)c1cccc2ccccc12 ; CC1(C)CCC(C)(C)c2c1cc1oc3ccccc3c1c2N(c1ccc(-c2ccc3ccccc3c2)cc1)c1cccc2ccccc12 ; CC1(C)CCC(C)(C)c2c1cc1oc3ccccc3c1c2N(c1cccc(-c2ccccc2)c1)c1cccc2ccccc12]

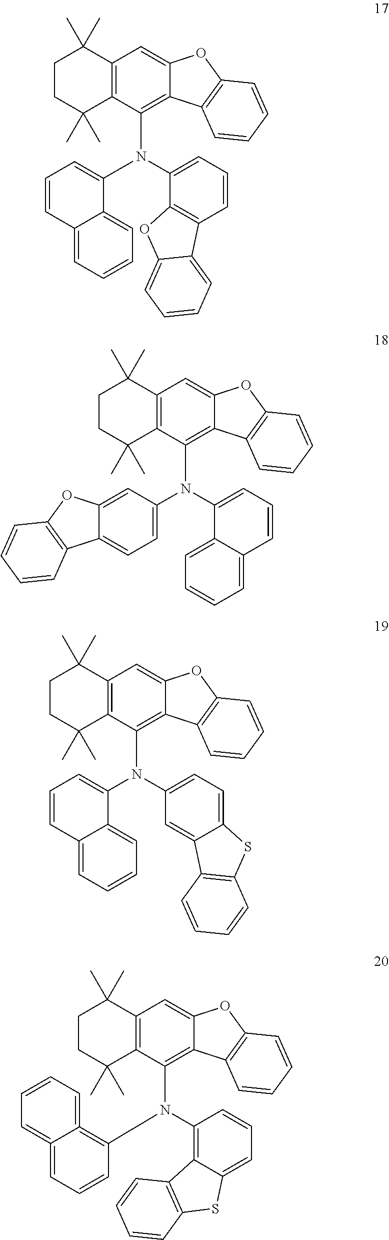

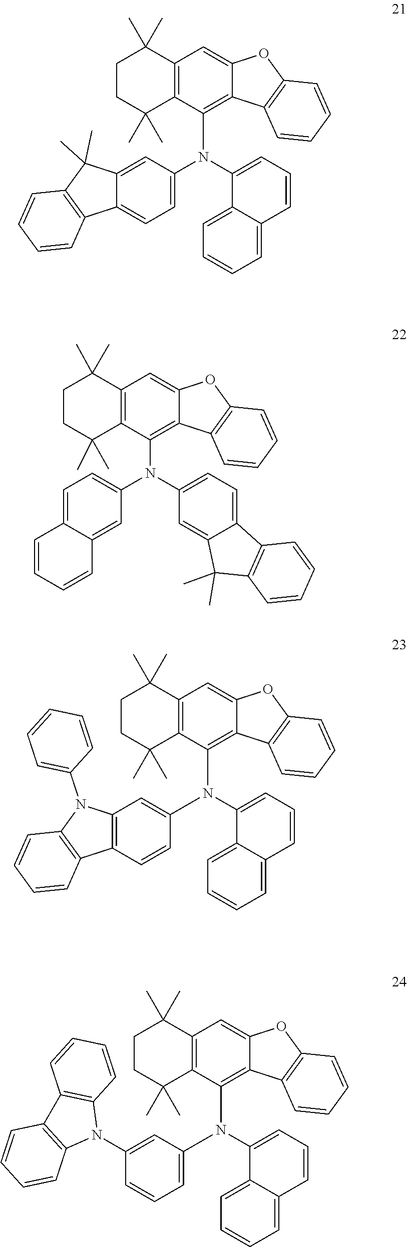 [SMILES: CC1(C)CCC(C)(C)c2c1cc1oc3ccccc3c1c2N(c1ccc2c(c1)C(C)(C)c1ccccc1-2)c1cccc2ccccc12 ; CC1(C)CCC(C)(C)c2c1cc1oc3ccccc3c1c2N(c1ccc2c(c1)C(C)(C)c1ccccc1-2)c1ccc2ccccc2c1 ; CC1(C)CCC(C)(C)c2c1cc1oc3ccccc3c1c2N(c1ccc2c3ccccc3n(-c3ccccc3)c2c1)c1cccc2ccccc12 ; CC1(C)CCC(C)(C)c2c1cc1oc3ccccc3c1c2N(c1cccc(-n2c3ccccc3c3ccccc32)c1)c1cccc2ccccc12]

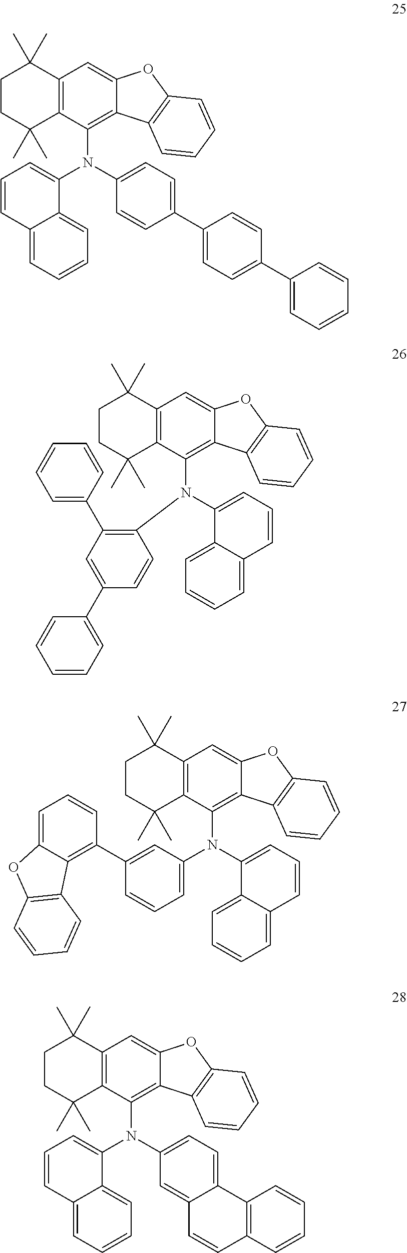 [SMILES: CC1(C)CCC(C)(C)c2c1cc1oc3ccccc3c1c2N(c1ccc(-c2ccc(-c3ccccc3)cc2)cc1)c1cccc2ccccc12 ; CC1(C)CCC(C)(C)c2c1cc1oc3ccccc3c1c2N(c1ccc(-c2ccccc2)cc1-c1ccccc1)c1cccc2ccccc12 ; CC1(C)CCC(C)(C)c2c1cc1oc3ccccc3c1c2N(c1cccc(-c2cccc3oc4ccccc4c23)c1)c1cccc2ccccc12 ; CC1(C)CCC(C)(C)c2c1cc1oc3ccccc3c1c2N(c1ccc2c(ccc3ccccc32)c1)c1cccc2ccccc12]

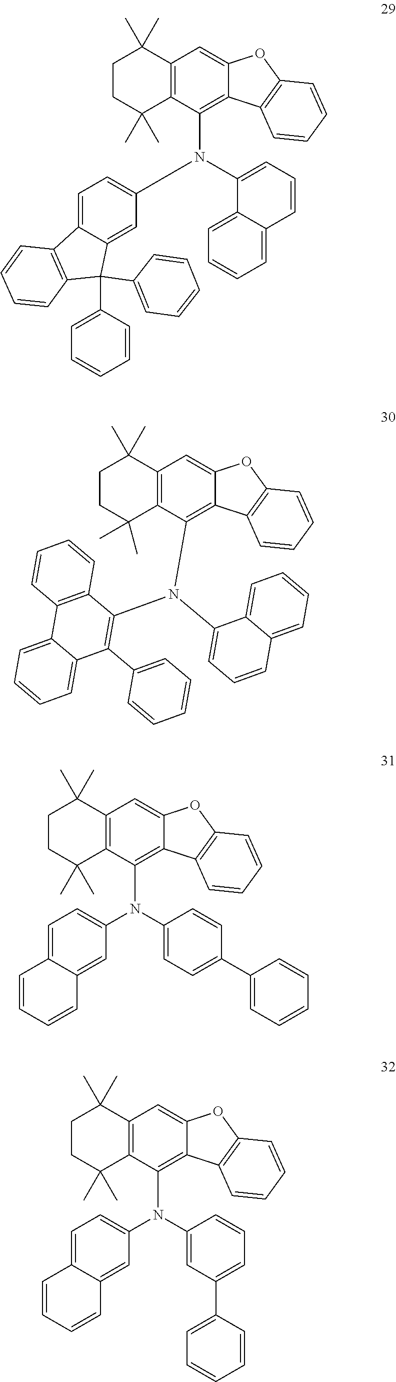 [SMILES: CC1(C)CCC(C)(C)c2c1cc1oc3ccccc3c1c2N(c1ccc2c(c1)C(c1ccccc1)(c1ccccc1)c1ccccc1-2)c1cccc2ccccc12 ; CC1(C)CCC(C)(C)c2c1cc1oc3ccccc3c1c2N(c1cccc2ccccc12)c1c(-c2ccccc2)c2ccccc2c2ccccc12 ; CC1(C)CCC(C)(C)c2c1cc1oc3ccccc3c1c2N(c1ccc(-c2ccccc2)cc1)c1ccc2ccccc2c1 ; CC1(C)CCC(C)(C)c2c1cc1oc3ccccc3c1c2N(c1cccc(-c2ccccc2)c1)c1ccc2ccccc2c1]

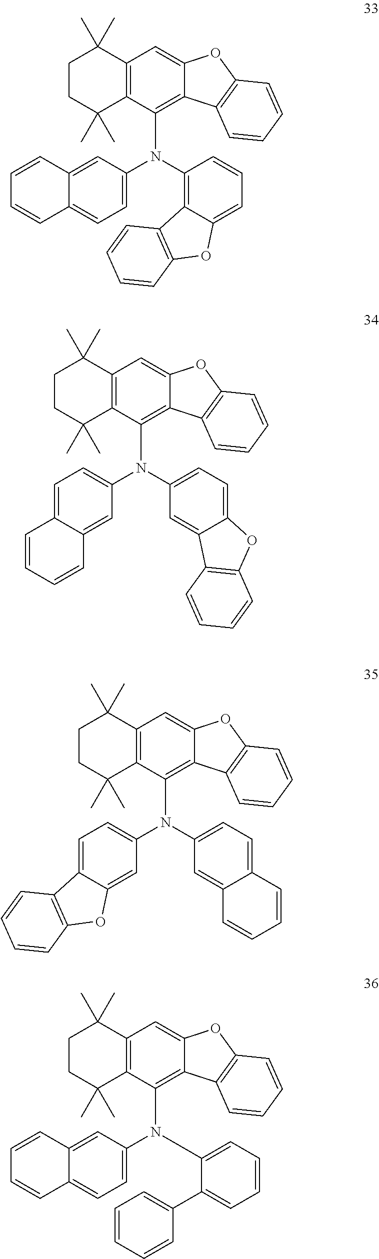 [SMILES: CC1(C)CCC(C)(C)c2c1cc1oc3ccccc3c1c2N(c1ccc2ccccc2c1)c1cccc2oc3ccccc3c12 ; CC1(C)CCC(C)(C)c2c1cc1oc3ccccc3c1c2N(c1ccc2ccccc2c1)c1ccc2oc3ccccc3c2c1 ; CC1(C)CCC(C)(C)c2c1cc1oc3ccccc3c1c2N(c1ccc2ccccc2c1)c1ccc2c(c1)oc1ccccc12 ; CC1(C)CCC(C)(C)c2c1cc1oc3ccccc3c1c2N(c1ccc2ccccc2c1)c1ccccc1-c1ccccc1]

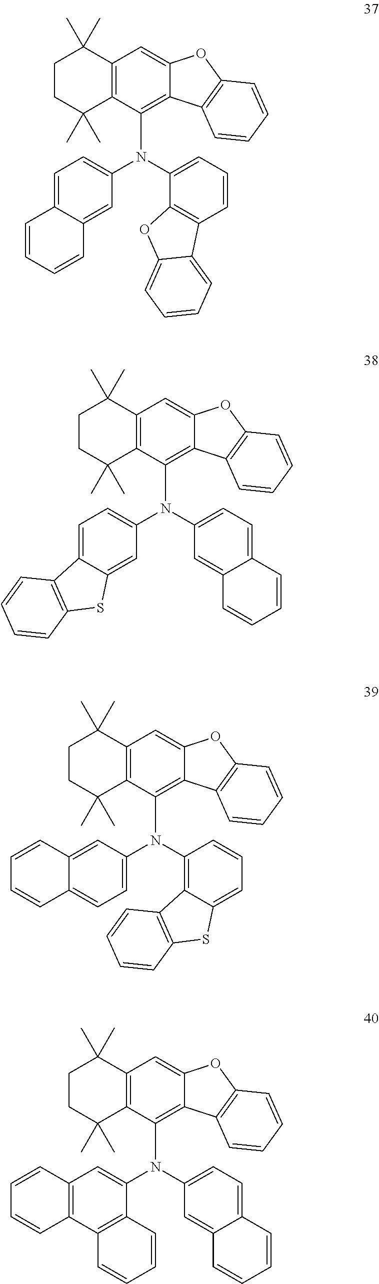 [SMILES: CC1(C)CCC(C)(C)c2c1cc1oc3ccccc3c1c2N(c1ccc2ccccc2c1)c1cccc2c1oc1ccccc12 ; CC1(C)CCC(C)(C)c2c1cc1oc3ccccc3c1c2N(c1ccc2ccccc2c1)c1ccc2c(c1)sc1ccccc12 ; CC1(C)CCC(C)(C)c2c1cc1oc3ccccc3c1c2N(c1ccc2ccccc2c1)c1cccc2sc3ccccc3c12 ; CC1(C)CCC(C)(C)c2c1cc1oc3ccccc3c1c2N(c1ccc2ccccc2c1)c1cc2ccccc2c2ccccc12]

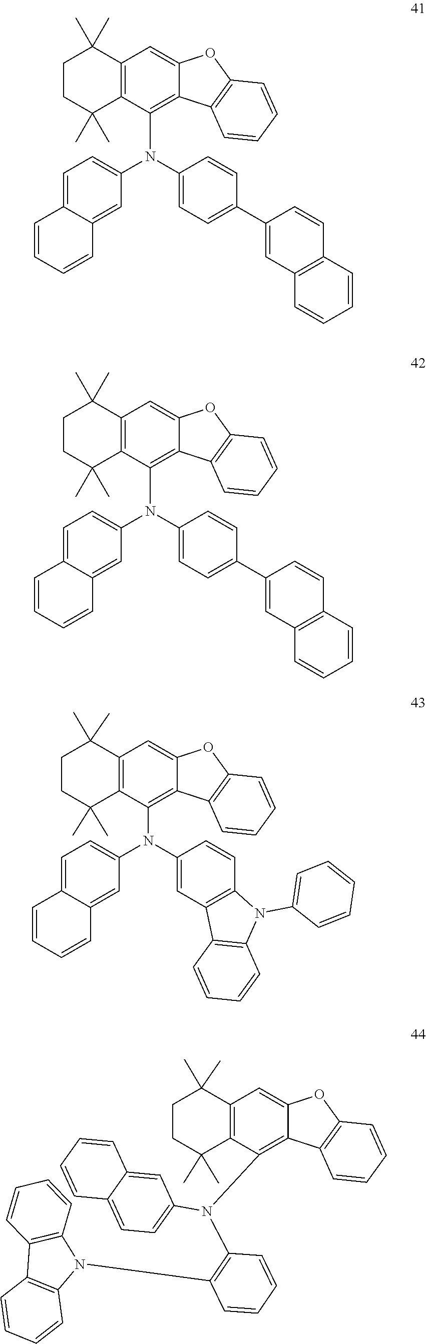 [SMILES: CC1(C)CCC(C)(C)c2c1cc1oc3ccccc3c1c2N(c1ccc(-c2ccc3ccccc3c2)cc1)c1ccc2ccccc2c1 ; CC1(C)CCC(C)(C)c2c1cc1oc3ccccc3c1c2N(c1ccc(-c2ccc3ccccc3c2)cc1)c1ccc2ccccc2c1 ; CC1(C)CCC(C)(C)c2c1cc1oc3ccccc3c1c2N(c1ccc2ccccc2c1)c1ccc2c(c1)c1ccccc1n2-c1ccccc1 ; CC1(C)CCC(C)(C)c2c1cc1oc3ccccc3c1c2N(c1ccc2ccccc2c1)c1ccccc1-n1c2ccccc2c2ccccc21]

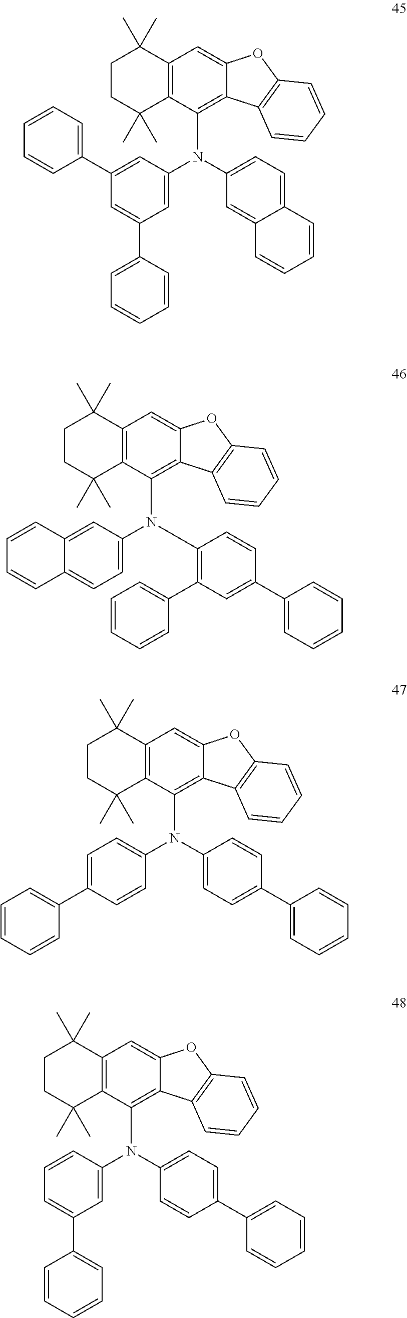 [SMILES: CC1(C)CCC(C)(C)c2c1cc1oc3ccccc3c1c2N(c1cc(-c2ccccc2)cc(-c2ccccc2)c1)c1ccc2ccccc2c1 ; CC1(C)CCC(C)(C)c2c1cc1oc3ccccc3c1c2N(c1ccc2ccccc2c1)c1ccc(-c2ccccc2)cc1-c1ccccc1 ; CC1(C)CCC(C)(C)c2c1cc1oc3ccccc3c1c2N(c1ccc(-c2ccccc2)cc1)c1ccc(-c2ccccc2)cc1 ; CC1(C)CCC(C)(C)c2c1cc1oc3ccccc3c1c2N(c1ccc(-c2ccccc2)cc1)c1cccc(-c2ccccc2)c1]

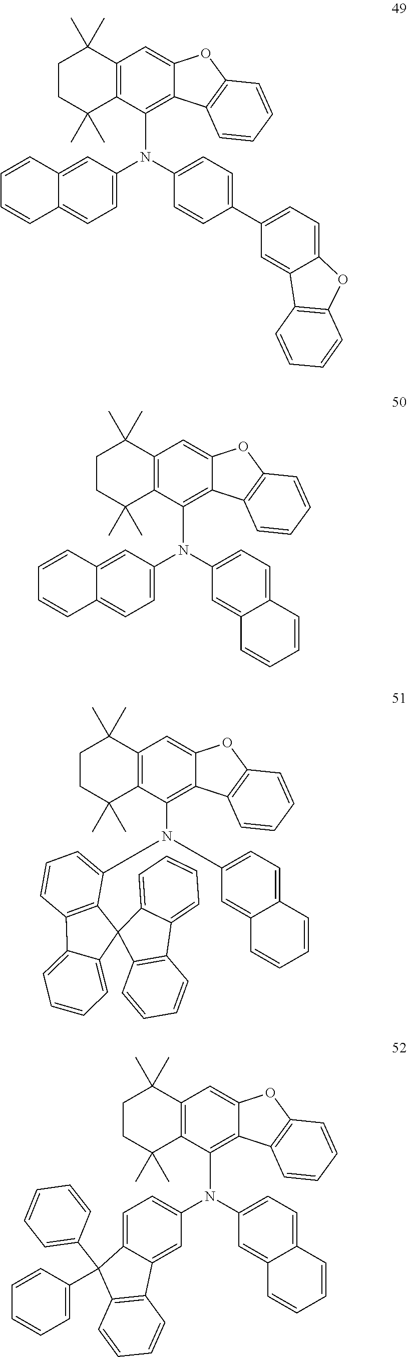

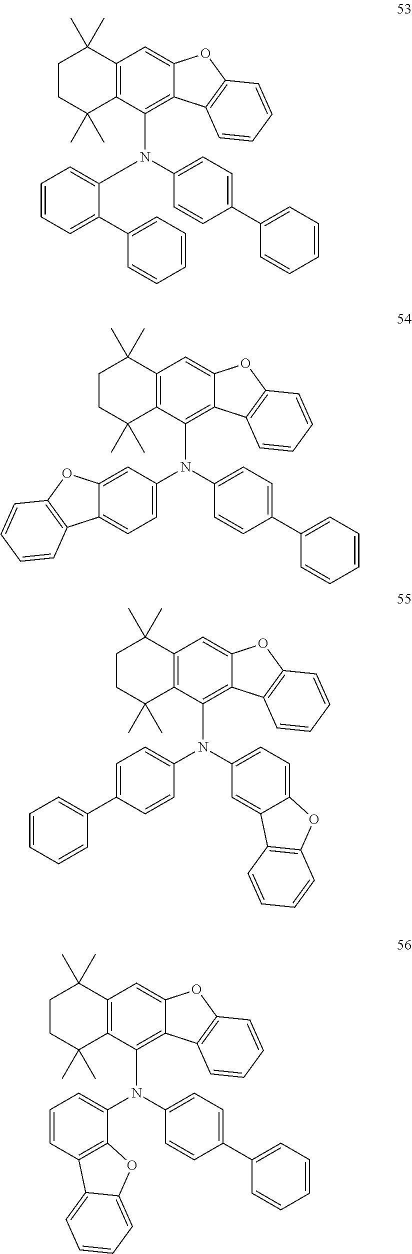

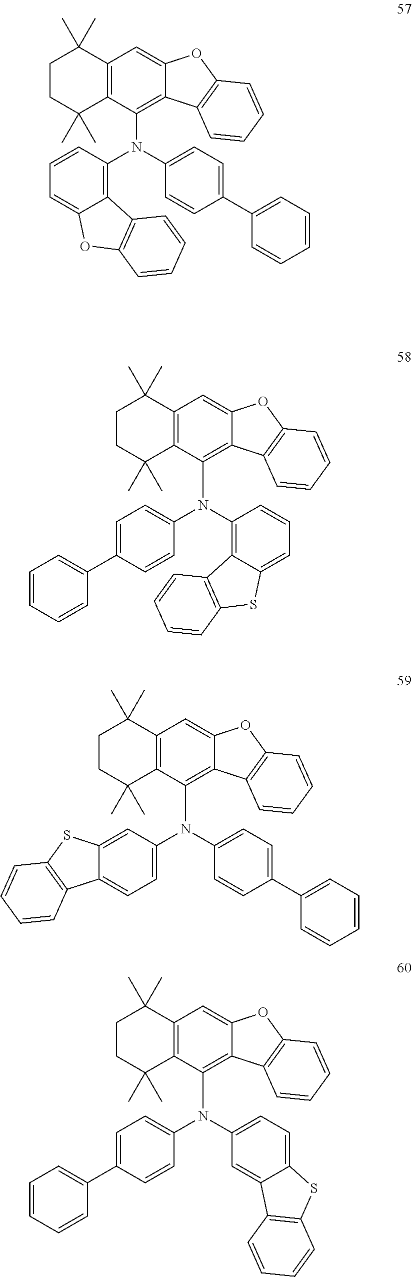 [SMILES: CC1(C)CCC(C)(C)c2c1cc1oc3ccccc3c1c2N(c1ccc(-c2ccccc2)cc1)c1cccc2oc3ccccc3c12 ; CC1(C)CCC(C)(C)c2c1cc1oc3ccccc3c1c2N(c1ccc(-c2ccccc2)cc1)c1cccc2sc3ccccc3c12 ; CC1(C)CCC(C)(C)c2c1cc1oc3ccccc3c1c2N(c1ccc(-c2ccccc2)cc1)c1ccc2c(c1)sc1ccccc12 ; CC1(C)CCC(C)(C)c2c1cc1oc3ccccc3c1c2N(c1ccc(-c2ccccc2)cc1)c1ccc2sc3ccccc3c2c1]

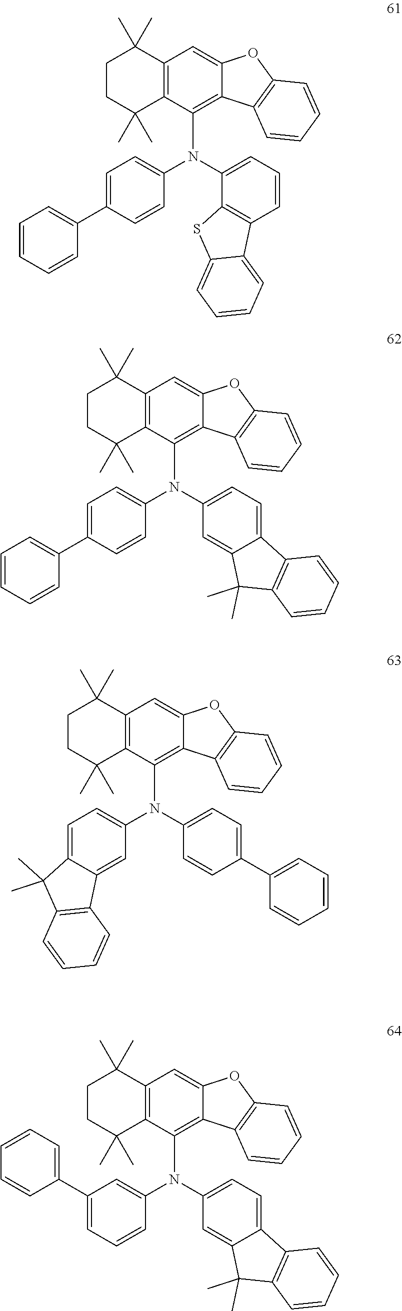 [SMILES: CC1(C)CCC(C)(C)c2c1cc1oc3ccccc3c1c2N(c1ccc(-c2ccccc2)cc1)c1cccc2c1sc1ccccc12 ; CC1(C)CCC(C)(C)c2c1cc1oc3ccccc3c1c2N(c1ccc(-c2ccccc2)cc1)c1ccc2c(c1)C(C)(C)c1ccccc1-2 ; CC1(C)CCC(C)(C)c2c1cc1oc3ccccc3c1c2N(c1ccc(-c2ccccc2)cc1)c1ccc2c(c1)-c1ccccc1C2(C)C ; CC1(C)CCC(C)(C)c2c1cc1oc3ccccc3c1c2N(c1cccc(-c2ccccc2)c1)c1ccc2c(c1)C(C)(C)c1ccccc1-2]

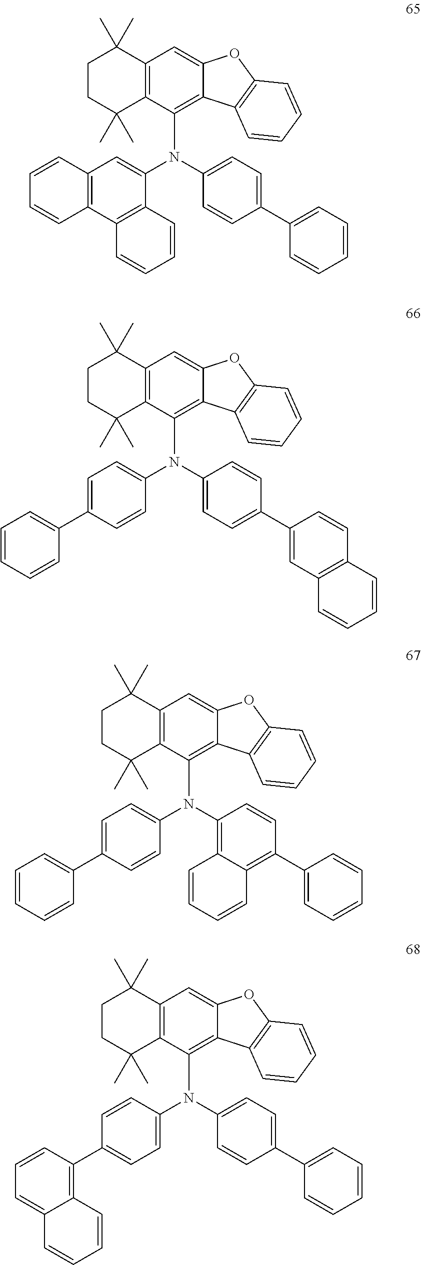 [SMILES: CC1(C)CCC(C)(C)c2c1cc1oc3ccccc3c1c2N(c1ccc(-c2ccccc2)cc1)c1cc2ccccc2c2ccccc12 ; CC1(C)CCC(C)(C)c2c1cc1oc3ccccc3c1c2N(c1ccc(-c2ccccc2)cc1)c1ccc(-c2ccc3ccccc3c2)cc1 ; CC1(C)CCC(C)(C)c2c1cc1oc3ccccc3c1c2N(c1ccc(-c2ccccc2)cc1)c1ccc(-c2ccccc2)c2ccccc12 ; CC1(C)CCC(C)(C)c2c1cc1oc3ccccc3c1c2N(c1ccc(-c2ccccc2)cc1)c1ccc(-c2cccc3ccccc23)cc1]

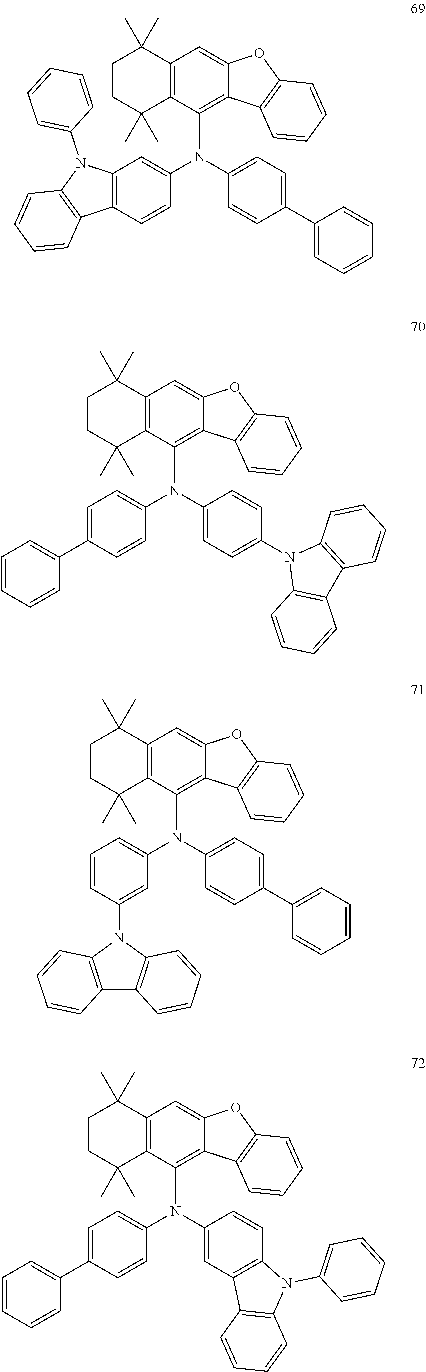 [SMILES: CC1(C)CCC(C)(C)c2c1cc1oc3ccccc3c1c2N(c1ccc(-c2ccccc2)cc1)c1ccc2c3ccccc3n(-c3ccccc3)c2c1 ; CC1(C)CCC(C)(C)c2c1cc1oc3ccccc3c1c2N(c1ccc(-c2ccccc2)cc1)c1ccc(-n2c3ccccc3c3ccccc32)cc1 ; CC1(C)CCC(C)(C)c2c1cc1oc3ccccc3c1c2N(c1ccc(-c2ccccc2)cc1)c1cccc(-n2c3ccccc3c3ccccc32)c1 ; CC1(C)CCC(C)(C)c2c1cc1oc3ccccc3c1c2N(c1ccc(-c2ccccc2)cc1)c1ccc2c(c1)c1ccccc1n2-c1ccccc1]

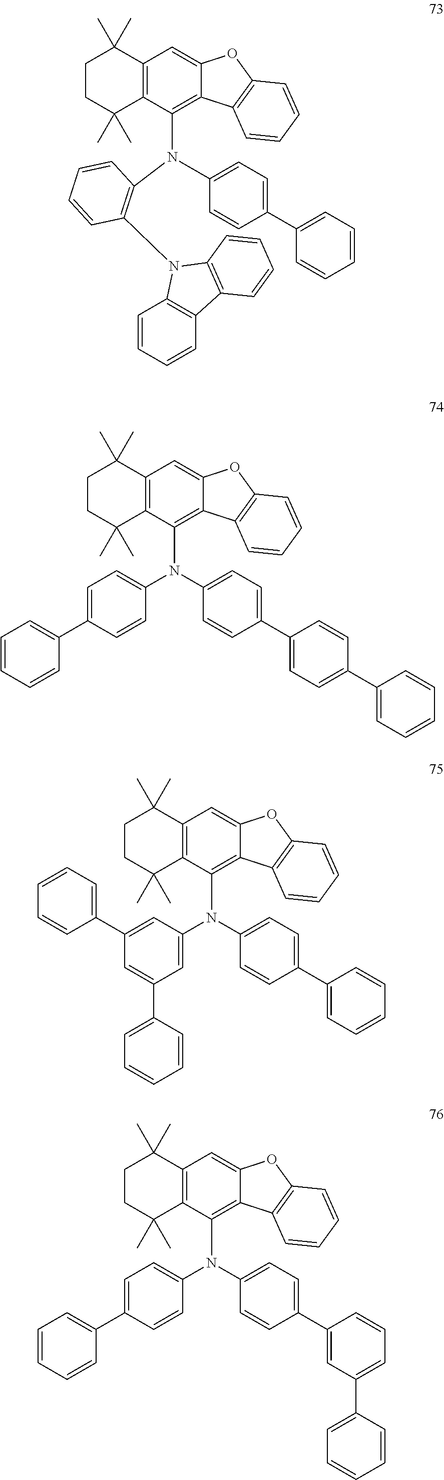

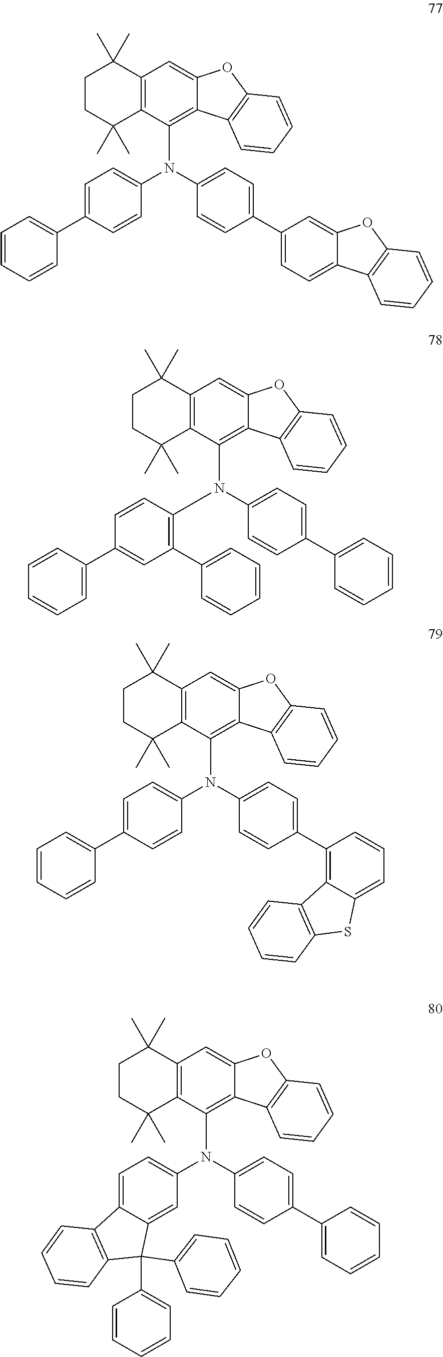 [SMILES: CC1(C)CCC(C)(C)c2c1cc1oc3ccccc3c1c2N(c1ccc(-c2ccccc2)cc1)c1ccc(-c2ccc3c(c2)oc2ccccc23)cc1 ; CC1(C)CCC(C)(C)c2c1cc1oc3ccccc3c1c2N(c1ccc(-c2ccccc2)cc1)c1ccc(-c2ccccc2)cc1-c1ccccc1 ; CC1(C)CCC(C)(C)c2c1cc1oc3ccccc3c1c2N(c1ccc(-c2ccccc2)cc1)c1ccc(-c2cccc3sc4ccccc4c23)cc1 ; CC1(C)CCC(C)(C)c2c1cc1oc3ccccc3c1c2N(c1ccc(-c2ccccc2)cc1)c1ccc2c(c1)C(c1ccccc1)(c1ccccc1)c1ccccc1-2]

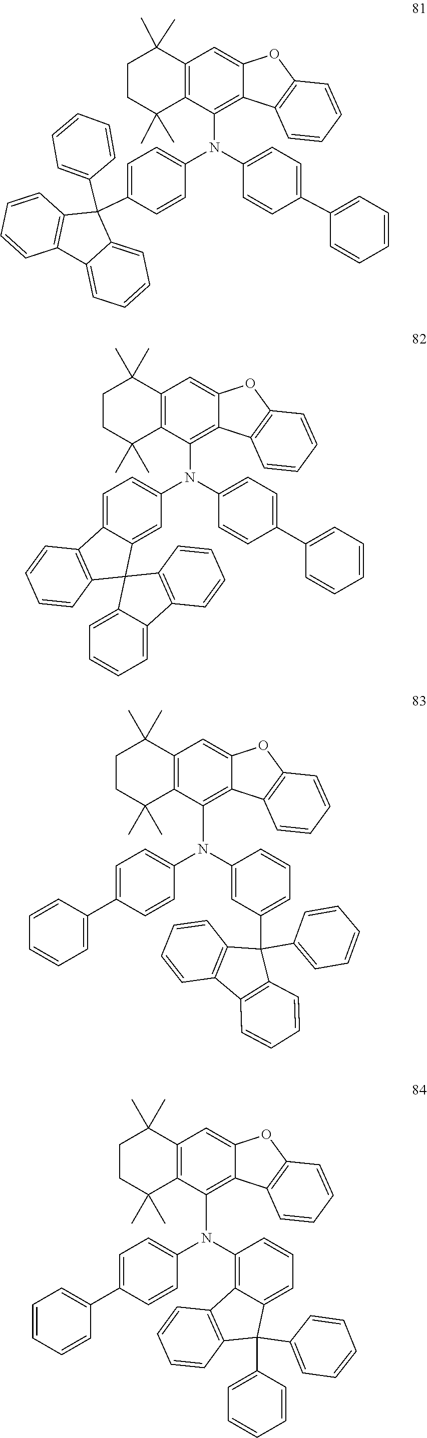

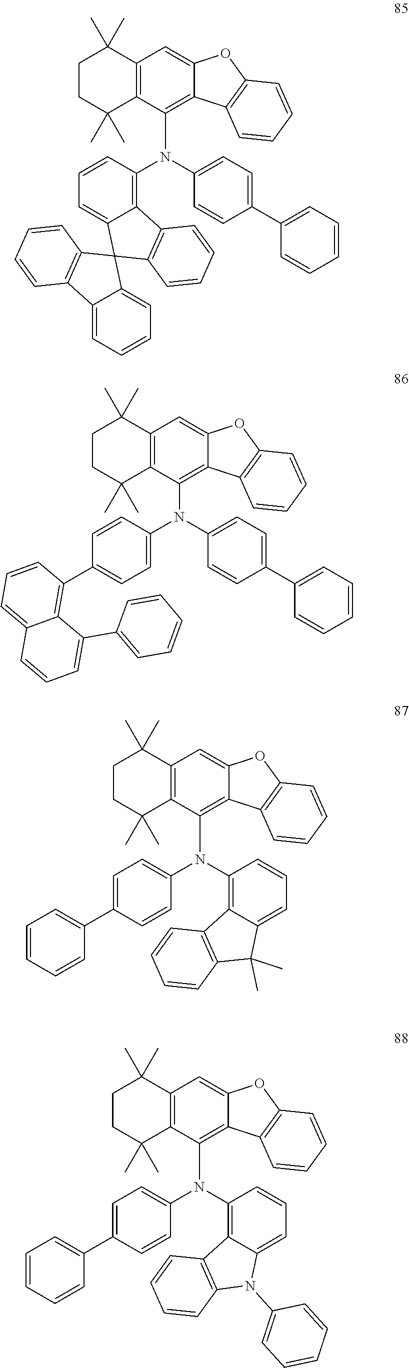

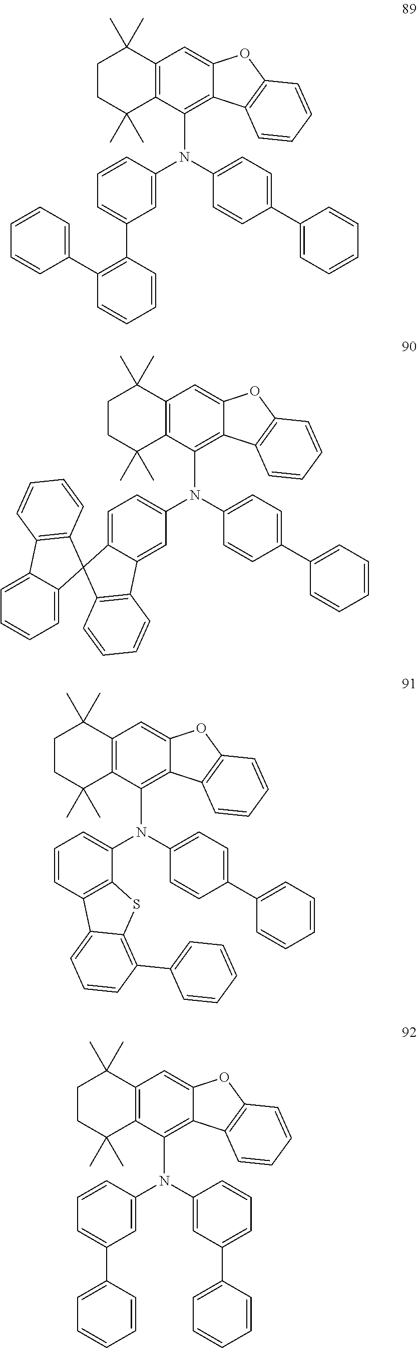

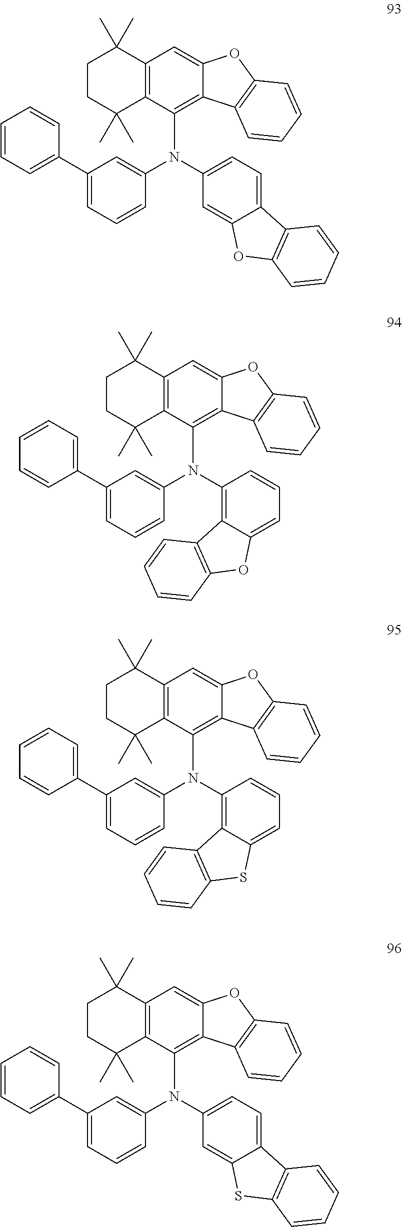

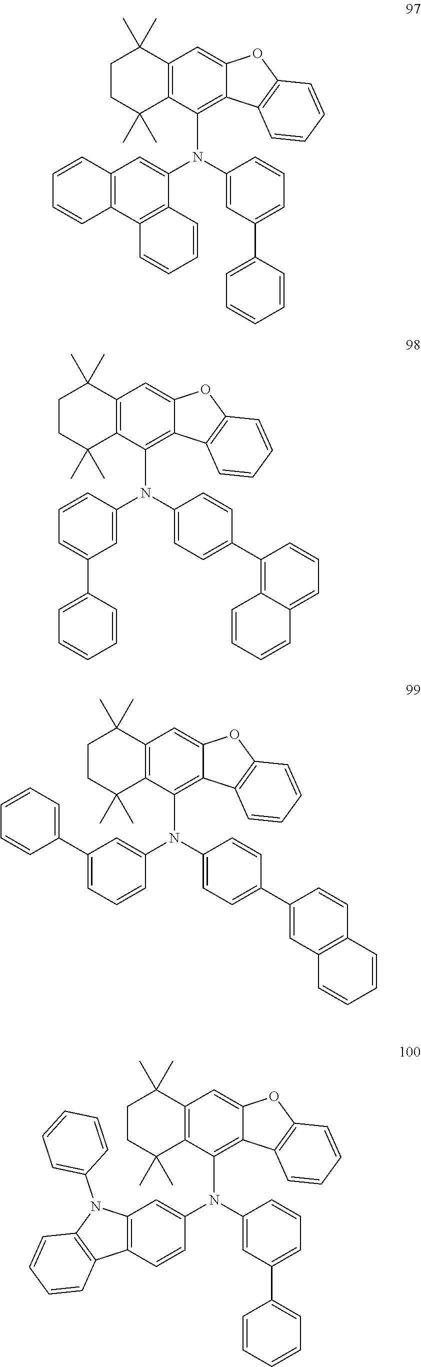

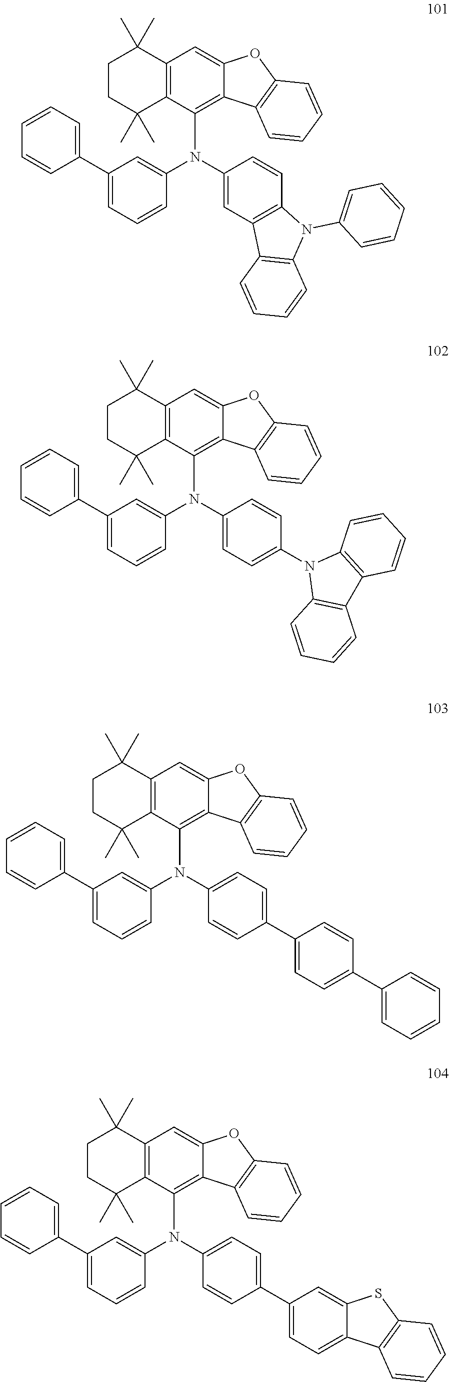 [SMILES: CC1(C)CCC(C)(C)c2c1cc1oc3ccccc3c1c2N(c1cccc(-c2ccccc2)c1)c1ccc2c(c1)c1ccccc1n2-c1ccccc1 ; CC1(C)CCC(C)(C)c2c1cc1oc3ccccc3c1c2N(c1ccc(-n2c3ccccc3c3ccccc32)cc1)c1cccc(-c2ccccc2)c1 ; CC1(C)CCC(C)(C)c2c1cc1oc3ccccc3c1c2N(c1ccc(-c2ccc(-c3ccccc3)cc2)cc1)c1cccc(-c2ccccc2)c1 ; CC1(C)CCC(C)(C)c2c1cc1oc3ccccc3c1c2N(c1ccc(-c2ccc3c(c2)sc2ccccc23)cc1)c1cccc(-c2ccccc2)c1]

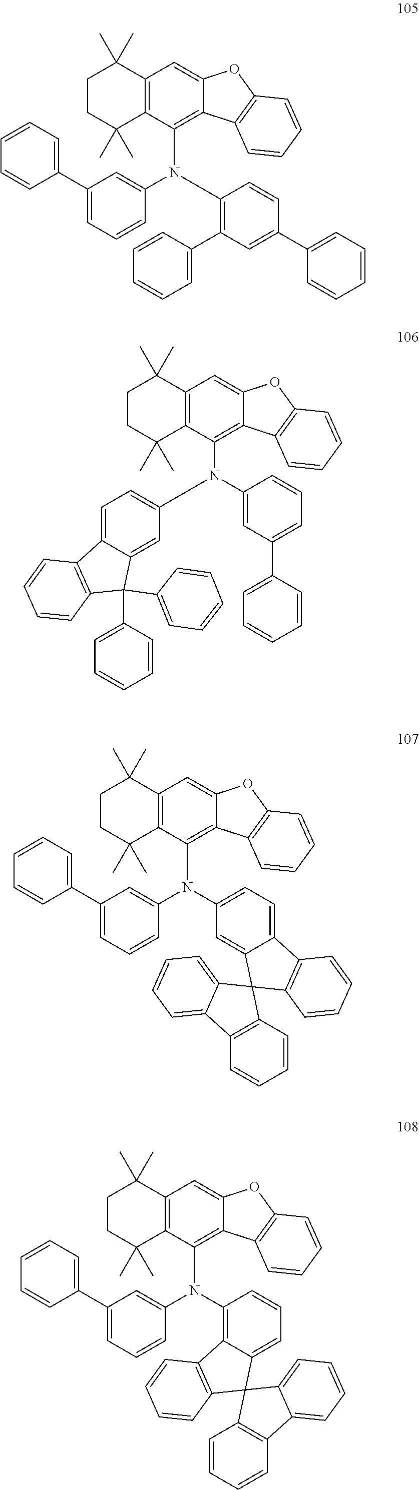 [SMILES: CC1(C)CCC(C)(C)c2c1cc1oc3ccccc3c1c2N(c1cccc(-c2ccccc2)c1)c1ccc(-c2ccccc2)cc1-c1ccccc1 ; CC1(C)CCC(C)(C)c2c1cc1oc3ccccc3c1c2N(c1cccc(-c2ccccc2)c1)c1ccc2c(c1)C(c1ccccc1)(c1ccccc1)c1ccccc1-2 ; CC1(C)CCC(C)(C)c2c1cc1oc3ccccc3c1c2N(c1cccc(-c2ccccc2)c1)c1ccc2c(c1)C1(c3ccccc3-c3ccccc31)c1ccccc1-2 ; CC1(C)CCC(C)(C)c2c1cc1oc3ccccc3c1c2N(c1cccc(-c2ccccc2)c1)c1cccc2c1-c1ccccc1C21c2ccccc2-c2ccccc21]

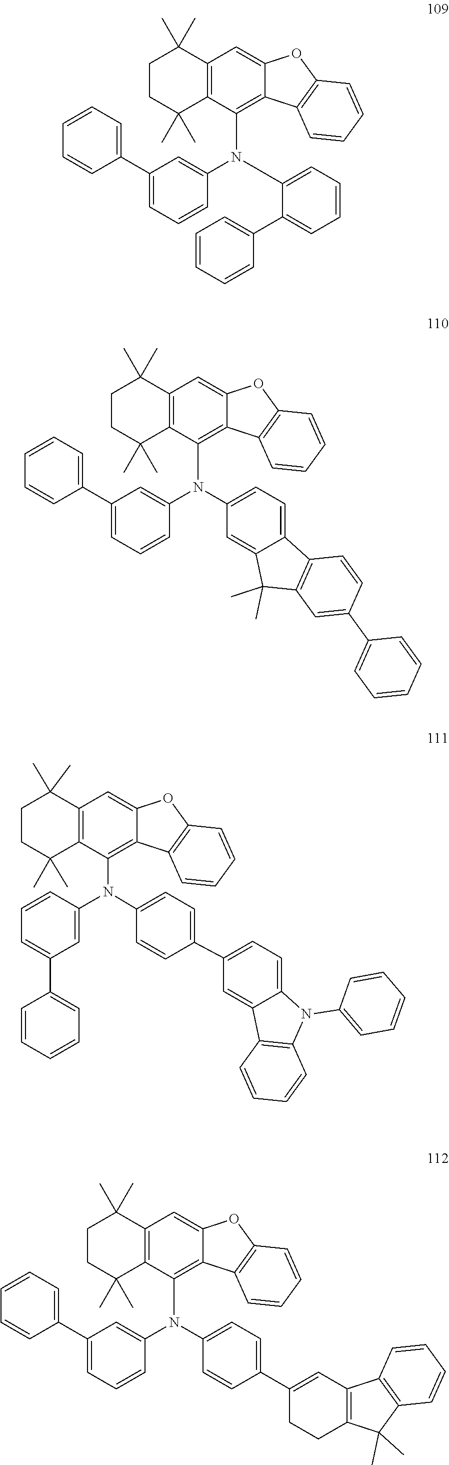 [SMILES: CC1(C)CCC(C)(C)c2c1cc1oc3ccccc3c1c2N(c1cccc(-c2ccccc2)c1)c1ccccc1-c1ccccc1 ; CC1(C)CCC(C)(C)c2c1cc1oc3ccccc3c1c2N(c1cccc(-c2ccccc2)c1)c1ccc2c(c1)C(C)(C)c1cc(-c3ccccc3)ccc1-2 ; CC1(C)CCC(C)(C)c2c1cc1oc3ccccc3c1c2N(c1ccc(-c2ccc3c(c2)c2ccccc2n3-c2ccccc2)cc1)c1cccc(-c2ccccc2)c1 ; CC1(C)CCC(C)(C)c2c1cc1oc3ccccc3c1c2N(c1ccc(C2=CC3=C(CC2)C(C)(C)c2ccccc23)cc1)c1cccc(-c2ccccc2)c1]

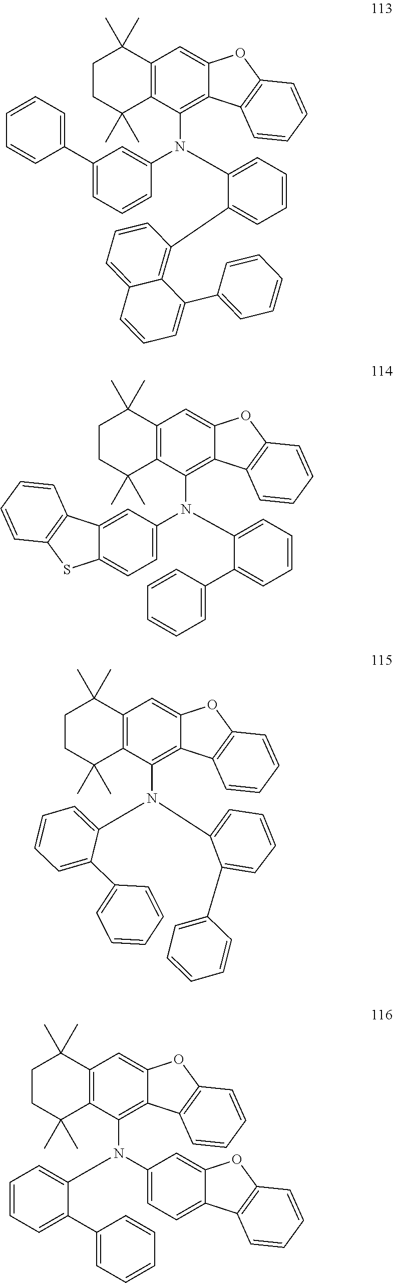

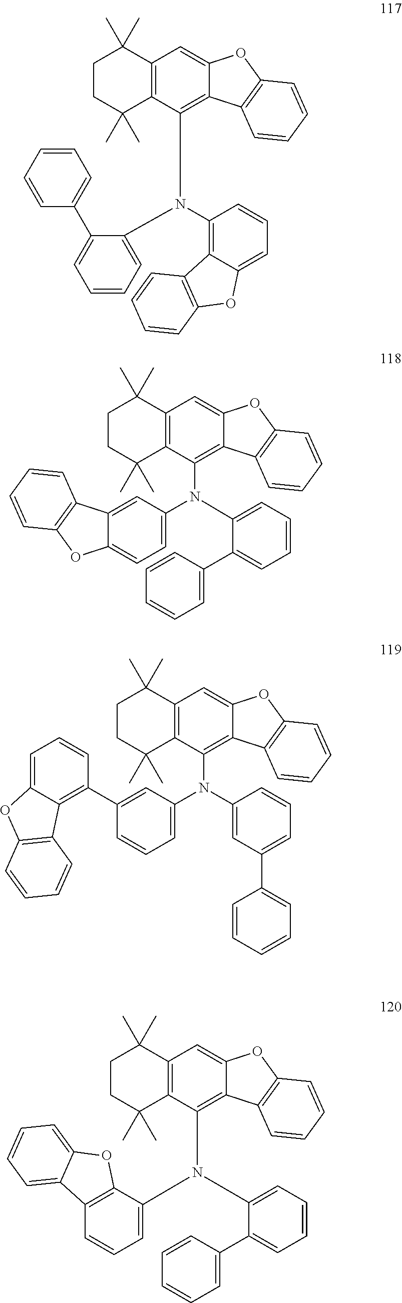

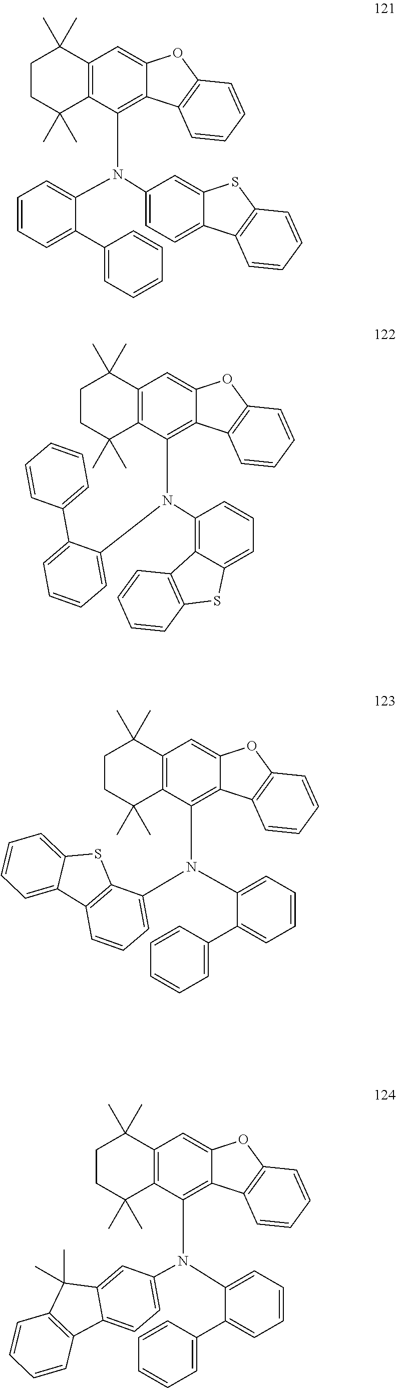 [SMILES: CC1(C)CCC(C)(C)c2c1cc1oc3ccccc3c1c2N(c1ccc2c(c1)sc1ccccc12)c1ccccc1-c1ccccc1 ; CC1(C)CCC(C)(C)c2c1cc1oc3ccccc3c1c2N(c1ccccc1-c1ccccc1)c1cccc2sc3ccccc3c12 ; CC1(C)CCC(C)(C)c2c1cc1oc3ccccc3c1c2N(c1ccccc1-c1ccccc1)c1cccc2c1sc1ccccc12 ; CC1(C)CCC(C)(C)c2c1cc1oc3ccccc3c1c2N(c1ccc2c(c1)C(C)(C)c1ccccc1-2)c1ccccc1-c1ccccc1]

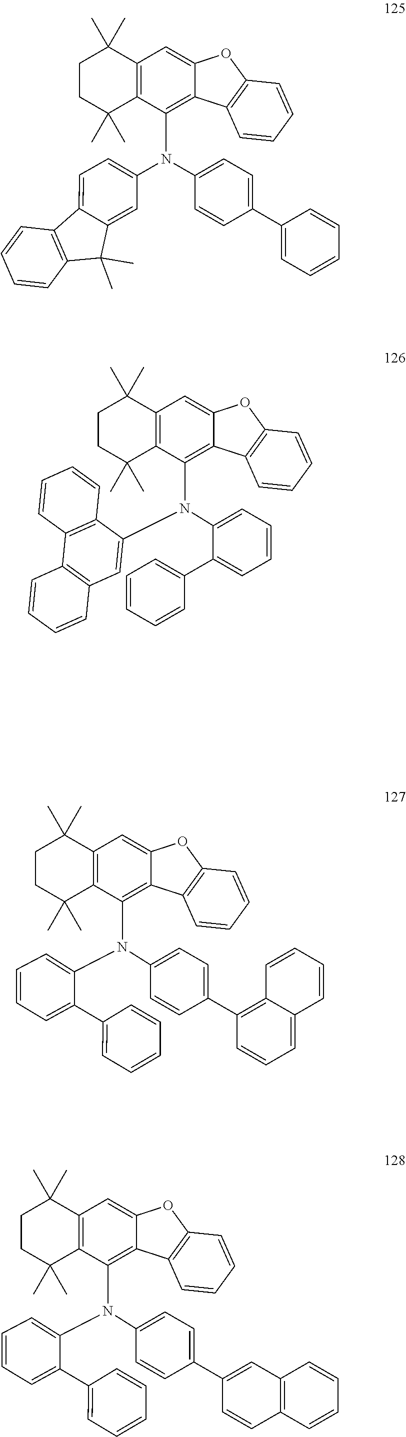 [SMILES: CC1(C)CCC(C)(C)c2c1cc1oc3ccccc3c1c2N(c1ccc(-c2ccccc2)cc1)c1ccc2c(c1)C(C)(C)c1ccccc1-2 ; CC1(C)CCC(C)(C)c2c1cc1oc3ccccc3c1c2N(c1ccccc1-c1ccccc1)c1cc2ccccc2c2ccccc12 ; CC1(C)CCC(C)(C)c2c1cc1oc3ccccc3c1c2N(c1ccc(-c2cccc3ccccc23)cc1)c1ccccc1-c1ccccc1 ; CC1(C)CCC(C)(C)c2c1cc1oc3ccccc3c1c2N(c1ccc(-c2ccc3ccccc3c2)cc1)c1ccccc1-c1ccccc1]

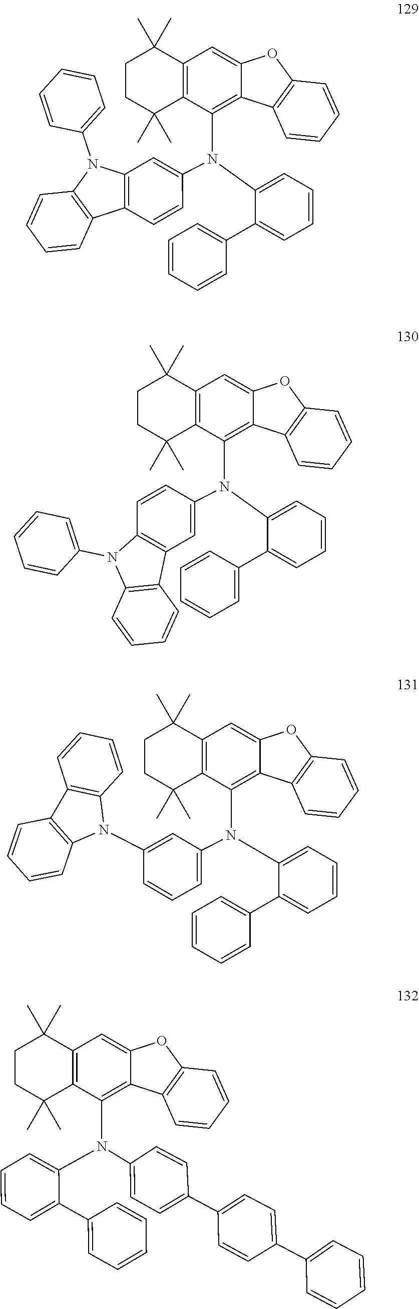 [SMILES: CC1(C)CCC(C)(C)c2c1cc1oc3ccccc3c1c2N(c1ccc2c3ccccc3n(-c3ccccc3)c2c1)c1ccccc1-c1ccccc1 ; CC1(C)CCC(C)(C)c2c1cc1oc3ccccc3c1c2N(c1ccc2c(c1)c1ccccc1n2-c1ccccc1)c1ccccc1-c1ccccc1 ; CC1(C)CCC(C)(C)c2c1cc1oc3ccccc3c1c2N(c1cccc(-n2c3ccccc3c3ccccc32)c1)c1ccccc1-c1ccccc1 ; CC1(C)CCC(C)(C)c2c1cc1oc3ccccc3c1c2N(c1ccc(-c2ccc(-c3ccccc3)cc2)cc1)c1ccccc1-c1ccccc1]

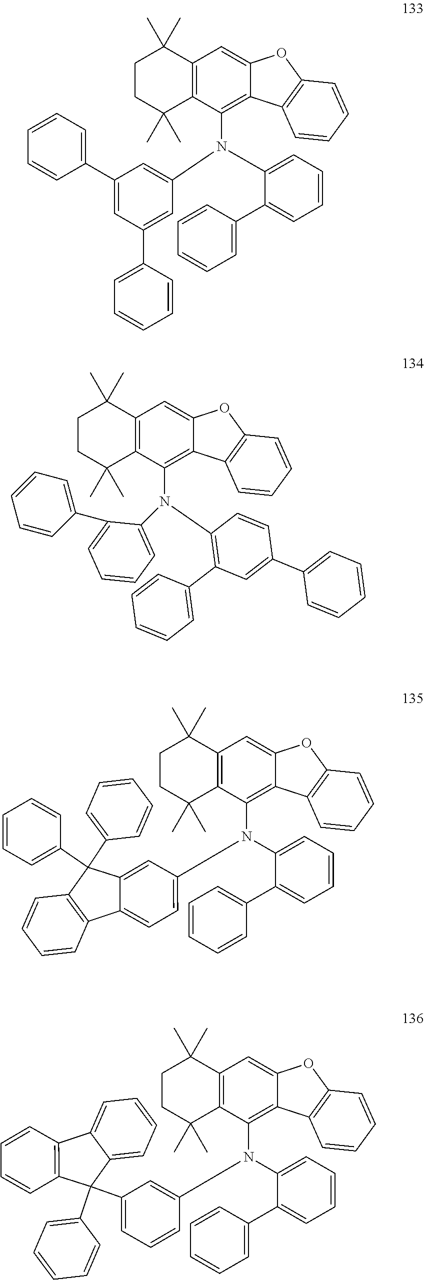 [SMILES: CC1(C)CCC(C)(C)c2c1cc1oc3ccccc3c1c2N(c1cc(-c2ccccc2)cc(-c2ccccc2)c1)c1ccccc1-c1ccccc1 ; CC1(C)CCC(C)(C)c2c1cc1oc3ccccc3c1c2N(c1ccccc1-c1ccccc1)c1ccc(-c2ccccc2)cc1-c1ccccc1 ; CC1(C)CCC(C)(C)c2c1cc1oc3ccccc3c1c2N(c1ccc2c(c1)C(c1ccccc1)(c1ccccc1)c1ccccc1-2)c1ccccc1-c1ccccc1 ; CC1(C)CCC(C)(C)c2c1cc1oc3ccccc3c1c2N(c1cccc(C2(c3ccccc3)c3ccccc3-c3ccccc32)c1)c1ccccc1-c1ccccc1]

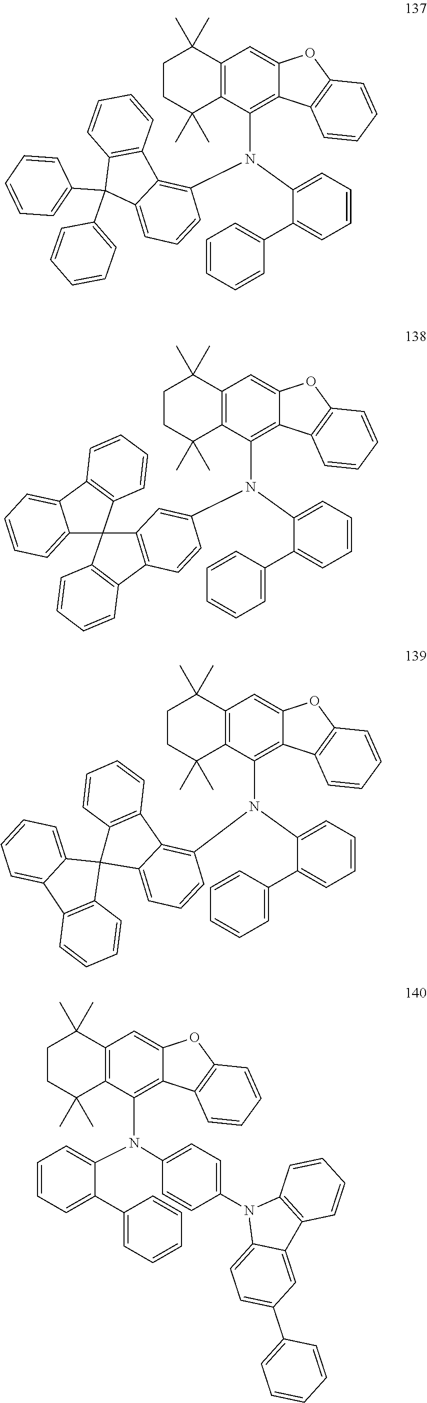

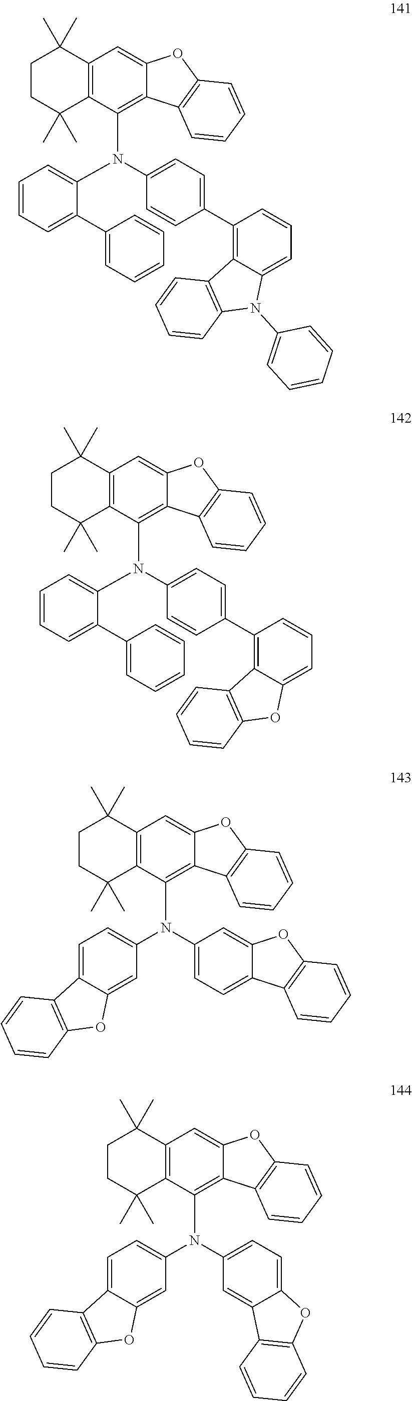 [SMILES: CC1(C)CCC(C)(C)c2c1cc1oc3ccccc3c1c2N(c1ccc(-c2cccc3c2c2ccccc2n3-c2ccccc2)cc1)c1ccccc1-c1ccccc1 ; CC1(C)CCC(C)(C)c2c1cc1oc3ccccc3c1c2N(c1ccc(-c2cccc3oc4ccccc4c23)cc1)c1ccccc1-c1ccccc1 ; CC1(C)CCC(C)(C)c2c1cc1oc3ccccc3c1c2N(c1ccc2c(c1)oc1ccccc12)c1ccc2c(c1)oc1ccccc12 ; CC1(C)CCC(C)(C)c2c1cc1oc3ccccc3c1c2N(c1ccc2c(c1)oc1ccccc12)c1ccc2oc3ccccc3c2c1]

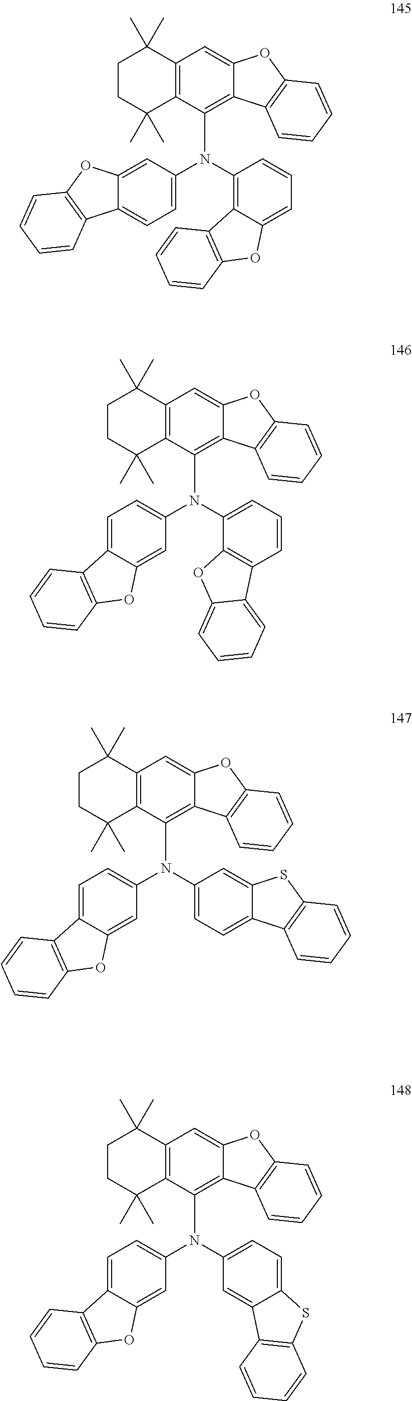 [SMILES: CC1(C)CCC(C)(C)c2c1cc1oc3ccccc3c1c2N(c1ccc2c(c1)oc1ccccc12)c1cccc2oc3ccccc3c12 ; CC1(C)CCC(C)(C)c2c1cc1oc3ccccc3c1c2N(c1ccc2c(c1)oc1ccccc12)c1cccc2c1oc1ccccc12 ; CC1(C)CCC(C)(C)c2c1cc1oc3ccccc3c1c2N(c1ccc2c(c1)oc1ccccc12)c1ccc2c(c1)sc1ccccc12 ; CC1(C)CCC(C)(C)c2c1cc1oc3ccccc3c1c2N(c1ccc2c(c1)oc1ccccc12)c1ccc2sc3ccccc3c2c1]

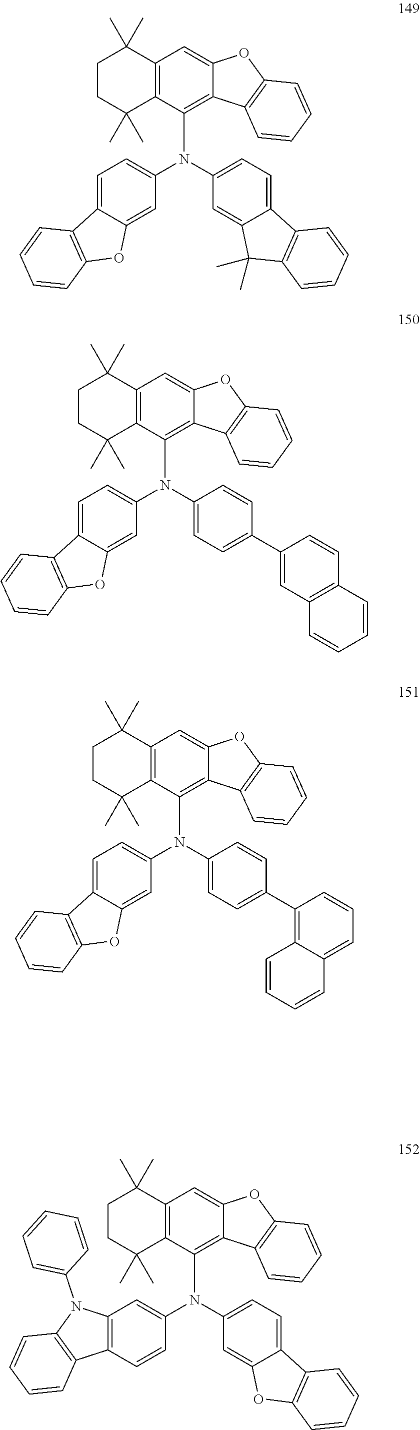 [SMILES: CC1(C)CCC(C)(C)c2c1cc1oc3ccccc3c1c2N(c1ccc2c(c1)C(C)(C)c1ccccc1-2)c1ccc2c(c1)oc1ccccc12 ; CC1(C)CCC(C)(C)c2c1cc1oc3ccccc3c1c2N(c1ccc(-c2ccc3ccccc3c2)cc1)c1ccc2c(c1)oc1ccccc12 ; CC1(C)CCC(C)(C)c2c1cc1oc3ccccc3c1c2N(c1ccc(-c2cccc3ccccc23)cc1)c1ccc2c(c1)oc1ccccc12 ; CC1(C)CCC(C)(C)c2c1cc1oc3ccccc3c1c2N(c1ccc2c(c1)oc1ccccc12)c1ccc2c3ccccc3n(-c3ccccc3)c2c1]

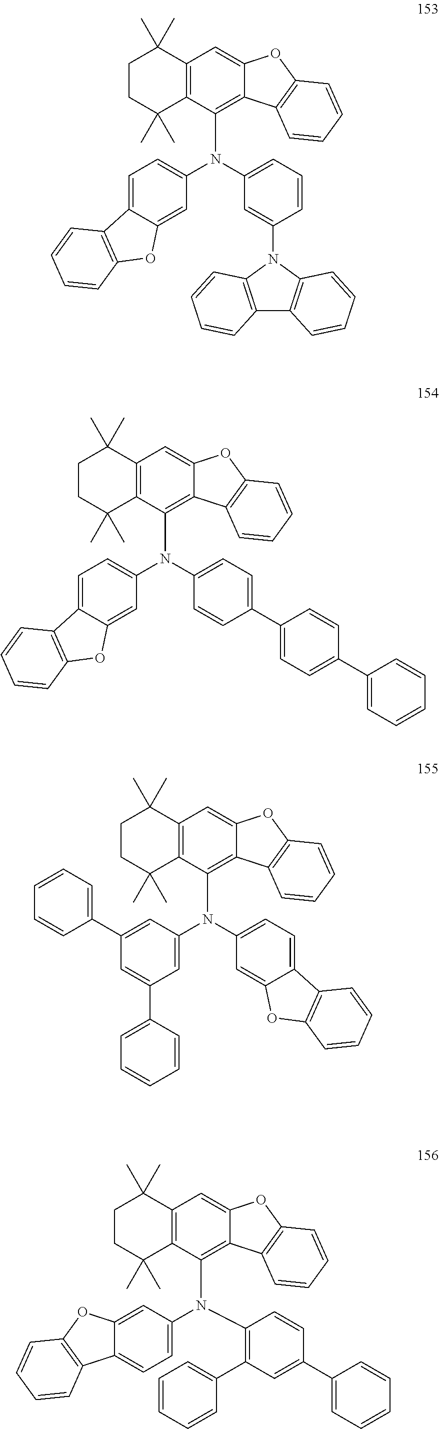 [SMILES: CC1(C)CCC(C)(C)c2c1cc1oc3ccccc3c1c2N(c1cccc(-n2c3ccccc3c3ccccc32)c1)c1ccc2c(c1)oc1ccccc12 ; CC1(C)CCC(C)(C)c2c1cc1oc3ccccc3c1c2N(c1ccc(-c2ccc(-c3ccccc3)cc2)cc1)c1ccc2c(c1)oc1ccccc12 ; CC1(C)CCC(C)(C)c2c1cc1oc3ccccc3c1c2N(c1cc(-c2ccccc2)cc(-c2ccccc2)c1)c1ccc2c(c1)oc1ccccc12 ; CC1(C)CCC(C)(C)c2c1cc1oc3ccccc3c1c2N(c1ccc2c(c1)oc1ccccc12)c1ccc(-c2ccccc2)cc1-c1ccccc1]

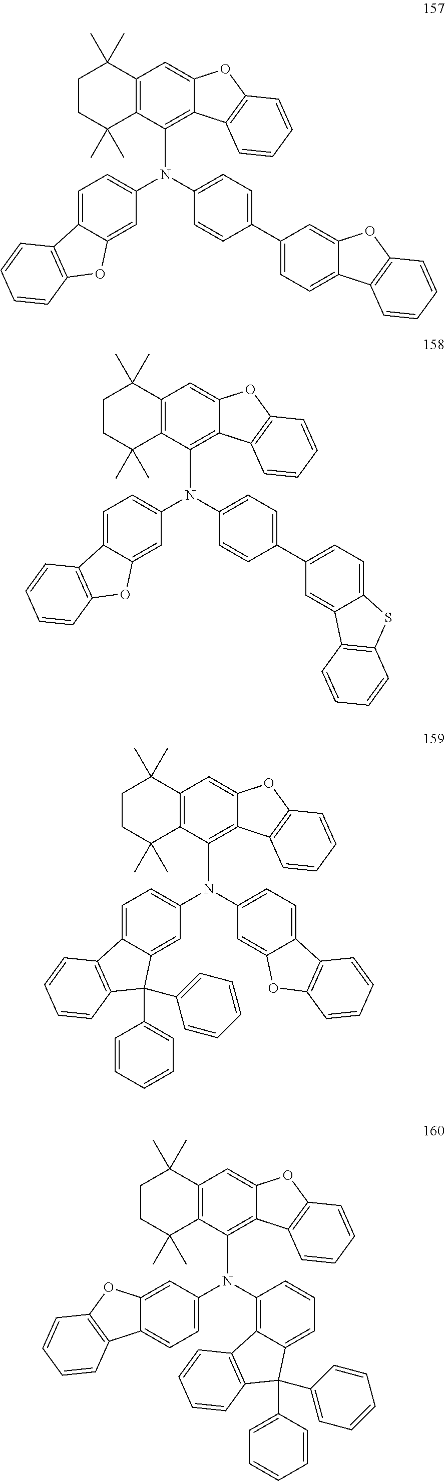 [SMILES: CC1(C)CCC(C)(C)c2c1cc1oc3ccccc3c1c2N(c1ccc(-c2ccc3c(c2)oc2ccccc23)cc1)c1ccc2c(c1)oc1ccccc12 ; CC1(C)CCC(C)(C)c2c1cc1oc3ccccc3c1c2N(c1ccc(-c2ccc3sc4ccccc4c3c2)cc1)c1ccc2c(c1)oc1ccccc12 ; CC1(C)CCC(C)(C)c2c1cc1oc3ccccc3c1c2N(c1ccc2c(c1)C(c1ccccc1)(c1ccccc1)c1ccccc1-2)c1ccc2c(c1)oc1ccccc12 ; CC1(C)CCC(C)(C)c2c1cc1oc3ccccc3c1c2N(c1ccc2c(c1)oc1ccccc12)c1cccc2c1-c1ccccc1C2(c1ccccc1)c1ccccc1]

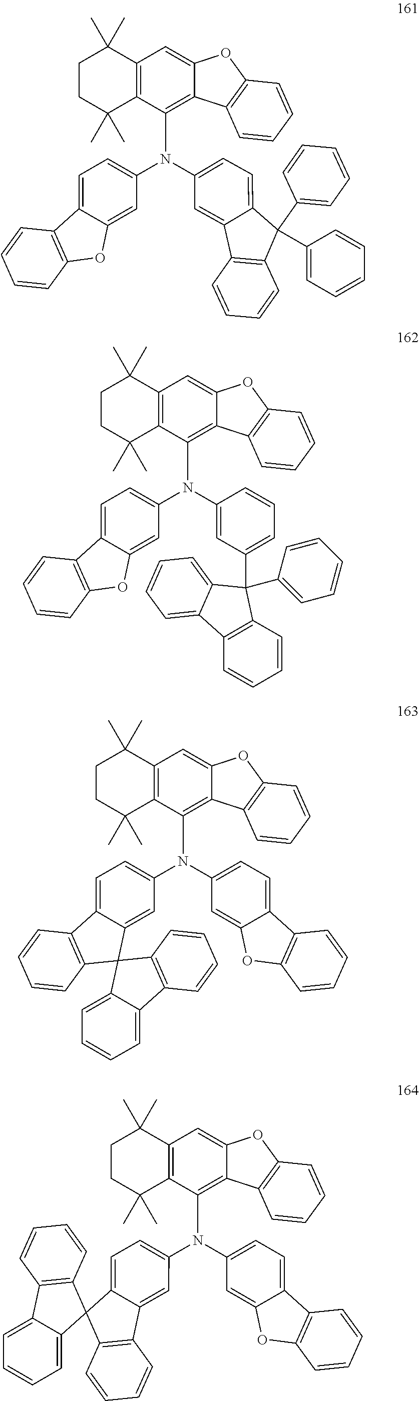

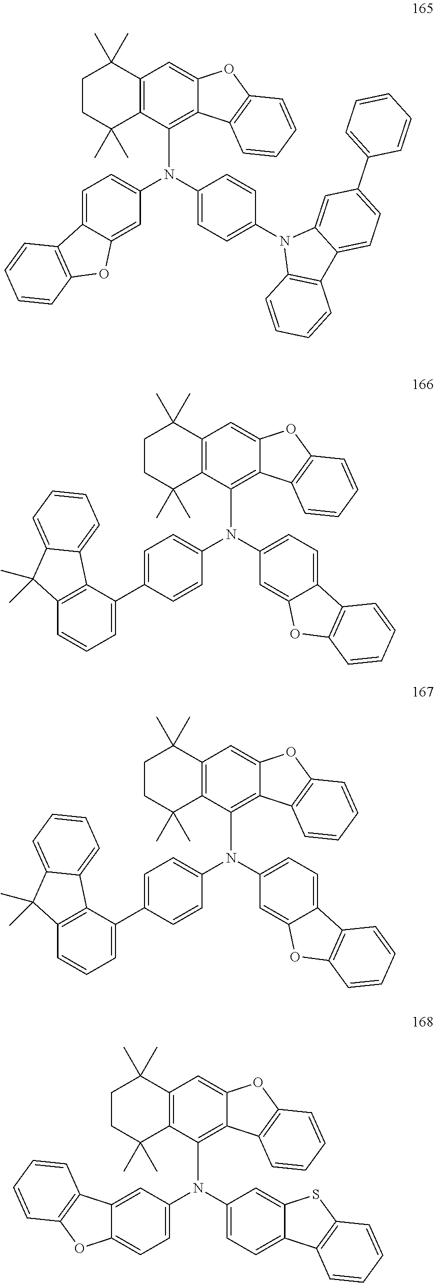 [SMILES: CC1(C)CCC(C)(C)c2c1cc1oc3ccccc3c1c2N(c1ccc(-n2c3ccccc3c3ccc(-c4ccccc4)cc32)cc1)c1ccc2c(c1)oc1ccccc12 ; CC1(C)CCC(C)(C)c2c1cc1oc3ccccc3c1c2N(c1ccc(-c2cccc3c2-c2ccccc2C3(C)C)cc1)c1ccc2c(c1)oc1ccccc12 ; CC1(C)CCC(C)(C)c2c1cc1oc3ccccc3c1c2N(c1ccc(-c2cccc3c2-c2ccccc2C3(C)C)cc1)c1ccc2c(c1)oc1ccccc12 ; CC1(C)CCC(C)(C)c2c1cc1oc3ccccc3c1c2N(c1ccc2c(c1)sc1ccccc12)c1ccc2oc3ccccc3c2c1]

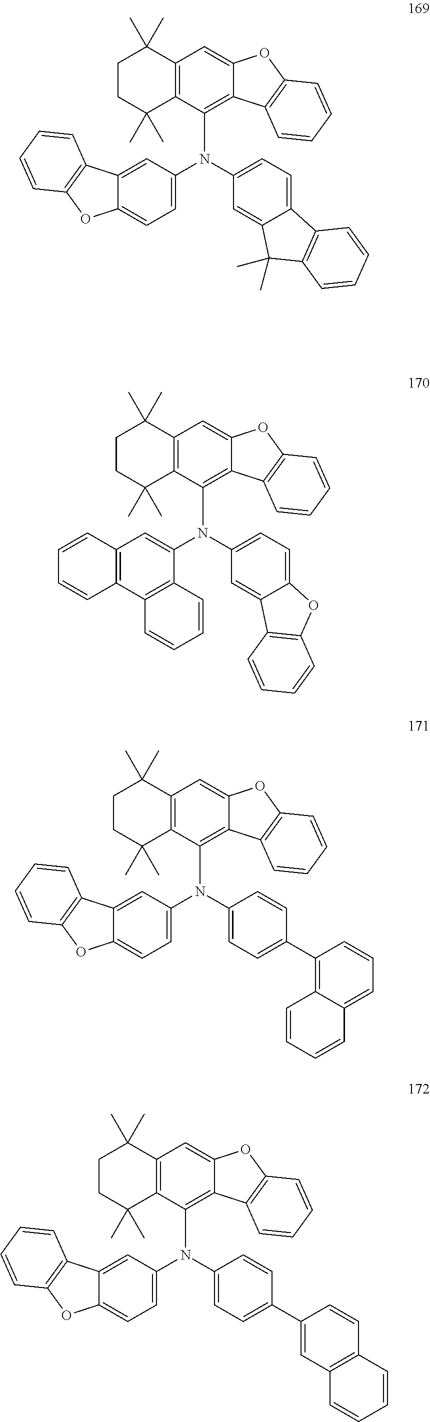 [SMILES: CC1(C)CCC(C)(C)c2c1cc1oc3ccccc3c1c2N(c1ccc2c(c1)C(C)(C)c1ccccc1-2)c1ccc2oc3ccccc3c2c1 ; CC1(C)CCC(C)(C)c2c1cc1oc3ccccc3c1c2N(c1ccc2oc3ccccc3c2c1)c1cc2ccccc2c2ccccc12 ; CC1(C)CCC(C)(C)c2c1cc1oc3ccccc3c1c2N(c1ccc(-c2cccc3ccccc23)cc1)c1ccc2oc3ccccc3c2c1 ; CC1(C)CCC(C)(C)c2c1cc1oc3ccccc3c1c2N(c1ccc(-c2ccc3ccccc3c2)cc1)c1ccc2oc3ccccc3c2c1]

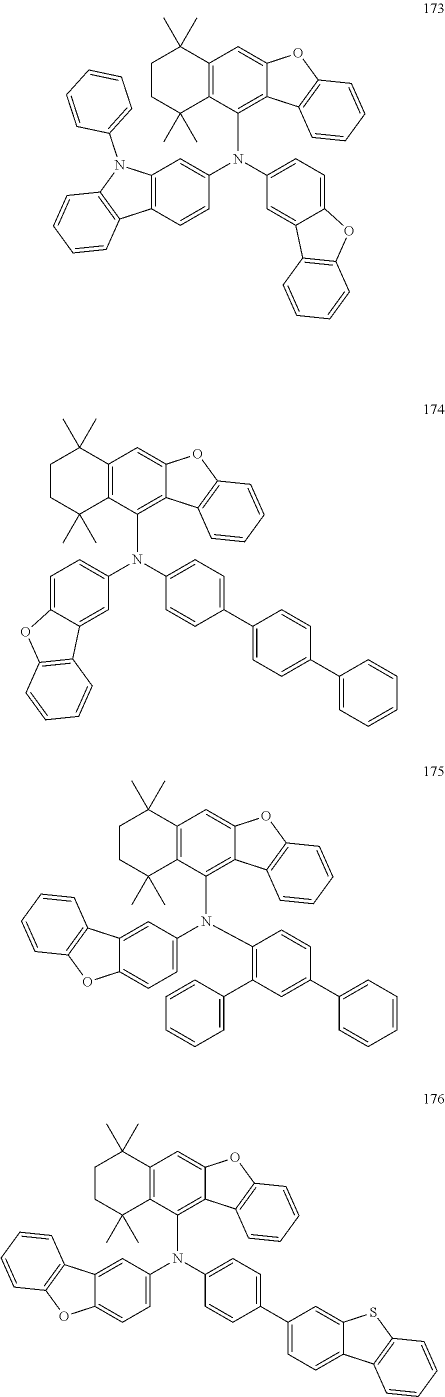 [SMILES: CC1(C)CCC(C)(C)c2c1cc1oc3ccccc3c1c2N(c1ccc2oc3ccccc3c2c1)c1ccc2c3ccccc3n(-c3ccccc3)c2c1 ; CC1(C)CCC(C)(C)c2c1cc1oc3ccccc3c1c2N(c1ccc(-c2ccc(-c3ccccc3)cc2)cc1)c1ccc2oc3ccccc3c2c1 ; CC1(C)CCC(C)(C)c2c1cc1oc3ccccc3c1c2N(c1ccc2oc3ccccc3c2c1)c1ccc(-c2ccccc2)cc1-c1ccccc1 ; CC1(C)CCC(C)(C)c2c1cc1oc3ccccc3c1c2N(c1ccc(-c2ccc3c(c2)sc2ccccc23)cc1)c1ccc2oc3ccccc3c2c1]

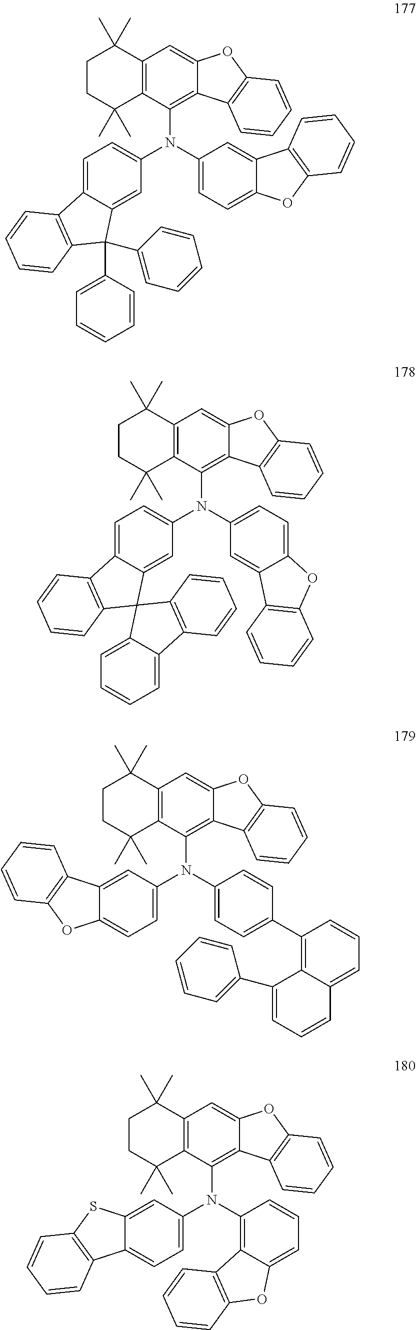

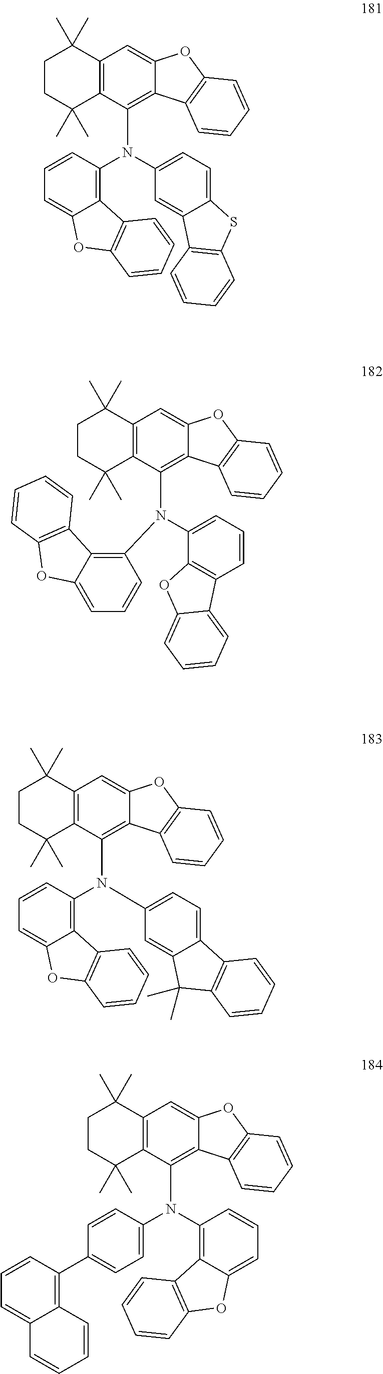 [SMILES: CC1(C)CCC(C)(C)c2c1cc1oc3ccccc3c1c2N(c1ccc2sc3ccccc3c2c1)c1cccc2oc3ccccc3c12 ; CC1(C)CCC(C)(C)c2c1cc1oc3ccccc3c1c2N(c1cccc2c1oc1ccccc12)c1cccc2oc3ccccc3c12 ; CC1(C)CCC(C)(C)c2c1cc1oc3ccccc3c1c2N(c1ccc2c(c1)C(C)(C)c1ccccc1-2)c1cccc2oc3ccccc3c12 ; CC1(C)CCC(C)(C)c2c1cc1oc3ccccc3c1c2N(c1ccc(-c2cccc3ccccc23)cc1)c1cccc2oc3ccccc3c12]

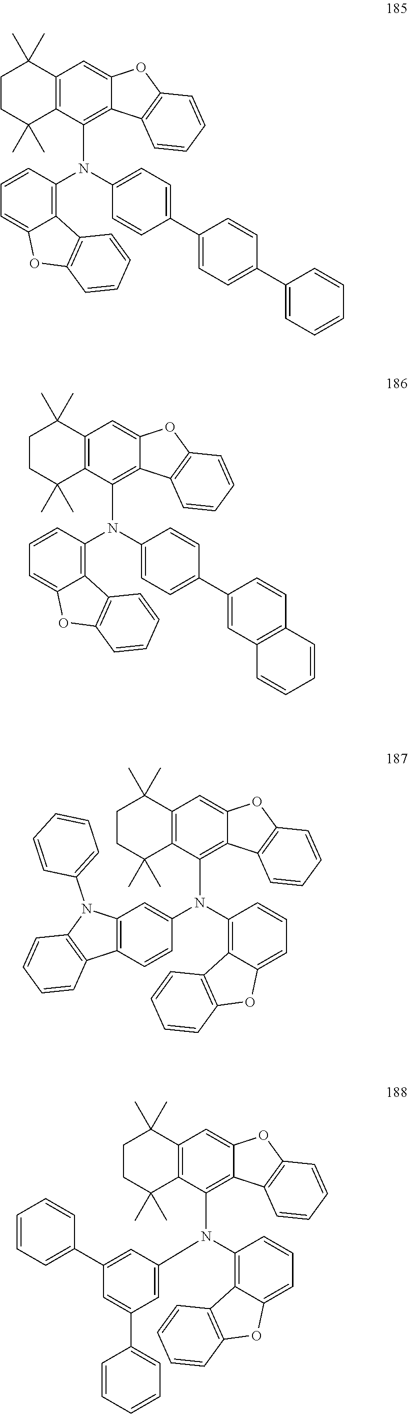 [SMILES: CC1(C)CCC(C)(C)c2c1cc1oc3ccccc3c1c2N(c1ccc(-c2ccc(-c3ccccc3)cc2)cc1)c1cccc2oc3ccccc3c12 ; CC1(C)CCC(C)(C)c2c1cc1oc3ccccc3c1c2N(c1ccc(-c2ccc3ccccc3c2)cc1)c1cccc2oc3ccccc3c12 ; CC1(C)CCC(C)(C)c2c1cc1oc3ccccc3c1c2N(c1ccc2c3ccccc3n(-c3ccccc3)c2c1)c1cccc2oc3ccccc3c12 ; CC1(C)CCC(C)(C)c2c1cc1oc3ccccc3c1c2N(c1cc(-c2ccccc2)cc(-c2ccccc2)c1)c1cccc2oc3ccccc3c12]

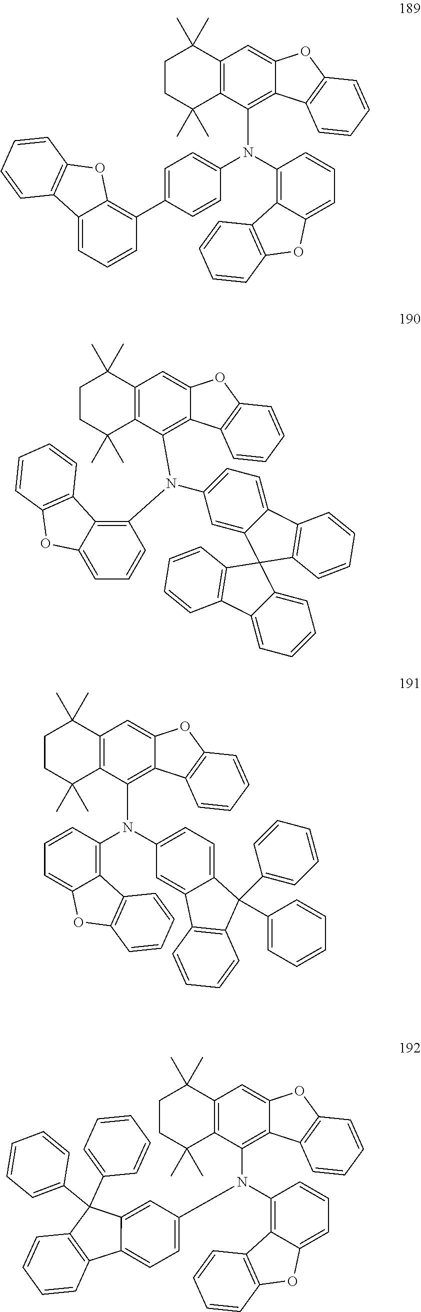 [SMILES: CC1(C)CCC(C)(C)c2c1cc1oc3ccccc3c1c2N(c1ccc(-c2cccc3c2oc2ccccc23)cc1)c1cccc2oc3ccccc3c12 ; CC1(C)CCC(C)(C)c2c1cc1oc3ccccc3c1c2N(c1ccc2c(c1)C1(c3ccccc3-c3ccccc31)c1ccccc1-2)c1cccc2oc3ccccc3c12 ; CC1(C)CCC(C)(C)c2c1cc1oc3ccccc3c1c2N(c1ccc2c(c1)-c1ccccc1C2(c1ccccc1)c1ccccc1)c1cccc2oc3ccccc3c12 ; CC1(C)CCC(C)(C)c2c1cc1oc3ccccc3c1c2N(c1ccc2c(c1)C(c1ccccc1)(c1ccccc1)c1ccccc1-2)c1cccc2oc3ccccc3c12]

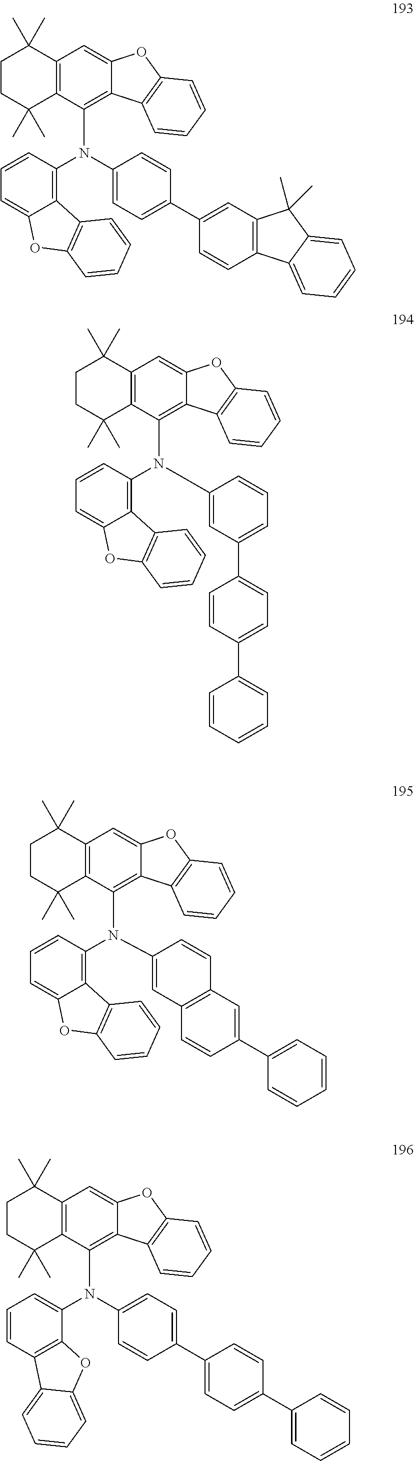 [SMILES: CC1(C)CCC(C)(C)c2c1cc1oc3ccccc3c1c2N(c1ccc(-c2ccc3c(c2)C(C)(C)c2ccccc2-3)cc1)c1cccc2oc3ccccc3c12 ; CC1(C)CCC(C)(C)c2c1cc1oc3ccccc3c1c2N(c1cccc(-c2ccc(-c3ccccc3)cc2)c1)c1cccc2oc3ccccc3c12 ; CC1(C)CCC(C)(C)c2c1cc1oc3ccccc3c1c2N(c1ccc2cc(-c3ccccc3)ccc2c1)c1cccc2oc3ccccc3c12 ; CC1(C)CCC(C)(C)c2c1cc1oc3ccccc3c1c2N(c1ccc(-c2ccc(-c3ccccc3)cc2)cc1)c1cccc2c1oc1ccccc12]

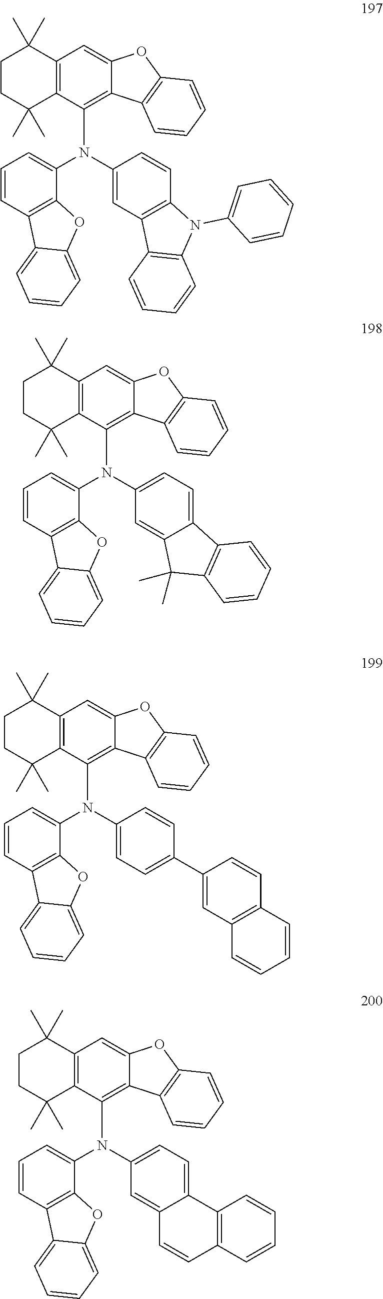

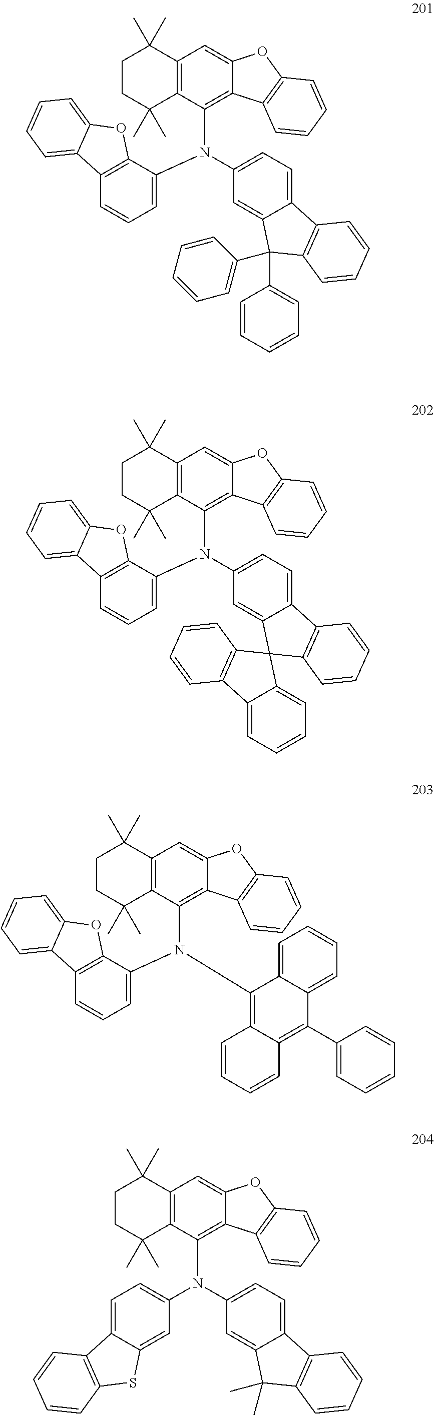 [SMILES: CC1(C)CCC(C)(C)c2c1cc1oc3ccccc3c1c2N(c1ccc2c(c1)C(c1ccccc1)(c1ccccc1)c1ccccc1-2)c1cccc2c1oc1ccccc12 ; CC1(C)CCC(C)(C)c2c1cc1oc3ccccc3c1c2N(c1ccc2c(c1)C1(c3ccccc3-c3ccccc31)c1ccccc1-2)c1cccc2c1oc1ccccc12 ; CC1(C)CCC(C)(C)c2c1cc1oc3ccccc3c1c2N(c1c2ccccc2c(-c2ccccc2)c2ccccc12)c1cccc2c1oc1ccccc12 ; CC1(C)CCC(C)(C)c2c1cc1oc3ccccc3c1c2N(c1ccc2c(c1)C(C)(C)c1ccccc1-2)c1ccc2c(c1)sc1ccccc12]

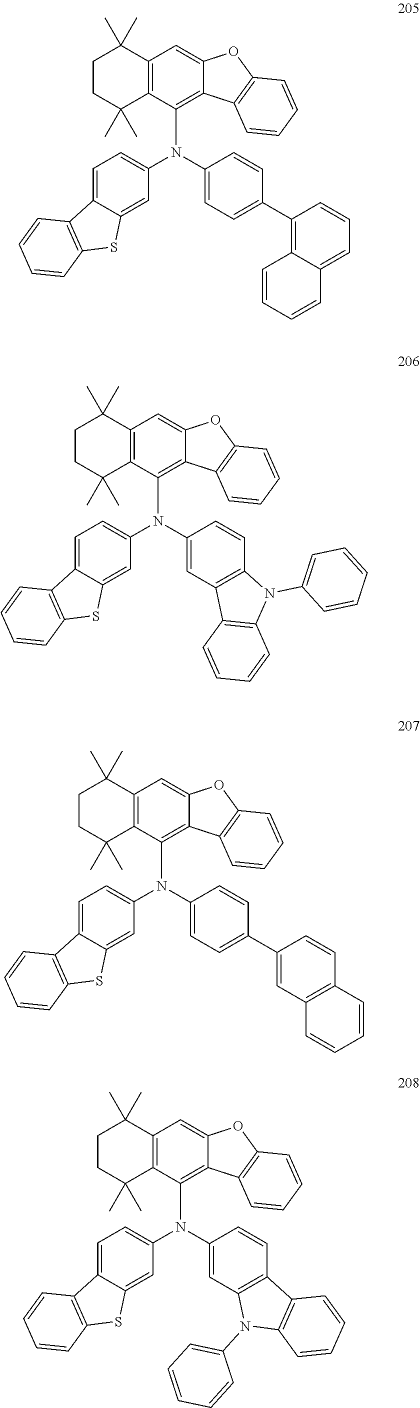 [SMILES: CC1(C)CCC(C)(C)c2c1cc1oc3ccccc3c1c2N(c1ccc(-c2cccc3ccccc23)cc1)c1ccc2c(c1)sc1ccccc12 ; CC1(C)CCC(C)(C)c2c1cc1oc3ccccc3c1c2N(c1ccc2c(c1)sc1ccccc12)c1ccc2c(c1)c1ccccc1n2-c1ccccc1 ; CC1(C)CCC(C)(C)c2c1cc1oc3ccccc3c1c2N(c1ccc(-c2ccc3ccccc3c2)cc1)c1ccc2c(c1)sc1ccccc12 ; CC1(C)CCC(C)(C)c2c1cc1oc3ccccc3c1c2N(c1ccc2c(c1)sc1ccccc12)c1ccc2c3ccccc3n(-c3ccccc3)c2c1]

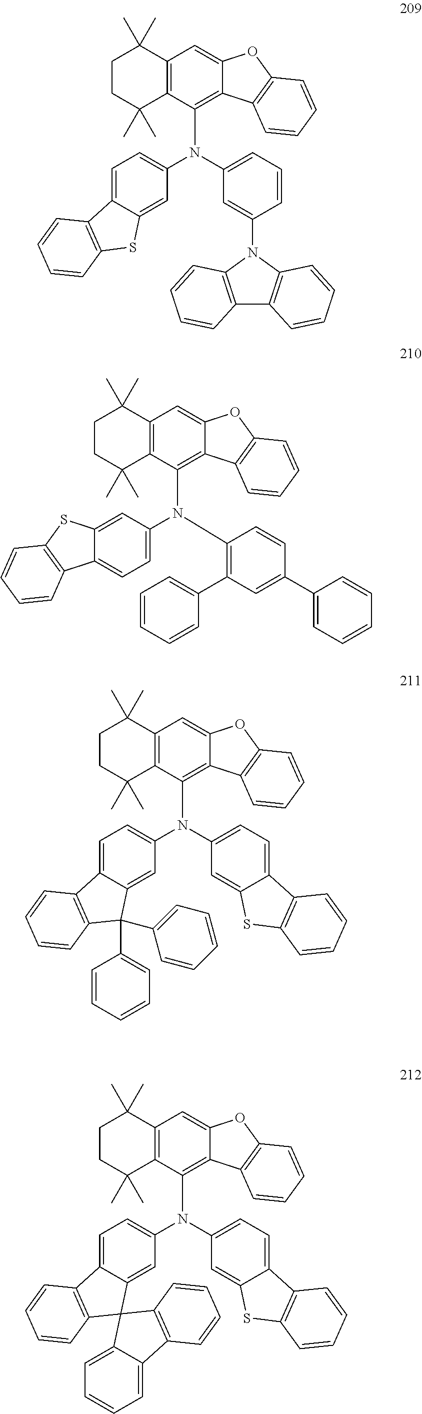 [SMILES: CC1(C)CCC(C)(C)c2c1cc1oc3ccccc3c1c2N(c1cccc(-n2c3ccccc3c3ccccc32)c1)c1ccc2c(c1)sc1ccccc12 ; CC1(C)CCC(C)(C)c2c1cc1oc3ccccc3c1c2N(c1ccc2c(c1)sc1ccccc12)c1ccc(-c2ccccc2)cc1-c1ccccc1 ; CC1(C)CCC(C)(C)c2c1cc1oc3ccccc3c1c2N(c1ccc2c(c1)C(c1ccccc1)(c1ccccc1)c1ccccc1-2)c1ccc2c(c1)sc1ccccc12 ; CC1(C)CCC(C)(C)c2c1cc1oc3ccccc3c1c2N(c1ccc2c(c1)C1(c3ccccc3-c3ccccc31)c1ccccc1-2)c1ccc2c(c1)sc1ccccc12]

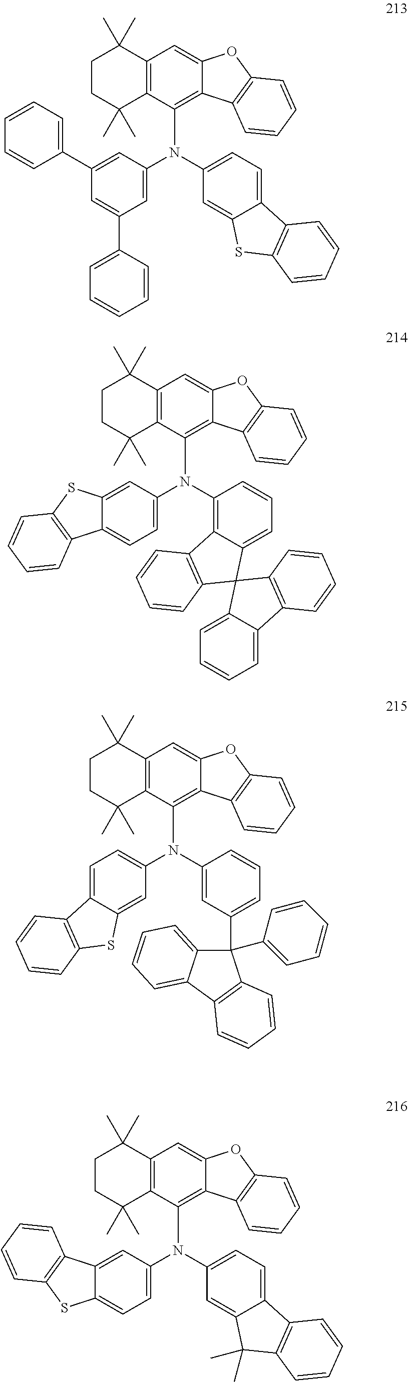

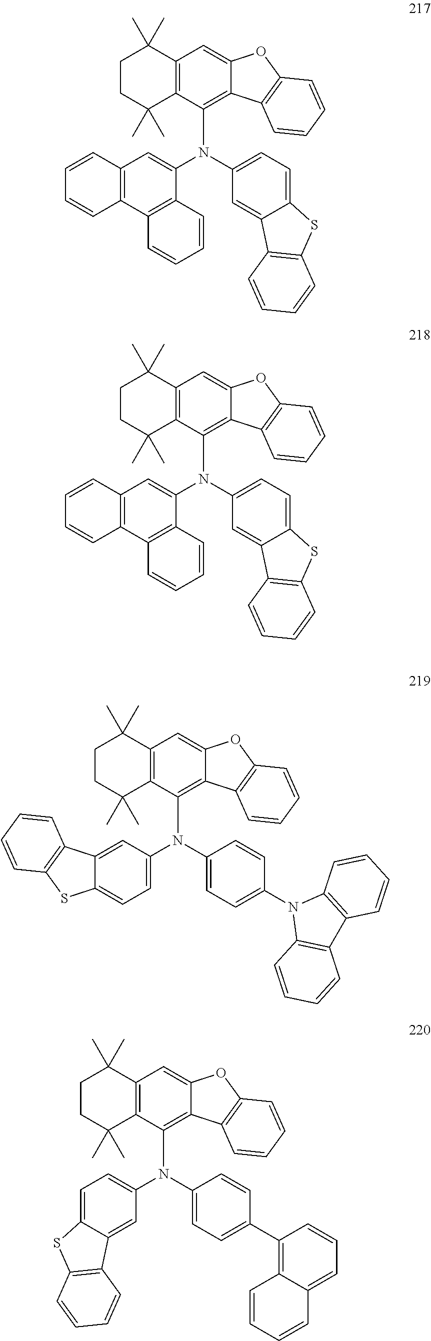 [SMILES: CC1(C)CCC(C)(C)c2c1cc1oc3ccccc3c1c2N(c1ccc2sc3ccccc3c2c1)c1cc2ccccc2c2ccccc12 ; CC1(C)CCC(C)(C)c2c1cc1oc3ccccc3c1c2N(c1ccc2sc3ccccc3c2c1)c1cc2ccccc2c2ccccc12 ; CC1(C)CCC(C)(C)c2c1cc1oc3ccccc3c1c2N(c1ccc(-n2c3ccccc3c3ccccc32)cc1)c1ccc2sc3ccccc3c2c1 ; CC1(C)CCC(C)(C)c2c1cc1oc3ccccc3c1c2N(c1ccc(-c2cccc3ccccc23)cc1)c1ccc2sc3ccccc3c2c1]

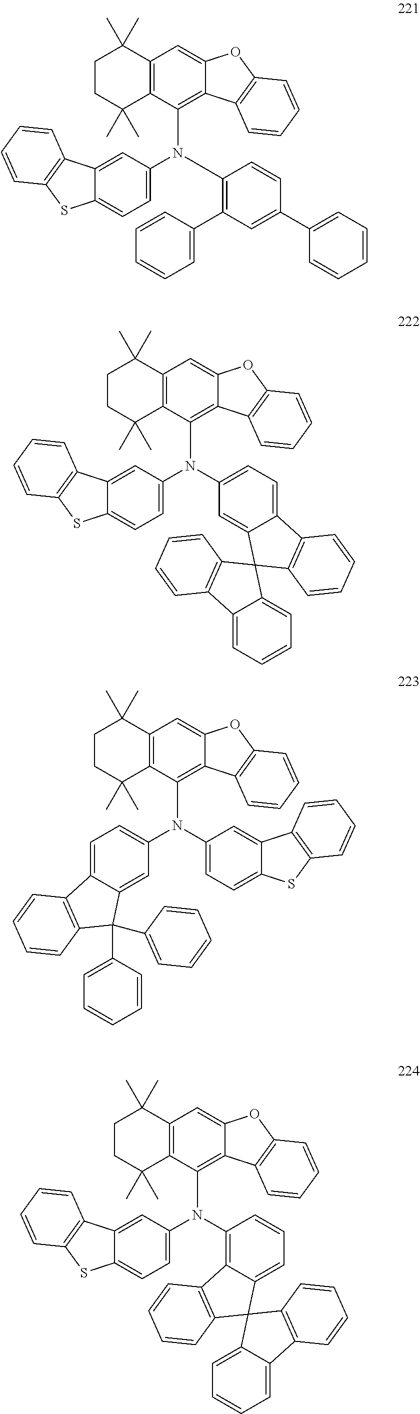 [SMILES: CC1(C)CCC(C)(C)c2c1cc1oc3ccccc3c1c2N(c1ccc2sc3ccccc3c2c1)c1ccc(-c2ccccc2)cc1-c1ccccc1 ; CC1(C)CCC(C)(C)c2c1cc1oc3ccccc3c1c2N(c1ccc2c(c1)C1(c3ccccc3-c3ccccc31)c1ccccc1-2)c1ccc2sc3ccccc3c2c1 ; CC1(C)CCC(C)(C)c2c1cc1oc3ccccc3c1c2N(c1ccc2c(c1)C(c1ccccc1)(c1ccccc1)c1ccccc1-2)c1ccc2sc3ccccc3c2c1 ; CC1(C)CCC(C)(C)c2c1cc1oc3ccccc3c1c2N(c1ccc2sc3ccccc3c2c1)c1cccc2c1-c1ccccc1C21c2ccccc2-c2ccccc21]

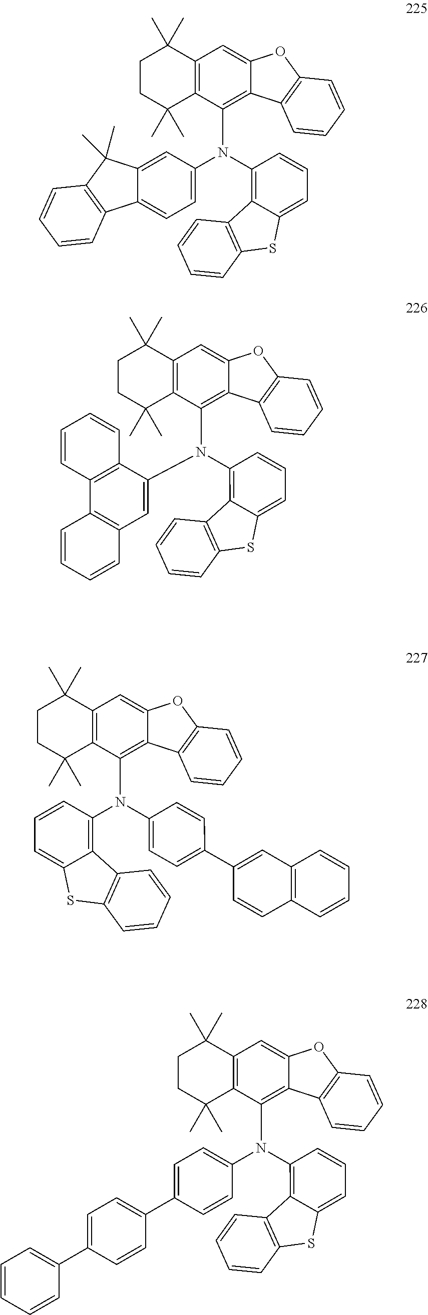

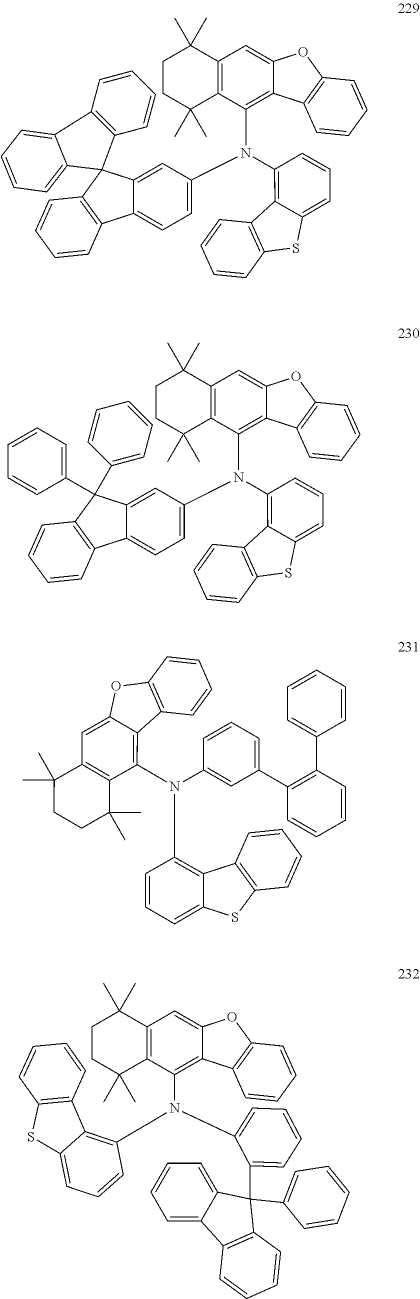 [SMILES: CC1(C)CCC(C)(C)c2c1cc1oc3ccccc3c1c2N(c1ccc2c(c1)C1(c3ccccc3-c3ccccc31)c1ccccc1-2)c1cccc2sc3ccccc3c12 ; CC1(C)CCC(C)(C)c2c1cc1oc3ccccc3c1c2N(c1ccc2c(c1)C(c1ccccc1)(c1ccccc1)c1ccccc1-2)c1cccc2sc3ccccc3c12 ; CC1(C)CCC(C)(C)c2c1cc1oc3ccccc3c1c2N(c1cccc(-c2ccccc2-c2ccccc2)c1)c1cccc2sc3ccccc3c12 ; CC1(C)CCC(C)(C)c2c1cc1oc3ccccc3c1c2N(c1ccccc1C1(c2ccccc2)c2ccccc2-c2ccccc21)c1cccc2sc3ccccc3c12]

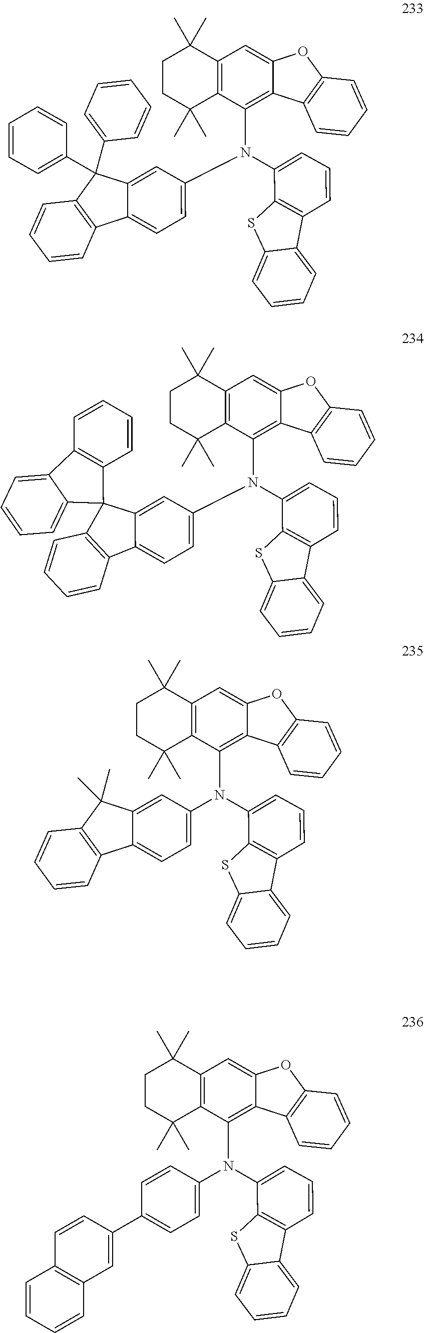 [SMILES: CC1(C)CCC(C)(C)c2c1cc1oc3ccccc3c1c2N(c1ccc2c(c1)C(c1ccccc1)(c1ccccc1)c1ccccc1-2)c1cccc2c1sc1ccccc12 ; CC1(C)CCC(C)(C)c2c1cc1oc3ccccc3c1c2N(c1ccc2c(c1)C1(c3ccccc3-c3ccccc31)c1ccccc1-2)c1cccc2c1sc1ccccc12 ; CC1(C)CCC(C)(C)c2c1cc1oc3ccccc3c1c2N(c1ccc2c(c1)C(C)(C)c1ccccc1-2)c1cccc2c1sc1ccccc12 ; CC1(C)CCC(C)(C)c2c1cc1oc3ccccc3c1c2N(c1ccc(-c2ccc3ccccc3c2)cc1)c1cccc2c1sc1ccccc12]

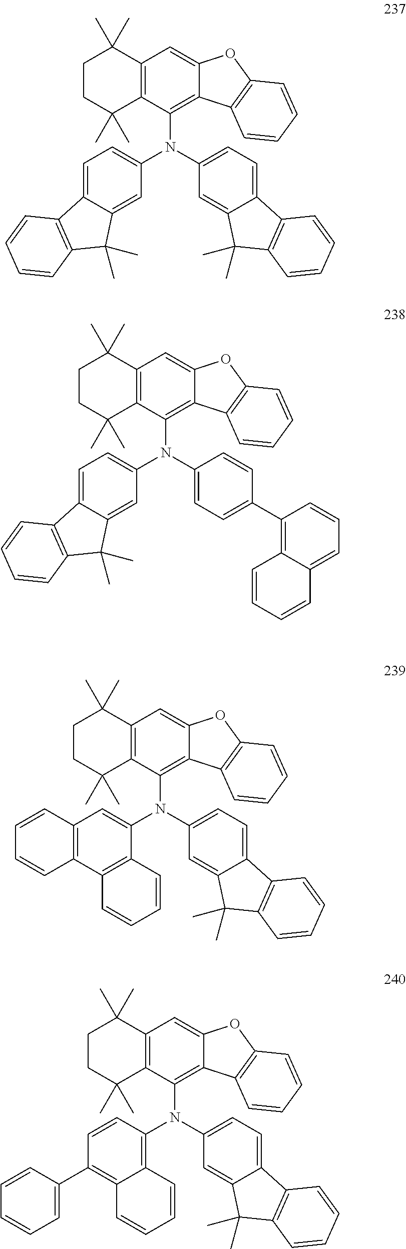

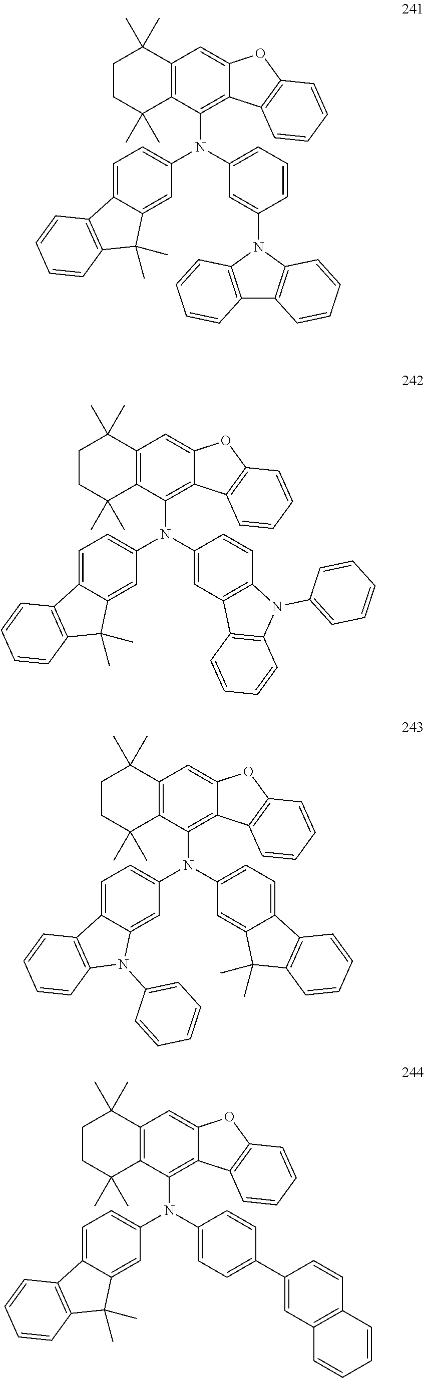

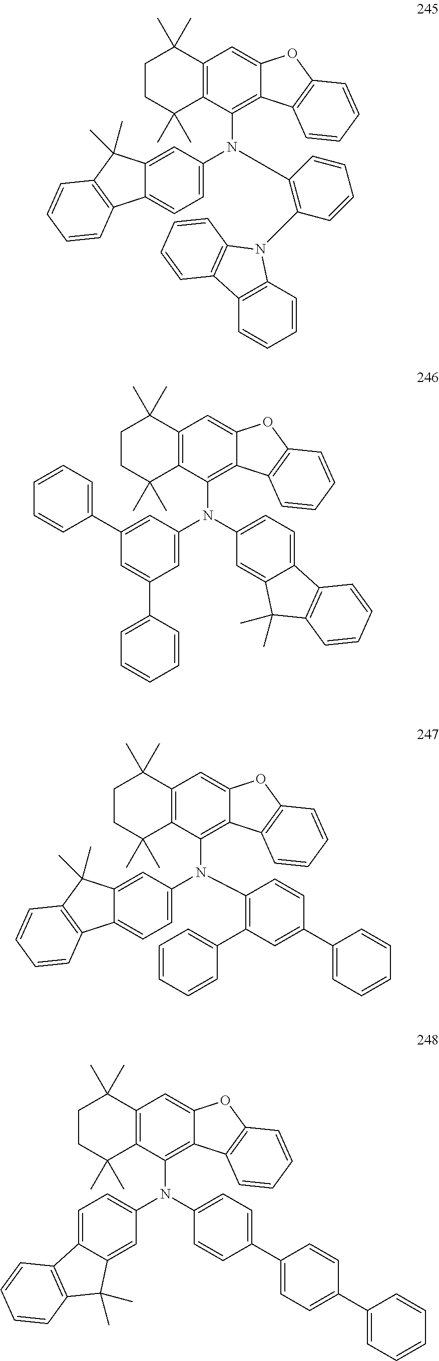 [SMILES: CC1(C)CCC(C)(C)c2c1cc1oc3ccccc3c1c2N(c1ccc2c(c1)C(C)(C)c1ccccc1-2)c1ccccc1-n1c2ccccc2c2ccccc21 ; CC1(C)CCC(C)(C)c2c1cc1oc3ccccc3c1c2N(c1cc(-c2ccccc2)cc(-c2ccccc2)c1)c1ccc2c(c1)C(C)(C)c1ccccc1-2 ; CC1(C)CCC(C)(C)c2c1cc1oc3ccccc3c1c2N(c1ccc2c(c1)C(C)(C)c1ccccc1-2)c1ccc(-c2ccccc2)cc1-c1ccccc1 ; CC1(C)CCC(C)(C)c2c1cc1oc3ccccc3c1c2N(c1ccc(-c2ccc(-c3ccccc3)cc2)cc1)c1ccc2c(c1)C(C)(C)c1ccccc1-2]

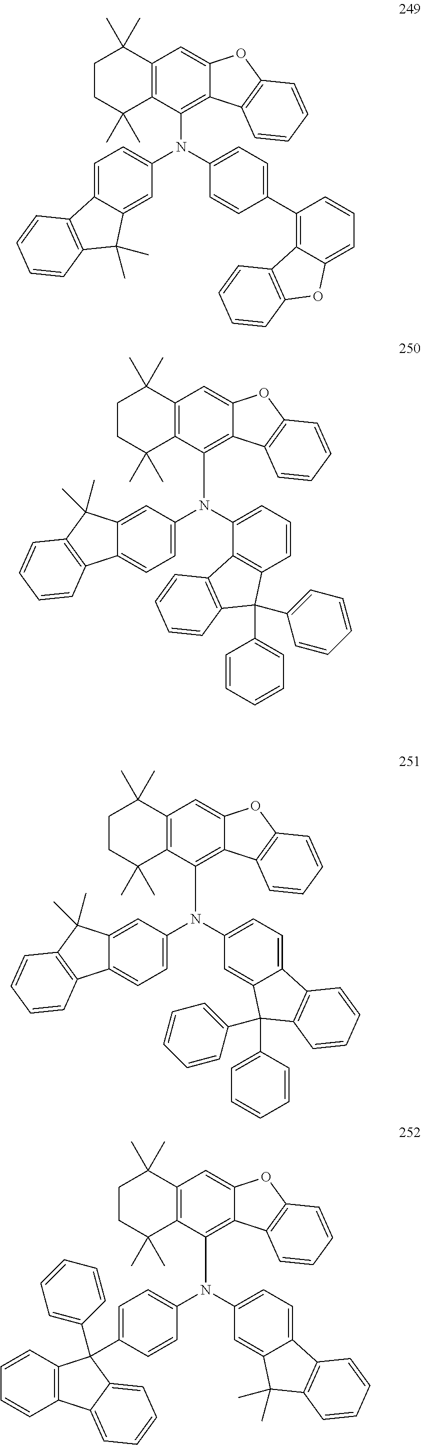 [SMILES: CC1(C)CCC(C)(C)c2c1cc1oc3ccccc3c1c2N(c1ccc(-c2cccc3oc4ccccc4c23)cc1)c1ccc2c(c1)C(C)(C)c1ccccc1-2 ; CC1(C)CCC(C)(C)c2c1cc1oc3ccccc3c1c2N(c1ccc2c(c1)C(C)(C)c1ccccc1-2)c1cccc2c1-c1ccccc1C2(c1ccccc1)c1ccccc1 ; CC1(C)CCC(C)(C)c2c1cc1oc3ccccc3c1c2N(c1ccc2c(c1)C(C)(C)c1ccccc1-2)c1ccc2c(c1)C(c1ccccc1)(c1ccccc1)c1ccccc1-2 ; CC1(C)CCC(C)(C)c2c1cc1oc3ccccc3c1c2N(c1ccc(C2(c3ccccc3)c3ccccc3-c3ccccc32)cc1)c1ccc2c(c1)C(C)(C)c1ccccc1-2]

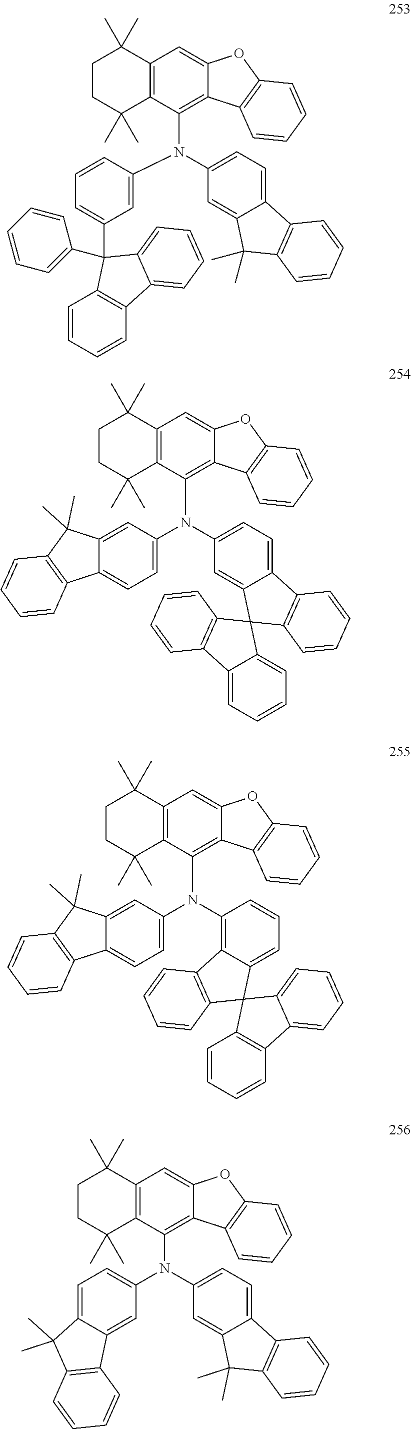

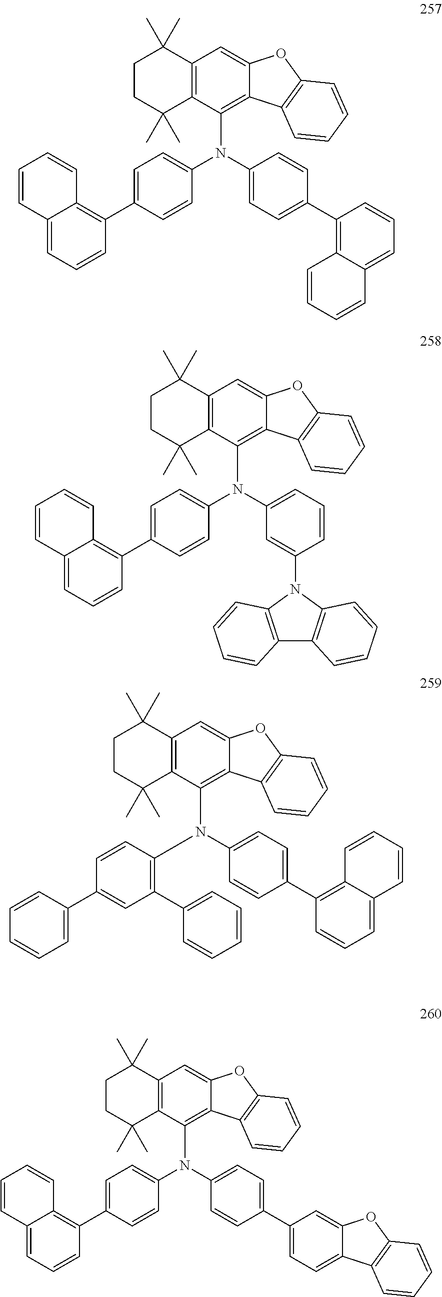 [SMILES: CC1(C)CCC(C)(C)c2c1cc1oc3ccccc3c1c2N(c1ccc(-c2cccc3ccccc23)cc1)c1ccc(-c2cccc3ccccc23)cc1 ; CC1(C)CCC(C)(C)c2c1cc1oc3ccccc3c1c2N(c1ccc(-c2cccc3ccccc23)cc1)c1cccc(-n2c3ccccc3c3ccccc32)c1 ; CC1(C)CCC(C)(C)c2c1cc1oc3ccccc3c1c2N(c1ccc(-c2cccc3ccccc23)cc1)c1ccc(-c2ccccc2)cc1-c1ccccc1 ; CC1(C)CCC(C)(C)c2c1cc1oc3ccccc3c1c2N(c1ccc(-c2ccc3c(c2)oc2ccccc23)cc1)c1ccc(-c2cccc3ccccc23)cc1]

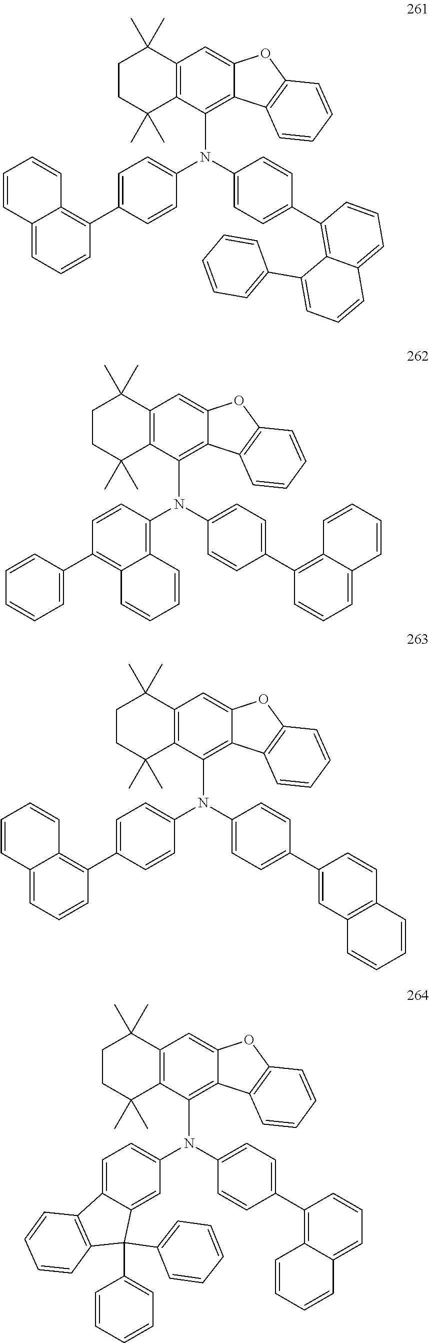

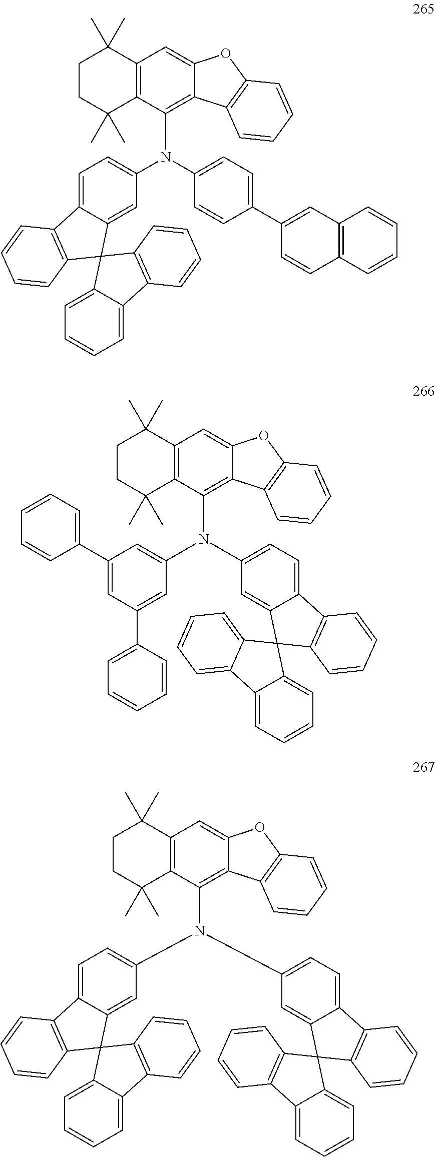 [SMILES: CC1(C)CCC(C)(C)c2c1cc1oc3ccccc3c1c2N(c1ccc(-c2ccc3ccccc3c2)cc1)c1ccc2c(c1)C1(c3ccccc3-c3ccccc31)c1ccccc1-2 ; CC1(C)CCC(C)(C)c2c1cc1oc3ccccc3c1c2N(c1cc(-c2ccccc2)cc(-c2ccccc2)c1)c1ccc2c(c1)C1(c3ccccc3-c3ccccc31)c1ccccc1-2 ; CC1(C)CCC(C)(C)c2c1cc1oc3ccccc3c1c2N(c1ccc2c(c1)C1(c3ccccc3-c3ccccc31)c1ccccc1-2)c1ccc2c(c1)C1(c3ccccc3-c3ccccc31)c1ccccc1-2]

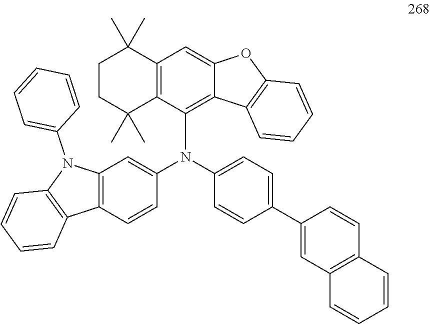 [SMILES: CC1(C)CCC(C)(C)c2c1cc1oc3ccccc3c1c2N(c1ccc(-c2ccc3ccccc3c2)cc1)c1ccc2c3ccccc3n(-c3ccccc3)c2c1]

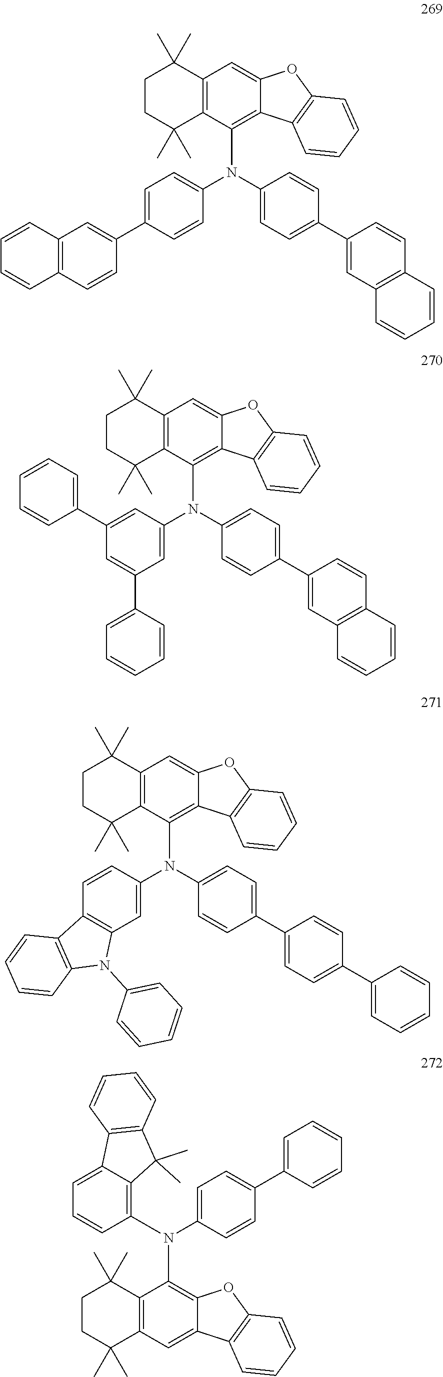

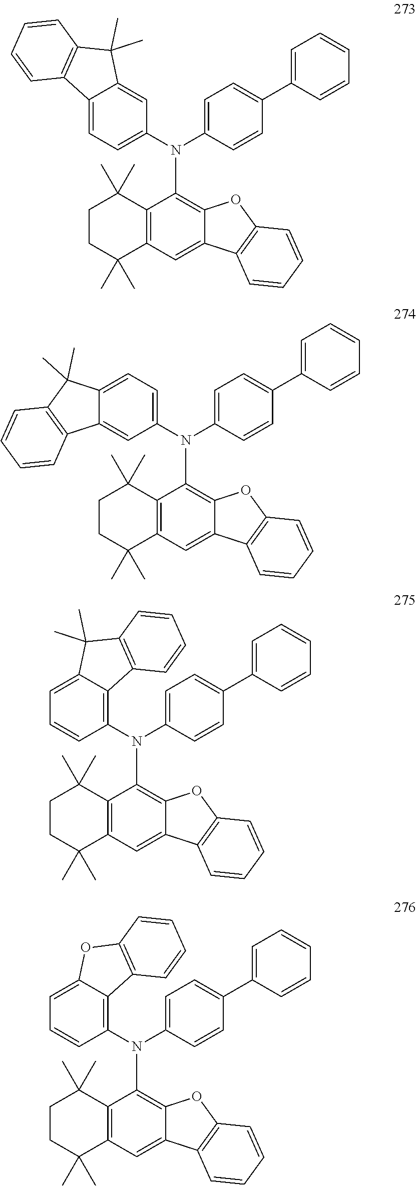

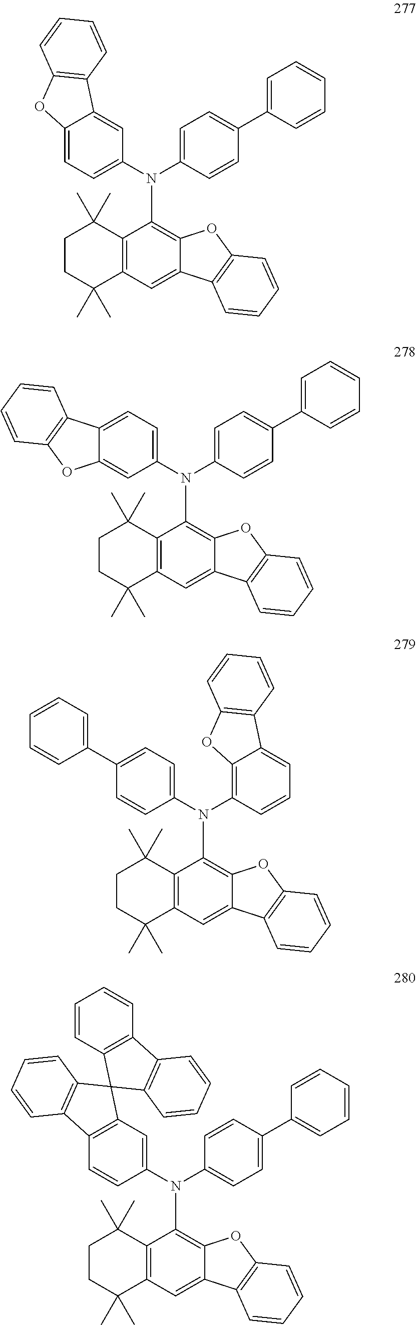

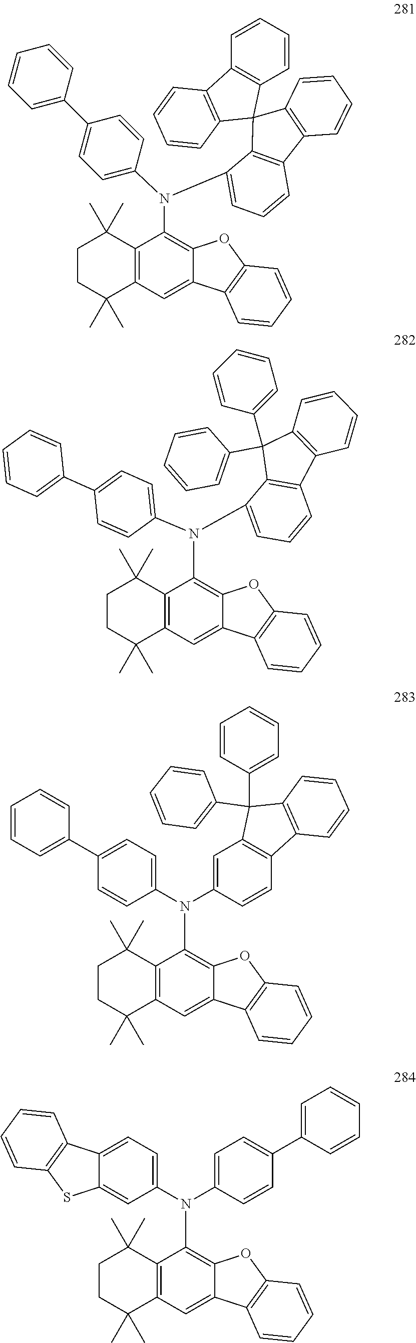

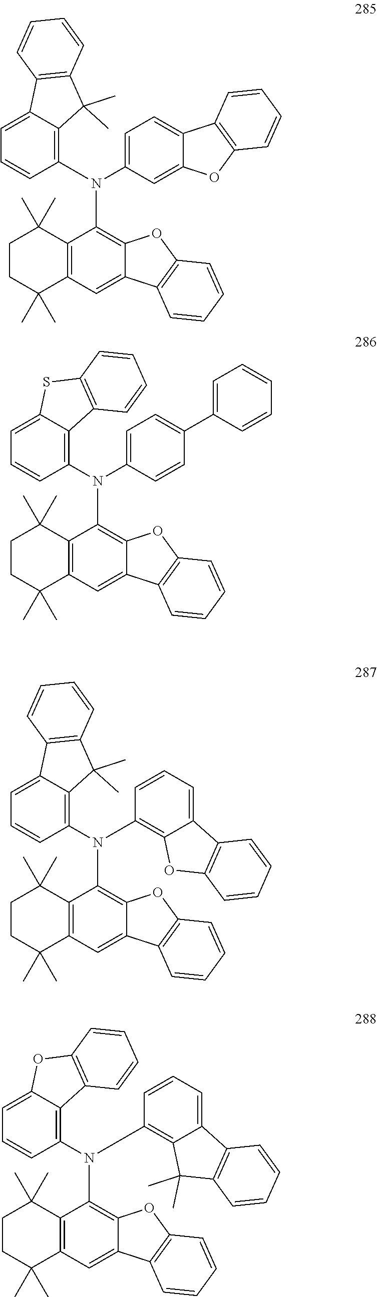 [SMILES: CC1(C)CCC(C)(C)c2c1cc1c(oc3ccccc31)c2N(c1ccc2c(c1)oc1ccccc12)c1cccc2c1C(C)(C)c1ccccc1-2 ; CC1(C)CCC(C)(C)c2c1cc1c(oc3ccccc31)c2N(c1ccc(-c2ccccc2)cc1)c1cccc2sc3ccccc3c12 ; CC1(C)CCC(C)(C)c2c1cc1c(oc3ccccc31)c2N(c1cccc2c1C(C)(C)c1ccccc1-2)c1cccc2c1oc1ccccc12 ; CC1(C)CCC(C)(C)c2c1cc1c(oc3ccccc31)c2N(c1cccc2c1C(C)(C)c1ccccc1-2)c1cccc2oc3ccccc3c12]

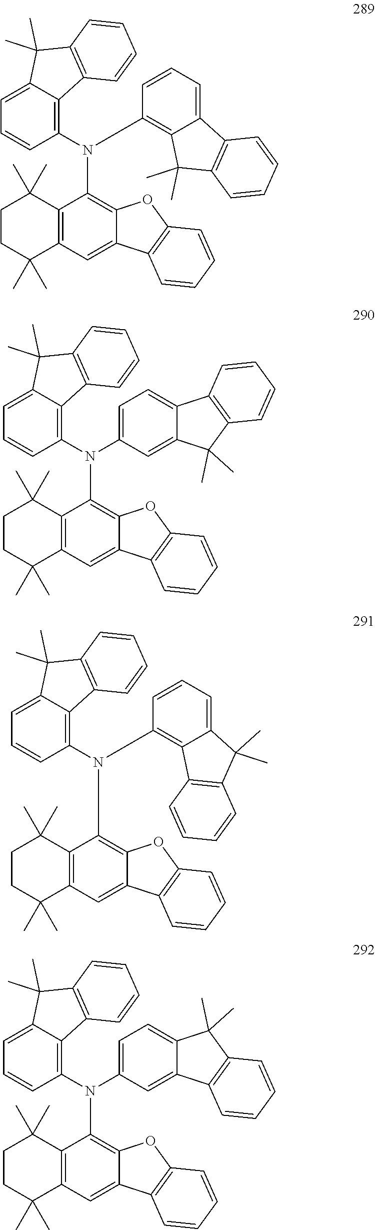

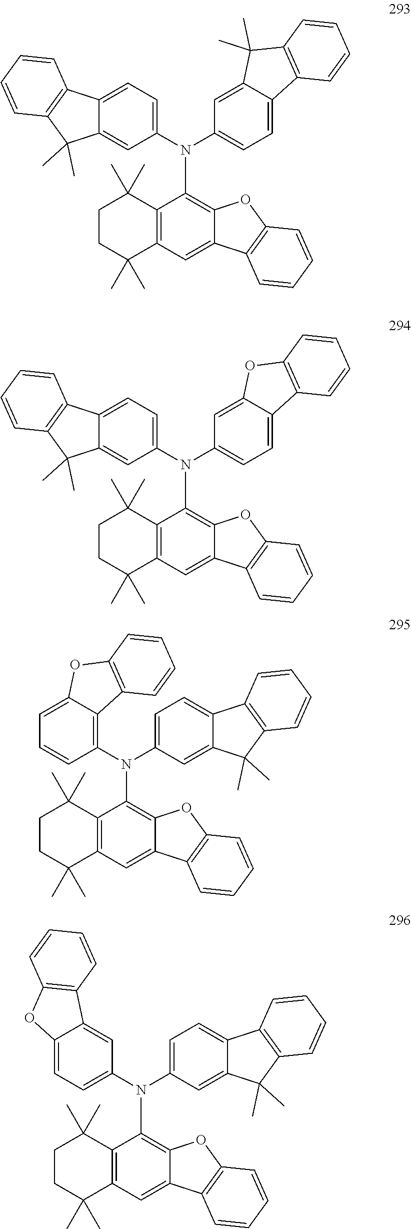

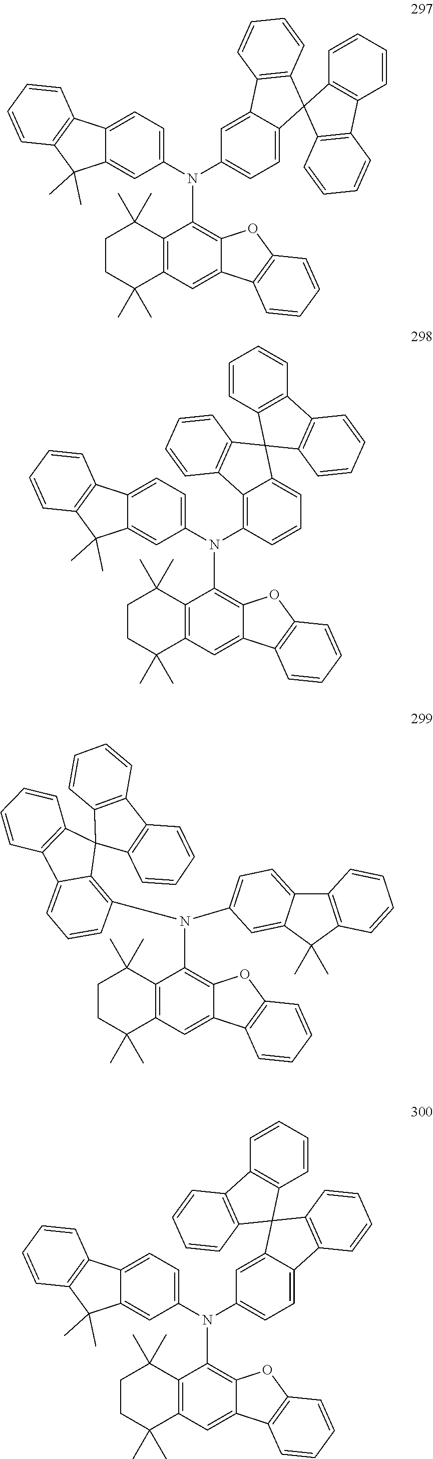

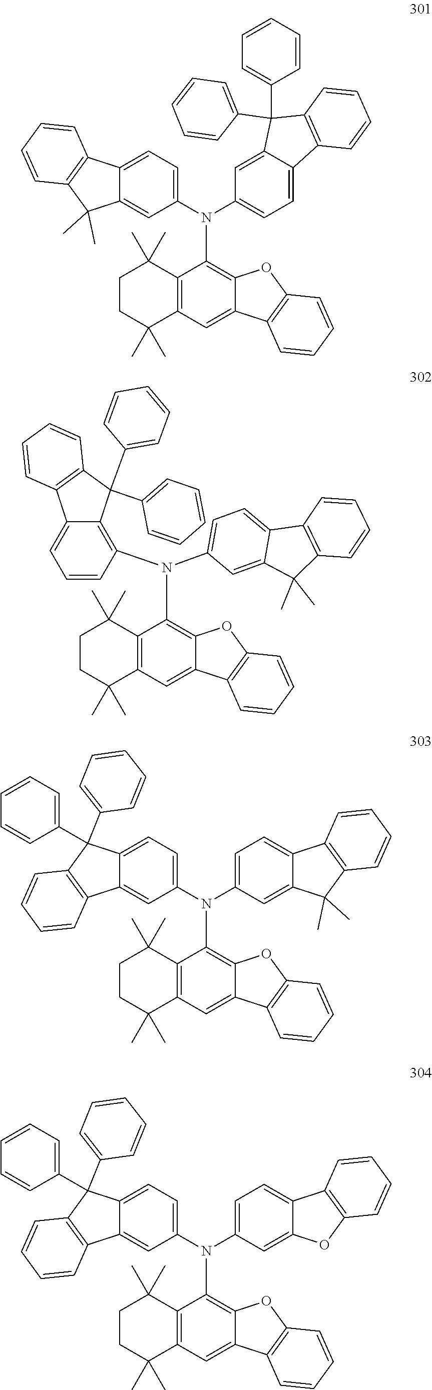

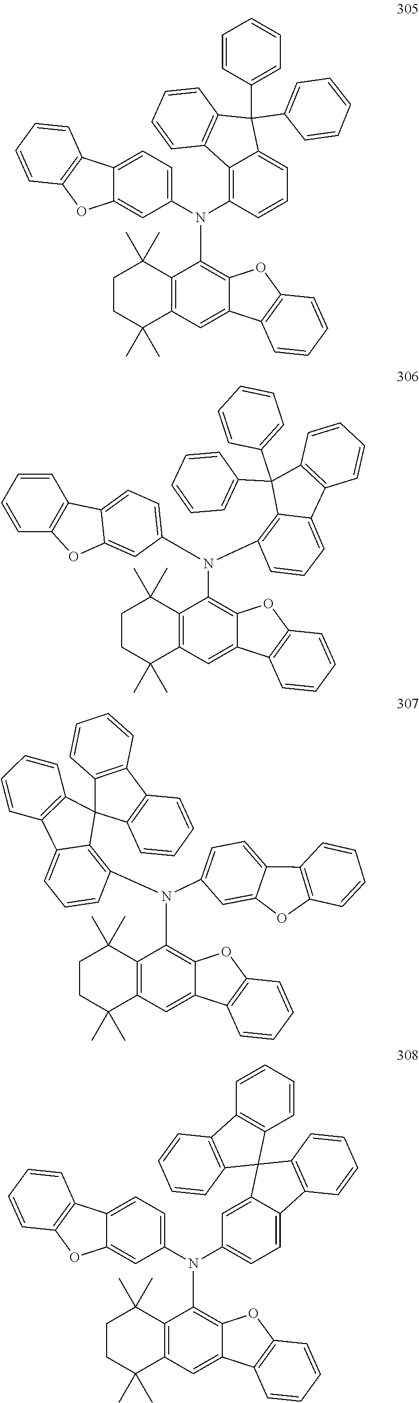

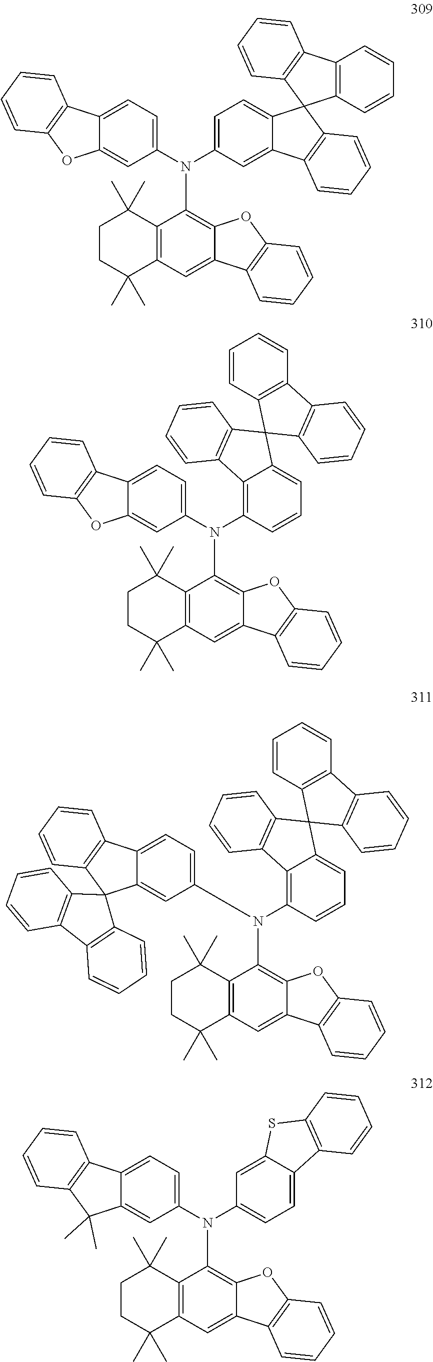

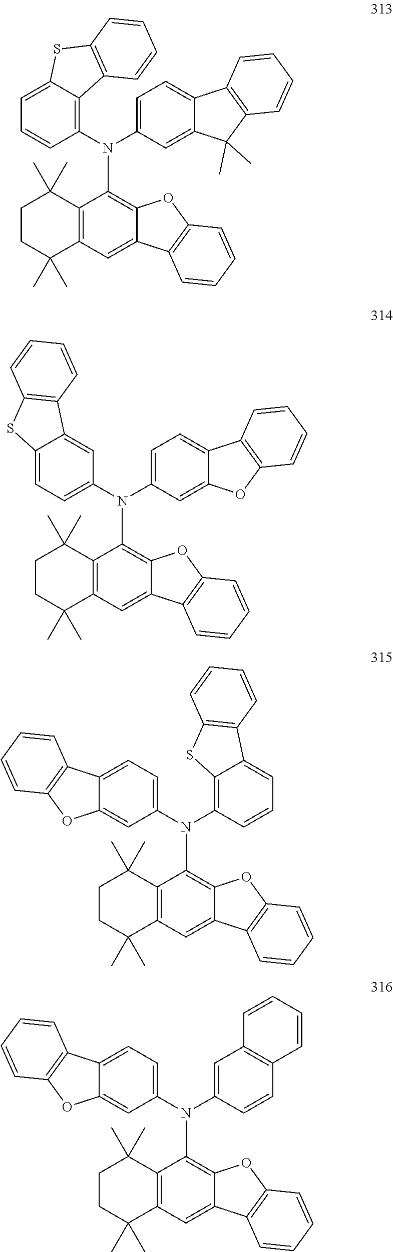

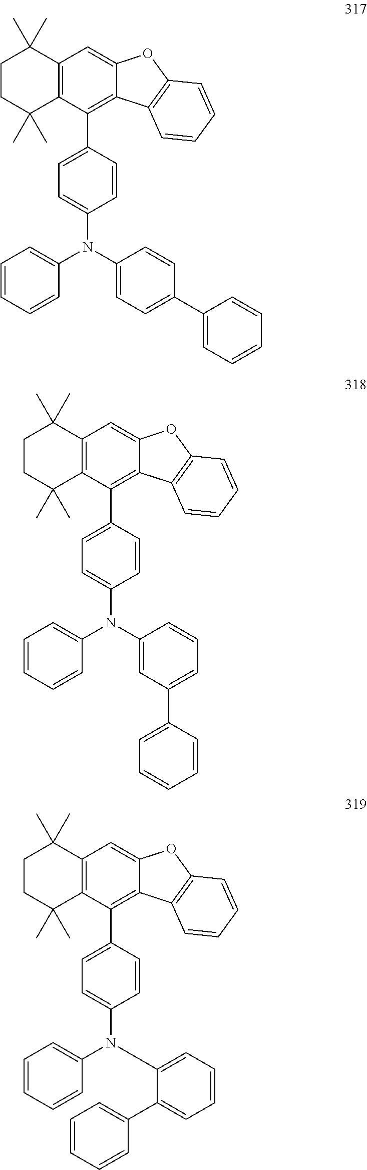 [SMILES: CC1(C)CCC(C)(C)c2c1cc1oc3ccccc3c1c2-c1ccc(N(c2ccccc2)c2ccc(-c3ccccc3)cc2)cc1 ; CC1(C)CCC(C)(C)c2c1cc1oc3ccccc3c1c2-c1ccc(N(c2ccccc2)c2cccc(-c3ccccc3)c2)cc1 ; CC1(C)CCC(C)(C)c2c1cc1oc3ccccc3c1c2-c1ccc(N(c2ccccc2)c2ccccc2-c2ccccc2)cc1]

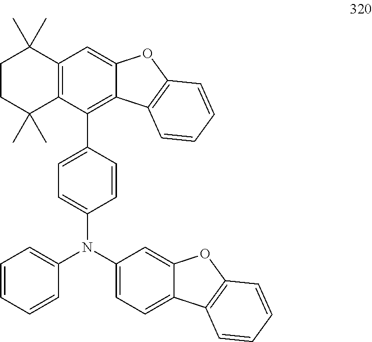 [SMILES: CC1(C)CCC(C)(C)c2c1cc1oc3ccccc3c1c2-c1ccc(N(c2ccccc2)c2ccc3c(c2)oc2ccccc23)cc1]

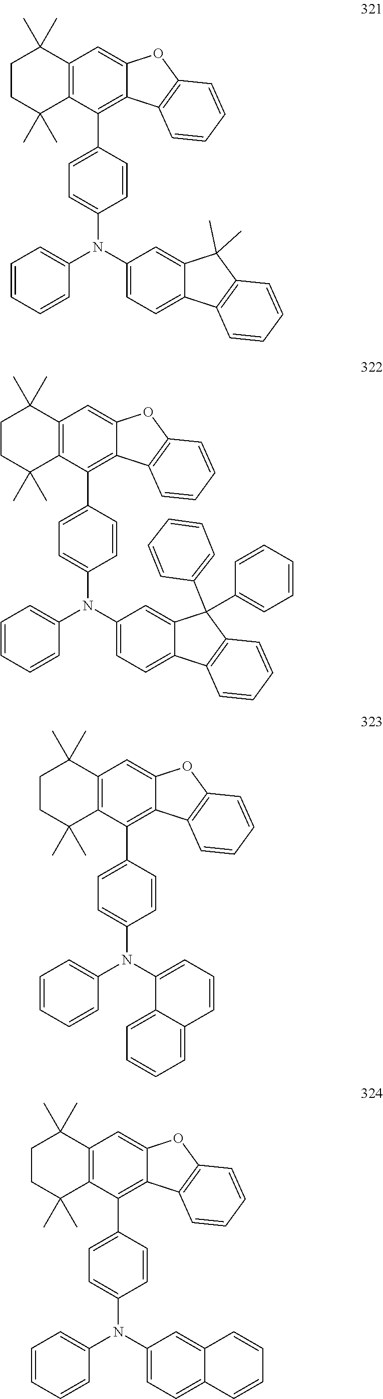 [SMILES: CC1(C)CCC(C)(C)c2c1cc1oc3ccccc3c1c2-c1ccc(N(c2ccccc2)c2ccc3c(c2)C(C)(C)c2ccccc2-3)cc1 ; CC1(C)CCC(C)(C)c2c1cc1oc3ccccc3c1c2-c1ccc(N(c2ccccc2)c2ccc3c(c2)C(c2ccccc2)(c2ccccc2)c2ccccc2-3)cc1 ; CC1(C)CCC(C)(C)c2c1cc1oc3ccccc3c1c2-c1ccc(N(c2ccccc2)c2cccc3ccccc23)cc1 ; CC1(C)CCC(C)(C)c2c1cc1oc3ccccc3c1c2-c1ccc(N(c2ccccc2)c2ccc3ccccc3c2)cc1]

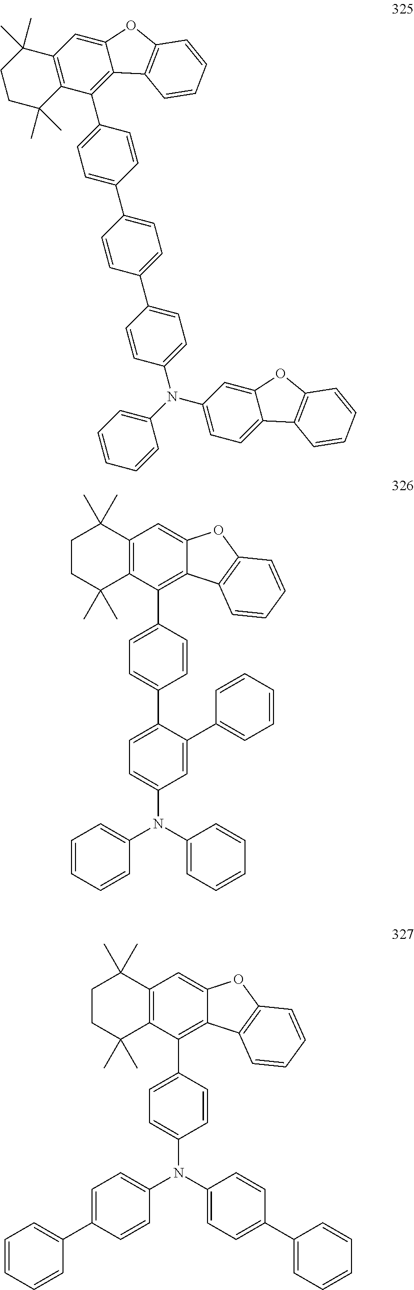

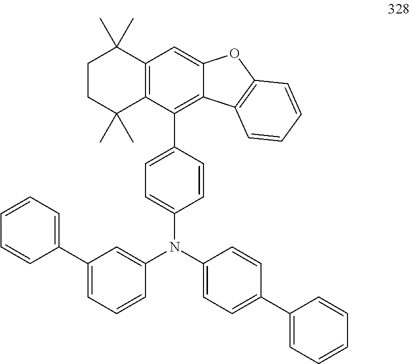 [SMILES: CC1(C)CCC(C)(C)c2c1cc1oc3ccccc3c1c2-c1ccc(N(c2ccc(-c3ccccc3)cc2)c2cccc(-c3ccccc3)c2)cc1]

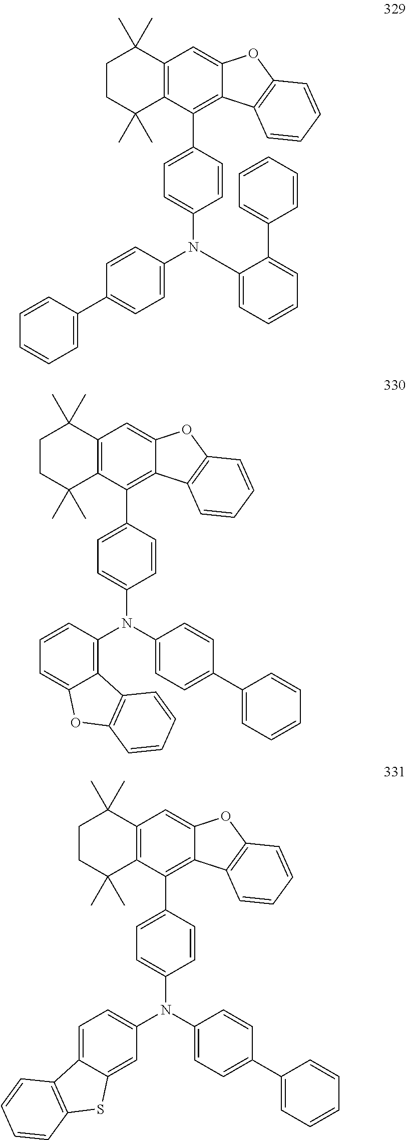

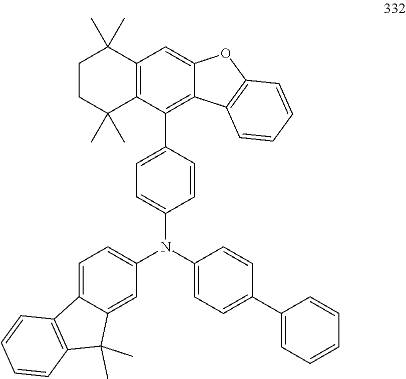 [SMILES: CC1(C)CCC(C)(C)c2c1cc1oc3ccccc3c1c2-c1ccc(N(c2ccc(-c3ccccc3)cc2)c2ccc3c(c2)C(C)(C)c2ccccc2-3)cc1]

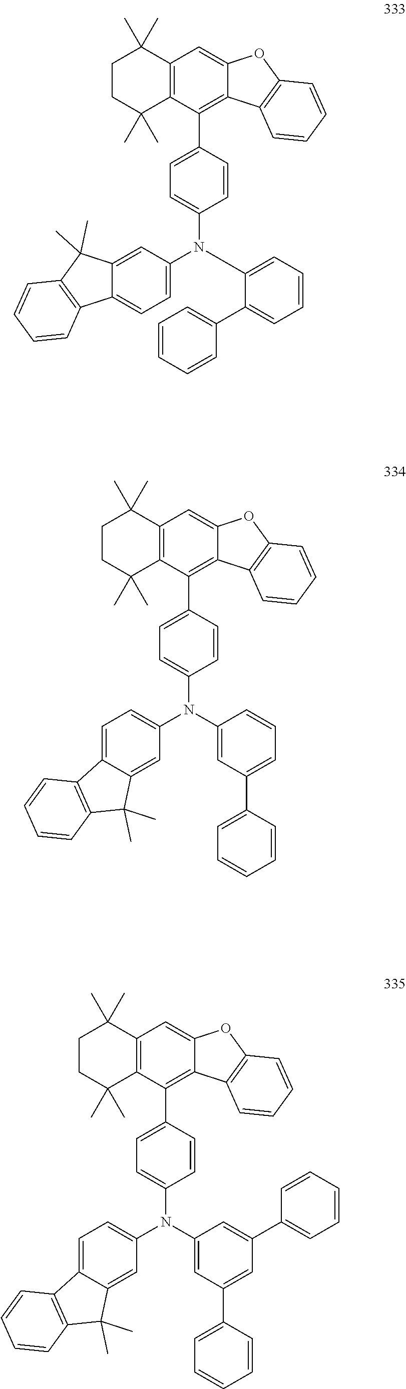 [SMILES: CC1(C)CCC(C)(C)c2c1cc1oc3ccccc3c1c2-c1ccc(N(c2ccc3c(c2)C(C)(C)c2ccccc2-3)c2ccccc2-c2ccccc2)cc1 ; CC1(C)CCC(C)(C)c2c1cc1oc3ccccc3c1c2-c1ccc(N(c2cccc(-c3ccccc3)c2)c2ccc3c(c2)C(C)(C)c2ccccc2-3)cc1 ; CC1(C)CCC(C)(C)c2c1cc1oc3ccccc3c1c2-c1ccc(N(c2cc(-c3ccccc3)cc(-c3ccccc3)c2)c2ccc3c(c2)C(C)(C)c2ccccc2-3)cc1]

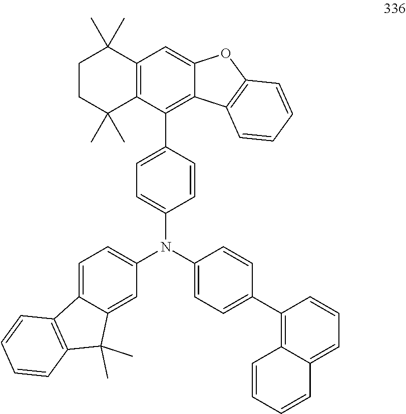 [SMILES: CC1(C)CCC(C)(C)c2c1cc1oc3ccccc3c1c2-c1ccc(N(c2ccc(-c3cccc4ccccc34)cc2)c2ccc3c(c2)C(C)(C)c2ccccc2-3)cc1]

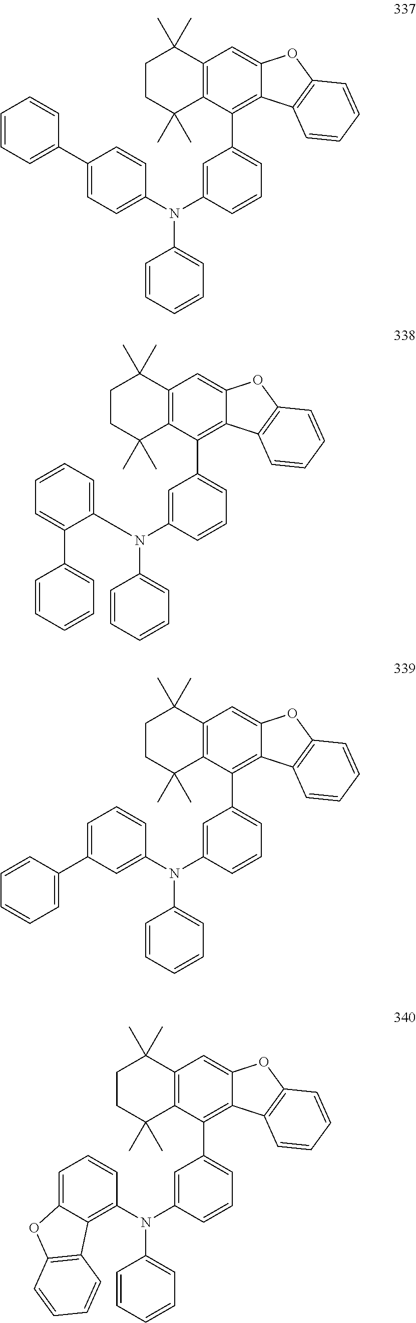

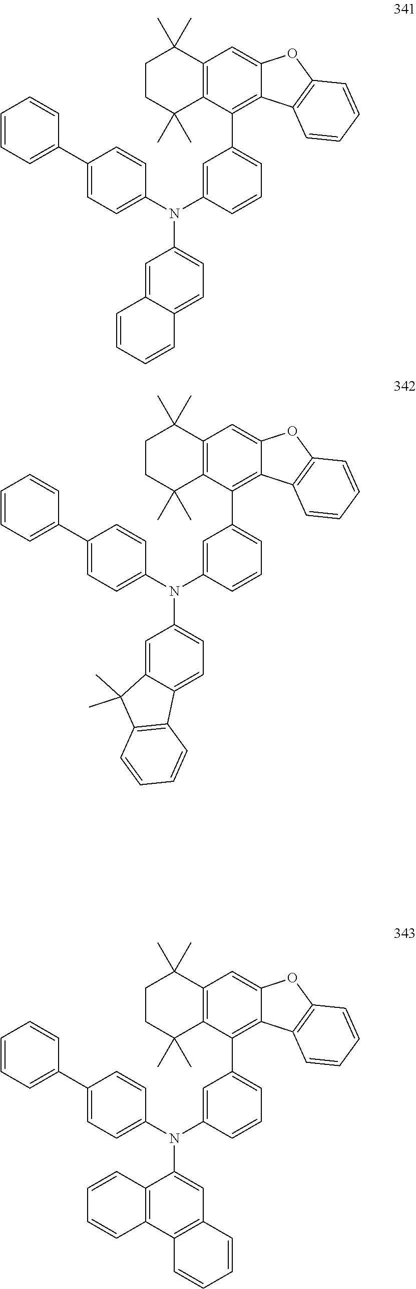

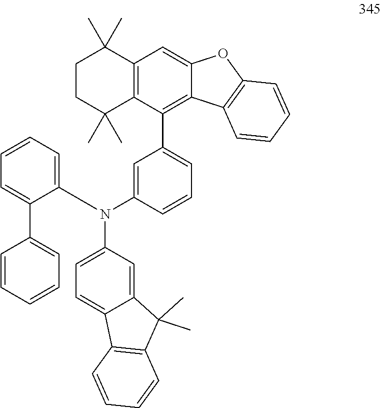 [SMILES: CC1(C)CCC(C)(C)c2c1cc1oc3ccccc3c1c2-c1cccc(N(c2ccc3c(c2)C(C)(C)c2ccccc2-3)c2ccccc2-c2ccccc2)c1]

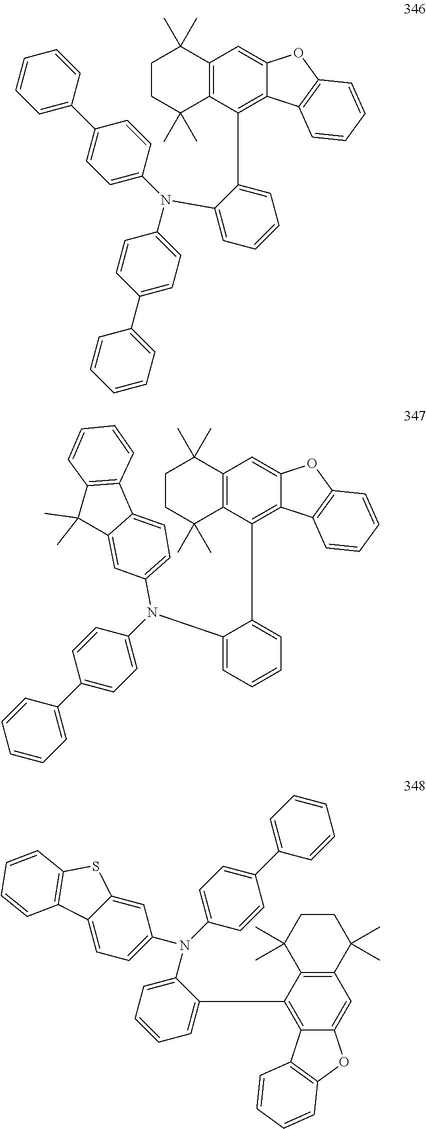

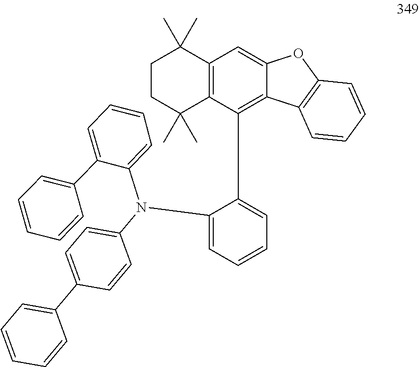 [SMILES: CC1(C)CCC(C)(C)c2c1cc1oc3ccccc3c1c2-c1ccccc1N(c1ccc(-c2ccccc2)cc1)c1ccccc1-c1ccccc1]

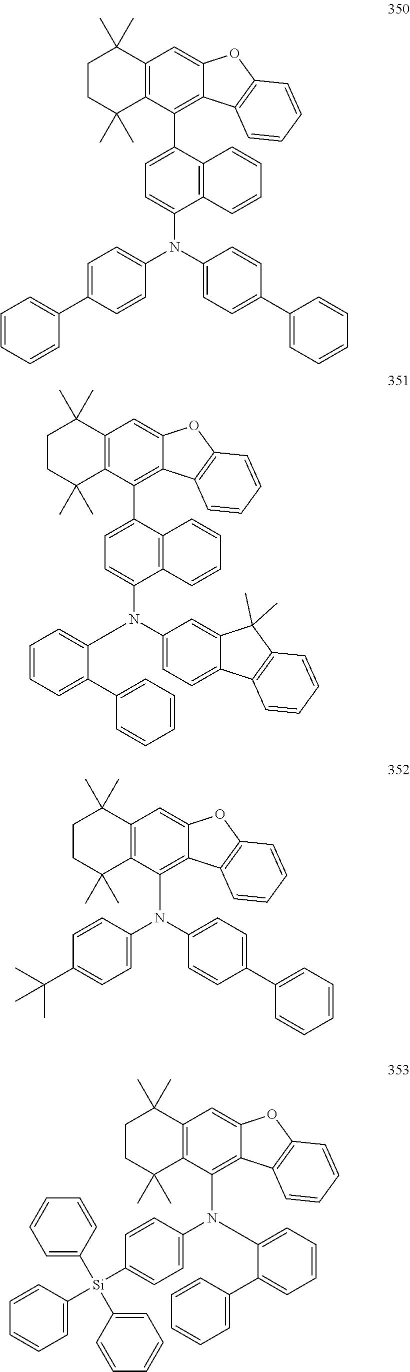

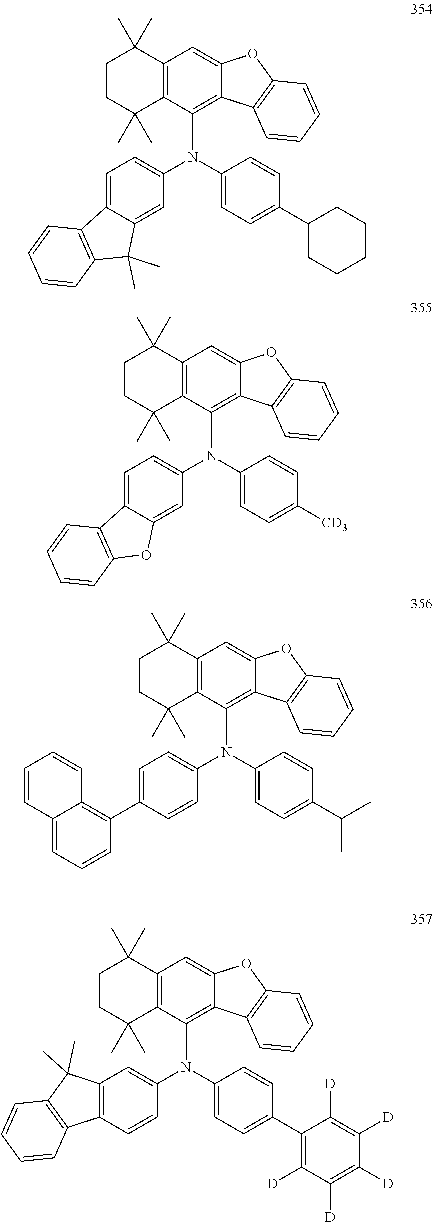

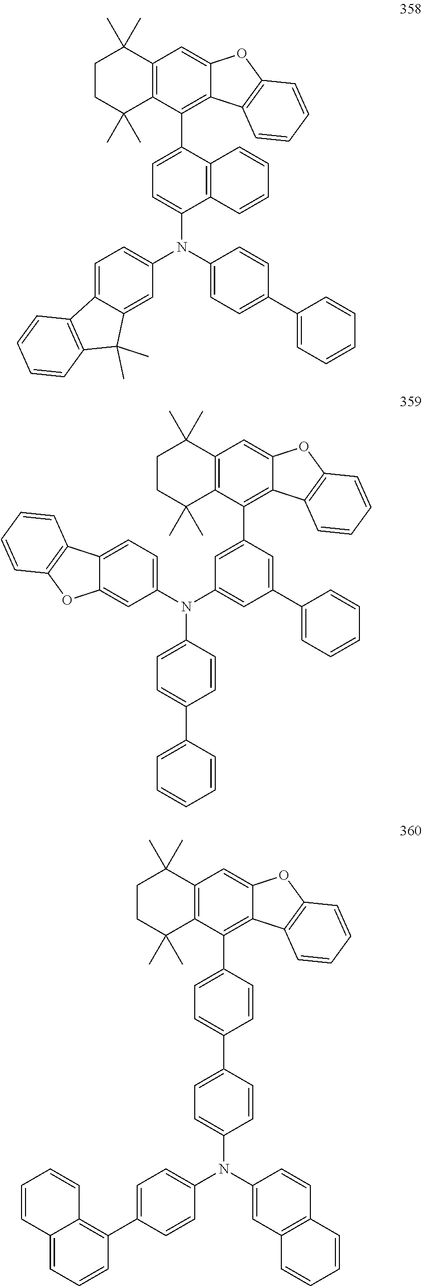

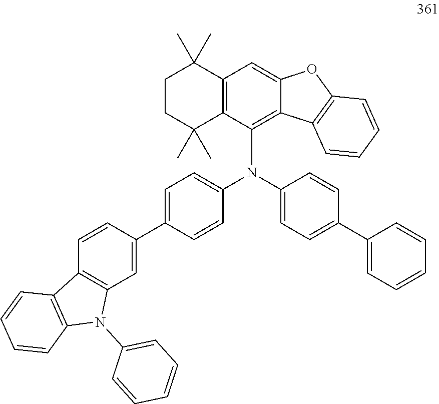 [SMILES: CC1(C)CCC(C)(C)c2c1cc1oc3ccccc3c1c2N(c1ccc(-c2ccccc2)cc1)c1ccc(-c2ccc3c4ccccc4n(-c4ccccc4)c3c2)cc1]

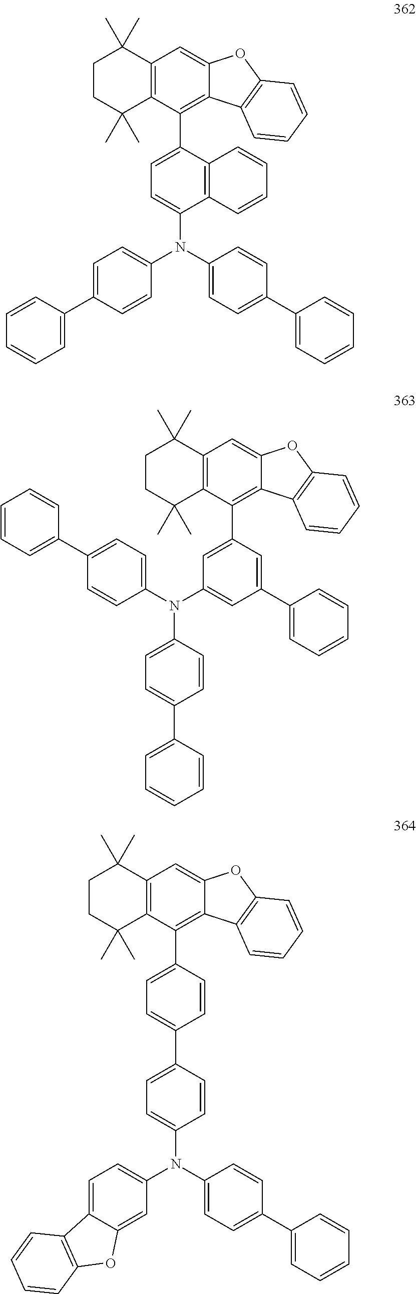 [SMILES: CC1(C)CCC(C)(C)c2c1cc1oc3ccccc3c1c2-c1ccc(N(c2ccc(-c3ccccc3)cc2)c2ccc(-c3ccccc3)cc2)c2ccccc12 ; CC1(C)CCC(C)(C)c2c1cc1oc3ccccc3c1c2-c1cc(-c2ccccc2)cc(N(c2ccc(-c3ccccc3)cc2)c2ccc(-c3ccccc3)cc2)c1 ; CC1(C)CCC(C)(C)c2c1cc1oc3ccccc3c1c2-c1ccc(-c2ccc(N(c3ccc(-c4ccccc4)cc3)c3ccc4c(c3)oc3ccccc34)cc2)cc1]

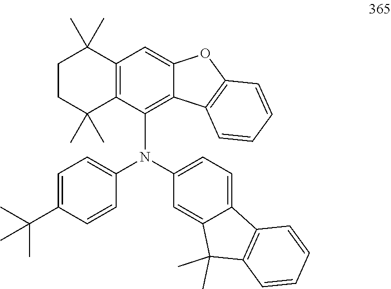 [SMILES: CC(C)(C)c1ccc(N(c2ccc3c(c2)C(C)(C)c2ccccc2-3)c2c3c(cc4oc5ccccc5c24)C(C)(C)CCC3(C)C)cc1]

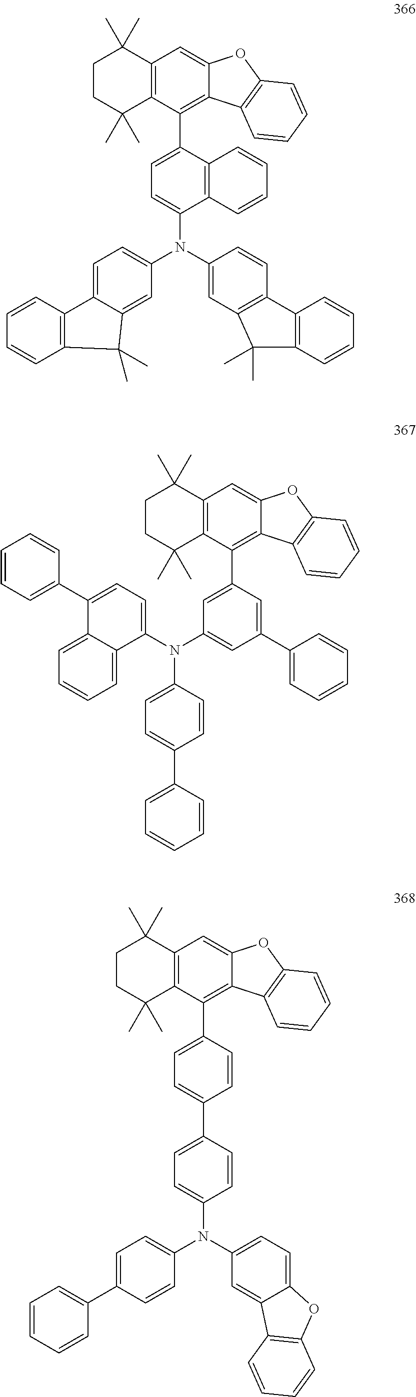 [SMILES: CC1(C)CCC(C)(C)c2c1cc1oc3ccccc3c1c2-c1ccc(N(c2ccc3c(c2)C(C)(C)c2ccccc2-3)c2ccc3c(c2)C(C)(C)c2ccccc2-3)c2ccccc12 ; CC1(C)CCC(C)(C)c2c1cc1oc3ccccc3c1c2-c1cc(-c2ccccc2)cc(N(c2ccc(-c3ccccc3)cc2)c2ccc(-c3ccccc3)c3ccccc23)c1 ; CC1(C)CCC(C)(C)c2c1cc1oc3ccccc3c1c2-c1ccc(-c2ccc(N(c3ccc(-c4ccccc4)cc3)c3ccc4oc5ccccc5c4c3)cc2)cc1]

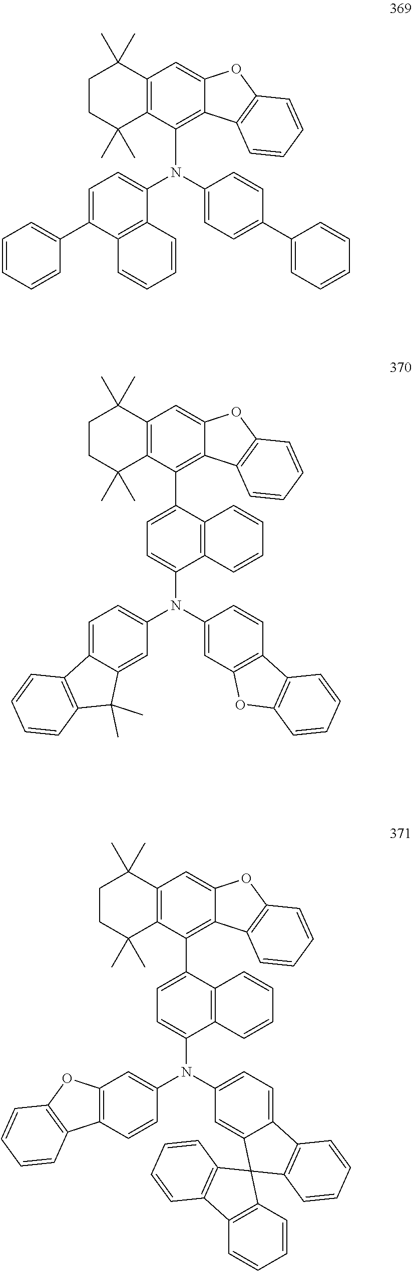 [SMILES: CC1(C)CCC(C)(C)c2c1cc1oc3ccccc3c1c2N(c1ccc(-c2ccccc2)cc1)c1ccc(-c2ccccc2)c2ccccc12 ; CC1(C)CCC(C)(C)c2c1cc1oc3ccccc3c1c2-c1ccc(N(c2ccc3c(c2)C(C)(C)c2ccccc2-3)c2ccc3c(c2)oc2ccccc23)c2ccccc12 ; CC1(C)CCC(C)(C)c2c1cc1oc3ccccc3c1c2-c1ccc(N(c2ccc3c(c2)C2(c4ccccc4-c4ccccc42)c2ccccc2-3)c2ccc3c(c2)oc2ccccc23)c2ccccc12]

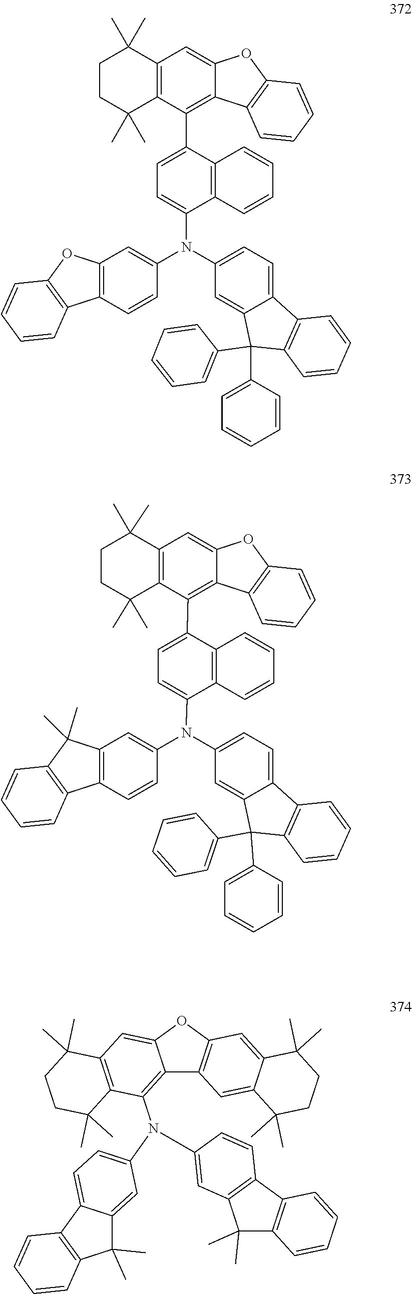 [SMILES: CC1(C)CCC(C)(C)c2c1cc1oc3ccccc3c1c2-c1ccc(N(c2ccc3c(c2)C(c2ccccc2)(c2ccccc2)c2ccccc2-3)c2ccc3c(c2)oc2ccccc23)c2ccccc12 ; CC1(C)CCC(C)(C)c2c1cc1oc3ccccc3c1c2-c1ccc(N(c2ccc3c(c2)C(C)(C)c2ccccc2-3)c2ccc3c(c2)C(c2ccccc2)(c2ccccc2)c2ccccc2-3)c2ccccc12 ; CC1(C)CCC(C)(C)c2cc3c(cc21)oc1cc2c(c(N(c4ccc5c(c4)C(C)(C)c4ccccc4-5)c4ccc5c(c4)C(C)(C)c4ccccc4-5)c13)C(C)(C)CCC2(C)C]

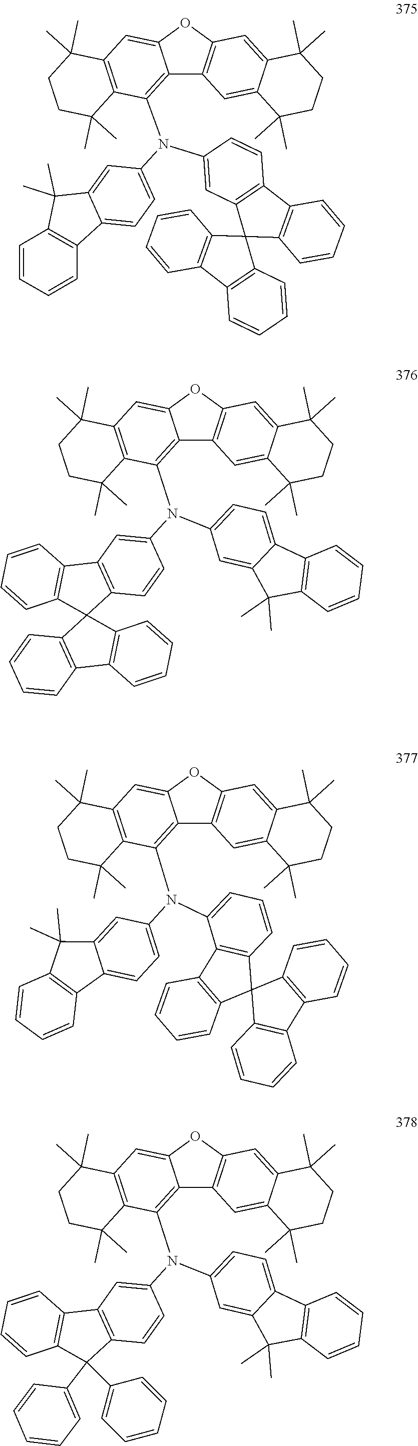

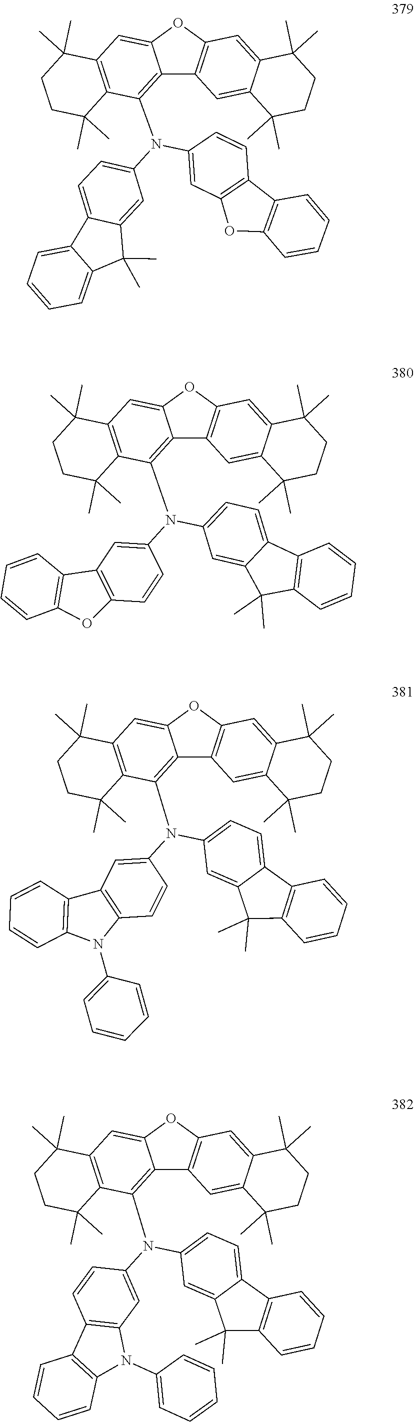 [SMILES: CC1(C)CCC(C)(C)c2cc3c(cc21)oc1cc2c(c(N(c4ccc5c(c4)C(C)(C)c4ccccc4-5)c4ccc5c(c4)oc4ccccc45)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5oc6ccccc6c5c4)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5c(c4)c4ccccc4n5-c4ccccc4)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5c6ccccc6n(-c6ccccc6)c5c4)c13)C(C)(C)CCC2(C)C]

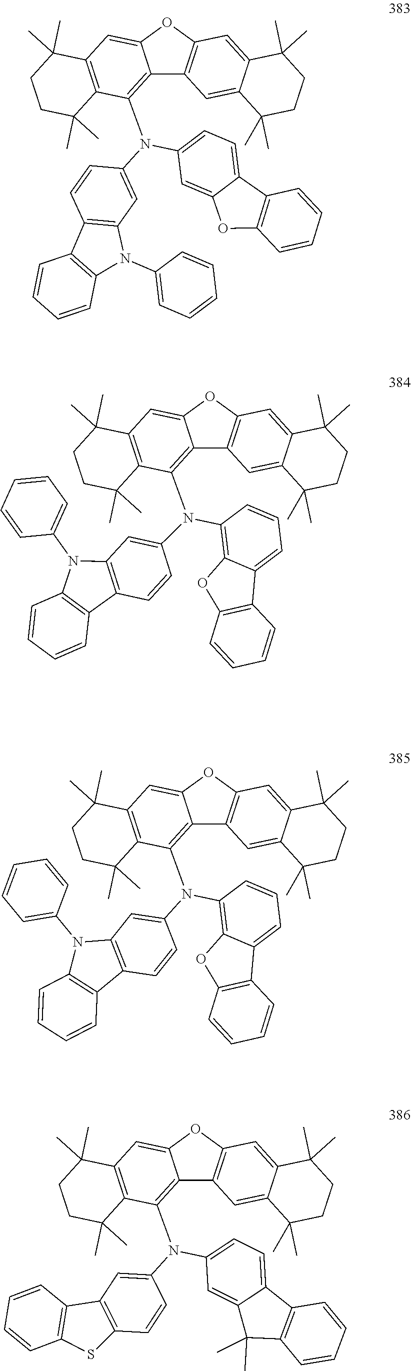 [SMILES: CC1(C)CCC(C)(C)c2cc3c(cc21)oc1cc2c(c(N(c4ccc5c(c4)oc4ccccc45)c4ccc5c6ccccc6n(-c6ccccc6)c5c4)c13)C(C)(C)CCC2(C)C ; CC1(C)CCC(C)(C)c2cc3c(cc21)oc1cc2c(c(N(c4ccc5c6ccccc6n(-c6ccccc6)c5c4)c4cccc5c4oc4ccccc45)c13)C(C)(C)CCC2(C)C ; CC1(C)CCC(C)(C)c2cc3c(cc21)oc1cc2c(c(N(c4ccc5c6ccccc6n(-c6ccccc6)c5c4)c4cccc5c4oc4ccccc45)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5sc6ccccc6c5c4)c13)C(C)(C)CCC2(C)C]

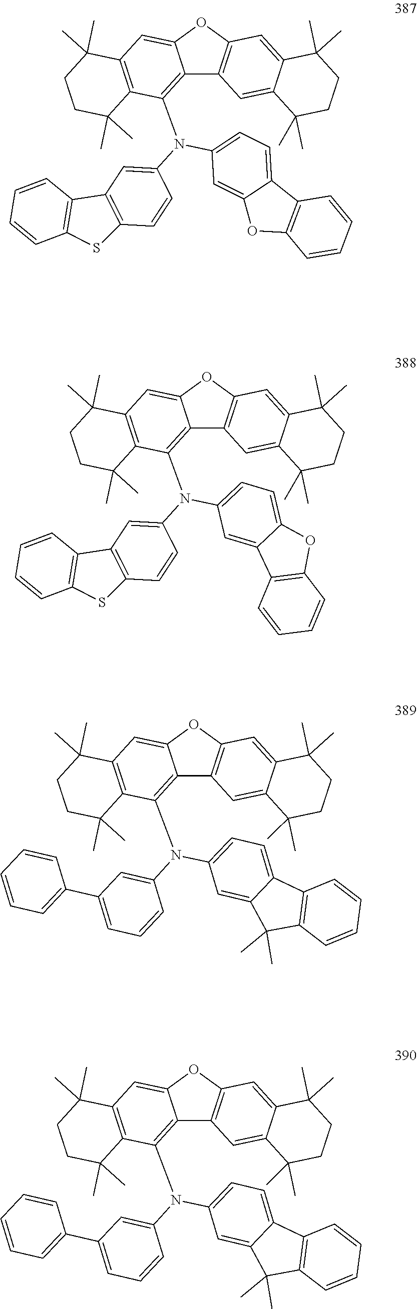 [SMILES: CC1(C)CCC(C)(C)c2cc3c(cc21)oc1cc2c(c(N(c4ccc5c(c4)oc4ccccc45)c4ccc5sc6ccccc6c5c4)c13)C(C)(C)CCC2(C)C ; CC1(C)CCC(C)(C)c2cc3c(cc21)oc1cc2c(c(N(c4ccc5oc6ccccc6c5c4)c4ccc5sc6ccccc6c5c4)c13)C(C)(C)CCC2(C)C ; CC1(C)CCC(C)(C)c2cc3c(cc21)oc1cc2c(c(N(c4cccc(-c5ccccc5)c4)c4ccc5c(c4)C(C)(C)c4ccccc4-5)c13)C(C)(C)CCC2(C)C ; CC1(C)CCC(C)(C)c2cc3c(cc21)oc1cc2c(c(N(c4cccc(-c5ccccc5)c4)c4ccc5c(c4)C(C)(C)c4ccccc4-5)c13)C(C)(C)CCC2(C)C]

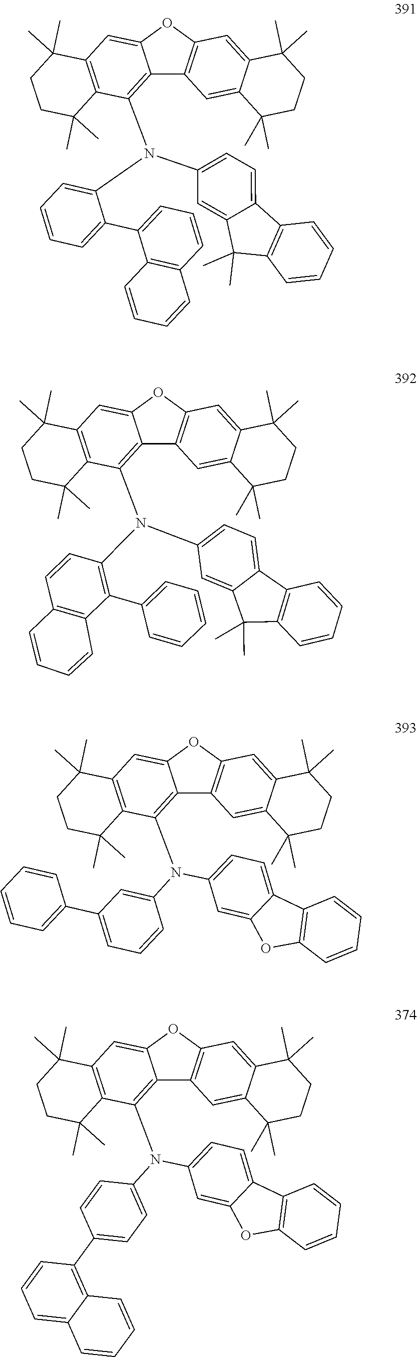 [SMILES: CC1(C)CCC(C)(C)c2cc3c(cc21)oc1cc2c(c(N(c4ccc5c(c4)C(C)(C)c4ccccc4-5)c4ccccc4-c4cccc5ccccc45)c13)C(C)(C)CCC2(C)C ; CC1(C)CCC(C)(C)c2cc3c(cc21)oc1cc2c(c(N(c4ccc5c(c4)C(C)(C)c4ccccc4-5)c4ccc5ccccc5c4-c4ccccc4)c13)C(C)(C)CCC2(C)C ; CC1(C)CCC(C)(C)c2cc3c(cc21)oc1cc2c(c(N(c4cccc(-c5ccccc5)c4)c4ccc5c(c4)oc4ccccc45)c13)C(C)(C)CCC2(C)C ; CC1(C)CCC(C)(C)c2cc3c(cc21)oc1cc2c(c(N(c4ccc(-c5cccc6ccccc56)cc4)c4ccc5c(c4)oc4ccccc45)c13)C(C)(C)CCC2(C)C]

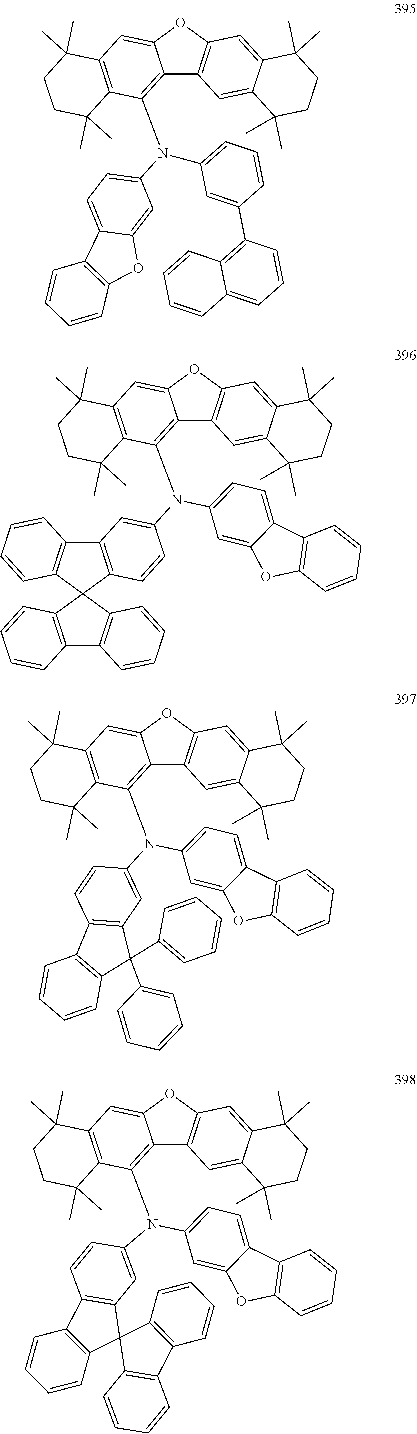

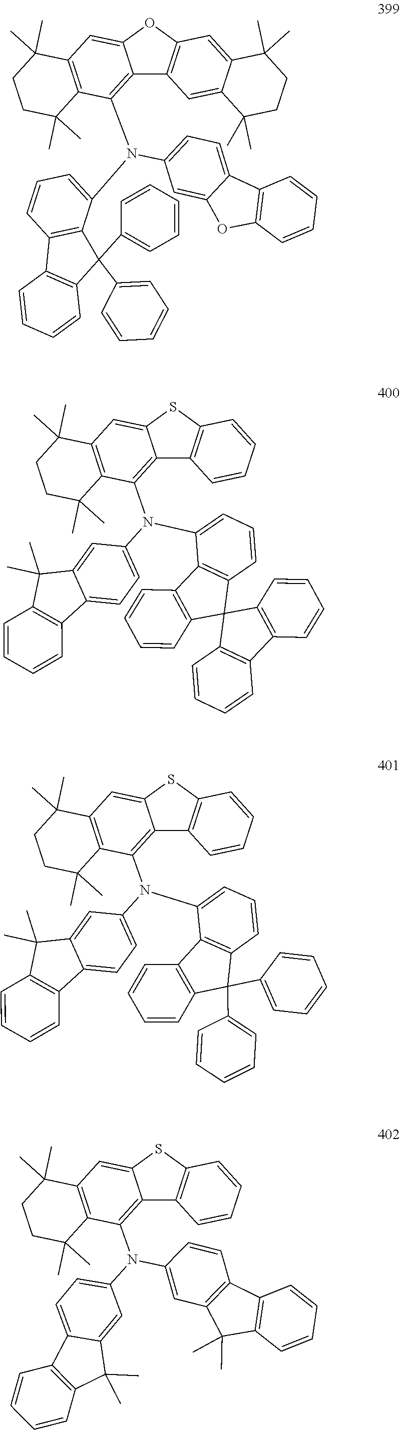 [SMILES: CC1(C)CCC(C)(C)c2cc3c(cc21)oc1cc2c(c(N(c4ccc5c(c4)oc4ccccc45)c4cccc5c4C(c4ccccc4)(c4ccccc4)c4ccccc4-5)c13)C(C)(C)CCC2(C)C ; CC1(C)CCC(C)(C)c2c1cc1sc3ccccc3c1c2N(c1ccc2c(c1)C(C)(C)c1ccccc1-2)c1cccc2c1-c1ccccc1C21c2ccccc2-c2ccccc21 ; CC1(C)CCC(C)(C)c2c1cc1sc3ccccc3c1c2N(c1ccc2c(c1)C(C)(C)c1ccccc1-2)c1cccc2c1-c1ccccc1C2(c1ccccc1)c1ccccc1 ; CC1(C)CCC(C)(C)c2c1cc1sc3ccccc3c1c2N(c1ccc2c(c1)C(C)(C)c1ccccc1-2)c1ccc2c(c1)C(C)(C)c1ccccc1-2]

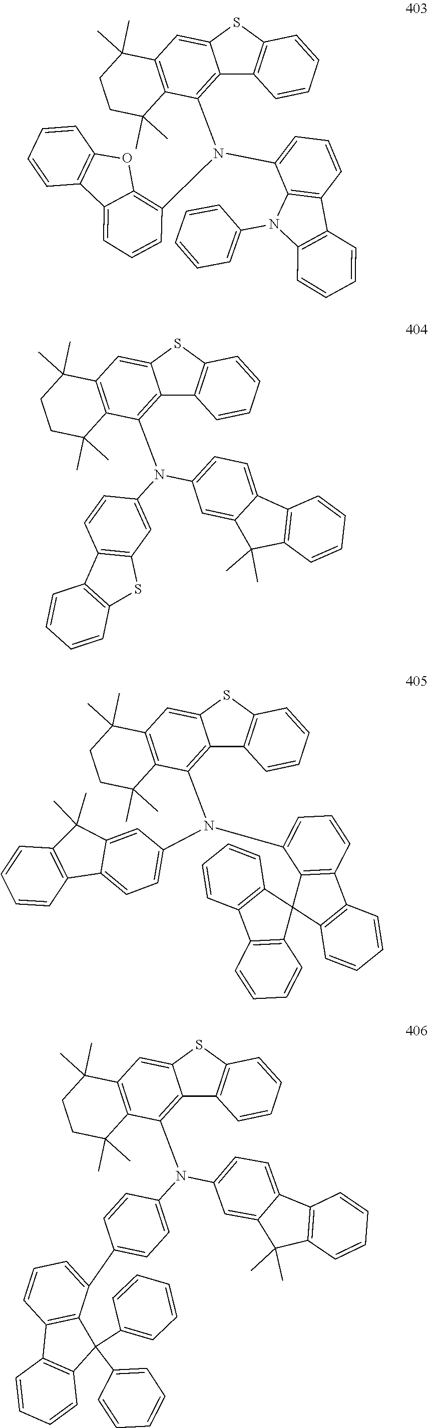 [SMILES: CC1(C)CCC(C)(C)c2c1cc1sc3ccccc3c1c2N(c1cccc2c1oc1ccccc12)c1cccc2c3ccccc3n(-c3ccccc3)c12 ; CC1(C)CCC(C)(C)c2c1cc1sc3ccccc3c1c2N(c1ccc2c(c1)C(C)(C)c1ccccc1-2)c1ccc2c(c1)sc1ccccc12 ; CC1(C)CCC(C)(C)c2c1cc1sc3ccccc3c1c2N(c1ccc2c(c1)C(C)(C)c1ccccc1-2)c1cccc2c1C1(c3ccccc3-c3ccccc31)c1ccccc1-2 ; CC1(C)CCC(C)(C)c2c1cc1sc3ccccc3c1c2N(c1ccc(-c2cccc3c2C(c2ccccc2)(c2ccccc2)c2ccccc2-3)cc1)c1ccc2c(c1)C(C)(C)c1ccccc1-2]

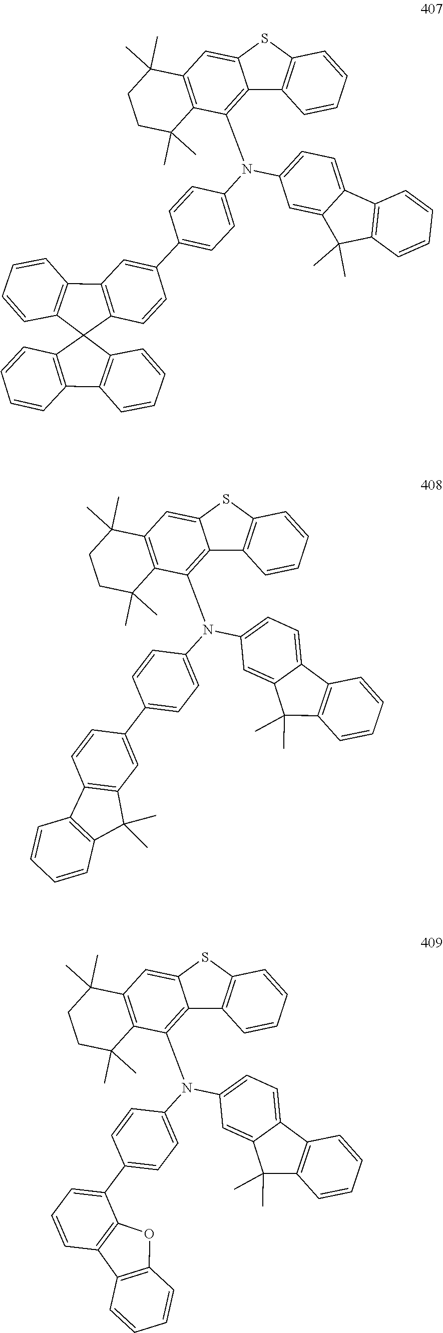 [SMILES: CC1(C)CCC(C)(C)c2c1cc1sc3ccccc3c1c2N(c1ccc(-c2ccc3c(c2)-c2ccccc2C32c3ccccc3-c3ccccc32)cc1)c1ccc2c(c1)C(C)(C)c1ccccc1-2 ; CC1(C)CCC(C)(C)c2c1cc1sc3ccccc3c1c2N(c1ccc(-c2ccc3c(c2)C(C)(C)c2ccccc2-3)cc1)c1ccc2c(c1)C(C)(C)c1ccccc1-2 ; CC1(C)CCC(C)(C)c2c1cc1sc3ccccc3c1c2N(c1ccc(-c2cccc3c2oc2ccccc23)cc1)c1ccc2c(c1)C(C)(C)c1ccccc1-2]

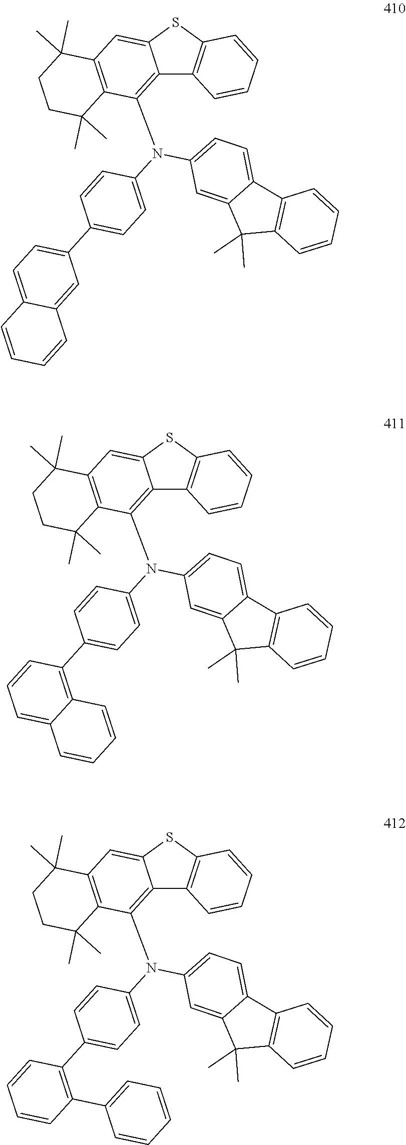 [SMILES: CC1(C)CCC(C)(C)c2c1cc1sc3ccccc3c1c2N(c1ccc(-c2ccc3ccccc3c2)cc1)c1ccc2c(c1)C(C)(C)c1ccccc1-2 ; CC1(C)CCC(C)(C)c2c1cc1sc3ccccc3c1c2N(c1ccc(-c2cccc3ccccc23)cc1)c1ccc2c(c1)C(C)(C)c1ccccc1-2 ; CC1(C)CCC(C)(C)c2c1cc1sc3ccccc3c1c2N(c1ccc(-c2ccccc2-c2ccccc2)cc1)c1ccc2c(c1)C(C)(C)c1ccccc1-2]

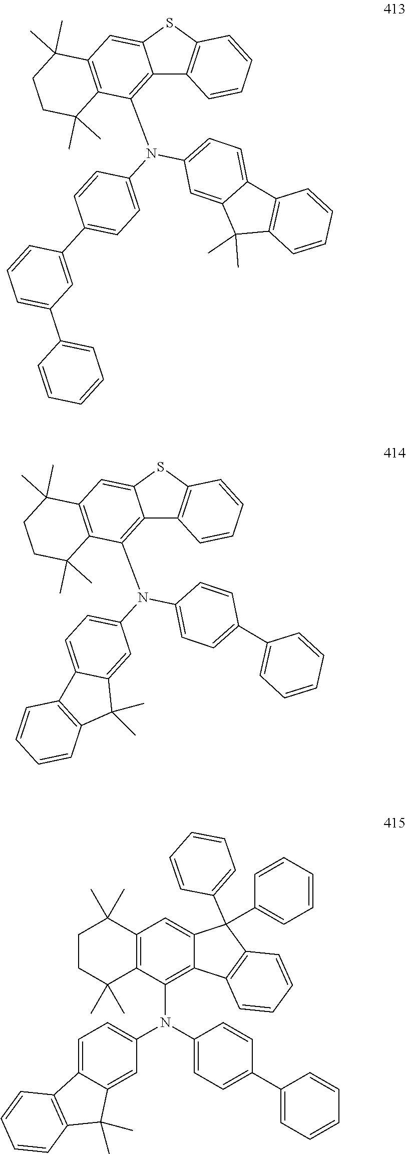 [SMILES: CC1(C)CCC(C)(C)c2c1cc1sc3ccccc3c1c2N(c1ccc(-c2cccc(-c3ccccc3)c2)cc1)c1ccc2c(c1)C(C)(C)c1ccccc1-2 ; CC1(C)CCC(C)(C)c2c1cc1sc3ccccc3c1c2N(c1ccc(-c2ccccc2)cc1)c1ccc2c(c1)C(C)(C)c1ccccc1-2 ; CC1(C)CCC(C)(C)c2c1cc1c(c2N(c2ccc(-c3ccccc3)cc2)c2ccc3c(c2)C(C)(C)c2ccccc2-3)-c2ccccc2C1(c1ccccc1)c1ccccc1]

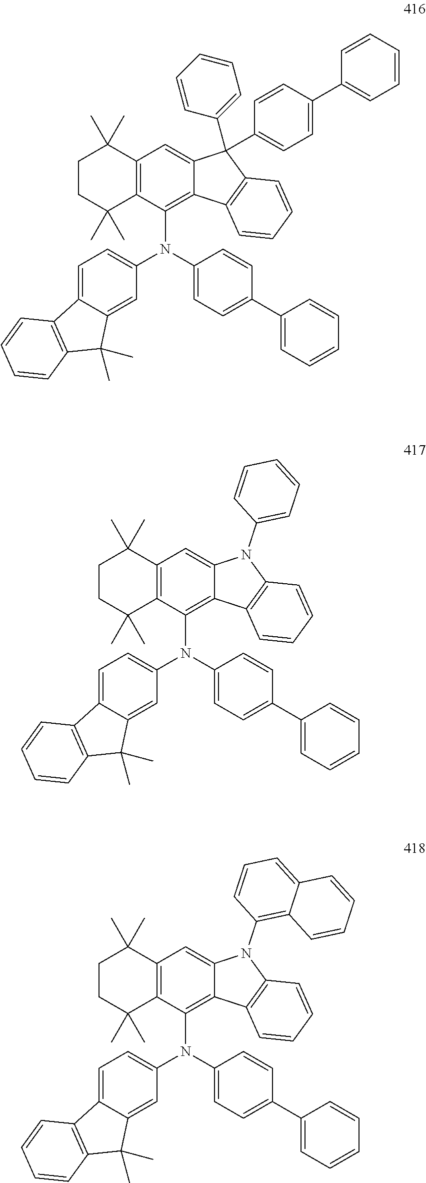 [SMILES: CC1(C)CCC(C)(C)c2c1cc1c(c2N(c2ccc(-c3ccccc3)cc2)c2ccc3c(c2)C(C)(C)c2ccccc2-3)-c2ccccc2C1(c1ccccc1)c1ccc(-c2ccccc2)cc1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1ccccc1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1cccc2ccccc12]

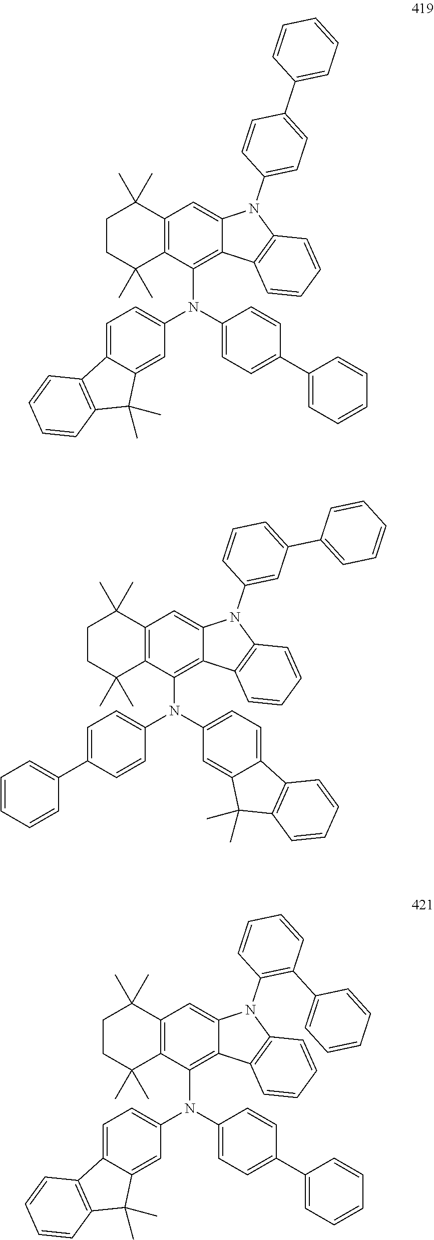 [SMILES: CC1(C)CCC(C)(C)c2c1cc1c(c2N(c2ccc(-c3ccccc3)cc2)c2ccc3c(c2)C(C)(C)c2ccccc2-3)c2ccccc2n1-c1ccc(-c2ccccc2)cc1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1cccc(-c2ccccc2)c1 ; CC1(C)CCC(C)(C)c2c1cc1c(c2N(c2ccc(-c3ccccc3)cc2)c2ccc3c(c2)C(C)(C)c2ccccc2-3)c2ccccc2n1-c1ccccc1-c1ccccc1]

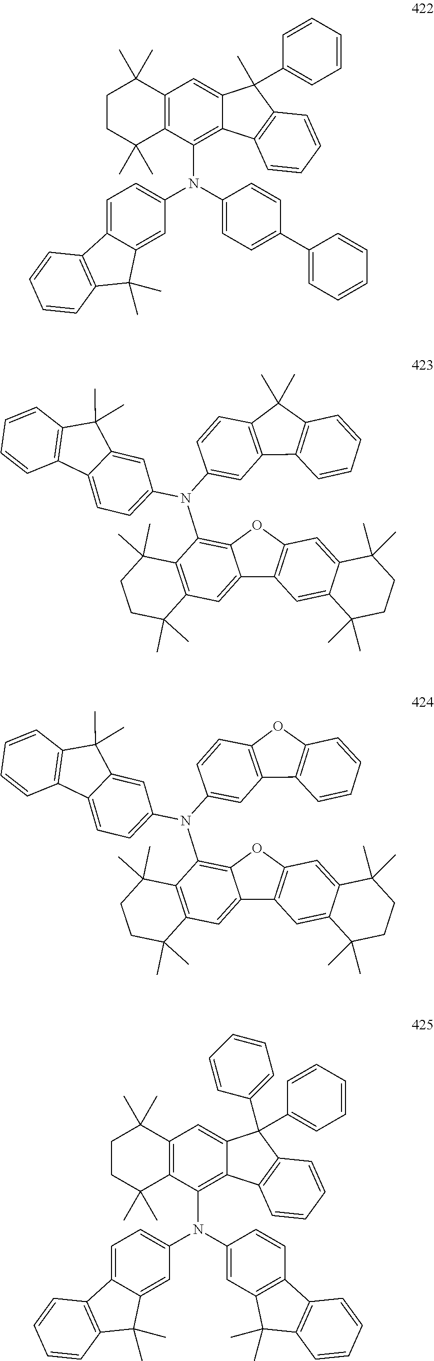 [SMILES: CC1(C)CCC(C)(C)c2c1cc1c(c2N(c2ccc(-c3ccccc3)cc2)c2ccc3c(c2)C(C)(C)c2ccccc2-3)-c2ccccc2C1(C)c1ccccc1 ; CC1(C)CCC(C)(C)c2cc3c(cc21)oc1c(N(c2ccc4c(c2)-c2ccccc2C4(C)C)c2ccc4c(c2)C(C)(C)c2ccccc2-4)c2c(cc13)C(C)(C)CCC2(C)C ; CC1(C)CCC(C)(C)c2cc3c(cc21)oc1c(N(c2ccc4c(c2)C(C)(C)c2ccccc2-4)c2ccc4oc5ccccc5c4c2)c2c(cc13)C(C)(C)CCC2(C)C ; CC1(C)CCC(C)(C)c2c1cc1c(c2N(c2ccc3c(c2)C(C)(C)c2ccccc2-3)c2ccc3c(c2)C(C)(C)c2ccccc2-3)-c2ccccc2C1(c1ccccc1)c1ccccc1]

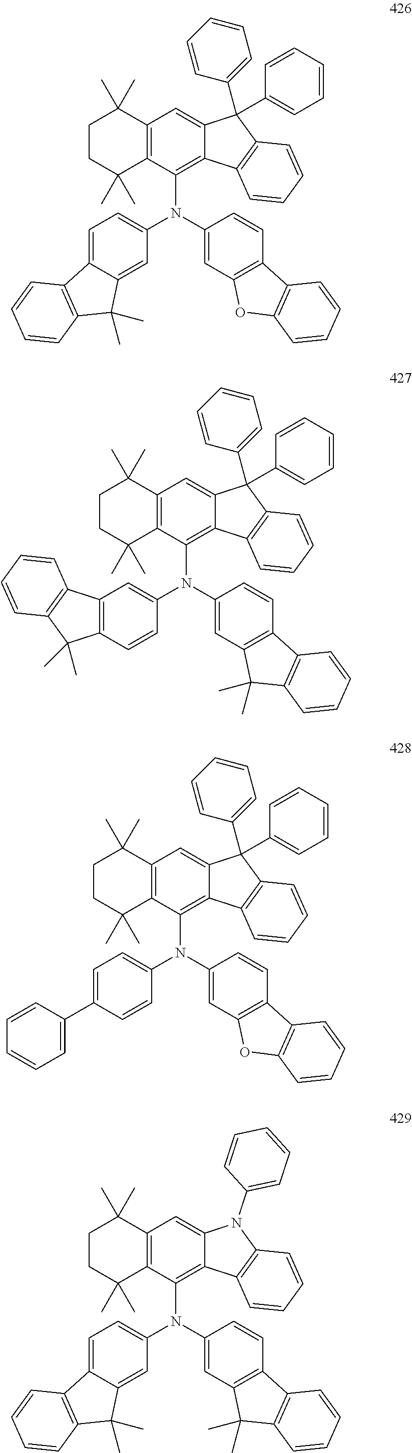 [SMILES: CC1(C)CCC(C)(C)c2c1cc1c(c2N(c2ccc3c(c2)C(C)(C)c2ccccc2-3)c2ccc3c(c2)oc2ccccc23)-c2ccccc2C1(c1ccccc1)c1ccccc1 ; CC1(C)CCC(C)(C)c2c1cc1c(c2N(c2ccc3c(c2)-c2ccccc2C3(C)C)c2ccc3c(c2)C(C)(C)c2ccccc2-3)-c2ccccc2C1(c1ccccc1)c1ccccc1 ; CC1(C)CCC(C)(C)c2c1cc1c(c2N(c2ccc(-c3ccccc3)cc2)c2ccc3c(c2)oc2ccccc23)-c2ccccc2C1(c1ccccc1)c1ccccc1 ; CC1(C)CCC(C)(C)c2c1cc1c(c2N(c2ccc3c(c2)C(C)(C)c2ccccc2-3)c2ccc3c(c2)C(C)(C)c2ccccc2-3)c2ccccc2n1-c1ccccc1]

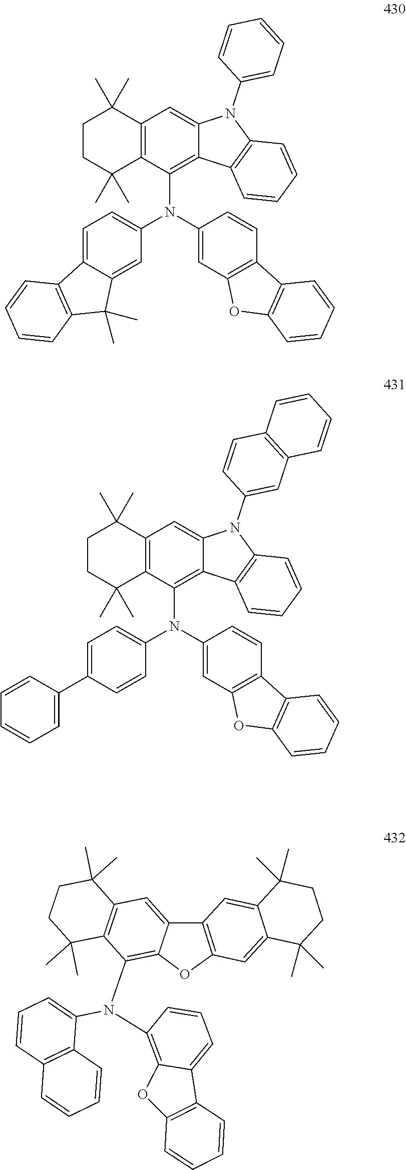 [SMILES: CC1(C)CCC(C)(C)c2c1cc1c(c2N(c2ccc3c(c2)C(C)(C)c2ccccc2-3)c2ccc3c(c2)oc2ccccc23)c2ccccc2n1-c1ccccc1 ; CC1(C)CCC(C)(C)c2c1cc1c(c2N(c2ccc(-c3ccccc3)cc2)c2ccc3c(c2)oc2ccccc23)c2ccccc2n1-c1ccc2ccccc2c1 ; CC1(C)CCC(C)(C)c2cc3c(cc21)oc1c(N(c2cccc4ccccc24)c2cccc4c2oc2ccccc24)c2c(cc13)C(C)(C)CCC2(C)C]

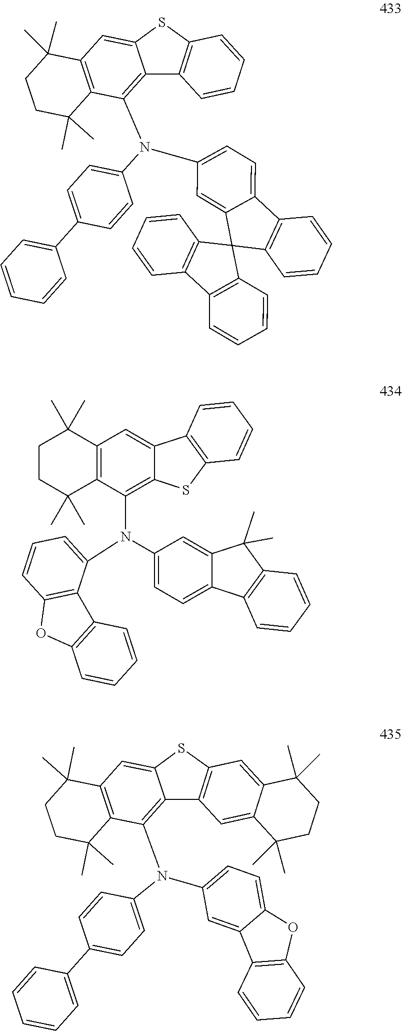 [SMILES: CC1(C)CCC(C)(C)c2c1cc1sc3ccccc3c1c2N(c1ccc(-c2ccccc2)cc1)c1ccc2c(c1)C1(c3ccccc3-c3ccccc31)c1ccccc1-2 ; CC1(C)CCC(C)(C)c2c1cc1c(sc3ccccc31)c2N(c1ccc2c(c1)C(C)(C)c1ccccc1-2)c1cccc2oc3ccccc3c12 ; CC1(C)CCC(C)(C)c2cc3c(cc21)sc1cc2c(c(N(c4ccc(-c5ccccc5)cc4)c4ccc5oc6ccccc6c5c4)c13)C(C)(C)CCC2(C)C]

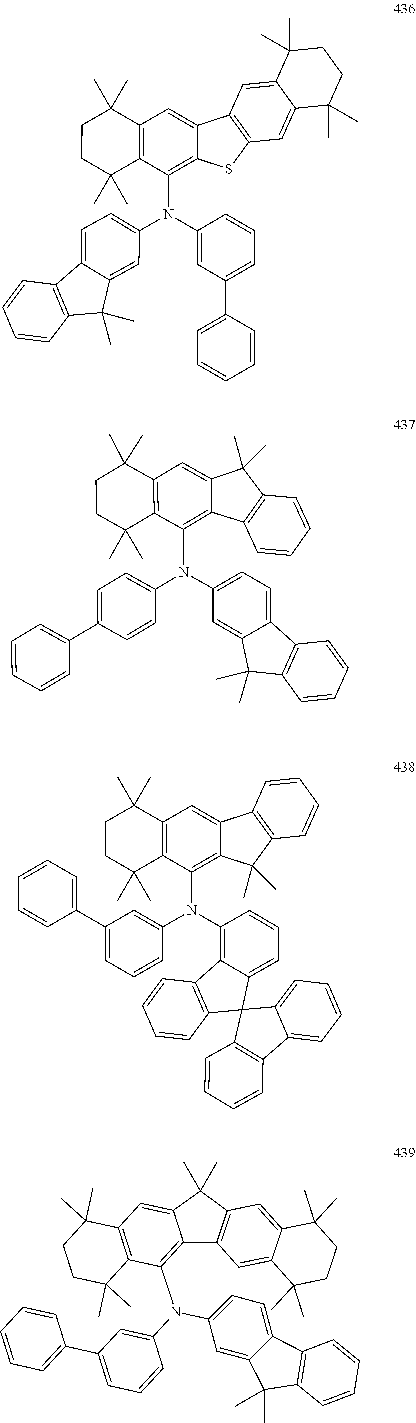 [SMILES: CC1(C)CCC(C)(C)c2cc3c(cc21)sc1c(N(c2cccc(-c4ccccc4)c2)c2ccc4c(c2)C(C)(C)c2ccccc2-4)c2c(cc13)C(C)(C)CCC2(C)C ; CC1(C)CCC(C)(C)c2c1cc1c(c2N(c2ccc(-c3ccccc3)cc2)c2ccc3c(c2)C(C)(C)c2ccccc2-3)-c2ccccc2C1(C)C ; CC1(C)CCC(C)(C)c2c1cc1c(c2N(c2cccc(-c3ccccc3)c2)c2cccc3c2-c2ccccc2C32c3ccccc3-c3ccccc32)C(C)(C)c2ccccc2-1 ; CC1(C)CCC(C)(C)c2cc3c(cc21)-c1c(cc2c(c1N(c1cccc(-c4ccccc4)c1)c1ccc4c(c1)C(C)(C)c1ccccc1-4)C(C)(C)CCC2(C)C)C3(C)C]

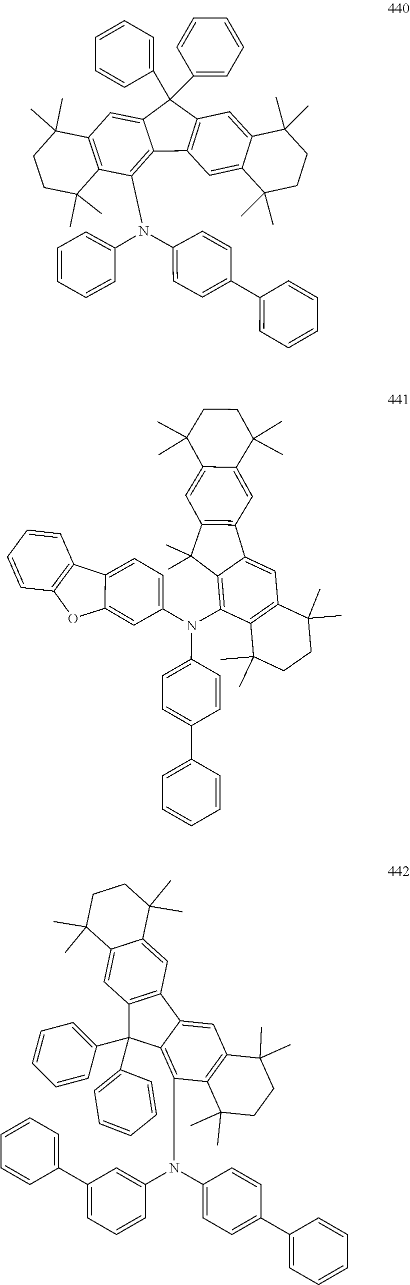 [SMILES: CC1(C)CCC(C)(C)c2cc3c(cc21)-c1c(cc2c(c1N(c1ccccc1)c1ccc(-c4ccccc4)cc1)C(C)(C)CCC2(C)C)C3(c1ccccc1)c1ccccc1 ; CC1(C)CCC(C)(C)c2cc3c(cc21)-c1cc2c(c(N(c4ccc(-c5ccccc5)cc4)c4ccc5c(c4)oc4ccccc45)c1C3(C)C)C(C)(C)CCC2(C)C ; CC1(C)CCC(C)(C)c2cc3c(cc21)-c1cc2c(c(N(c4ccc(-c5ccccc5)cc4)c4cccc(-c5ccccc5)c4)c1C3(c1ccccc1)c1ccccc1)C(C)(C)CCC2(C)C]

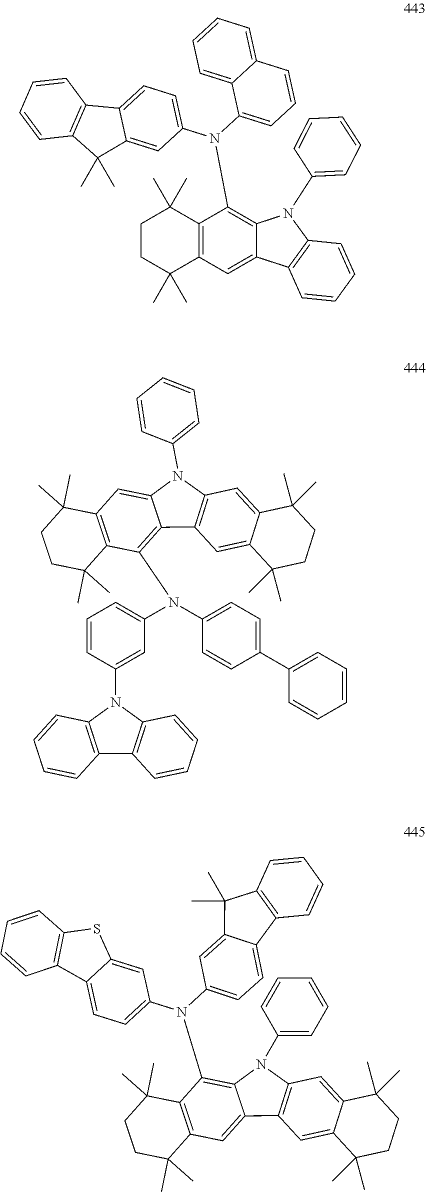 [SMILES: CC1(C)CCC(C)(C)c2c1cc1c3ccccc3n(-c3ccccc3)c1c2N(c1ccc2c(c1)C(C)(C)c1ccccc1-2)c1cccc2ccccc12 ; CC1(C)CCC(C)(C)c2cc3c(cc21)c1c(N(c2ccc(-c4ccccc4)cc2)c2cccc(-n4c5ccccc5c5ccccc54)c2)c2c(cc1n3-c1ccccc1)C(C)(C)CCC2(C)C ; CC1(C)CCC(C)(C)c2cc3c(cc21)c1cc2c(c(N(c4ccc5c(c4)C(C)(C)c4ccccc4-5)c4ccc5c(c4)sc4ccccc45)c1n3-c1ccccc1)C(C)(C)CCC2(C)C]

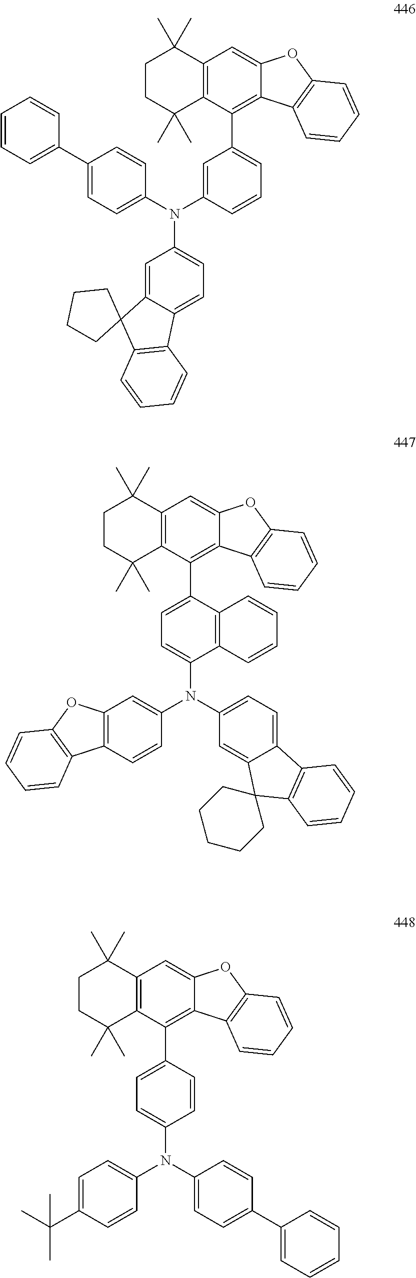 [SMILES: CC1(C)CCC(C)(C)c2c1cc1oc3ccccc3c1c2-c1cccc(N(c2ccc(-c3ccccc3)cc2)c2ccc3c(c2)C2(CCCC2)c2ccccc2-3)c1 ; CC1(C)CCC(C)(C)c2c1cc1oc3ccccc3c1c2-c1ccc(N(c2ccc3c(c2)C2(CCCCC2)c2ccccc2-3)c2ccc3c(c2)oc2ccccc23)c2ccccc12 ; CC(C)(C)c1ccc(N(c2ccc(-c3ccccc3)cc2)c2ccc(-c3c4c(cc5oc6ccccc6c35)C(C)(C)CCC4(C)C)cc2)cc1]

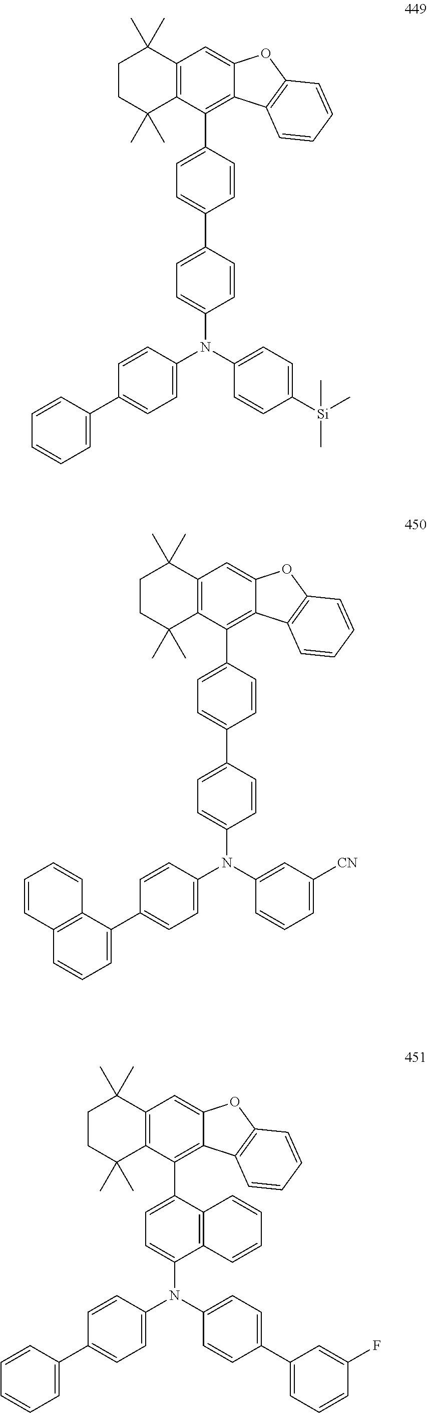 [SMILES: CC1(C)CCC(C)(C)c2c1cc1oc3ccccc3c1c2-c1ccc(-c2ccc(N(c3ccc(-c4ccccc4)cc3)c3ccc([Si](C)(C)C)cc3)cc2)cc1 ; CC1(C)CCC(C)(C)c2c1cc1oc3ccccc3c1c2-c1ccc(-c2ccc(N(c3ccc(-c4cccc5ccccc45)cc3)c3cccc(C#N)c3)cc2)cc1 ; CC1(C)CCC(C)(C)c2c1cc1oc3ccccc3c1c2-c1ccc(N(c2ccc(-c3ccccc3)cc2)c2ccc(-c3cccc(F)c3)cc2)c2ccccc12]

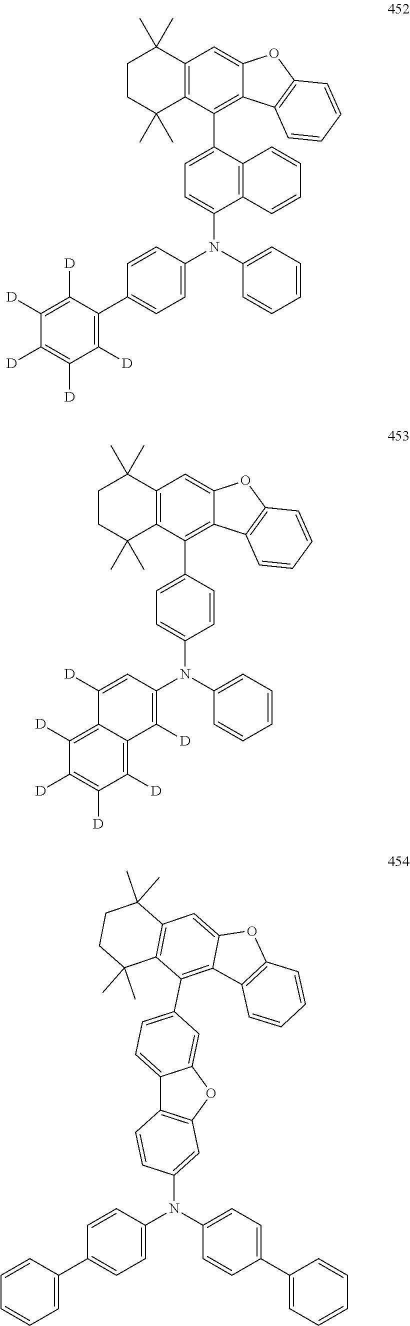 [SMILES: [2H]c1c([2H])c([2H])c(-c2ccc(N(c3ccccc3)c3ccc(-c4c5c(cc6oc7ccccc7c46)C(C)(C)CCC5(C)C)c4ccccc34)cc2)c([2H])c1[2H] ; [2H]c1c([2H])c([2H])c2c([2H])c(N(c3ccccc3)c3ccc(-c4c5c(cc6oc7ccccc7c46)C(C)(C)CCC5(C)C)cc3)cc([2H])c2c1[2H] ; CC1(C)CCC(C)(C)c2c1cc1oc3ccccc3c1c2-c1ccc2c(c1)oc1cc(N(c3ccc(-c4ccccc4)cc3)c3ccc(-c4ccccc4)cc3)ccc12]

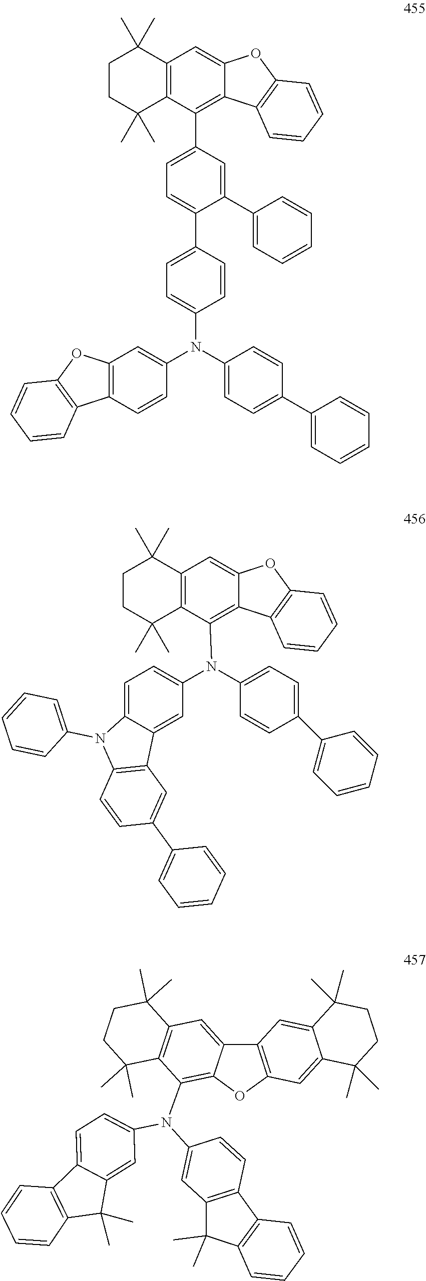 [SMILES: CC1(C)CCC(C)(C)c2c1cc1oc3ccccc3c1c2-c1ccc(-c2ccc(N(c3ccc(-c4ccccc4)cc3)c3ccc4c(c3)oc3ccccc34)cc2)c(-c2ccccc2)c1 ; CC1(C)CCC(C)(C)c2c1cc1oc3ccccc3c1c2N(c1ccc(-c2ccccc2)cc1)c1ccc2c(c1)c1cc(-c3ccccc3)ccc1n2-c1ccccc1 ; CC1(C)CCC(C)(C)c2cc3c(cc21)oc1c(N(c2ccc4c(c2)C(C)(C)c2ccccc2-4)c2ccc4c(c2)C(C)(C)c2ccccc2-4)c2c(cc13)C(C)(C)CCC2(C)C]

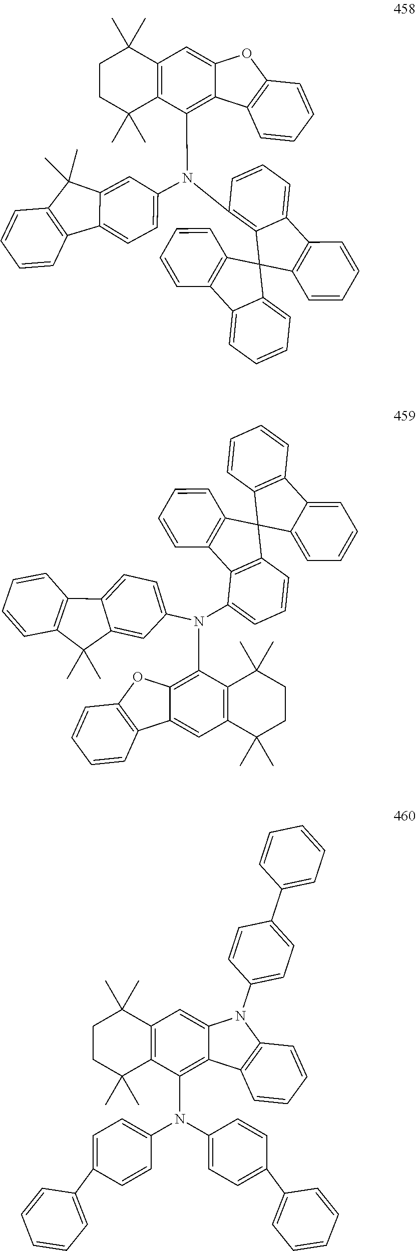 [SMILES: CC1(C)CCC(C)(C)c2c1cc1oc3ccccc3c1c2N(c1ccc2c(c1)C(C)(C)c1ccccc1-2)c1cccc2c1C1(c3ccccc3-c3ccccc31)c1ccccc1-2 ; CC1(C)CCC(C)(C)c2c1cc1c(oc3ccccc31)c2N(c1ccc2c(c1)C(C)(C)c1ccccc1-2)c1cccc2c1-c1ccccc1C21c2ccccc2-c2ccccc21 ; CC1(C)CCC(C)(C)c2c1cc1c(c2N(c2ccc(-c3ccccc3)cc2)c2ccc(-c3ccccc3)cc2)c2ccccc2n1-c1ccc(-c2ccccc2)cc1]

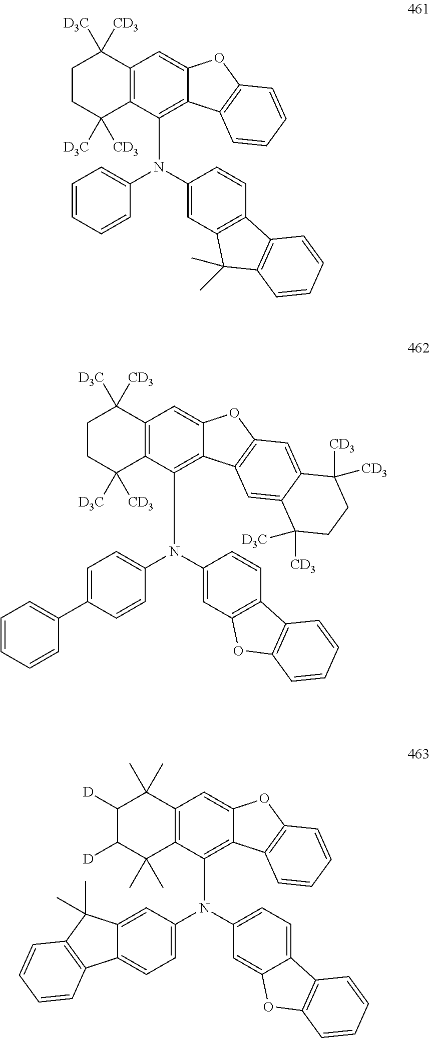 [SMILES: [2H]C([2H])([2H])C1(C([2H])([2H])[2H])CCC(C([2H])([2H])[2H])(C([2H])([2H])[2H])c2c1cc1oc3ccccc3c1c2N(c1ccccc1)c1ccc2c(c1)C(C)(C)c1ccccc1-2 ; [2H]C([2H])([2H])C1(C([2H])([2H])[2H])CCC(C([2H])([2H])[2H])(C([2H])([2H])[2H])c2cc3c(cc21)oc1cc2c(c(N(c4ccc(-c5ccccc5)cc4)c4ccc5c(c4)oc4ccccc45)c13)C(C([2H])([2H])[2H])(C([2H])([2H])[2H])CCC2(C([2H])([2H])[2H])C([2H])([2H])[2H] ; [2H]C1C([2H])C(C)(C)c2c(cc3oc4ccccc4c3c2N(c2ccc3c(c2)C(C)(C)c2ccccc2-3)c2ccc3c(c2)oc2ccccc23)C1(C)C]

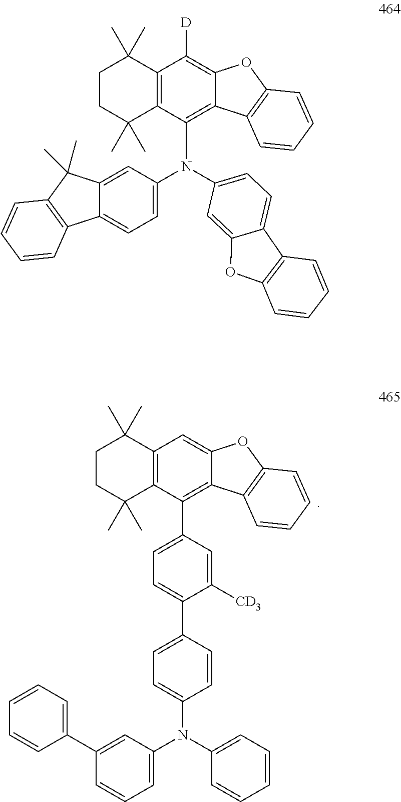 [SMILES: [2H]c1c2c(c(N(c3ccc4c(c3)C(C)(C)c3ccccc3-4)c3ccc4c(c3)oc3ccccc34)c3c1oc1ccccc13)C(C)(C)CCC2(C)C ; [2H]C([2H])([2H])c1cc(-c2c3c(cc4oc5ccccc5c24)C(C)(C)CCC3(C)C)ccc1-c1ccc(N(c2ccccc2)c2cccc(-c3ccccc3)c2)cc1]

13. An organic electroluminescent device, comprising an anode and a cathode disposed opposite to each other, and a functional layer disposed between the anode and the cathode; wherein the functional layer comprises the organic compound of claim 1.

14. The organic electroluminescent device according to claim 13, wherein the functional layer comprises a hole auxiliary layer, and the hole auxiliary layer comprises the organic compound;
optionally, the organic electroluminescent device is a green organic electroluminescent device.

15. An electronic apparatus, comprising the organic electroluminescent device of claim 13.
